(19) United States
(12) Patent Application Publication
(10) Pub. No.: US 2022/0249504 A1
(43) Pub. Date: Aug. 11, 2022

(54) LONGEVITY SIGNATURES AND THEIR APPLICATIONS

(71) Applicant: The Brigham and Women's Hospital, Inc., Boston, MA (US)

(72) Inventors: Vadim GLADYSHEV, Boston, MA (US); Alexander TYSHKOVSKIY, Boston, MA (US); Anastasia SHINDYAPINA, Boston, MA (US)

(21) Appl. No.: 17/625,425

(22) PCT Filed: Jul. 10, 2020
(86) PCT No.: PCT/US2020/041681

§ 371 Date: Jan. 7, 2022

Related U.S. Application Data
(60) Provisional application No. 63/014,256, filed on Apr. 23, 2020.
(60) Provisional application No. 62/872,499, filed on Jul. 10, 2019.

(51) Int. Cl. A61K 31/5377; A61P 39/00; A61K 31/4184; A61K 31/19

(52) U.S. Cl. CPC A61K 31/5377; A61K 31/19; A61K 31/4184; A61P 39/00

(57) ABSTRACT

The present disclosure relates to compositions and methods useful for elongating the lifespan of a subject (e.g., a mammalian subject, such as a human). Additionally or alternatively, the compositions and methods of the disclosure can be used to treat, prevent, and/or delay the onset of various geriatric syndromes in such a subject. The disclosure also provides compositions and methods that can be used to identify new interventions, such as chemical agents, lifestyle changes, or diets, that can be used to increase lifespan and to treat, prevent, and/or delay the onset of geriatric syndromes.

### CROSS-REFERENCE TO RELATED APPLICATIONS

This application claims benefit of U.S. Provisional Application Nos. 62/872,499 and 63/014,256, filed Jul. 10, 2019 and Apr. 23, 2020, respectively, the contents of which are incorporated herein by reference in their entirety.

### GOVERNMENT LICENSE RIGHTS

This invention was made with government support under grant AG047745 awarded by the National Institutes of Health. The government has certain rights in the invention.

### BACKGROUND

Several pharmacological, dietary and genetic interventions that increase lifespan in mammals are known, but the general principles of lifespan control have long been unclear. There remains a need for compositions and methods that can be used to increase the lifespan of a subject, as well as methodologies for identifying new interventions that have this beneficial biological activity.

### SUMMARY OF THE INVENTION

The present disclosure features compositions and methods that can be used to increase the lifespan of a subject (e.g., a mammalian subject, such as a human), as well as to treat, prevent, and/or delay the onset of various geriatric syndromes in such a subject. The disclosure also provides compositions and methods that can be used to identify new interventions, such as chemical agents, lifestyle changes, or diets, that can be used to increase lifespan and to treat, prevent, and/or delay the onset of geriatric syndromes.

The compositions and methods of the disclosure are based, in part, on the discovery of gene signatures that are characteristic of lifespan longevity. It has presently been discovered that certain genes, such as those recited in Tables 1-10 herein, are expressed in cells (e.g., mammalian cells, such as human cells) that have a relatively long lifespan, while other genes, such as those recited in Tables 2-20 herein, are down-regulated or expressed to a lower extent in cells (e.g., mammalian cells, such as human cells) that have a relatively short lifespan. This discovery provides a series of therapeutic and prophylactic benefits. Particularly, using these gene signatures, one can screen for new agents (e.g., small molecules, peptides, peptidomimetics, interfering ribonucleic acids (RNA), antibodies, aptamers, or gene therapies) that elevate the expression of one or more genes set forth in Tables 1-10 and/or that suppress the expression of one or more genes set forth in Tables 2-20 so as to identify interventions capable of increasing lifespan and delaying the onset of age-related pathologies. Additionally, guided by the gene signatures described herein, one can use existing agents that elevate the expression of one or more genes set forth in Tables 1-10 and/or that suppress the expression of one or more genes set forth in Tables 2-20 in order to increase the lifespan of a subject (e.g., a mammalian subject, such as a human) and/or delay the onset of age-related pathologies in such a subject.

In a first aspect, the disclosure features a method of identifying an agent capable of increasing the lifespan of a mammalian subject (e.g., a human). The method may include contacting the agent with a cell containing one or more genes set forth in any of Tables 1-20, wherein a finding that the agent (i) increases expression of one or more genes in any of Tables 1-10 and/or (ii) decreases expression of one or more genes in any of Tables 11-20 identifies the agent as being capable of increasing the lifespan of a mammalian subject.

In some embodiments, the cell contains one or more genes set forth in any of Tables 1-6 or Tables 11-16, and a finding that the agent (i) increases expression of one or more genes in any of Tables 1-6 and/or (ii) decreases expression of one or more genes in any of Tables 11-16 identifies the agent as being capable of increasing the lifespan of the mammalian subject. In some embodiments, the cell contains two, three, four, five, six, seven, eight, nine, ten, or more genes set forth in Table 1 and/or Table 11. In some embodiments, the cell contains two, three, four, five, six, seven, eight, nine, ten, or more genes set forth in Table 2 and/or Table 12. In some embodiments, the cell contains two, three, four, five, six, seven, eight, nine, ten, or more genes set forth in Table 3 and/or Table 13. In some embodiments, the cell contains two, three, four, five, six, seven, eight, nine, ten, or more genes set forth in Table 4 and/or Table 14. In some embodiments, the cell contains two, three, four, five, six, seven, eight, nine, ten, or more genes set forth in Table 5 and/or Table 15. In some embodiments, the cell contains two, three, four, five, six, seven, eight, nine, ten, or more genes set forth in Table 6 and/or Table 16.

In some embodiments, the cell contains one or more genes set forth in Table 7 or Table 17, and a finding that the agent (i) increases expression of one or more genes in Table 7 and/or (ii) decreases expression of one or more genes in Table 17 identifies the agent as being capable of increasing the lifespan of the mammalian subject. In some embodiments, the cell contains two, three, four, five, six, seven, eight, nine, ten, or more genes set forth in Table 7 and/or Table 17.

In some embodiments, the cell contains one or more genes set forth in any of Tables 8-10 or Tables 18-20, and a finding that the agent (i) increases expression of one or more genes in any of Tables 8-10 and/or (ii) decreases expression of one or more genes in any of Tables 18-20 identifies the agent as being capable of increasing the lifespan of the mammalian subject. In some embodiments, the cell contains two, three, four, five, six, seven, eight, nine, ten, or more genes set forth in Table 8 and/or Table 18. In some embodiments, the cell contains two, three, four, five, six, seven, eight, nine, ten, or more genes set forth in Table 9 and/or Table 19. In some embodiments, the cell contains two, three, four, five, six, seven, eight, nine, ten, or more genes set forth in Table 10 and/or Table 20.

In some embodiments, the agent is contacted with the cell by administering the agent to a test subject containing the cell. The test subject may be a mammal, such as a mouse. In some embodiments, expression of the one or more genes in the cell is determined by RNA-seq.

In some embodiments, the method further includes administering the identified agent to a mammalian subject to increase the lifespan of the subject and/or to treat an age-related disease.

In another aspect, the disclosure features a collection of (i) gene expression signatures as set forth in any of Tables 1-10, or a combination thereof, that are upregulated, and (ii) gene expression signatures as set forth in any of Tables 11-20, or a combination thereof, that are downregulated.

In a further aspect, the disclosure features a composition containing a biological sample and a plurality of nucleic acid primers suitable for amplification of one or more genes set forth in any of Tables 1-10 and/or Tables 11-20. In some embodiments, the nucleic acid primers are at least 85% complementary (e.g., 85%, 86%, 87%, 88%, 89%, 90%, 91%, 92%, 93%, 94%, 95%, 96%, 97%, 98%, 99%, 99.9%, or 100% complementary) to a portion of one or more of the genes set forth in any of Tables 1-10 and/or Tables 11-20. In some embodiments, the nucleic acid primers are at least 90% complementary (e.g., 90%, 91%, 92%, 93%, 94%, 95%, 96%, 97%, 98%, 99%, 99.9%, or 100% complementary) to a portion of one or more of the genes set forth in any of Tables 1-10 and/or Tables 11-20. In some embodiments, the nucleic acid primers are at least 95% complementary (e.g., 95%, 96%, 97%, 98%, 99%, 99.9%, or 100% complementary) to a portion of one or more of the genes set forth in any of Tables 1-10 and/or Tables 11-20. In some embodiments, the nucleic acid primers are 100% complementary to a portion of one or more of the genes set forth in any of Tables 1-10 and/or Tables 11-20.

In some embodiments, the nucleic acid primers are suitable for amplification of one or more genes set forth in any of Tables 1-6 or Tables 11-16. For example, the nucleic acid primers may be suitable for amplification of two, three, four, five, six, seven, eight, nine, ten, or more genes set forth in Table 1 and/or Table 11. In some embodiments, the nucleic acid primers are suitable for amplification of two, three, four, five, six, seven, eight, nine, ten, or more genes set forth in Table 2 and/or Table 12. In some embodiments, the nucleic acid primers are suitable for amplification of two, three, four, five, six, seven, eight, nine, ten, or more genes set forth in Table 3 and/or Table 13. In some embodiments, the nucleic acid primers are suitable for amplification of two, three, four, five, six, seven, eight, nine, ten, or more genes set forth in Table 4 and/or Table 14. In some embodiments, the nucleic acid primers are suitable for amplification of two, three, four, five, six, seven, eight, nine, ten, or more genes set forth in Table 5 and/or Table 15. In some embodiments, the nucleic acid primers are suitable for amplification of two, three, four, five, six, seven, eight, nine, ten, or more genes set forth in Table 6 and/or Table 16.

In some embodiments, the nucleic acid primers are suitable for amplification of one or more genes set forth in Table 7 or Table 17. For example, the nucleic acid primers may be suitable for amplification of two, three, four, five, six, seven, eight, nine, ten, or more genes set forth in Table 7 and/or Table 17.

In some embodiments, the nucleic acid primers are suitable for amplification of one or more genes set forth in any of Tables 8-10 or Tables 18-20. For example, the nucleic acid primers are suitable for amplification of two, three, four, five, six, seven, eight, nine, ten, or more genes set forth in Table 8 and/or Table 18. In some embodiments, the nucleic acid primers are suitable for amplification of two, three, four, five, six, seven, eight, nine, ten, or more genes set forth in Table 9 and/or Table 19. In some embodiments, the nucleic acid primers are suitable for amplification of two, three, four, five, six, seven, eight, nine, ten, or more genes set forth in Table 10 and/or Table 20.

In an additional aspect, the disclosure features a method of increasing the lifespan of a mammalian subject by providing the subject with a treatment that (i) increases expression of one or more genes set forth in any of Tables 1-10 and/or (ii) decreases expression of one or more genes set forth in any of Tables 11-20.

In a further aspect, the disclosure features a method of reducing the frailty index in a mammalian subject by providing the subject with a treatment that (i) increases expression of one or more genes set forth in any of Tables 1-10 and/or (ii) decreases expression of one or more genes set forth in any of Tables 11-20.

In yet another aspect, the disclosure features a method of improving learning ability in a mammalian subject by providing the subject with a treatment that (i) increases expression of one or more genes set forth in any of Tables 1-10 and/or (ii) decreases expression of one or more genes set forth in any of Tables 11-20.

In an additional aspect, the disclosure features a method of delaying onset of a geriatric syndrome in a mammalian subject by providing the subject with a treatment that (i) increases expression of one or more genes set forth in any of Tables 1-10 and/or (ii) decreases expression of one or more genes set forth in any of Tables 11-20.

In another aspect, the disclosure features a method of increasing the lifespan of a mammalian subject by administering to the subject a therapeutically effective amount of KU-0063794 (rel-5-[2-[(2R,6S)-2,6-dimethyl-4-morpholinyl]-4-(4-morpholinyl)pyrido[2,3-d]pyrimidin-7-yl]-2-methoxybenzenemethanol), Ascorbyl Palmitate ([(2S)-2-[(2R)-4,5-Dihydroxy-3-oxo-2-furyl]-2-hydroxy-ethyl] hexadecanoate), Celastrol (3-Hydroxy-9β,13α-dimethyl-2-oxo-24,25,26-trinoroleana-1(10),3,5,7-tetraen-29-oic acid), Oligomycin-a ((1R,4E,5'S,6S,6'S,7R,8S,10R,11R,12S,14R,15S,16R,18E,20E,22R,25S,27R,28S,29R)-22-ethyl-7,11,14,15-tetrahydroxy-6′-[(2R)-2-hydroxypropyl]-5′,6,8,10,12,14,16,28,29-nonamethyl-3′,4′,5′,6′-tetrahydro-3H,9H,13H-spiro[2,26-dioxabicyclo[23.3.1]nonacosa-4,18,20-triene-27,2′-pyran]-3,9,13-trione), NVP-BEZ235 (2-Methyl-2-{4-[3-methyl-2-oxo-8-(quinolin-3-yl)-2,3-dihydro-1H-imidazo[4,5-c]quinolin-1-yl]phenyl}propanenitrile), AZD-8055 (5-[2,4-bis[(3S)-3-methyl-4-morpholinyl]pyrido[2,3-d]pyrimidin-7-yl]-2-methoxy-benzenemethanol), Importazole (N-(1-Phenylethyl)-2-(pyrrolidin-1-yl)quinazolin-4-amine), Ryuvidine (2-methyl-5-[(4-methylphenyl)amino]-4,7-benzothiazoledione), NSC-663284 (6-Chloro-7-[[2-(4-morpholinyl)ethyl]amino]-5,8-quinolinedione), PI-828 (2-(4-Morpholinyl)-8-(4-aminopheny)l-4H-1-benzopyran-4-one), Pyrvinium pamoate (6-(Dimethylamino)-2-[2-(2,5-dimethyl-1-phenyl-1H-pyrrol-3-yl)ethenyl]-1-methyl-4,4′-methylenebis[3-hydroxy-2-naphthalenecarboxylate] (2:1)-quinolinium), PI-103 (3-[4-(4-morpholinyl)pyrido[3′,2′:4,5]furo[3,2-d]pyrimidin-2-yl]-phenol), YM-155 (4,9-dihydro-1-(2-methoxyethyl)2-methyl-4,9-dioxo-3-(2-pyrazinylmethyl)-1H-naphth[2,3-d]imidazolium, bromide), Prostratin ((1aR,1bS,4aR,7aS,7bR,8R,9aS)-4a,7b-dihydroxy-3-(hydroxymethyl)-1,1,6,8-tetramethyl-5-oxo-1,1a,1b,4,4a,5,7a,7b,8,9-decahydro-9aH-cyclopropa[3,4]benzo[1,2-e]azulen-9a-yl acetate), BCI hydrochloride (3-(cyclohexylamino)-2,3-dihydro-2-(phenylmethylene)-1H-inden-1-one, monohydrochloride), Dorsomorphin-Compound C (6-[4-[2-(1-Piperidinyl)ethoxy]phenyl]-3-(4-pyridinyl)pyrazolo[1,5-a]pyrimidine), VU-0418947-2 (6-Phenyl-N-[(3-phenylphenyl)methyl]-3-pyridin-2-yl-1,2,4-triazin-5-amine), JNK-9L (4-[3-fluoro-5-(4-morpholinyl)phenyl]-N-[4-[3-(4-morpholinyl)-1,2,4-triazol-1-yl]phenyl]-2-pyrimidinamine), Phloretin (3-(4-Hydroxyphenyl)-1-(2,4,6-trihydroxyphenyl)propan-1-one), ZG-10 ((E)-4-(4-(dimethylamino)but-2-enamido)-N-(3-((4-(pyridin-3-yl)pyrimidin-2-yl)amino)phenyl)benzamide), Proscillaridin (5-[(3S,8R,9S,10R,13R,14S,17R)-14-Hydroxy-10,13-dimethyl-3-((2R,3R,4R,5R,6R)-3,4,5-trihydroxy-6-methyltetrahydro-2H-pyran-2-yloxy)-2,3,6,7,8,9,10,11,12,13,14,15,16,17-tetradecahydro-1H-cyclopenta[a]phenanthren-17-yl]-2H-pyran-2-one), YC-1 (3-(5′-Hydroxymethyl-2′-furyl)-1-benzyl indazole), IKK-2-inhibitor-V (N-(3,5-Bis-trifluoromethylphenyl)-5-chloro-2-hydroxybenzamide), Anisomycin ((2R,3S,4S)-4-hydroxy-2-(4-methoxybenzyl)-pyrrolidin-3-yl acetate), LY294002 (2-Morpholin-4-yl-8-phenylchromen-4-one), Colforsin ([(3R,4aR,5S,6S,6aS,10S,10aR,10bS)-5-acetyloxy-3-ethenyl-10,10b-dihydroxy-3,4a,7,7,10a-Pentamethyl-1-oxo-5,6,6a,8,9,10-hexahydro-2H-benzo[f]chromen-6-yl] 3-d imethylaminopropanoate), Rilmenidine (N-(Dicyclopropylmethyl)-4,5-dihydro-1,3-oxazol-2-amine), Selumetinib (6-(4-Bromo-2-chloroanilino)-7-fluoro-N-(2-hydroxyethoxy)-3-methylbenzimidazole-5-carboxamide), GDC-0941 (Pictilisib, 4-(2-(1H-Indazol-4-yl)-6-((4-(methylsulfonyl)piperazin-1-yl)methyl)thieno[3,2-d]pyrimidin-4-yl)morpholine), Valdecoxib (4-(5-methyl-3-phenylisoxazol-4-yl)benzenesulfonamide), Myricetin (3,5,7-Trihydroxy-2-(3,4,5-trihydroxyphenyl)-4-chromenone), Cyproheptadine (4-(5H-Dibenzo[a,d]cyclohepten-5-ylidene)-1-methylpiperidine), Vorinostat (N-Hydroxy-N′-phenyloctanediamide), Nifedipine (3,5-Dimethyl 2,6-dimethyl-4-(2-nitrophenyl)-1,4-dihydropyridine-3,5-dicarboxylate), Phylloquinone (2-Methyl-3-[(E)-3,7,11,15-tetramethylhexadec-2-enyl]naphthalene-1,4-dione), Withaferin-A ((4β,5β,6β,22R)-4,27-Dihydroxy-5,6:22,26-diepoxyergosta-2,24-diene-1,26-dione), Temsirolimus ((1R,2R,4S)-4-{(2R)-2-[(3S,6R,7E,9R,10R,12R,14S,15E,17E,19E,21S,23S,26R,27R,34aS)-9,27-dihydroxy-10,21-dimethoxy-6,8,12,14,20,26-hexamethyl-1,5,11,28,29-pentaoxo-1,4,5,6,9,10,11,12,13,14,21,22,23,24,25,26,27,28,29,31,32,33,34,34a-tetracosahydro-3H-23,27-epoxypyrido[2,1-c][1,4]oxazacyclohentriacontin-3-yl]propyl}-2-methoxycyclohexyl 3-hydroxy-2-(hydroxymethyl)-2-methylpropanoate), SN-38 (4,11-diethyl-4,9-dihydroxy-(4S)-1H-pyrano[3′,4′:6,7]indolizino[1,2-b]quinoline-3,14(4H,12H)-dione), GSK-1059615 (5-[[4-(4-Pyridinyl)-6-quinolinyl]methylene]-2,4-thiazolidenedione), Tipifarnib (6-[(R)-amino-(4-chlorophenyl)-(3-methylimidazol-4-yl)methyl]-4-(3-chlorophenyl)-1-methylquinolin-2-one), Linifanib (1-[4-(3-amino-1H-indazol-4-yl)phenyl]-3-(2-fluoro-5-methylphenyl)urea), WYE-354 (4-[6-[4-[(methoxycarbonyl)amino]phenyl]-4-(4-morpholinyl)-1H-pyrazolo[3,4-d]pyrimidin-1-yl-]methyl ester-1-piperidinecarboxylic acid), MK-212 (6-Chloro-2-(1-piperazinyl)pyrazine hydrochloride), and/or Enzastaurin (3-(1-Methylindol-3-yl)-4-[1-[1-(pyridin-2-ylmethyl)piperidin-4-yl]indol-3-yl]pyrrole-2,5-dione), thereby increasing the lifespan of the subject.

In some embodiments, the method of increasing the lifespan of the mammalian subject includes administering to the subject a therapeutically effective amount of KU-0063794, Ascorbyl Palmitate, Celastrol, NVP-BEZ235, AZD-8055, Pyrvinium pamoate, LY294002, Colforsin, Rilmenidine, Selumetinib, GDC-0941, Valdecoxib, Myricetin, Vorinostat, Nifedipine, Phylloquinone, Linifanib, and/or Enzastaurin.

In some embodiments, the method of increasing the lifespan of the mammalian subject includes administering to the subject a therapeutically effective amount of Selumetinib, LY294002, AZD-8055, KU-0063794, and/or Celastrol. In some embodiments, the method of increasing the lifespan of the mammalian subject includes administering to the subject a therapeutically effective amount of Selumetinib.

In another aspect, the disclosure features a method of reducing the frailty index of a mammalian subject by administering to the subject a therapeutically effective amount of KU-0063794, Ascorbyl Palmitate, Celastrol, Oligomycin-a, NVP-BEZ235, AZD-8055, Importazole, Ryuvidine, NSC-663284, PI-828, Pyrvinium pamoate, P1-103, YM-155, Prostratin, BCI hydrochloride, Dorsomorphin-Compound C, VU-0418947-2, JNK-9L, Phloretin, ZG-10, Proscillaridin, YC-1, IKK-2-inhibitor-V, Anisomycin, LY294002, Colforsin, Rilmenidine, Selumetinib, GDC-0941, Valdecoxib, Myricetin, Cyproheptadine, Vorinostat, Nifedipine, Phylloquinone, Withaferin-A, Temsirolimus, SN-38, GSK-1059615, Tipifarnib, Linifanib, WYE-354, MK-212, and/or Enzastaurin, thereby reducing the frailty index of the subject.

In some embodiments, the method of reducing the frailty index of the mammalian subject includes administering to the subject a therapeutically effective amount of KU-0063794, Ascorbyl Palmitate, Celastrol, NVP-BEZ235, AZD-8055, Pyrvinium pamoate, LY294002, Colforsin, Rilmenidine, Selumetinib, GDC-0941, Valdecoxib, Myricetin, Vorinostat, Nifedipine, Phylloquinone, Linifanib, and/or Enzastaurin.

In some embodiments, the method of reducing the frailty index of the mammalian subject includes administering to the subject a therapeutically effective amount of Selumetinib, LY294002, AZD-8055, KU-0063794, and/or Celastrol. In some embodiments, the method of reducing the frailty index of the mammalian subject includes administering to the subject a therapeutically effective amount of Selumetinib.

In an additional aspect, the disclosure features a method of improving learning ability in a mammalian subject by administering to the subject a therapeutically effective amount of KU-0063794, Ascorbyl Palmitate, Celastrol, Oligomycin-a, NVP-BEZ235, AZD-8055, Importazole, Ryuvidine, NSC-663284, PI-828, Pyrvinium pamoate, PI-103, YM-155, Prostratin, BCI hydrochloride, Dorsomorphin-Compound C, VU-0418947-2, JNK-9L, Phloretin, ZG-10, Proscillaridin, YC-1, IKK-2-inhibitor-V, Anisomycin, LY294002, Colforsin, Rilmenidine, Selumetinib, GDC-0941, Valdecoxib, Myricetin, Cyproheptadine, Vorinostat, Nifedipine, Phylloquinone, Withaferin-A, Temsirolimus, SN-38, GSK-1059615, Tipifarnib, Linifanib, WYE-354, MK-212, and/or Enzastaurin, thereby improving the learning ability of the subject.

In some embodiments, the method of improving learning ability in the mammalian subject includes administering to the subject a therapeutically effective amount of KU-0063794, Ascorbyl Palmitate, Celastrol, NVP-BEZ235, AZD-8055, Pyrvinium pamoate, LY294002, Colforsin, Rilmenidine, Selumetinib, GDC-0941, Valdecoxib, Myricetin, Vorinostat, Nifedipine, Phylloquinone, Linifanib, and/or Enzastaurin.

In some embodiments, the method of improving learning ability in the mammalian subject includes administering to the subject a therapeutically effective amount of Selumetinib, LY294002, AZD-8055, KU-0063794, and/or Celastrol. In some embodiments, the method of improving learning ability in the mammalian subject includes administering to the subject a therapeutically effective amount of Selumetinib.

In another aspect, the disclosure features a method of delaying onset of a geriatric syndrome in a mammalian subject by administering to the subject a therapeutically effective amount of KU-0063794, Ascorbyl Palmitate, Celastrol, Oligomycin-a, NVP-BEZ235, AZD-8055, Importazole, Ryuvidine, NSC-663284, PI-828, Pyrvinium pamoate, PI-103, YM-155, Prostratin, BCI hydrochloride, Dorsomorphin-Compound C, VU-0418947-2, JNK-9L, Phloretin, ZG-10, Proscillaridin, YC-1, IKK-2-inhibitor-V, Anisomycin, LY294002, Colforsin, Rilmenidine, Selumetinib, GDC-0941, Valdecoxib, Myricetin, Cyproheptadine, Vorinostat, Nifedipine, Phylloquinone, Withaferin-A, Temsirolimus, SN-38, GSK-1059615, Tipifarnib, Linifanib, WYE-354, MK-212, and/or Enzastaurin, thereby delaying the onset of a geriatric syndrome in the subject.

In some embodiments, the method of delaying onset of a geriatric syndrome in the mammalian subject includes administering to the subject a therapeutically effective amount of KU-0063794, Ascorbyl Palmitate, Celastrol, NVP-BEZ235, AZD-8055, Pyrvinium pamoate, LY294002, Colforsin, Rilmenidine, Selumetinib, GDC-0941, Valdecoxib, Myricetin, Vorinostat, Nifedipine, Phylloquinone, Linifanib, and/or Enzastaurin.

In some embodiments, the method of delaying onset of a geriatric syndrome in the mammalian subject includes administering to the subject a therapeutically effective amount of Selumetinib, LY294002, AZD-8055, KU-0063794, and/or Celastrol. In some embodiments, the method of delaying onset of a geriatric syndrome in the mammalian subject includes administering to the subject a therapeutically effective amount of Selumetinib.

In some embodiments of any of the eight preceding aspects of the disclosure, the subject is a human.

In some embodiments, the treatment includes administration of an agent, a lifestyle change, a change in disease status, or a combination thereof. In some embodiments, the treatment includes administration of an agent, such as an agent that contains a small molecule, a peptide, a peptidomimetic, an interfering ribonucleic acid (RNA), an antibody, an aptamer, or a gene therapy.

In some embodiments, the agent contains a small molecule, such as a compound represented by formula (I)

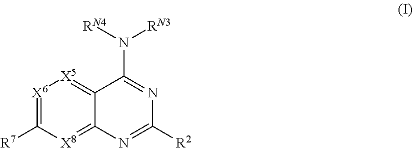

wherein one or two of X⁵, X⁶ and X⁸ is N, and the other(s) is/are CH;

R⁷ is selected from halo, OR⁰¹, SR^{S1}, NR^{N1}R^{N2}, NR^{N7a}C(═O)R^{C1}, NR^{N7b}SO₂R^{S2a}, an optionally substituted C₅₋₂₀ heteroaryl group, and an optionally substituted C₅₋₂₀ aryl group;

R⁰¹ and R^{S1} are selected from H, an optionally substituted C₅₋₂₀ aryl group, an optionally substituted C₅₋₂₀ heteroaryl group, and an optionally substituted C₁₋₇ alkyl group;

R^{N1} and R^{N2} are independently selected from H, an optionally substituted C₁₋₇ alkyl group, an optionally substituted C₅₋₂₀ heteroaryl group, and an optionally substituted C₅₋₂₀ aryl group, or R^{N1} and R^{N2}, together with the nitrogen to which they are bound, form a heterocyclic ring containing from 3 to 8 ring atoms;

R^{C1} is selected from H, an optionally substituted C₅₋₂₀ aryl group, an optionally substituted C₅₋₂₀ heteroaryl group, an optionally substituted C₁₋₇ alkyl group;

NR^{N8}R^{N9}, wherein R^{N8} and R^{N9} are independently selected from H, an optionally substituted C₁₋₇ alkyl group, an optionally substituted C₅₋₂₀ heteroaryl group, and an optionally substituted C₅₋₂₀ aryl group, or R^{N8} and R^{N9}, together with the nitrogen to which they are bound, form a heterocyclic ring containing from 3 to 8 ring atoms;

R^{S2a} is selected from H, an optionally substituted C₅₋₂₀ aryl group, an optionally substituted C₅₋₂₀ heteroaryl group, and an optionally substituted C₁₋₇ alkyl group; R^{N7a} and R^{N7b} are selected from H and a C₁₋₄ alkyl group;

R^{N3} and R^{N4}, together with the nitrogen to which they are bound, form an optionally substituted heterocyclic ring containing from 3 to 8 ring atoms;

R² is selected from H, halo, OR⁰², SR^{S2b}, NR^{N5}R^{N6}, an optionally substituted C₅₋₂₀ heteroaryl group, and an optionally substituted C₅₋₂₀ aryl group, wherein R⁰² and R^{S2b} are selected from H, an optionally substituted C₅₋₂₀ aryl group, an optionally substituted C₅₋₂₀ heteroaryl group, and an optionally substituted C₁₋₇ alkyl group; and

R^{N5} and R^{N6} are independently selected from H, an optionally substituted C₁₋₇ alkyl group, an optionally substituted C₅₋₂₀ heteroaryl group, and an optionally substituted C₅₋₂₀ aryl group, or R^{N5} and R^{N6}, together with the nitrogen to which they are bound, form an optionally substituted heterocyclic ring containing from 3 to 8 ring atoms,

or a pharmaceutically acceptable salt thereof.

In some embodiments, the agent contains KU-0063794, represented by formula (1)

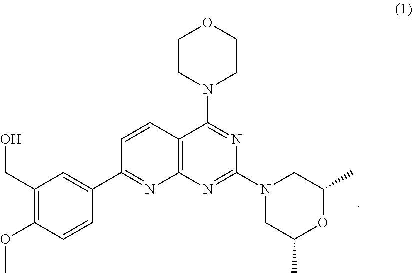 [SMILES: COc1ccc(-c2ccc3c(N4CCOCC4)nc(N4C[C@@H](C)O[C@@H](C)C4)nc3n2)cc1CO]

In some embodiments, the agent contains ascorbyl palmitate.

In some embodiments, the agent contains Selumetinib, LY294002, AZD-8055, KU-0063794, and/or Celastrol.

In some embodiments, the agent contains Selumetinib.

In some embodiments, the treatment contains a lifestyle chang, such as a dietary change.

In some embodiments, the agent is administered to the subject orally, intraarticularly, intravenously, intramuscularly, rectally, cutaneously, subcutaneously, topically, transdermally, sublingually, nasally, intravesicularly, intrathecally, epidurally, or transmucosally.

In some embodiments, the agent is administered to the subject orally, and may optionally be formulated as a tablet, capsule, gel cap, powder, liquid solution, or liquid suspension.

In some embodiments, the method further includes monitoring the subject for (i) an increase in expression of one or more genes set forth in Tables 1-10 and/or (ii) a decrease in expression of one or more genes set forth in Tables 11-20 following the treatment.

In yet another aspect, the disclosure features a pharmaceutical composition containing a compound represented by formula (I)

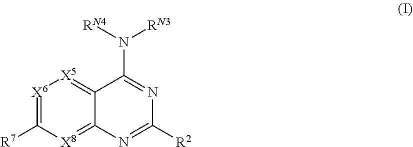

wherein one or two of X⁵, X⁶ and k is N, and the other(s) is/are CH;

R⁷ is selected from halo, OR⁰¹, SR^{S1}, NR^{N1}R^{N2}, NR^{N7a}C(═O)R^{C1}, NR^{N7b}SO₂R^{S2a}, an optionally substituted C₅₋₂₀ heteroaryl group, and an optionally substituted C₅₋₂₀ aryl group;

R⁰¹ and R^{S1} are selected from H, an optionally substituted C₅₋₂₀ aryl group, an optionally substituted C₅₋₂₀ heteroaryl group, and an optionally substituted C₁₋₇ alkyl group;

R^{N1} and R^{N2} are independently selected from H, an optionally substituted C₁₋₇ alkyl group, an optionally substituted C₅₋₂₀ heteroaryl group, and an optionally substituted C₅₋₂₀ aryl group, or R^{N1} and R^{N2}, together with the nitrogen to which they are bound, form a heterocyclic ring containing from 3 to 8 ring atoms;

R^{C1} is selected from H, an optionally substituted C₅₋₂₀ aryl group, an optionally substituted C₅₋₂₀ heteroaryl group, an optionally substituted C₁₋₇ alkyl group;

NR^{N8}R^{N9}, wherein R^{N8} and R^{N9} are independently selected from H, an optionally substituted C₁₋₇ alkyl group, an optionally substituted C₅₋₂₀ heteroaryl group, and an optionally substituted C₅₋₂₀ aryl group, or R^{N8} and R^{N9}, together with the nitrogen to which they are bound, form a heterocyclic ring containing from 3 to 8 ring atoms;

R^{S2a} is selected from H, an optionally substituted C₅₋₂₀ aryl group, an optionally substituted C₅₋₂₀ heteroaryl group, and an optionally substituted C₁₋₇ alkyl group;

R^{N7}a and R^{N7b} are selected from H and a C₁₋₄ alkyl group; R^{N3} and R^{N4}, together with the nitrogen to which they are bound, form an optionally substituted heterocyclic ring containing from 3 to 8 ring atoms;

R² is selected from H, halo, OR⁰², SR^{S2b}, NR^{N5}R^{N6}, an optionally substituted C₅₋₂₀ heteroaryl group, and an optionally substituted C₅₋₂₀ aryl group, wherein R⁰² and R^{S2b} are selected from H, an optionally substituted C₅₋₂₀ aryl group, an optionally substituted C₅₋₂₀ heteroaryl group, and an optionally substituted C₁₋₇ alkyl group; and

R^{N5} and R^{N6} are independently selected from H, an optionally substituted C₁₋₇ alkyl group, an optionally substituted C₅₋₂₀ heteroaryl group, and an optionally substituted C₅₋₂₀ aryl group, or R^{N5} and R^{N6}, together with the nitrogen to which they are bound, form an optionally substituted heterocyclic ring containing from 3 to 8 ring atoms,

or a pharmaceutically acceptable salt thereof.

In some embodiments, the composition contains one or more pharmaceutically acceptable excipients and/or is formulated for administration to a subject in combination with a meal.

In some embodiments, the compound is KU-0063794, represented by formula (1)

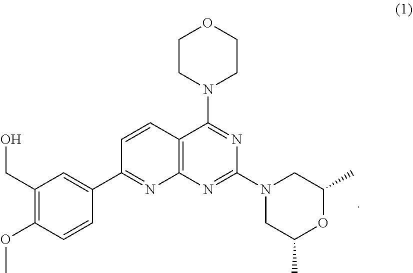 [SMILES: COc1ccc(-c2ccc3c(N4CCOCC4)nc(N4C[C@@H](C)O[C@@H](C)C4)nc3n2)cc1CO]

In another aspect, the disclosure features a pharmaceutical composition containing ascorbyl palmitate. The pharmaceutical composition may further contain one or more pharmaceutically acceptable excipients and/or be formulated for administration to a subject in combination with a meal.

In a further aspect, the disclosure features a pharmaceutical composition containing KU-0063794, Ascorbyl Palmitate, Celastrol, Oligomycin-a, NVP-BEZ235, AZD-8055, Importazole, Ryuvidine, NSC-663284, PI-828, Pyrvinium pamoate, PI-103, YM-155, Prostratin, BCI hydrochloride, Dorsomorphin-Compound C, VU-0418947-2, JNK-9L, Phloretin, ZG-10, Proscillaridin, YC-1, IKK-2-inhibitor-V, Anisomycin, LY294002, Colforsin, Rilmenidine, Selumetinib, GDC-0941, Valdecoxib, Myricetin, Cyproheptadine, Vorinostat, Nifedipine, Phylloquinone, Withaferin-A, Temsirolimus, SN-38, GSK-1059615, Tipifarnib, Linifanib, WYE-354, MK-212, and/or Enzastaurin. The pharmaceutical composition may further contain one or more pharmaceutically acceptable excipients and/or be formulated for administration to a subject in combination with a meal.

In yet another aspect, the disclosure features a pharmaceutical composition containing Selumetinib, LY294002, AZD-8055, KU-0063794, and/or Celastrol. The pharmaceutical composition may further contain one or more pharmaceutically acceptable excipients and/or be formulated for administration to a subject in combination with a meal.

In some embodiments of any of the four preceding aspects of the disclosure, the composition is a tablet, capsule, gel cap, powder, liquid solution, or liquid suspension. In some embodiments, the composition is formulated for administration to a subject by way of intraarticular, intravenous, intramuscular, rectal, cutaneous, subcutaneous, topical, transdermal, sublingual, nasal, intravesicular, intrathecal, epidural, or transmucosal delivery.

In some embodiments, the subject is a mammal, such as a human.

In an additional aspect, the disclosure features a dietary supplement containing KU-0063794, Ascorbyl Palmitate, Celastrol, Oligomycin-a, NVP-BEZ235, AZD-8055, Importazole, Ryuvidine, NSC-663284, PI-828, Pyrvinium pamoate, PI-103, YM-155, Prostratin, BCI hydrochloride, Dorsomorphin-Compound C, VU-0418947-2, JNK-9L, Phloretin, ZG-10, Proscillaridin, YC-1, IKK-2-inhibitor-V, Anisomycin, LY294002, Colforsin, Rilmenidine, Selumetinib, GDC-0941, Valdecoxib, Myricetin, Cyproheptadine, Vorinostat, Nifedipine, Phylloquinone, Withaferin-A, Temsirolimus, SN-38, GSK-1059615, Tipifarnib, Linifanib, WYE-354, MK-212, or Enzastaurin, or a combination thereof.

In an additional aspect, the disclosure features a dietary supplement containing Selumetinib, LY294002, AZD-8055, KU-0063794, or Celastrol, or a combination thereof.

In some embodiments, the dietary supplement is a tablet, capsule, gel cap, powder, liquid solution, or liquid suspension. The dietary supplement may be formulated for administration to a subject (e.g., a mammalian subject, such as a human) in combination with a meal.

### Definitions

As used herein, an agent (e.g., a therapeutic or prophylactic agent) is considered to be “provided” to a subject if the subject is directly administered the agent or if the subject is administered a substance that is processed or metabolized in vivo so as to yield the agent endogenously. For example, a subject, such as a subject having or at risk of developing a geriatric syndrome, may be provided an agent of the disclosure by direct administration of the agent or by administration of a substance that is processed or metabolized in vivo so as to yield the desired agent endogenously.

As used herein, the terms “effective amount,” “therapeutically effective amount,” and the like, when used in reference to a therapeutic or prophylactic composition, refer to a quantity sufficient to, when administered to the subject, including a mammal, for example a human, effect beneficial or desired results. Exemplary beneficial or desired results include the elongation of lifespan, as well as the treatment and/or prevention of geriatric syndromes, among other beneficial or desired results described herein. The quantity of a given composition described herein that will correspond to an effective amount may vary depending upon various factors, such as the given agent, the pharmaceutical formulation, the route of administration, the type of disease or disorder, the identity of the subject (e.g., age, sex, weight) being treated, and the like.

As used herein in the context of a gene or protein, the term “expression” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. In the context of a gene that encodes a protein product, the terms “gene expression” and the like are used interchangeably with the terms “protein expression” and the like. Expression of a gene or protein of interest in a subject can manifest, for example, by detecting: an increase in the quantity or concentration of mRNA encoding corresponding protein (as assessed, e.g., using RNA detection procedures described herein or known in the art, such as quantitative polymerase chain reaction (qPCR) and RNA seq techniques), an increase in the quantity or concentration of the corresponding protein (as assessed, e.g., using protein detection methods described herein or known in the art, such as enzyme-linked immunosorbent assays (ELISA), among others), and/or an increase in the activity of the corresponding protein (e.g., in the case of an enzyme, as assessed using an enzymatic activity assay described herein or known in the art) in a sample obtained from the subject. As used herein, a cell is considered to “express” a gene or protein of interest if one or more, or all, of the above events can be detected in the cell or in a medium in which the cell resides. For example, a gene or protein of interest is considered to be “expressed” by a cell or population of cells if one can detect (i) production of a corresponding RNA transcript, such as an mRNA template, by the cell or population of cells (e.g., using RNA detection procedures described herein); (ii) processing of the RNA transcript (e.g., splicing, editing, 5′ cap formation, and/or 3′ end processing, such as using RNA detection procedures described herein); (iii) translation of the RNA template into a protein product (e.g., using protein detection procedures described herein); and/or (iv) post-translational modification of the protein product (e.g., using protein detection procedures described herein).

As used herein, the term “frailty index” refers to a system used to assess the risk of frailty in a subject (e.g., a mammalian subject, such as a human). Frailty indices may be numerical scales, such as the 0-10 scale described in Tocchi, Best Practices in Nursing Care to Older Adults (The Hartford Institute for Geriatric Nursing, New York University, College of Nursing, 34, 2016), the disclosure of which is incorporated herein by reference.

As used herein, the term “geriatric syndrome” refers to a clinical pathology that is exhibited with an increasing frequency in a population of subjects (e.g., mammalian subjects, such as human subjects) as the age of the subjects in the population increases. While heterogeneous, geriatric syndromes share many common features. Geriatric syndromes are multifactorial health conditions that occur when the accumulated effects of impairments in multiple systems render an older person vulnerable to situational challenges. Examples of geriatric syndromes and criteria used to define this class of diseases are provided in Inouye et al., J. Am. Geriatr. Soc. 55:780-791 (2007), the disclosure of which is incorporated herein by reference.

As used herein, the terms “interfering ribonucleic acid” and “interfering RNA” refer to a RNA, such as a short interfering RNA (siRNA), micro RNA (miRNA), or short hairpin RNA (shRNA) that suppresses the expression of a target RNA transcript by way of (i) annealing to the target RNA transcript, thereby forming a nucleic acid duplex; and (ii) promoting the nuclease-mediated degradation of the RNA transcript and/or (iii) slowing, inhibiting, or preventing the translation of the RNA transcript, such as by sterically precluding the formation of a functional ribosome-RNA transcript complex or otherwise attenuating formation of a functional protein product from the target RNA transcript. Interfering RNAs as described herein may be provided to a patient in the form of, for example, a single- or double-stranded oligonucleotide, or in the form of a vector (e.g., a viral vector) containing a transgene encoding the interfering RNA. Exemplary interfering RNA platforms are described, for example, in Lam et al., Molecular Therapy—Nucleic Acids 4:e252 (2015); Rao et al., Advanced Drug Delivery Reviews 61:746-769 (2009); and Borel et al., Molecular Therapy 22:692-701 (2014), the disclosures of each of which are incorporated herein by reference in their entirety.

As used herein, the term “pharmaceutical composition” means a mixture containing a therapeutic compound to be administered to a patient, such as a mammal, e.g., a human, in order elongate the lifespan of the patient and/or prevent, treat or control a particular disease or condition affecting the patient.

As used herein, the term “pharmaceutically acceptable” refers to those compounds, materials, compositions and/or dosage forms, which are suitable for contact with the tissues of a patient, such as a mammal (e.g., a human) without excessive toxicity, irritation, allergic response and other problem complications commensurate with a reasonable benefit/risk ratio.

“Percent (%) sequence complementarity” with respect to a reference polynucleotide sequence is defined as the percentage of nucleic acids in a candidate sequence that are complementary to the nucleic acids in the reference polynucleotide sequence, after aligning the sequences and introducing gaps, if necessary, to achieve the maximum percent sequence complementarity. A given nucleotide is considered to be “complementary” to a reference nucleotide as described herein if the two nucleotides form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal complementarity over the full length of the sequences being compared. As an illustration, the percent sequence complementarity of a given nucleic acid sequence, A, to a given nucleic acid sequence, B, (which can alternatively be phrased as a given nucleic acid sequence, A that has a certain percent complementarity to a given nucleic acid sequence, B) is calculated as follows:

100multiplied by(the fraction X/Y)

where X is the number of complementary base pairs in an alignment (e.g., as executed by computer software, such as BLAST) of A and B, and where Y is the total number of nucleic acids in B. It will be appreciated that where the length of nucleic acid sequence A is not equal to the length of nucleic acid sequence B, the percent sequence complementarity of A to B will not equal the percent sequence complementarity of B to A. As used herein, a query nucleic acid sequence is considered to be “completely complementary” to a reference nucleic acid sequence if the query nucleic acid sequence has 100% sequence complementarity to the reference nucleic acid sequence.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100multiplied by(the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from an organism (e.g., a mammal, such as a human).

As used herein, the terms “subject′ and “patient” are used interchangeably and refer to an organism, such as a mammal (e.g., a human) that receives treatment so as to increase the lifespan of the subject and/or to prevent, treat, or control a disease or condition that is affecting the subject (e.g., a disease or condition described herein, such as a geriatric syndrome).

As used herein, the terms “treat” or “treatment” refer to therapeutic or prophylactic treatment, in which the object is to prevent or slow down (lessen) an undesired physiological change or disorder in a subject (e.g., a mammalian subject, such as a human).

As used herein, abbreviations of gene names refer to a wild-type version of the corresponding gene, as well as variants (e.g., splice variants, truncations, concatemers, and fusion constructs, among others) thereof. Examples of such variants are genes having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the nucleic acid sequences of a wild-type version of the gene.

As used herein, the term “antibody” (Ab) refers to an immunoglobulin molecule that specifically binds to, or is immunologically reactive with, a particular antigen, and includes polyclonal, monoclonal, genetically engineered, and otherwise modified forms of antibodies, including, but not limited to, chimeric antibodies, humanized antibodies, heteroconjugate antibodies (e.g., bi- tri- and quad-specific antibodies, diabodies, triabodies, and tetrabodies), and antigen-binding fragments of antibodies, including e.g., Fab′, F(ab′)₂, Fab, Fv, rlgG, and scFv fragments. In some embodiments, two or more portions of an immunoglobulin molecule are covalently bound to one another, e.g., via an amide bond, a thioether bond, a carbon-carbon bond, a disulfide bridge, or by a linker, such as a linker described herein or known in the art. Antibodies also include antibody-like protein scaffolds, such as the tenth fibronectin type III domain (¹⁰Fn3), which contains BC, DE, and FG structural loops similar in structure and solvent accessibility to antibody complementarity-determining regions (CDRs). The tertiary structure of the ¹⁰Fn3 domain resembles that of the variable region of the IgG heavy chain, and one of skill in the art can graft, e.g., the CDRs of a reference antibody onto the fibronectin scaffold by replacing residues of the BC, DE, and FG loops of ¹⁰Fn3 with residues from the CDR-H1, CDR-H2, or CDR-H3 regions, respectively, of the reference antibody.

As used herein, the term “aryl” refers to an unsaturated aromatic carbocyclic group of from 6 to 14 carbon atoms having a single ring (e.g., optionally substituted phenyl) or multiple condensed rings (e.g., optionally substituted naphthyl). Exemplary aryl groups include phenyl, naphthyl, phenanthrenyl, and the like.

As used herein, the term “cycloalkyl” refers to a monocyclic cycloalkyl group having from 3 to 8 carbon atoms, such as cyclopropyl, cyclobutyl, cyclopentyl, cyclohexyl, cycloheptyl, cyclooctyl, and the like.

As used herein, the term “halogen atom” refers to a fluorine atom, a chlorine atom, a bromine atom, or an iodine atom.

As used herein, the term “heteroaryl” refers to a monocyclic heteroaromatic, or a bicyclic or a tricyclic fused-ring heteroaromatic group. Exemplary heteroaryl groups include optionally substituted pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadia-zolyl, 1,2,5-oxadiazolyl, 1,3,4-oxadiazolyl, 1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxazolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl, benzoquinolyl, and the like.

As used herein, the term “heterocycloalkyl” refers to a 3 to 8-membered heterocycloalkyl group having 1 or more heteroatoms, such as a nitrogen atom, an oxygen atom, a sulfur atom, and the like, and optionally having 1 or 2 oxo groups such as pyrrolidinyl, piperidinyl, oxopiperidinyl, morpholinyl, piperazinyl, oxopiperazinyl, thiomorpholinyl, azepanyl, diazepanyl, oxazepanyl, thiazepanyl, dioxothiazepanyl, azokanyl, tetrahydrofuranyl, tetrahydropyranyl, and the like.

As used herein, the terms “lower alkyl” and “Cis alkyl” refer to an optionally branched alkyl moiety having from 1 to 6 carbon atoms, such as methyl, ethyl, propyl, isopropyl, butyl, isobutyl, sec-butyl, tert-butyl, pentyl, isopentyl, neopentyl, tert-pentyl, hexyl, and the like.

As used herein, the term “lower alkylene” refers to an optionally branched alkylene group having from 1 to 6 carbon atoms, such as methylene, ethylene, methylmethylene, trimethylene, dimethylmethylene, ethylmethylene, methylethylene, propylmethylene, isopropylmethylene, dimethylethylene, butylmethylene, ethylmethylmethylene, pentamethylene, diethylmethylene, dimethyltrimethylene, hexamethylene, diethylethylene and the like.

As used herein, the term “lower alkenyl” refers to an optionally branched alkenyl moiety having from 2 to 6 carbon atoms, such as vinyl, allyl, 1-propenyl, isopropenyl, 1-butenyl, 2-butenyl, 2-methylallyl, and the like.

As used herein, the term “lower alkynyl” refers to an optionally branched alkynyl moiety having from 2 to 6 carbon atoms, such as ethynyl, 2-propynyl, and the like.

As used herein, the term “optionally substituted” refers to a chemical moiety that may have one or more (e.g., 1, 2, 3, 4, 5, 6, 7, 8, 9, 10, or more chemical substituents, such as lower alkyl, lower alkenyl, lower alkynyl, cycloalkyl, heterocyclolalkyl, aryl, alkylaryl, heteroaryl, alkylheteroaryl, amino, ammonium, acyl, acyloxy, acylamino, aminocarbonyl, alkoxycarbonyl, ureido, carbamate, sulfinyl, sulfonyl, alkoxy, sulfanyl, halogen, carboxy, trihalomethyl, cyano, hydroxy, mercapto, nitro, and the like. An optionally substituted chemical moiety may contain, e.g., neighboring substituents that have undergone ring closure, such as ring closure of vicinal functional substituents, thus forming, e.g., lactams, lactones, cyclic anhydrides, acetals, thioacetals, or aminals formed by ring closure, for instance, in order to generate protecting group.

As used herein, the term “sulfinyl” refers to the chemical moiety “—S(O)—R” in which R represents, e.g., hydrogen, aryl, heteroaryl, optionally substituted alkyl, optionally substituted alkenyl, or optionally substituted alkynyl.

As used herein, the term “sulfonyl” refers to the chemical moiety “—SO₂—R” in which R represents, e.g., hydrogen, aryl, heteroaryl, optionally substituted alkyl, optionally substituted alkenyl, or optionally substituted alkynyl.

As used herein, the term “pharmaceutically acceptable salt” refers to a salt, such as a salt of a compound described herein, that retains the desired biological activity of the non-ionized parent compound from which the salt is formed. Examples of such salts include, but are not restricted to acid addition salts formed with inorganic acids (e.g., hydrochloric acid, hydrobromic acid, sulfuric acid, phosphoric acid, nitric acid, and the like), and salts formed with organic acids such as acetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalene sulfonic acid, naphthalene disulfonic acid, and poly-galacturonic acid. The compounds can also be administered as pharmaceutically acceptable quaternary salts, such as quaternary ammonium salts of the formula —NR,R′,R″ ⁺Z⁻, wherein each of R, R′, and R″ may independently be, for example, hydrogen, alkyl, benzyl, C₁-C₆-alkyl, C₂-C₆-alkenyl, C₂-C₆-alkynyl, C₁-C₆-alkyl aryl, C₁-C₆-alkyl heteroaryl, cycloalkyl, heterocycloalkyl, or the like, and Z is a counterion, such as chloride, bromide, iodide, —O-alkyl, toluenesulfonate, methyl sulfonate, sulfonate, phosphate, carboxylate (such as benzoate, succinate, acetate, glycolate, maleate, malate, fumarate, citrate, tartrate, ascorbate, cinnamoate, mandeloate, and diphenylacetate), or the like.

The structural compositions described herein also include the tautomers, geometrical isomers (e.g., E/Z isomers and cis/trans isomers), enantiomers, diastereomers, and racemic forms, as well as pharmaceutically acceptable salts thereof. Such salts include, e.g., acid addition salts formed with pharmaceutically acceptable acids like hydrochloride, hydrobromide, sulfate or bisulfate, phosphate or hydrogen phosphate, acetate, benzoate, succinate, fumarate, maleate, lactate, citrate, tartrate, gluconate, methanesulfonate, benzenesulfonate, and para-toluenesulfonate salts.

As used herein, chemical structural formulas that do not depict the stereochemical configuration of a compound having one or more stereocenters will be interpreted as encompassing any one of the stereoisomers of the indicated compound, or a mixture of one or more such stereoisomers (e.g., any one of the enantiomers or diastereomers of the indicated compound, or a mixture of the enantiomers (e.g., a racemic mixture) or a mixture of the diastereomers). As used herein, chemical structural formulas that do specifically depict the stereochemical configuration of a compound having one or more stereocenters will be interpreted as referring to the substantially pure form of the particular stereoisomer shown. “Substantially pure” forms refer to compounds having a purity of greater than 85%, such as a purity of from 85% to 99%, 85% to 99.9%, 85% to 99.99%, or 85% to 100%, such as a purity of 85%, 86%, 87%, 88%, 89%, 90%, 91%, 92%, 93%, 94%, 95%, 96%, 97%, 98%, 99%, 99.9%, 99.99%, 99.999%, or 100%, as assessed, for example, using chromatography and nuclear magnetic resonance techniques known in the art.

### BRIEF DESCRIPTION OF THE DRAWINGS

FIG. 1. RNAseq analysis of hepatic gene expression in mice subjected to lifespan-extending interventions.
(A) RNAseq dataset. Mice were subjected to indicated lifespan-extending interventions for several months (from 2 to 12 for different interventions and age groups; n=3 for each control and treatment group within each sex and age setting resulting in 78 samples in total). Interventions, which have not been previously analyzed at the level of gene expression, are colored in green. Sex and age of mice corresponding to each intervention is shown with X marks. Cases where an intervention failed to extend lifespan with statistical significance in females are shown with grey X mark.
(B) Overlap of gene expression changes in response to longevity interventions. Overlap of differentially expressed genes (BH adjusted p-value <0.05 and FC >1.5 in any direction) in response to MR in males, CR in males and females and in Snell dwarf males is shown. 44.3% of upregulated and 41.8% of downregulated genes in response to MR are shared with at least one other lifespan-extending intervention.
(C) Heatmap of functions enriched by gene changes in response to lifespan-extending interventions. Normalized enrichment score (NES) of functions are shown for every intervention. All functions enriched by at least one intervention are presented. FDR threshold of 0.1 was used to filter out functions nonsignificant for every individual intervention. Clustering has been performed with hierarchical average approach and Spearman correlation distance.
(D) Functions enriched by upregulated (up) and downregulated (down) genes across different interventions based on GSEA. Significance score, calculated as log₁₀(FDR q-value) corrected by the sign of regulation, is plotted on the y axis. FDR threshold of 0.1 is shown by dotted lines. Shown functions were selected manually. Ribosome: Ribosome (KEGG); Cytochrome P450: Drug metabolism by cytochrome P450 (KEGG); Glutathione: Glutathione metabolism (KEGG); Ox Phosph: Oxidative phosphorylation (KEGG); TCA cycle: Citrate Cycle/TCA Cycle (KEGG); FA oxidation: Fatty acid β-oxidation (GO); Mito Translation: Mitochondrial translation (GO); MR: Methionine Restriction; CR: Caloric Restriction; Snell: Snell dwarf mice; F: Females; M: Males.

FIG. 2. Feminizing effect of lifespan-extending interventions.
(A) Overlap of genes differentially expressed between males and females and in response to feminizing lifespan-extending interventions. More than 66% of genes differentially expressed between males and females are also differentially expressed in response to feminizing lifespan-extending interventions in males (such as GHRKO and CR at 6 months age). Notably, the overlap with gene expression changes in response to interventions in females (3.1% of upregulated and 2.6% of downregulated genes in CR females are shared with the feminizing phenotype) is about 2-fold smaller than in males (9.3% of upregulated and 8% of downregulated genes in CR males and 7.3% of upregulated and 8.1% of downregulated genes in GHRKO males are shared with the feminizing phenotype). Fisher exact test BH adjusted p-value <4.1·10⁻⁴ for overlap of all presented interventions with sex-associated genes.
(B) Feminizing effect of gene expression changes across interventions. Genetic (GHRKO, Snell dwarf mice) and dietary (CR, MR) interventions together with acarbose at 12 months and rapamycin at 6 months show significant feminizing effect in males. For all interventions and age groups, except for Protandim, males show significantly higher feminizing effect than females (Spearman correlation test adjusted p-value <2.6·10⁻⁶ for all interventions and age groups). The feminizing effect is defined as correlation of log₂FC of gender-associated genes between sexes and in response to certain interventions. Error bars represent 90% confidence intervals.
(C) Diminution of gender gene expression differences by lifespan-extending interventions. Gene expression distance between males and females is significantly decreased by the majority of lifespan-extending interventions, except for Protandim at 6 months and rapamycin at 12 months (BH adjusted Mann-Whitney test p-value <0.024). Each dot represents Manhattan distance between the expression of sex-specific genes in 2 samples (corresponding to male and female). All pairwise comparisons between single samples are shown on the plot. All individual distances are centered around the average distance between control samples. M: Males; F: Females. * P.adjusted <0.1; ** P.adjusted <0.05; *** P.adjusted <0.01.
(D) Feminizing effect of metabolite changes across interventions. The majority of interventions in males show a significant feminizing effect, except for rapamycin given at 12 months based on our RNAseq data. Males, except for rapamycin from the previously obtained data, also show a significantly higher feminizing effect compared to females (Spearman correlation test adjusted p-value <9.8·10⁻²). The feminizing effect is defined as correlation of log₂FC of gender-associated metabolites between sexes and in response to certain intervention. Error bars represent 90% confidence intervals.
(E) Diminution of gender metabolome differences by lifespan-extending interventions. Metabolic distance between males and females is significantly decreased by the majority of lifespan-extending interventions, except for rapamycin at 12 months from the new dataset (BH adjusted Mann-Whitney test p-value <0.011). Each dot represents Manhattan distance between the level of sex-specific metabolites in 2 samples (corresponding to male and female). All pairwise comparisons between single samples are shown on the plot. All individual distances are centered around the average distance between control samples. M: Males; F: Females. * P.adjusted <0.1; ** P.adjusted <0.05; *** P.adjusted <0.01.
(F) Heatmap with log₂FC of genes differentially expressed between females and males (Fem changes) and in response to different interventions within each sex. log₂FC of genes differentially expressed between females and males (BH adjusted p-value <0.05 and FC >1.5 in any direction) aggregated across age groups are shown.
(G) Functional enrichment of feminizing genes significantly associated with feminizing effect across interventions. Drug metabolism, fatty acid metabolism and complement and coagulation cascades are annotated by KEGG database; major urinary proteins are annotated by INTERPRO database.
Estradiol: 17-α-estradiol; GHRKO: Growth hormone receptor knockout; MR: Methionine Restriction; CR: Caloric Restriction; Snell: Snell dwarf mice; F: Females; M: Males; 12 m: 12 months; 6 m: 6 months; 5 m: 5 months.

FIG. 3. Genes significantly changed in response to CR, rapamycin and GH-deficiency across multiple datasets.
(A) Genes identified as significantly up- and downregulated in response to CR, rapamycin and GH deficiency. FDR threshold of 0.01 and p-value LOO threshold of 0.01 were used to select significant genes. There is significant overlap between the genes changed in response to CR and GH deficiency (Fisher exact test p-value <2.2·10⁻¹⁶)
(B) Functions enriched by upregulated and downregulated genes in response to CR, rapamycin and GH deficiency based on GSEA. Significance score, calculated as log₁₀(FDR q-value) corrected by the sign of regulation, is plotted on y axis. q-value threshold of 0.1 is shown by dotted lines. Presented functions were selected manually. Ox Phosph: Oxidative phosphorylation (KEGG); TCA cycle: Citrate Cycle/TCA Cycle (KEGG); Parkinsons: Parkinson's Disease (KEGG); Huntingtons: Huntington's Disease (KEGG); Ribosome: Ribosome (KEGG); Amino Acid Catabolism: Cellular Amino Acid Catabolic Process (GO); Glycolysis: Glycolysis/Gluconeogenesis (KEGG); Metabolism by P450: Drug metabolism by cytochrome P450 (KEGG).
(C) Overlap of transcription factors IDs enriched by genes differentially expressed in response to CR, rapamycin and GH deficiency. Permutation FDR of 0.01 was used to obtain the list of overrepresented IDs. Transcription factors specified in the text were selected manually.
(D) Interventions included into meta-analysis. 17 interventions associated with increased lifespan or healthspan are included in the aggregated dataset. Two interventions (metformin and resveratrol, shown in grey) are included into the dataset despite their inability to significantly increase lifespan in healthy mice as shown by the ITP program.
(E) Fold changes of genes up- and downregulated in response to CR, rapamycin and GH deficiency across different lifespan- and healthspan-extending interventions. GH-deficiency interventions form a tight cluster with similar transcriptome profile behavior, pointing to the same molecular mechanisms. Union of genes differentially expressed in response to CR, rapamycin and GH-deficiency interventions (BH adjusted p-value <0.01 and p-value LOO <0.01) and log₂FC scale was used to create the heatmap. Complete hierarchical clustering approach was employed.
(F) Spearman correlation between genes differentially expressed in response to CR, rapamycin and GH deficiency. The major cluster is formed by GH deficiency (Snell and Ames dwarf mice, GHRKO, Little mice), dietary interventions (CR, MR, EOD), FGF21 overexpression and others. Spearman correlation coefficient was calculated based on gene logFC aggregated across different datasets for every intervention. Complete hierarchical clustering approach was employed.
Snell: Snell dwarf mice; Ames: Ames dwarf mice; Little: Little mice; CR: Caloric Restriction; GH: Growth Hormone; GHRKO: Growth Hormone Receptor Knockout; FGF21 over: FGF21 overexpression.

FIG. 4. Mutual organization of gene expression profiles of lifespan-extending interventions.
(A) GSEA enrichment of interventions by genes regulated by CR, rapamycin and GH deficiency. Each cell represents adjusted p-value calculated based on GSEA against subsets of genes significantly affected by CR, rapamycin and GH-deficient interventions. Only statistically significant associations (BH adjusted p-value <0.1) are colored.
(B) Spearman correlation coefficient distribution between gene expression profiles of CR and other interventions. At the level of gene expression, CR showed statistically significant (BH adjusted Mann-Whitney test p-value <0.1) positive correlation with the majority of interventions, including itself (median Spearman correlation coefficient=0.32; BH adjusted Mann-Whitney test p-value=2.9·10⁻⁹³). For every intervention, violinplot shows distribution of Spearman correlation coefficients between gene expression changes of every dataset of CR and the indicated interventions. 250 genes with the lowest p-value were used for the calculation.
(C) Gene expression profile correlation matrix aggregated for every intervention pair. The majority of lifespan-extending interventions show significant positive correlation at the level of gene expression changes. For each pair of interventions, the matrix represents median Spearman correlation value across all possible comparisons of datasets representing corresponding interventions from different sources. 250 genes with the lowest p-value were used for the calculation. To make results unbiased, only data from different sources was used. For this reason, correlation couldn't be estimated for interventions, for which no independent pair of datasets from different sources was available. This missing data is shown by grey boxes. For the same reason, correlation coefficient of intervention with itself is not equal to 1 and, in some cases, could not be calculated (when only one source for certain intervention was available).
(D) Network of interventions based on similarity of their gene expression profiles. Protandim, rapamycin, MYC +/− and S6K1 −/− didn't show statistically significant positive association with any other intervention. The width of edge is defined by BH adjusted Mann-Whitney test p-value of Spearman correlation between interventions (in logarithmic scale). Only statistically significant (BH adjusted Mann-Whitney test p-value <0.1) connections are shown.
Estradiol: 17-α-estradiol; Snell: Snell dwarf mice; Ames: Ames dwarf mice; CR: Caloric restriction; MR: Methionine Restriction; EOD: Every-other-day feeding; FGF21 over: FGF21 overexpression; Little: Little mice; GHRKO: Growth Hormone Receptor Knockout.

FIG. 5. Common signatures of lifespan-extending interventions.
(A) Fold change of genes commonly regulated in response to lifespan-extending interventions. 166 upregulated and 134 downregulated genes were identified as common signatures of lifespan-extending interventions. Genes significantly regulated across interventions (BH adjusted robust p-value <0.01) were included in the heatmap. Individual control-intervention datasets are shown on the x axis.
(B) Cth fold change across different lifespan-extending interventions (upper panel) and across individual datasets used in the analysis (lower panel). Cystathionine gamma-lyase (Cth) gene is significantly upregulated across different lifespan-extending interventions (BH adjusted robust p-value=0.0033) and within 7 individual interventions. On the upper barplot, red asterisk denotes interventions with the BH adjusted p-value <0.05. On the lower plot, dots representing gene fold change within each individual dataset are colored based on the intervention type. Estradiol: 17-α-estradiol; Snell: Snell dwarf mice; Ames: Ames dwarf mice; Little: Little mice; CR: Caloric restriction; MR: Methionine Restriction; EOD: Every-other-day feeding; FGF21 over: FGF21 overexpression; GHRKO: Growth Hormone Receptor Knockout.
(C) GSEA functional enrichment of genes up- (red) and downregulated (blue) in response to lifespan-extending interventions in liver. Statistically significantly enriched functions (FDR q-value <0.1) are shown. Significance score, calculated as log₁₀(FDR q-value) corrected by the sign of regulation, is presented on x-axis. Presented functions were selected manually.
(D) Number of common up- (left) and downregulated (right) genes across lifespan-extending interventions in different tissues. Almost no individual genes are commonly changed in response to lifespan-extending interventions in liver, skeletal muscle and white adipose tissue. Genes were considered significantly associated if BH adjusted p-value <0.05 and p-value LOO <0.05.
(E) GSEA functional enrichment of genes up- (upper) and downregulated (lower) in response to lifespan-extending interventions across tissues. Although almost no common signatures were identified across tissues at the level of individual genes, a number of molecular functions were shared between liver, skeletal muscle and white adipose tissue. They include upregulated oxidative phosphorylation, amino acid metabolism and ribosome structural genes along with downregulated immune response genes. Functions statistically significantly associated with at least one lifespan extension metric (FDR q-value <0.1) are shown. Cells are colored based on significance scores, calculated as log₁₀(FDR q-value) corrected by sign of regulation. Presented functions were selected manually. Muscle: Skeletal Muscle; WAT: White Adipose Tissue.

FIG. 6. Gene expression signatures associated with the degree of lifespan extension.
(A) Fold change of genes associated with the maximum lifespan effect across different datasets. Genes identified as significantly associated with maximum lifespan effect (BH adjusted p-value <0.05 and p-value LOO <0.05), calculated as In(maximum lifespan ratio), are shown in the heatmap. 351 and 264 genes were found to have positive and negative association with maximum lifespan effect, respectively. Plot on the top shows maximum lifespan effect for corresponding dataset.
(B) Association of Dgat1 fold change with maximum lifespan. Although Dgat1 deletion is associated with lifespan extension in female mice, its fold change shows a slight positive association with the maximum lifespan ratio (slope coefficient=0.38 and BH adjusted p-value=0.007).
(C-F) Association of Hint1 (C), Irf2 (D), Eci1 (E) and Ndufab1 (F) fold change with maximum (left) and median (right) lifespan ratio. All specified genes show statistically significant associations with both maximum and median lifespan.
   CR: Caloric Restriction; FGF21 over: FGF21 overexpression; EOD: Every-Other-Day Feeding; Snell: Snell dwarf mice; Ames: Ames dwarf mice; Little: Little mice; GHRKO: Growth Hormone Receptor Knockout.

FIG. 7. Analysis of signatures associated with lifespan extension effect and identification of candidate lifespan-extending interventions.
(A-B) Fold change of Nqo1 (A) and Slc15a4 (B) across different interventions and their association with the maximum lifespan extension effect. Nqo1 (coding for NADH dehydrogenase 1) and Slc15a4 (coding for lysosomal amino acid transporter) are examples of genes both significantly shared by lifespan-extending interventions (BH adjusted robust p-value=0.011 and 0.008, respectively) and positively associated with the lifespan extension effect (BH adjusted p-value=0.002 and 0.02, respectively). Red asterisk denotes interventions with BH adjusted p-value <0.1. Estradiol: 17-α-estradiol; Snell: Snell dwarf mice; Ames: Ames dwarf mice; Little: Little mice; CR: Caloric restriction; MR: Methionine Restriction; EOD: Every-other-day feeding; FGF21 over: FGF21 overexpression; GHRKO: Growth Hormone Receptor Knockout.
(C) GSEA functional enrichment of genes positively (upper) and negatively (lower) associated with the lifespan extension effect. Generally, results are consistent across different metrics. Functions statistically significantly associated with at least one lifespan extension metric (FDR q-value <0.1) are shown. Cells are colored based on significance score, calculated as log₁₀(FDR q-value) corrected by the sign of regulation. Presented functions were selected manually.
(D) Number of genes showing positive (left) and negative (right) association with different metrics of the lifespan extension effect. Generally, different metrics show significant overlap in genes significantly associated with them (Fisher exact test p-value <10⁻¹⁸ in all cases). Genes were considered significantly associated if BH adjusted p-value <0.05 and p-value LOO <0.05.
(E) Association of identified longevity signatures with hepatic gene expression changes induced by individual interventions from publicly available datasets and those predicted by CMap. Longevity signatures include genes aggregated across individual interventions (CR, rapamycin and GH deficiency interventions), common signatures (Interventions common) and signatures associated with the lifespan extension effect (Maximum and median lifespan). Cells are colored based on significance score, calculated as log₁₀(BH adjusted p-value) corrected by sign of regulation. IL-6 Injection: GSE21060; Mat1a Knockout: GSE77082; Hypoxia: GSE15891; Keap1 Knockout: GSE11287; SRT2104: GSE49000; Sirt6 Over: Sirt6 Overexpression (PMID 22367546); Long-lived Strain: GSE10421; CR Rhesus Monkey: GSE104234.

FIG. 8. Functional enrichment of methionine restriction (A) and the feminizing effect of GHRKO (B).
(A) Significantly enriched functions in response to MR based on GSEA. Statistically significantly enriched functions (FDR q-value <0.1) are shown. Significance score, calculated as log₁₀(FDR q-value) corrected by the sign of regulation, is presented on x-axis. Presented functions were selected manually.
(B) Correlation between feminizing changes and changes induced by GHRKO in males. log₂FC of genes differentially expressed between males and females (BH adjusted p-value <0.05 and FC >1.5 in any direction) aggregated across age groups are shown. Genes statistically significantly changed in response to GHRKO (BH adjusted p-value <0.05 and FC >1,5 in any direction) are colored in red. Regression and identity lines are shown as grey and black dotted line, respectively.

FIG. 9. Igf1 (A) and Igfbp2 (B) fold change across different lifespan-extending interventions.
(A) Igf1 fold change across lifespan-extending interventions. Insulin-like growth factor 1 (Igf1) is significantly downregulated in response to all GH deficiency interventions (Ames and Snell dwarf mice, GHRKO and Little mice) as well as FGF21 overexpression and methionine restriction (BH adjusted p-value <0.1). Red asterisk denotes interventions with BH adjusted p-value <0.1.
(B) Igfbp2 fold change across lifespan-extending interventions. Insulin-like growth factor binding protein 2 (Igfbp2), being Igf1 inhibitor, is significantly upregulated in response to GH deficiency interventions (Ames dwarf mice, GHRKO and Little mice) as well as dietary interventions (MR and CR) and acarbose (BH adjusted p-value <0.1). Red asterisk denotes interventions with BH adjusted p-value <0.1.
Estradiol: 17-α-estradiol; Snell: Snell dwarf mice; Ames: Ames dwarf mice; Little: Little mice; CR: Caloric restriction; MR: Methionine Restriction; EOD: Every-other-day feeding; FGF21 over: FGF21 overexpression; GHRKO: Growth Hormone Receptor Knockout.

FIG. 10. Pathway enrichment analysis of individual interventions based on iPANDA.
(A) Pathways enriched by genes regulated in response to CR. Enrichment of similar pathways, such as activation of TCA cycle, respiratory electron transport, lipid biosynthesis, mitochondrial biogenesis, response to xenobiotics and glycolysis/gluconeogenesis along with inhibition of complement, mTOR and insulin signaling pathway, was discovered using an iPANDA approach. Statistically significantly enriched functions (BH adjusted p-value <0.1) are shown. Significance score, calculated as log₁₀(BH adjusted p-value) corrected by the sign of regulation, is presented on x-axis. The dotted line shows the border between up- and downregulated functions. The pathways shown were chosen manually.
(B) Pathways enriched by genes regulated in response to GH deficiency. Enrichment of similar pathways, such as activation of GSK3 signaling, respiratory electron transport and TCA cycle along with inhibition of complement and interferon, MAPK signaling, estrogen, mTOR and IGF1R pathways, was discovered using iPANDA approach. Statistically significantly enriched functions (BH adjusted p-value <0.1) are shown. Significance score, calculated as log₁₀(BH adjusted p-value) corrected by the sign of regulation, is presented on x-axis. The dotted line shows the border between up- and downregulated functions. Shown pathways were chosen manually.

FIG. 11. Amplitude of gene expression changes induced by different types of interventions.
(A) Standard deviations of gene expression changes (log₂FC) across three main types of interventions. Different intervention types lead to a different scale of gene expression changes, with pharmacological interventions being the mildest and genetic interventions being the most affected. All differences are statistically significant (Mann-Whitney test p-value is equal to 1.71·10⁻⁶ between pharmacological and dietary and 0.003 between dietary and genetic).
(B) Medians of gene expression changes (log₂FC) across three main types of interventions. Medians of gene expression changes are distributed similarly across different types of interventions (Mann-Whitney test p-value >0.05 for all three comparisons).

FIG. 12. Spearman correlation coefficient distribution between gene expression profiles of rapamycin and other interventions.

At the level of gene expression change, rapamycin shows significant positive correlation only with itself (median Spearman correlation coefficient=0.088; BH adjusted Mann-Whitney test p-value=2.8·10⁻³). Although thought to be CR mimetic, rapamycin shows slight (median Spearman correlation coefficient=−0.049) but significant (BH adjusted Mann-Whitney test p-value=2·10⁻³) negative correlation with CR at the level of gene expression. For every intervention, violinplot shows the distribution of Spearman correlation coefficient between gene expression changes of every dataset of rapamycin and the corresponding intervention. 250 genes consisting of 125 genes with the lowest p-value in each pair of datasets were used for calculation.
Estradiol: 17-α-estradiol; Snell: Snell dwarf mice; Ames: Ames dwarf mice; Little: Little mice; CR: Caloric restriction; MR: Methionine Restriction; EOD: Every-other-day feeding; FGF21 over: FGF21 overexpression; GHRKO: Growth Hormone Receptor Knockout.

FIG. 13. Distribution of the number of differentially expressed genes shared across interventions (A) and Gsta4 fold change across lifespan-extending interventions (B).
(A) Number of genes identified as statistically significantly up- (red) and downregulated (blue) in response to different lifespan-extending interventions. Genes affected by the largest number of individual interventions encode cytochrome P450s and glutathione metabolism proteins. FDR threshold of 0.1 was used to select significant genes within each intervention.
(B) Gsta4 fold change across lifespan-extending interventions. Glutathione S-transferase A4 (Gsta4) gene is one of significant commonly upregulated genes across lifespan-extending interventions (BH adjusted robust p-value=0.013). In addition to being common signature, it is significantly upregulated in response to 9 individual interventions (BH adjusted p-value <0.1). Red asterisk denotes interventions with BH adjusted p-value <0.1.
Estradiol: 17-α-estradiol; Snell: Snell dwarf mice; Ames: Ames dwarf mice; Little: Little mice; CR: Caloric restriction; MR: Methionine Restriction; EOD: Every-other-day feeding; FGF21 over: FGF21 overexpression; GHRKO: Growth Hormone Receptor Knockout.

FIG. 14. Expression change of genes, whose alterations lead to lifespan extension or shortening in mouse models.
(A) Brca1 is one of commonly upregulated genes across lifespan-extending interventions (BH adjusted p-value=0.04). Snell: Snell dwarf mice; Ames: Ames dwarf mice; Little: Little mice; CR: Caloric restriction; MR: Methionine Restriction; EOD: Every-other-day feeding; FGF21 over: FGF21 overexpression; GHRKO: Growth Hormone Receptor Knockout.
(B) Overlap of gene signatures associated with lifespan extension and genes, whose alteration affects mouse lifespan. Overlap of longevity signatures and genes with the effect on lifespan is not significant for all pairwise comparisons (Fisher exact test p-value >0.33 for all comparisons). Common: Common signatures; Max Lifespan: signatures associated with maximum lifespan increase; Pro Longevity: Genes, whose overexpression extends lifespan; Anti Longevity: Genes, whose depletion extends lifespan.

FIG. 15. Identification of candidate lifespan-extending interventions based on longevity signatures.

Gene expression changes in response to interventions are scored against longevity signatures to identify candidate compounds with lifespan-extending effects. Statistical significance of association with longevity signatures is calculated using permutation test and adjusted with Benjamini-Hochberg procedure.

FIG. 16. Association of gene expression profiles of interventions from public sources (upper) and predicted by CMap (lower) with identified longevity signatures.

The latter include gene signatures of individual interventions (CR, rapamycin and GH deficiency), common signatures (Interventions common) and signatures associated with the effect on lifespan (Maximum and median lifespan). Cells are colored based on significance score, calculated as log₁₀(adjusted p-value) corrected by sign of regulation. Sirt6 Over: Sirt6 Overexpression.

FIG. 17. Validation of predicted interventions.

CMap is used for prediction of perspective compounds. Mouse and human primary hepatocytes and mouse in vivo models are used for validation.

FIG. 18. Validation of predicted compounds in mouse liver.

Four-month old UM-HET3 mice were subjected to diets for 1 month, followed by gene expression analyses. The figure shows the significance of associations between longevity signatures and gene expression changes in response to predicted compounds.

FIGS. 19A-19C. Schemes outlining the design of lifespan and healthspan experiments described in Example 21, below.

FIG. 19A shows the general design of the experiments described in Example 21, the results of which are reported in FIGS. 20-33, summarized below. Briefly, 24-28 mice per intervention were used, with an approximately equal number of males and females. Mice were first assessed with regard to frailty index and gait speed, and then randomized to make sure the experimental and control groups had the same average frailty index and gait speed. Mice were then given a diet containing a compound of interest. Control mice were treated identically, except that their diet did not have the compound of interest. Mice were monitored daily until they died. A separate cohort of old mice was assessed for frailty index and gait speed. Compounds that exhibited a lifespan-extending effect are discussed below.

FIG. 20. Effect of AZD-8055 on lifespan.

AZD-8055 extends lifespan of C57BL/6 male mice when given late in life. Arrow indicates the treatment onset. n=15 in the AZD-8055 group, and n=14 in the Control group.

FIGS. 21A-21B. Effect of AZD-8055 on frailty index and gait speed.

Left panel: AZD-8055 was given to 31-month-old C57BL/6 mice, n=10 for males and n=4 for females the AZD-8055 group, and n=18 for males and n=8 for females for the Control group. Right panel: same as in left panel, but gait speed was assessed in males. AZD-8055 was given to 31-month-old C57BL/6 mice, n=6 for the AZD-8055 group, and n=17 for the Control group.

FIG. 22. Effect of AZD-8055 on glucose tolerance.

The treatment does not lead to glucose intolerance in old C57BL/6 mice. 23-month-old mice were treated for 2.5 months prior to analyses.

FIGS. 23A-23B. Effect of Selumetinib on lifespan.

Selumetinib extends lifespan of C57BL/6 mice when given late in life. Left: survival of males and females combined (n=20 per group). Right: an independent cohort of female mice (n=15 per group), until they were sacrificed for biochemical experiments. Arrows indicate the onset of treatment.

FIGS. 24A-24B. Effect of Selumetinib on frailty index and gait speed.

Left: Selumetinib improves frailty index of 31-month-old C57BL/6 female mice (n=8 for Selumetinib, n=10 for Control). Right: Gait speed.

FIG. 25. Effect of Selumetinib on the immune system.

Population of immune cells in the spleen is not altered by Selumetinib (n=7 for Control, n=12 for Selumetinib). 27-month-old C57BI/6 females were used. Cells were analyzed by FACS: B-cells are CD45+CD19+, T-cells are CD45+CD3+, and myeloid cells are CD45+CD11b+.

FIG. 26. Celastrol may extend lifespan of C57BL/6 mice when given late in life.

Arrow indicates the onset of treatment. n=19 for the Celastrol group, and n=20 for the Control group.

FIGS. 27A-27B. Effect of Celastrol on frailty index and gait speed.

Celastrol does not affect frailty index or gait speed. n=10 per sex per treatment.

FIG. 28. Effect of LY294002 on lifespan of C57BI/6 male mice when given in late life.

Arrow indicates the onset of treatment. n=14 for the LY294002 group, and n=14 for the Control group.

FIGS. 29A-29B. Effect of LY294002 on frailty index and gait speed.

Both frailty index and gait speed are improved by this compound in 31-month-old C57BL/6 male mice. Left: frailty index: n=9 for males and n=3 for females for the LY294002 group, and n=18 for males and n=8 for females for the Control group. Right: gait speed: n=9 for the LY294002 group, and n=17 for the Control group.

FIG. 30. Effect of LY294002 on glucose tolerance.

No effect was observed. ns: not significant.

FIG. 31. Effect of KU-0063794 on lifespan of C57BL/6 male mice when given in late life.

Arrow indicates the onset of treatment. n=14 for the KU-0063794, and n=14 for the Control group.

FIGS. 32A-32B. Effect of KU-0063794 on gait speed and frailty index.

Both frailty index and gait speed are improved by this compound in 31-month-old C57BL/6 male mice. Left: frailty index: n=13 for males and n=2 for females for the KU-0063794 group, and n=18 for males and n=8 for females for the Control group. Right: gait speed: n=7 for the KU-0063794 group, and n=17 for the Control group.

FIG. 33. Effect of KU-0063794 on glucose tolerance.

No effect of this compound was observed. ns: not significant.

### DETAILED DESCRIPTION

The potential to live shorter or longer life is defined by the metabolic state of cells, and, in turn, is reflected in their gene expression patterns. The transition from a shorter- to a longer-lived state is observed when comparing the transcriptomes of (i) particular organs of mice subjected to interventions known to extend lifespan; (ii) cell types widely differing in lifespan, a parameter referred to as “cell turnover;” and (iii) particular organs between shorter- and longer-lived mammals.

Based on gene expression analyses of these models, transcriptomic patterns associated with lifespan have been identified, and an approach for identification of new lifespan-extending interventions has been developed. This approach was then applied to predict candidate longevity interventions. The present disclosure describes this approach and the validation of candidate prediction using different biological models.

### Identification of Gene Expression Longevity Signatures

The gene expression patterns that reflect the transition from shorter to longer lived states are designated throughout the present disclosure as “longevity signatures.” A total of 10 longevity signatures have been developed based on the transcriptomes of (i) mice treated with 17 different lifespan-extending interventions (6 “intervention-based signatures”); (ii) 20 organs and cell types differing in cell turnover (1 “turnover-based signature”); and (iii) liver, kidney, and brain of 41 species of mammals differing 30-fold in lifespan (3 “organ-specific signatures”). Each of these gene signatures contains a set of genes that is up-regulated in longer-living cells, as well as a set of genes that is down-regulated in longer-living cells. The 6 intervention-based signatures are shown in Tables 1-6 (up-regulated genes) and in Tables 11-16 (down-regulated genes), below. The 1 turnover-based signature is shown in Table 7 (up-regulated genes) and Table 17 (down-regulated genes), below. The 3 organ-specific signatures are shown in Tables 8-10 (up-regulated genes) and Tables 18-20 (down-regulated genes), below.

As described in further detail in the working examples, below, the genes within the foregoing signatures were identified as having an expression pattern associated with lifespan by various metrics. For example, the intervention-based signatures were identified by analyzing gene expression patterns that are observed in mammals upon treatment with agents known to have a lengthening effect on lifespan. The intervention-based signatures include 3 signatures corresponding to the genes perturbed in response to individual longevity interventions (calorie restriction, rapamycin and growth hormone deficient mutants), 1 signature corresponding to the genes commonly perturbed by all interventions and 2 signatures corresponding to the genes, which expression change in response to interventions is associated with the effect on median or maximum lifespan. The turnover-based signature was identified by analyzing gene expression patterns across different cell types and tissues in humans and correlating genes that are up-regulated or down-regulated with cell lifespan. The organ-specific signatures were identified by analyzing the gene expression patterns in particular organs (liver, kidney, and brain) across 41 species of mammals and correlating genes that are up-regulated or down-regulated with the lifespan of the corresponding mammal.

In sum, the above procedures enabled the identification of 10 longevity signatures, captured by Tables 1-10 (up-regulated genes) and Tables 11-20 (down-regulated genes), that are characteristic of elevated lifespan. The sections that follow describe the procedures used to identify these signatures in further detail. The following sections also describe methods that can be used to screen for interventions (e.g., chemical agents and/or lifestyle changes, among others) capable of up-regulating one or more genes in Tables 1-10 and/or down-regulating one or more genes in Tables 11-20. Such interventions can be used to increase lifespan of a subject (e.g., a mammalian subject, such as a human), as well as to reduce the risk of frailty in a subject, improve the learning ability of the subject, and treat, prevent, and/or delay the onset of geriatric syndromes in a subject.

### Identification of Candidate Lifespan-Extending Interventions Based on Longevity Signatures

This section provides an example of how the gene signatures described above can be used to screen for lifespan-extending interventions. Briefly, the gene signatures described above were screened for candidate longevity interventions across 3,300 compounds using the Connectivity Map (CMap) database. CMap aggregates gene expression data related to the response of several human cell lines to different drugs. This platform was utilized to identify compounds with the most significant positive association with the above longevity signatures. To account for possible differences in mechanisms behind different signatures, each of these signatures was analyzed separately. To search for other interventions potentially effecting lifespan, including genetic, pharmacological, and environmental interventions, the GEO database was also utilized, which contains gene expression datasets corresponding to the effect of many interventions on different biological models.

To statistically estimate the association of certain gene expression profiles with the above signatures, a gene set enrichment analysis (GSEA)-based approach was also developed, which examines whether a certain gene set is enriched among up- or down-regulated genes (FIG. 15). For this purpose, the genes in the above longevity signatures were ranked from the most statistically significantly up-regulated genes to the most statistically significant down-regulated genes in response to certain interventions. The final GSEA score was calculated as a mean of similarity scores determined separately for sets of up- and down-regulated genes. To calculate the statistical significance of this score, a permutation test was performed, and to adjust for multiple comparisons, a Benjamini-Hochberg procedure was used. The resulting adjusted p-value was considered as a measure of association with longevity signatures.

### Validation of Predicted Interventions

To validate the above approach and predictions, specific gene expression datasets were chosen from the GEO database that correspond to the effect of certain interventions considered to be health- and lifespan-extending or shortening on mouse liver (FIG. 16). 4 compounds were chosen as most significantly associated with signatures obtained across longevity interventions in mice. UM-HET3 mice were then subjected to these compounds for 1 month, at which point the mice were sacrificed and subjected to gene expression analysis. Finally, the association GSEA-based test was applied to validate the findings (FIG. 16).

A significant positive association was detected with the majority of longevity signatures for all compounds predicted via CMap. Additionally, significant associations were detected for datasets from GEO, consistent with the predictions described above. For example, as described in the working examples below, mild hypoxia and Keap1 knockout perturbed gene expression in the same way as longevity interventions, whereas interleukin-6 injection and Mat1a knockout led to the opposite changes.

This approach was then expanded and compounds with the most significant positive association with different longevity associations using CMap were selected. These hits were verified using various biological models (FIG. 17).

First, the identified hits were applied to human and mouse primary hepatocytes, and ensuing gene expression profiles were obtained. To treat human cells, 3 different doses of each agent were used, whereas for mice, a single dose of each agent was used. Using the GSEA-based approach, statistically significant (permutation test adjusted p-value <0.1) associations were identified with at least one longevity signature for 10 (25% of tested compounds) and 31 (44.3% of tested compounds) drugs in human and mouse hepatocytes, respectively.

Second, diets were prepared for 24 of the identified compounds. These diets were administered to mice for 1 month, and ensuing gene expression changes were monitored. Using the GSEA-based association test, 18 drugs (69% of tested compounds) were identified as having a statistically significant association with at least one longevity signature. Moreover, on average, every compound had significant associations with 2.8 different signatures, supporting the robustness of this approach (FIG. 18).

Taken together, the above findings substantiate a method for unbiased identification of candidate longevity interventions and show how this method was validated in both cell culture and in vivo models. These findings are described in further detail in the working examples below.

### Methods of Measuring Gene Expression

The expression level of a gene described herein (e.g., a gene set forth in one or more of the longevity signatures recited in Tables 1-20) can be ascertained, for example, by evaluating the concentration or relative abundance of mRNA transcripts derived from transcription of the gene. Additionally or alternatively, gene expression can be determined by evaluating the concentration or relative abundance of encoded protein produced by transcription and translation of the corresponding gene. Protein concentrations can also be assessed using functional assays. The sections that follow describe exemplary techniques that can be used to measure the expression level of a gene of interest. Gene expression can be evaluated by a number of methodologies known in the art, including, but not limited to, nucleic acid sequencing, microarray analysis, proteomics, in-situ hybridization (e.g., fluorescence in-situ hybridization (FISH)), amplification-based assays, in situ hybridization, fluorescence activated cell sorting (FACS), northern analysis and/or PCR analysis of mRNAs.

### Nucleic Acid Detection

Nucleic acid-based methods for determining gene expression include imaging-based techniques (e.g., Northern blotting or Southern blotting). Northern blot analysis is a conventional technique well known in the art and is described, for example, in Molecular Cloning, a Laboratory Manual, second edition, 1989, Sambrook, Fritch, Maniatis, Cold Spring Harbor Press, 10 Skyline Drive, Plainview, N.Y. 11803-2500. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis).

Gene detection techniques that may be used in conjunction with the compositions and methods described herein further include microarray sequencing experiments (e.g., Sanger sequencing and next-generation sequencing methods, also known as high-throughput sequencing or deep sequencing). Exemplary next generation sequencing technologies include, without limitation, Illumina sequencing, Ion Torrent sequencing, 454 sequencing, SOLiD sequencing, and nanopore sequencing platforms. Additional methods of sequencing known in the art can also be used. For instance, gene expression at the mRNA level may be determined using RNA-Seq (e.g., as described in Mortazavi et al., Nat. Methods 5:621-628 (2008) the disclosure of which is incorporated herein by reference in their entirety). RNA-Seq is a robust technology for monitoring expression by direct sequencing the RNA molecules in a sample. Briefly, this methodology may involve fragmentation of RNA to an average length of 200 nucleotides, conversion to cDNA by random priming, and synthesis of double-stranded cDNA (e.g., using the Just cDNA DoubleStranded cDNA Synthesis Kit from Agilent Technology). Then, the cDNA is converted into a molecular library for sequencing by addition of sequence adapters for each library (e.g., from Illumina®/Solexa), and the resulting 50-100 nucleotide reads are mapped onto the genome.

Gene expression levels may be determined using microarray-based platforms (e.g., single-nucleotide polymorphism arrays), as microarray technology offers high resolution. Details of various microarray methods can be found in the literature. See, for example, U.S. Pat. No. 6,232,068 and Pollack et al., Nat. Genet. 23:41-46 (1999), the disclosures of each of which are incorporated herein by reference in their entirety. Using nucleic acid microarrays, mRNA samples are reverse transcribed and labeled to generate cDNA. The probes can then hybridize to one or more complementary nucleic acids arrayed and immobilized on a solid support. The array can be configured, for example, such that the sequence and position of each member of the array is known. Hybridization of a labeled probe with a particular array member indicates that the sample from which the probe was derived expresses that gene. Expression level may be quantified according to the amount of signal detected from hybridized probe-sample complexes. A typical microarray experiment involves the following steps: 1) preparation of fluorescently labeled target from RNA isolated from the sample, 2) hybridization of the labeled target to the microarray, 3) washing, staining, and scanning of the array, 4) analysis of the scanned image and 5) generation of gene expression profiles. One example of a microarray processor is the Affymetrix GENECHIP® system, which is commercially available and comprises arrays fabricated by direct synthesis of oligonucleotides on a glass surface. Other systems may be used as known to one skilled in the art.

Amplification-based assays also can be used to measure the expression level of a gene described herein. In such assays, the nucleic acid sequences of the gene act as a template in an amplification reaction (for example, PCR, such as qPCR). In a quantitative amplification, the amount of amplification product is proportional to the amount of template in the original sample. Comparison to appropriate controls provides a measure of the expression level of the gene, corresponding to the specific probe used, according to the principles described herein. Methods of real-time qPCR using TaqMan probes are well known in the art. Detailed protocols for real-time qPCR are provided, for example, in Gibson et al., Genome Res. 6:995-1001 (1996), and in Heid et al., Genome Res. 6:986-994 (1996), the disclosures of each of which are incorporated herein by reference in their entirety. Levels of gene expression as described herein can be determined by RT-PCR technology. Probes used for PCR may be labeled with a detectable marker, such as, for example, a radioisotope, fluorescent compound, bioluminescent compound, a chemiluminescent compound, metal chelator, or enzyme.

### Protein Detection

Gene expression can additionally be determined by measuring the concentration or relative abundance of a corresponding protein product. Protein levels can be assessed using standard detection techniques known in the art. Protein expression assays suitable for use with the compositions and methods described herein include proteomics approaches, immunohistochemical and/or western blot analysis, immunoprecipitation, molecular binding assays, ELISA, enzyme-linked immunofiltration assay (ELIFA), mass spectrometry, mass spectrometric immunoassay, and biochemical enzymatic activity assays. In particular, proteomics methods can be used to generate large-scale protein expression datasets in multiplex. Proteomics methods may utilize mass spectrometry to detect and quantify polypeptides (e.g., proteins) and/or peptide microarrays utilizing capture reagents (e.g., antibodies) specific to a panel of target proteins to identify and measure expression levels of proteins expressed in a sample (e.g., a single cell sample or a multi-cell population).

Exemplary peptide microarrays have a substrate-bound plurality of polypeptides, the binding of an oligonucleotide, a peptide, or a protein to each of the plurality of bound polypeptides being separately detectable. Alternatively, the peptide microarray may include a plurality of binders, including, but not limited to, monoclonal antibodies, polyclonal antibodies, phage display binders, yeast two-hybrid binders, aptamers, which can specifically detect the binding of specific oligonucleotides, peptides, or proteins. Examples of peptide arrays may be found in U.S. Pat. Nos. 6,268,210, 5,766,960, and 5,143,854, the disclosures of each of which are incorporated herein by reference in their entirety.

Mass spectrometry (MS) may be used in conjunction with the methods described herein to identify and characterize gene expression. Any method of MS known in the art may be used to determine, detect, and/or measure a protein or peptide fragment of interest, e.g., LC-MS, ESI-MS, ESI-MS/MS, MALDI-TOF-MS, MALDI-TOF/TOF-MS, tandem MS, and the like. Mass spectrometers generally contain an ion source and optics, mass analyzer, and data processing electronics. Mass analyzers include scanning and ion-beam mass spectrometers, such as time-of-flight (TOF) and quadruple (Q), and trapping mass spectrometers, such as ion trap (IT), Orbitrap, and Fourier transform ion cyclotron resonance (FT-ICR), may be used in the methods described herein. Details of various MS methods can be found in the literature. See, for example, Yates et al., Annu. Rev. Biomed. Eng. 11:49-79, 2009, the disclosure of which is incorporated herein by reference in its entirety.

Prior to MS analysis, proteins in a sample obtained from the patient can be first digested into smaller peptides by chemical (e.g., via cyanogen bromide cleavage) or enzymatic (e.g., trypsin) digestion. Complex peptide samples also benefit from the use of front-end separation techniques, e.g., 2D-PAGE, HPLC, RPLC, and affinity chromatography. The digested, and optionally separated, sample is then ionized using an ion source to create charged molecules for further analysis. Ionization of the sample may be performed, e.g., by electrospray ionization (ESI), atmospheric pressure chemical ionization (APCI), photoionization, electron ionization, fast atom bombardment (FAB)/liquid secondary ionization (LSIMS), matrix assisted laser desorption/ionization (MALDI), field ionization, field desorption, thermospray/plasmaspray ionization, and particle beam ionization. Additional information relating to the choice of ionization method is known to those of skill in the art.

After ionization, digested peptides may then be fragmented to generate signature MS/MS spectra. Tandem MS, also known as MS/MS, may be particularly useful for analyzing complex mixtures. Tandem MS involves multiple steps of MS selection, with some form of ion fragmentation occurring in between the stages, which may be accomplished with individual mass spectrometer elements separated in space or using a single mass spectrometer with the MS steps separated in time. In spatially separated tandem MS, the elements are physically separated and distinct, with a physical connection between the elements to maintain high vacuum. In temporally separated tandem MS, separation is accomplished with ions trapped in the same place, with multiple separation steps taking place over time. Signature MS/MS spectra may then be compared against a peptide sequence database (e.g., SEQUEST). Post-translational modifications to peptides may also be determined, for example, by searching spectra against a database while allowing for specific peptide modifications.

### Pharmaceutical Compositions

Using the compositions and methods of the disclosure, one can screen for interventions (e.g., chemical agents, dietary supplements, diets, and/or lifestyle changes, among others) that are capable of effectuating a change in gene expression consistent with the longevity signatures set forth in one or more of Tables 1-20. For example, one may screen for an intervention that is capable of (i) up-regulating one or more of the genes set forth in Tables 1-10 and/or (ii) down-regulating one or more of the genes set forth in Tables 11-20. Such interventions are expected to enhance lifespan and promote the overall wellbeing of the subject, e.g., by reducing the risk of frailty in the subject, improving the learning ability of the subject, and/or preventing or delaying the onset of a geriatric syndrome in the subject.

Examples of agents that up-regulate one or more genes set forth in the longevity signatures shown in Tables 1-10 and/or down-regulate one or more gens set forth in the longevity signatures shown in Tables 11-20 include the following compounds. As described herein, such compounds may be used to enhance lifespan and promote the overall wellbeing of the subject, e.g., by reducing the risk of frailty in the subject, improving the learning ability of the subject, and/or preventing or delaying the onset of a geriatric syndrome in the subject. Examples of these compounds are KU-0063794 (rel-5-[2-[(2R,6S)-2,6-dimethyl-4-morpholinyl]-4-(4-morpholinyl)pyrido[2,3-d]pyrimidin-7-yl]-2-methoxybenzenemethanol), Ascorbyl Palmitate ([(2S)-2-[(2R)-4,5-Dihydroxy-3-oxo-2-furyl]-2-hydroxy-ethyl] hexadecanoate), Celastrol (3-Hydroxy-9β,13α-dimethyl-2-oxo-24,25,26-trinoroleana-1(10),3,5,7-tetraen-29-oic acid), Oligomycin-a ((1R,4E,5'S,6S,6'S,7R,8S,10R,11R,12S,14R,15S,16R,18E,20E,22R,25S,27R,28S,29R)-22-ethyl-7,11,14,15-tetrahydroxy-6′-[(2R)-2-hydroxypropyl]-5′,6,8,10,12,14,16,28,29-nonamethyl-3′,4′,5′,6′-tetrahydro-3H,9H,13H-spiro[2,26-dioxabicyclo[23.3.1]nonacosa-4,18,20-triene-27,2′-pyran]-3,9,13-trione), NVP-BEZ235 (2-Methyl-2-{4-[3-methyl-2-oxo-8-(quinolin-3-yl)-2,3-dihydro-1H-imidazo[4,5-c]quinolin-1-yl]phenyl}propanenitrile), AZD-8055 (5-[2,4-bis[(3S)-3-methyl-4-morpholinyl]pyrido[2,3-d]pyrimidin-7-yl]-2-methoxy-benzenemethanol), Importazole (N-(1-Phenylethyl)-2-(pyrrolidin-1-yl)quinazolin-4-amine), Ryuvidine (2-methyl-5-[(4-methylphenyl)amino]-4,7-benzothiazoledione), NSC-663284 (6-Chloro-7-[[2-(4-morpholinyl)ethyl]amino]-5,8-quinolinedione), PI-828 (2-(4-Morpholinyl)-8-(4-aminopheny)l-4H-1-benzopyran-4-one), Pyrvinium pamoate (6-(Dimethylamino)-2-[2-(2,5-dimethyl-1-phenyl-1H-pyrrol-3-yl)ethenyl]-1-methyl-4,4′-methylenebis[3-hydroxy-2-naphthalenecarboxylate] (2:1)-quinolinium), PI-103 (3-[4-(4-morpholinyl)pyrido[3′,2′:4,5]furo[3,2-d]pyrimidin-2-yl]-phenol), YM-155 (4,9-dihydro-1-(2-methoxyethyl)2-methyl-4,9-dioxo-3-(2-pyrazinylmethyl)-1H-naphth[2,3-d]imidazolium, bromide), Prostratin ((1aR,1bS,4aR,7aS,7bR,8R,9aS)-4a,7b-dihydroxy-3-(hydroxymethyl)-1,1,6,8-tetramethyl-5-oxo-1,1a,1b,4,4a,5,7a,7b,8,9-decahydro-9aH-cyclopropa[3,4]benzo[1,2-e]azulen-9a-yl acetate), BCI hydrochloride (3-(cyclohexylamino)-2,3-dihydro-2-(phenylmethylene)-1H-inden-1-one, monohydrochloride), Dorsomorphin-Compound C (6-[4-[2-(1-Piperidinyl)ethoxy]phenyl]-3-(4-pyridinyl)pyrazolo[1,5-a]pyrimidine), VU-0418947-2 (6-Phenyl-N-[(3-phenylphenyl)methyl]-3-pyridin-2-yl-1,2,4-triazin-5-amine), JNK-9L (4-[3-fluoro-5-(4-morpholinyl)phenyl]-N-[4-[3-(4-morpholinyl)-1,2,4-triazol-1-yl]phenyl]-2-pyrimidinamine), Phloretin (3-(4-Hydroxyphenyl)-1-(2,4,6-trihydroxyphenyl)propan-1-one), ZG-10 ((E)-4-(4-(dimethylamino)but-2-enamido)-N-(3-((4-(pyridin-3-yl)pyrimidin-2-yl)amino)phenyl)benzamide), Proscillaridin (5-[(3S,8R,9S,10R,13R,14S,17R)-14-Hydroxy-10,13-dimethyl-3-((2R,3R,4R,5R,6R)-3,4,5-trihydroxy-6-methyltetrahydro-2H-pyran-2-yloxy)-2,3,6,7,8,9,10,11,12,13,14,15,16,17-tetradecahydro-1H-cyclopenta[a]phenanthren-17-yl]-2H-pyran-2-one), YC-1 (3-(5′-Hydroxymethyl-2′-furyl)-1-benzyl indazole), IKK-2-inhibitor-V (N-(3,5-Bis-trifluoromethylphenyl)-5-chloro-2-hydroxybenzamide), Anisomycin ((2R,3S,4S)-4-hydroxy-2-(4-methoxybenzyl)-pyrrolidin-3-yl acetate), LY294002 (2-Morpholin-4-yl-8-phenylchromen-4-one), Colforsin ([(3R,4aR,5S,6S,6aS,10S,10aR,10b5)-5-acetyloxy-3-ethenyl-10,10b-dihydroxy-3,4a,7,7,10a-Pentamethyl-1-oxo-5,6,6a,8,9,10-hexahydro-2H-benzo[f]chromen-6-yl] 3-d imethylaminopropanoate), Rilmenidine (N-(Dicyclopropylmethyl)-4,5-dihydro-1,3-oxazol-2-amine), Selumetinib (6-(4-Bromo-2-chloroanilino)-7-fluoro-N-(2-hydroxyethoxy)-3-methylbenzimidazole-5-carboxamide), GDC-0941 (Pictilisib, 4-(2-(1H-Indazol-4-yl)-6-((4-(methylsulfonyl)piperazin-1-yl)methyl)thieno[3,2-d]pyrimidin-4-yl)morpholine), Valdecoxib (4-(5-methyl-3-phenylisoxazol-4-yl)benzenesulfonamide), Myricetin (3,5,7-Trihydroxy-2-(3,4,5-trihydroxyphenyl)-4-chromenone), Cyproheptadine (4-(5H-Dibenzo[a,d]cyclohepten-5-ylidene)-1-methylpiperidine), Vorinostat (N-Hydroxy-N′-phenyloctanediamide), Nifedipine (3,5-Dimethyl 2,6-dimethyl-4-(2-nitrophenyl)-1,4-dihydropyridine-3,5-dicarboxylate), Phylloquinone (2-Methyl-3-[(E)-3,7,11,15-tetramethylhexadec-2-enyl]naphthalene-1,4-dione), Withaferin-A ((4β,5β,6β,22R)-4,27-Dihydroxy-5,6:22,26-diepoxyergosta-2,24-diene-1,26-dione), Temsirolimus ((1R,2R,4S)-4-{(2R)-2-[(3S,6R,7E,9R,10R,12R,14S,15E,17E,19E,21 S,23S,26R,27R,34aS)-9,27-dihydroxy-10,21-dimethoxy-6,8,12,14,20,26-hexamethyl-1,5,11,28,29-pentaoxo-1,4,5,6,9,10,11,12,13,14,21,22,23,24,25,26,27,28,29,31,32,33,34,34a-tetracosahydro-3H-23,27-epoxypyrido[2,1-c][1,4]oxazacyclohentriacontin-3-yl]propyl}-2-methoxycyclohexyl 3-hydroxy-2-(hydroxymethyl)-2-methylpropanoate), SN-38 (4,11-diethyl-4,9-dihydroxy-(4S)-1H-pyrano[3′,4′:6,7]indolizino[1,2-b]quinoline-3,14(4H,12H)-dione), GSK-1059615 (5-[[4-(4-Pyridinyl)-6-quinolinyl]methylene]-2,4-thiazolidenedione), Tipifarnib (6-[(R)-amino-(4-chlorophenyl)-(3-methylimidazol-4-yl)methyl]-4-(3-chlorophenyl)-1-methylquinolin-2-one), Linifanib (1-[4-(3-amino-1H-indazol-4-yl)phenyl]-3-(2-fluoro-5-methylphenyl)urea), WYE-354 (4-[6-[4-[(methoxycarbonyl)amino]phenyl]-4-(4-morpholinyl)-1H-pyrazolo[3,4-d]pyrimidin-1-yl-]methyl ester-1-piperidinecarboxylic acid), MK-212 (6-Chloro-2-(1-piperazinyl)pyrazine hydrochloride), and Enzastaurin (3-(1-Methylindol-3-yl)-4-[1-[1-(pyridin-2-ylmethyl)piperidin-4-yl]indol-3-yl]pyrrole-2,5-dione).

### Formulations

The therapeutic or prophylactic agents described herein may be incorporated into a vehicle for administration into a patient (e.g., a mammal, such as a human). Pharmaceutical compositions can be prepared using, e.g., physiologically acceptable carriers, excipients or stabilizers (Remington's Pharmaceutical Sciences 16th edition, Osol, A. Ed. (1980); incorporated herein by reference), and in a desired form, e.g., in the form of lyophilized formulations or aqueous solutions.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regards as their invention.

### Experimental Procedures

### Example 1

### Animals and Diets

Mice were subjected for methionine restriction (MR) as described in (Ables et al., 2012, 2015). Seven-weeks old male C57BL/6J mice were purchased from The Jackson Laboratory (Stock #000664, Bar Harbor, Me., USA) and housed in a conventional animal facility maintained at 20±2° C. and 50±10% relative humidity with a 12 h light: 12 h dark photoperiod. During a 1-week acclimatization, mice were fed Purina Lab Chow #5001 (St. Louis, Mo., USA). Mice were then weight matched and fed either a control (CF; 0.86% methionine w/w) or MR (0.12% methionine w/w) diet consisting of 14% kcal protein, 76% kcal carbohydrate, and 10% kcal fat (Research Diets, New Brunswick, N.J., USA) for 52 weeks. Body weight and food consumption were monitored twice weekly. Young mice were 8 weeks old (2 months) at the initiation of the experiments and 60 weeks old (14 months) upon termination. On the day of sacrifice, animals were fasted for 4 hours at the beginning of the light cycle. After mice were sacrificed by CO₂ asphyxiation, liver samples were collected, flash frozen, and stored at −80° C. until analyzed.

Other mice used in this study were obtained from the colonies at University of Michigan Medical School and were subjected to interventions as described in (Harrison et al., 2014; Miller et al., 2011, 2014; Strong et al., 2016). Liver samples corresponding to lifespan-extending interventions for RNA-seq and metabolome analysis were taken at 6 and 12 months of age from male and female mice treated by drugs or exposed to caloric restriction (CR) diet from 4 months of age along with control mice, which were untreated littermate mice matched by age and sex. The design of experiment was, therefore, in accordance with intervention testing program (ITP) studies, which confirmed the lifespan-extending effect of these interventions. Interventions analyzed at 6 months of age included 40% CR, Protandim™ (1,200 ppm, as in (Strong et al., 2016)), rapamycin (42 ppm, as in (Miller et al., 2014)), 17-α-estradiol (14.4 ppm, as in (Strong et al., 2016)) and acarbose (1000 ppm, as in (Harrison et al., 2014)), while interventions analyzed at 12 months of age included 40% CR, acarbose (1000 ppm, as in (Harrison et al., 2014)) and rapamycin (14 ppm, as in (Miller et al., 2011, 2014)). All organisms received the same diet (Purina 5LG6) made in the same commercial diet kitchen (TestDiet, Richmond, Ind., USA). All mice, except for those subjected to CR, were fed ad libitum. Genetically heterogenous UM-HET3 strain, in which each mouse had unique genetic background but shared the same set of inbred grandparents (C57BL/6J, BALB/cByJ, C3H/HeJ, and DBA/2J), was used in this setting. This cross produces a set of genetically diverse animals, which minimizes the possibility that the identified signatures represent gene expression patterns specific to inbred lines. Moreover, this strain was used by ITP to test the lifespan extension potential of the compounds analyzed in this study.

Liver samples from Snell dwarf (Flurkey et al., 2001) and GHRKO (Coschigano et al., 2003) males, and their sex- and age-matched littermate controls, were taken from mice at 5 months of age belonging to (PW/J×C3H/HeJ)/F2 and (C57BL/6J×BALB/cByJ)/F2 strains, respectively.

Liver samples corresponding to tested compounds predicted with the longevity gene expression signatures via Connectivity Map (CMap) were taken at 4 months of age from UM-HET3 males given diets containing KU-0063794 (10 ppm, as in (Yongxi et al., 2015)), AZD-8055 (20 ppm, as in (García-Martínez et al., 2011)), ascorbyl-palmitate (6.3 ppm, as in (Veurink et al., 2003)) and rilmenidine (10 ppm, as in (Jackson et al., 2014)) for 1 month along with untreated littermate control mice of the same age and sex, which were fed ad libitum.

In all cases, interventions continued until the animals were sacrificed. For RNA-seq analysis corresponding to lifespan-extending interventions, 3 biological replicates were used for each experimental group, including both treated and control mice, resulting in the total of 78 samples. For metabolome analysis, we utilized at least 5 and 8 biological replicates per experimental group for treated and control mice, respectively, resulting in the total of 39 samples. For RNA-seq analysis corresponding to drugs predicted with longevity signatures, we used 4 and 8 biological replicates per experimental group for treated and control mice, respectively, resulting in the total of 24 samples. RNA was extracted from liver tissues with PureLink RNA Mini Kit as described in the protocol and passed to sequencing.

### Example 2

### RNAseq Data Processing and Analysis

For samples corresponding to lifespan-extending interventions, paired-end sequencing with 100 bp read length was performed on illumine HiSeq2000 platform. For samples corresponding to predicted compounds, libraries were prepared as described in (Hashimshony et al., 2016) and sequenced with 100 bp read length option on the Illumina HiSeq2500. Quality filtering and adapter removal were performed using Trimmomatic version 0.32. Processed/cleaned reads were then mapped with STAR (version 2.5.2b) (Dobin et al., 2013) and counted via featureCounts (Liao et al., 2014). To filter out genes with low number of reads, we left only genes with at least 6 reads in at least 66.6% of samples, which resulted in 12,861 and 9,352 detected genes according to Entrez annotation for RNAseq corresponding to lifespan-extending interventions and compounds predicted by CMap, respectively. Filtered data was then passed to RLE normalization (Anders and Huber, 2010).

Differential expression analysis was performed with R package edgeR (Robinson et al., 2009). For individual interventions, we declared gene expression to be significantly changed, if p-value, adjusted by Benjamini-Hochberg procedure (Benjamini and Hochberg, 1995), was smaller than 0.05 and fold change (FC) was bigger than 1.5 in any direction. When several doses and age groups were presented, we added separate factors accounting for that to the model and looked for genes significantly changed across these settings. As dose and age groups experiments were run separately and had their own controls, such factors allowed us to adjust for possible batch effect. The effects of certain interventions on different sexes were investigated separately. To determine the statistical significance of overlap between differentially expressed genes corresponding to certain interventions, we performed Fisher exact test separately for up- and downregulated genes, considering 12,861 detected genes as a background.

When performing analysis of the feminizing effect, gene expression differences were identified between control males and females from UM-HET3 strains for each age group. Gene was declared significant if p-value, adjusted by Benjamini-Hochberg procedure, was smaller than 0.05 and FC was bigger than 1.5 in any direction. The intersection of these gene sets was used for subsequent calculation of the feminizing effect and distances between sexes. The statistical significance of correlation between sex-associated differences and response to certain intervention (“feminizing effect”) was calculated using Spearman correlation test and adjusted for multiple comparisons with Benjamini-Hochberg procedure. When calculating correlation between response to certain intervention in specific age group (6 or 12 months) and female-associated differences, the latter were calculated using gene expression data for control males and females from the other age group (12 or 6 months, respectively). This approach provided us with unbiased correlations, based on different control samples and, therefore, free of regression to the mean effect. In case of MR, GHRKO and Snell dwarf mice, which possess their own controls, the feminizing effect was calculated using both age groups.

Differences in the feminizing effect of interventions in certain age groups between males and females was tested by Spearman correlation test, applied to the difference in log₂FC of gender-associated genes in response to the specified conditions between males and females, and female-associated differences based on the other age group, with the following Benjamini-Hochberg adjustment. Manhattan distance between male and female gene expression profiles was calculated for individual samples in a pairwise manner using intersection of sex-specific gene sets across age groups. Unpaired Mann-Whitney test and Benjamini-Hochberg adjustment were used to assess statistical significance of difference between gender gene expression distances of control mice and animals subjected to interventions. Overlap between statistically significant sex-associated genes and genes differentially expressed in response to interventions was estimated by Fisher exact test similarly to comparison of individual interventions.

Heatmap of feminizing genes was created based on feminizing changes, aggregated across age groups, and log₂FC of corresponding genes in response to individual interventions, aggregated across age groups as well (using edgeR). Only genes differentially expressed between control males and females (637 genes) were used to build the heatmap. Clustering was performed with average hierarchical approach and Spearman correlation distance.

To investigate genes responsible for the feminizing effect, we used single edgeR model to identify genes with changes associated with the feminizing effect, calculated within unbiased correlation analysis. We declared a gene to be significantly changed, if its Benjamini-Hochberg adjusted p-value was smaller than 0.05. We then took an intersection of sex-associated genes, aggregated across age groups, and genes associated with the feminizing effect, separately for up- and downregulated genes, to obtain the final list of common genes. This resulted in 164 upregulated and 153 downregulated genes.

### Example 3

### Metabolome Data Processing and Analysis

Metabolite profiling using four complimentary liquid chromatography-mass spectrometry (LC-MS) methods (Paynter et al., 2018) was applied to characterize metabolites and lipids of male and female UMHET-3 mice subjected to control diet, acarbose and rapamycin (Data S1A). The samples were homogenates of freshly frozen tissues of sacrificed animals, matched by age and sex. To filter out metabolites with low coverage, only metabolites detected in at least 66.6% of the samples were remained. Afterwards, filtered data were log10-transformed and scaled (Data S1B). The data were further aggregated with our previous metabolome dataset on acarbose, rapamycin, CR, GHRKO and Snell dwarf mice models together with the corresponding controls, obtained using similar experimental procedure (Ma et al., 2015). The second dataset was preprocessed in the same way as the first one. Genetic background, age groups and treatment doses in both datasets were consistent with those used for gene expression analysis.

Analysis of the feminizing effect was performed similarly to that described for gene expression. First, metabolites that differ between control males and females were identified for each dataset using limma. Metabolite was declared significant if p-value, adjusted by Benjamini-Hochberg procedure, was less than 0.1. Then, statistical significance of the feminizing effect was calculated using Spearman correlation test and adjusted for multiple comparisons with Benjamini-Hochberg. For unbiased analysis, when calculating correlation between the response to certain interventions in specific datasets (new or published one) and female-associated differences, the latter were used from the metabolite data corresponding to the other dataset (the published or the new one, respectively) together with the set of metabolites identified for that dataset. In the case of GHRKO and Snell dwarf mice, which had their own controls, the feminizing effect was calculated using both datasets.

Differences in the feminizing effect of certain interventions in certain datasets between males and females was tested by Spearman correlation test, applied to the difference in log₂FC of gender-associated metabolites (identified based on the other dataset) in response to the specified conditions between males and females, and female-associated differences from the other dataset, with the following Benjamini-Hochberg adjustment. Manhattan distance between male and female metabolite profiles was calculated for individual samples in a pairwise manner using intersection of sex-specific metabolite sets across datasets. Unpaired Mann-Whitney test and Benjamini-Hochberg adjustment were used to assess statistical significance of difference between gender-associated metabolite profile distances of control mice and animals subjected to interventions.

### Example 4

### Functional Enrichment Analysis

For identification of functions enriched by genes differentially expressed in response to individual interventions within our RNAseq data and aggregated across datasets (CR, rapamycin and GH deficiency interventions), commonly changed across interventions (common signatures) as well as associated with the effect on lifespan, we performed GSEA (Subramanian et al., 2005) on a pre-ranked list of genes based on log₁₀(p-value) corrected by the sign of regulation, calculated as:

log₁₀(pv)×sgn(lfc),

where pv and lfc are p-value and logFC of certain gene, respectively, obtained from edgeR output, and sgn is signum function (is equal to 1, −1 and 0 if value is positive, negative and equal to 0, respectively). REACTOME, BIOCARTA, KEGG and GO biological process and molecular function from Molecular Signature Database (MSigDB) have been used as gene sets for GSEA (Subramanian et al., 2005). q-value cutoff of 0.1 was used to select statistically significant functions. Significance scores of enriched functions were calculated as:

significance score=−log₁₀(qv)×sgn(NES),

where NES and qv are normalized enrichment score and q-value, respectively.

Horizontal and vertical barplots were shown for manually chosen statistically significant functions with size of barplot being dependent on value of significance score. For functions associated with the lifespan effect and common signatures across tissues, heatmap colored based on significance scores was used. Clustering of functions enriched by individual interventions within RNAseq data was performed based on NES of functions with statistically significant enrichment (q-value <0.1) by at least one intervention. Clustering has been performed with hierarchical average approach and Spearman correlation distance.

To identify functions enriched by genes shared by differences between males and females along with changes in response to lifespan-extending interventions in males, we performed Fisher exact test using Database for Annotation, Visualization and Integrated Discovery (DAVID) (Huang et al., 2009a, 2009b). INTERPRO, KEGG and GO BP and MF databases were used. We declared functions to be enriched if their Benjamini-Hochberg adjusted Fisher exact test p-value was smaller than 0.1.

To perform further functional enrichment analysis of molecular pathways by CR and GH deficiency, we applied iPANDA method (Ozerov et al., 2016) to every individual dataset related to these interventions and obtained corresponding pathway activation scores (PAS) for each of them. PAS is based on both statistical significance and the strength of activation of the certain pathway. As some of the individual datasets measure response to certain intervention using the same control sampling, to calculate the aggregated PAS together with its p-value for the certain intervention, we used mixed-effect model, based on all single PAS values obtained from individual datasets with random term corresponding to the use of the same control sampling for calculation of gene expression change. Mixed-effect model was built with R package metafor (Viechtbauer, 2010). Obtained p-values were adjusted for multiple comparisons with Benjamini-Hochberg procedure. Functions were considered to be significantly enriched if their adjusted p-value was smaller than 0.1. Barplots with manually chosen enriched functions were built with the size of bars corresponding to the value of significance score, calculated as:

significance score=−log₁₀(adj.pv)×sgn(agPAS),

where adj. pv and agPAS are BH adjusted p-value and aggregated PAS obtained from mixed-effect model output, respectively.

### Example 5

### Aggregation of RNAseq and Microarray Datasets for Meta-Analysis

To identify signatures associated with lifespan extension and the effect of certain interventions, we expanded our data with publicly available datasets from Gene Expression Omnibus (GEO) (Edgar, 2002) and ArrayExpress (Kolesnikov et al., 2015) databases. For the analysis of signatures associated with certain interventions (CR, rapamycin, GH deficiency), we integrated available gene expression data obtained from liver of mice from healthy genetic strains on standard diets subjected to CR, rapamycin and mutations associated with GH deficiency (Ames dwarf mice, GHRKO, Little mice, Snell dwarf mice). For the analysis of signatures shared across lifespan-extending interventions, we included only the data with the experimental design statistically confirmed to extend lifespan. Finally, for the analysis of signatures associated with the lifespan extension effect, we integrated datasets on interventions with available and reliable survival data corresponding to the same experimental design (sex, strain, dose, age when the intervention started). In total, our hepatic meta-analysis covered 17 different interventions presented in 77 control-intervention datasets from 22 different sources (including ours) (FIG. 3D). The same approach was used to obtain microarray data corresponding to white adipose tissue (WAT) (9 control-intervention datasets from 5 sources) and skeletal muscle (13 control-intervention datasets from 9 sources).

To aggregate data across different platforms and studies, we developed the following method. First, data within each study was preprocessed independently and log-transformed to conform to normal distribution if needed. Then, filtering of low-covered genes was performed with soft threshold. Then, all identifiers were mapped to Entrez ID gene format, and genes not detected in our RNAseq data were filtered out. This resulted in the coverage of 12,861 genes or less if some of these genes were filtered out because of the low coverage. Afterwards, samples within every study were normalized by quantile normalization and scaling, followed by multiplication by the certain value to make it on the same scale as RNAseq data with more natural interpretation. Finally, mean and standard error of logFC of every gene for every response to intervention was calculated together with p-value (along with Benjamini-Hochberg adjusted p-value) estimated by edgeR (Robinson et al., 2009) and limma (Ritchie et al., 2015) for RNAseq and microarrays datasets, respectively. This resulted in 2 values representing every gene from every dataset. Importantly, one study may include several datasets if several interventions or settings have been analyzed there, and sometimes, different interventions or doses share the same control samples. This may be a source of batch effect, which we removed during subsequent steps of the analysis.

Scaling of genes within every sample, performed before calculation of logFC, results in similar and comparable distribution of gene changes across different studies and platforms. Importantly, scaling is not performed after calculation of logFC as different interventions may lead to different size of gene expression profile perturbation. Indeed, lifespan-extending genetic manipulations generally lead to bigger perturbation of transcriptome compared to diets and compounds (FIG. 8). To demonstrate this effect, we calculated median and standard deviation of logFC distribution across the whole transcriptome for every individual dataset. Median may be interpreted as imbalance between up- and downregulated changes whereas standard deviation corresponds to the scale of perturbation. To visualize distribution of specified metrics for different kinds of interventions (pharmacological, dietary and genetic manipulations), we used violinplots. Unpaired Mann-Whitney test was used to compare medians and standard deviations of logFC distributions corresponding to different kinds of interventions.

### Example 6

Identification of genes associated with individual longevity interventions logFC calculated for every dataset were further used as inputs to the statistical tests for meta-analysis. To account for standard error of logFC and remove batch effect related to the belonging of several datasets to the same study or same control sampling within the study, we applied mixed-effect model using R package metafor (Viechtbauer, 2010). As an input, we used both mean and standard error of logFC. Such approach allowed us to account for the size of the effect and variance of estimated gene expression change within each individual dataset, which provides a more sensitive and accurate analysis compared to previous studies focused on the comparison of lists of differentially expressed genes.

When calculating gene expression changes of individual interventions across different sources (such as CR and rapamycin), to remove batch effect, belonging to the same study or control group was considered as a random term. When calculating such changes for GH deficiency interventions, we also included type of intervention as a random term. Using this procedure, we obtained aggregated logFC and corresponding p-value for every gene. Besides standard p-value, we also calculated leave-one-out (LOO) and robust p-value. ‘LOO p-value’ is defined as the highest p-value after removal of every study one by one. On the other hand, ‘robust p-value’ is the lowest p-value after the same procedure. Benjamini-Hochberg procedure was used to adjust every type of p-value for multiple comparisons. We declared genes to be differentially expressed in response to CR, rapamycin and GH deficiency across datasets if adjusted p-value was smaller than 0.01 and their LOO p-value was smaller than 0.01. The significance of overlap between the lists of differentially expressed genes obtained from meta-analysis was estimated by Fisher exact test separately for up- and downregulated genes, considering 12,861 detected genes as background.

Similarly, aggregated logFC together with p-values were calculated for all interventions presented in our data by multiple sources. For interventions presented as a single dataset, logFC and p-values were obtained from individual datasets as described previously. For interventions measured in several datasets from the same source, single edgeR or limma model was used depending on the origin of the data (RNAseq or microarray). This resulted in the matrix containing aggregated log₂FC values of every gene in response to different interventions. To visualize change of each gene within each individual intervention, we built barplots representing aggregated log2FC of a certain gene in response to all intervention where it has been detected. Statistically significant changes were defined based on Benjamini-Hochberg adjusted p-value.

To identify upstream regulators of the detected gene expression response to CR, rapamycin and GH deficiency, we applied the Biobase Transfac platform (Matys, 2006). First, for every individual dataset, we identified transcription factor binding to sequences enriched in the promoters of differentially expressed genes using the platform. This resulted in a matrix, where every transcription factor was either enriched (1) or not (0) for the certain dataset. At this step, we excluded redundant IDs corresponding to different binding patterns of the same factor by considering factor to be enriched if at least one of its patterns is enriched. This resulted in 1,466 different upstream regulators. To identify factors overrepresented across different datasets of the same intervention, we applied permutation version of binomial statistical test as described in (Plank et al., 2012). Briefly, to identify the p-value threshold corresponding to the desired FDR (equal to 0.01), permutation test is performed, where 1 and 0 (corresponding to enrichment of different transcription factors) are shuffled within each dataset and number of false positives for different binomial test p-value thresholds are calculated. Based on the obtained numbers, p-value threshold ensuring FDR threshold of 0.01 is determined. The significance of overlap between enriched upstream regulators of different interventions was estimated by Fisher exact test, considering 1,466 non-redundant transcription factors as background.

### Example 7

### Analysis of Mutual Organization of Interventions

To assess similarity of gene expression response across interventions, we built a heatmap of aggregated log₂FC of genes significantly changed in response to CR, rapamycin and GH deficiency interventions (2507 genes in total). Complete hierarchical clustering was employed for the heatmap. Correlation matrix representing similarity between aggregated logFC of different interventions was calculated based on Spearman correlation coefficient.

To calculate correlations between interventions in unbiased way, we applied the following approach. For every pair of interventions, including comparison of intervention with itself, we examined all pairs of datasets from different sources. For each such pair we selected 250 genes consisting of 125 genes with the most significant expression change (with the lowest p-values) from each dataset and calculated Spearman correlation coefficient within the pair. We reiterated this algorithm and, as a result, for every pair of interventions obtained distribution of Spearman correlation coefficients, calculated between datasets from different sources. For CR and rapamycin, we visualized these distributions using violinplot. One-sample Mann-Whitney test and Benjamini-Hochberg adjustment were used to check if means of correlation coefficients are different from 0 with statistical significance. We declared correlation coefficient to be significant if adjusted p-value was smaller than 0.1.

For correlation matrix we employed median values of Spearman correlation coefficients. By filtering out comparisons of datasets from the same source, we removed possible batch effect and ended up with independent and unbiased comparison of interventions. However, as some interventions were presented only within the same source, we couldn't estimate unbiased correlation for such cases. This missing data was visualized by grey boxes. The same was sometimes true for comparison of intervention with itself, as in this case we also employed only datasets from different sources. For this reason, correlation coefficient of intervention with itself was not equal to 1 in resulted unbiased correlation matrix. Complete hierarchical clustering approach was employed for visualization of correlation matrix.

To demonstrate similarities between different interventions in network mode, we employed Cytoscape (Shannon et al., 2003). Only edges between interventions with significant positive correlation coefficients (median Spearman correlation coefficient >0 and adjusted Mann-Whitney p-value <0.1) were shown. The width of edge was defined by the log₁₀(adjusted p-value). Smaller p-value led to wider edge.

### Example 8

Identification of Common Signatures and Genes Associated with the Lifespan Effect

To identify hepatic genes, whose expression change is shared across lifespan-extending interventions, we filtered out all interventions and settings with unproven lifespan extension effects. To account for possible differences in the intervention effect on lifespan across different sexes, ages, strains and doses, we only considered the datasets, whose experimental settings were shown to produce a statistically significant extension of lifespan. Therefore, for example, 40% CR in C57BL/6 females was excluded from the analysis as this setting doesn't lead to a statistically significant lifespan extension, contrary to 20% CR applied to the same mouse strain (Mitchell et al., 2016). In the case of drugs, we also filtered out the datasets containing the response to compounds, which had not been confirmed by ITP studies (such as metformin and resveratrol).

First, for every single gene we calculated number of interventions, where it is differentially expressed based on adjusted aggregated p-value estimated as described previously. We considered gene to be differentially expressed if its adjusted aggregated p-value was smaller than 0.1. However, this approach overfits genes changed in response to similar interventions (such as GH deficiency interventions) and doesn't take into account possible consistent changes, which may be, however, not significant due to low sampling size or high variance. To overcome this problem, we applied single mixed-effect model to every gene as described previously and looked for genes, whose aggregated logFC across lifespan-extending interventions is significantly different from 0. Here, however, we also included the type of intervention as a random term together with correlation matrix specifying similarities between general response of the interventions. This correlation matrix was taken from unbiased mutual organization analysis described previously. We declared genes to be significantly shared across interventions if Benjamini-Hochberg adjusted robust p-value, obtained after removal of every type of intervention one by one, was smaller than 0.05. The same approach was used to identify genes shared across lifespan-extending interventions in the skeletal muscle and WAT. Heatmap with expression changes of significant genes across individual datasets was clustered using a complete hierarchical approach.

To identify genes associated with the lifespan effect, first, we estimated three main metrics of lifespan for every available setting, including median lifespan ratio (in logarithmic scale), maximum lifespan ratio (in logarithmic scale), defined as ratio of average lifespan of 10% most survived individuals, and median hazard ratio, defined as ratio of slopes of survival curves at the median point (timepoint where 50% of cohort is remained survived). These metrics were obtained from published survival data for the corresponding interventions. To account for heterogeneity of our data, we integrated gene expression and longevity studies only if they were associated with the same experimental design (sex, dose, strain, age when intervention started). We then calculated average metric values across the studies to obtain most consistent and reliable estimates. Interventions or settings, for which no appropriate longevity study was available, were excluded.

Afterwards, we applied mixed-effect model as described previously to identify genes associated with each of the 3 numeric metrics of the lifespan effect. Control group and type of intervention were considered as random term, and correlation matrix between interventions was used to define covariance matrix. We declared genes to be significantly associated with the lifespan effect if Benjamini-Hochberg adjusted p-value and LOO p-value, obtained after removal of every intervention one by one, were both smaller than 0.05. The significance of overlap between lists of genes associated with different metrics of the lifespan effect was estimated by Fisher exact test separately for genes with positive and negative association, considering 12,861 detected genes as a background. Complete hierarchical clustering was used to sort genes on heatmap, representing logFC of genes with significant association across individual datasets. Individual datasets were sorted there based on their effect on maximum lifespan.

Overlap between gene signatures associated with lifespan extension and genes, whose manipulation was demonstrated to extend or shorten mouse lifespan, was estimated by Fisher exact test, as described previously. The latter set was obtained from GenAge database and included 84 and 44 genes with pro- and anti-longevity effects, respectively (De Magalhães and Toussaint, 2004).

### Example 9

Test for Association with Longevity Signatures

To test association of interventions with longevity signatures related to individual interventions (CR, rapamycin and GH deficiency), common changes and lifespan effect association, we employed GSEA-based approach. First, for every signature we specified 250 genes with the lowest p-values and divided them into up- and downregulated genes. These lists were considered as gene sets. Then we ranked genes related to interventions of interest based on their p-values, calculated as described in functional enrichment section. When running association test for lifespan-extending interventions (FIG. 4A), we used p-values obtained from the aggregated analysis as described earlier.

For interventions from publicly available sources (FIG. 7E (upper)), we downloaded them from GEO under the following accession numbers: GSE21060 (Ramadoss et al., 2010), GSE77082 (Alonso et al., 2017), GSE15891 (Baze et al., 2010), GSE11287 (Osburn et al., 2008), GSE49000 (Mercken et al., 2014), GSE10421 (Kautz et al., 2008) and GSE104234 (Rhoads et al., 2018). Data corresponding to Sirt6 overexpression (Kanfi et al., 2012) were downloaded via the link provided in the original paper. When running association test for the rhesus monkey data, we converted monkey genes to mouse orthologs using Ensembl BioMart platform. We preprocessed each dataset, performed quantile normalization and Entrez ID transformation and applied limma model for calculation of p-values, which were converted to log₁₀(p-value) corrected by the sign of regulation as explained earlier.

For compounds predicted with the longevity signatures via CMap, we calculated p-values of gene expression changes compared to control independently for every drug using edgeR. We then converted them to log₁₀(p-value) corrected by the sign of regulation as described earlier and proceeded to GSEA-based analysis.

We calculated GSEA scores separately for up- and downregulated lists of gene set as described in (Lamb et al., 2006) and defined final GSEA score as a mean of the two. To calculate statistical significance of obtained GSEA score, we performed permutation test where we randomly assigned genes to the lists of gene set maintaining their size. To get p-value of association between certain intervention and longevity signature, we calculated the frequency of real final GSEA score being bigger by absolute value than random final GSEA scores obtained as results of 3,000 permutations. To adjust for multiple comparisons, we performed Benjamini-Hochberg procedure. Resulted adjusted p-values were converted into significance scores as:

significance score=−log₁₀(adj.pv)×sgn(GSEA score),

where adj. pv and GSEA score are BH adjusted p-value and final GSEA score, respectively. Heatmaps were colored based on values of significance scores.

### Example 10

### RNAseq Analysis Across Lifespan-Extending Interventions

We subjected 78 young adult mice to 8 interventions previously established to extend lifespan, including acarbose, 17-α-estradiol, rapamycin, Protandim, CR (40%), MR (0.12% methionine w/w), GHRKO and Pit1 knockout (Snell dwarf mice) (3 biological replicates were used in each experimental group; FIG. 1A). This set included three interventions that have never been analyzed at the gene expression level (acarbose, 17-α-estradiol and Protandim). All compounds and diets were applied to genetically heterogeneous UM-HET3 mice and started at 4 months of age, as in ITP studies (Harrison et al., 2014; Miller et al., 2011, 2014; Strong et al., 2016), except for MR, which was applied to 2-month-old C57BL6/J mice, as in (Ables et al., 2012, 2015). Duration of treatment exceeded 8 months in at least one age group for each compound or diet and was equal to 5 months for the long-lived mutants. We then performed RNAseq on liver samples of these mice, together with sex- and age-matched littermate controls, analyzing both males and females, except for MR, GHRKO and Snell dwarf mice, where only males were examined. Since some of these interventions are known to be effective when used at different concentrations and different ages (Harrison et al., 2009, 2014; Mercken et al., 2014a; Miller et al., 2014; Mitchell et al., 2016; Strong et al., 2016), we used 2 different age groups for CR, rapamycin and acarbose (6- and 12-month-old, representing 2 and 8 months of treatment, respectively), and 2 different effective concentrations of rapamycin (14 and 42 ppm) (FIG. 1A). As age- and lifespan-associated patterns may or may not correlate with each other, and we were interested in the identification of signatures associated with the effect of interventions apart from the changes related to the consequences of slowed down aging, all mice utilized in these experiments were young and middle-aged. This allowed us to attribute the observed gene expression changes to the direct effect of lifespan-extending interventions and to analyze longevity patterns independent from the aging process.

Differentially expressed genes associated with each intervention were initially examined separately for males and females. Many differentially expressed genes were found to be common to interventions. For example, almost half of MR genes (44.3% upregulated and 41.8% downregulated genes) were altered significantly and in the same direction in Snell dwarf males and CR males and females (FIG. 1B). Moreover, genes affected only in Snell dwarfs covered 37% of MR upregulated and 36.5% of MR downregulated genes (Fisher exact test p-value <2.210⁻¹⁶ in both cases). This observation supports the idea of common molecular mechanisms shared by MR and other interventions such as CR and Snell dwarf mice. It is also consistent with the previous findings that the lifespan extension effect of CR in flies is highly dependent on the presence of methionine in the diet and can be abrogated by the addition of amino acids only if they include methionine (Grandison et al., 2009).

Analysis of enriched functions using gene set enrichment analysis (GSEA) (Subramanian et al., 2005) revealed many similarities among the interventions (FIG. 1D). For example, ribosomal protein genes were upregulated in response to all interventions except MR (q-value <0.001), and other commonly upregulated functions included drug metabolism by cytochrome P450, glutathione metabolism, oxidative phosphorylation and TCA cycle. These patterns are consistent with the reports on individual lifespan-extending interventions, including Ames and Snell dwarf mice, Little mice, GHRKO, CR and rapamycin (Amador-Noguez et al., 2004; Miller et al., 2014; Steinbaugh et al., 2012).

In addition to common strategies, we detected some distinct signatures. For example, 17-α-estradiol in females and MR resulted in downregulation of oxidative phosphorylation. Although ribosomal protein genes, in general, represented the most common upregulated pattern across the interventions, this was not the case for mitochondrial ribosomal protein genes. Some interventions, including CR, GHRKO, Snell dwarf mice and acarbose in males, showed significant upregulation of these genes, whereas 17-α-estradiol in both sexes and MR showed their downregulation. Finally, fatty acid oxidation, which is known to be positively associated with the lifespan extension effect of several interventions (Amador-Noguez et al., 2004; Plank et al., 2012; Tsuchiya et al., 2004), was significantly downregulated when applied to females (FIG. 1D). 17-α-estradiol, acarbose and CR showed significant downregulation of fatty acid oxidation genes in females, whereas in males we observed an opposite effect for acarbose and CR.

Interestingly, although MR mice resemble CR mice in stress resistance and endocrine changes, and MR mice share many differentially expressed genes with CR and growth hormone (GH) deficiency-associated interventions (i.e. GHRKO and Snell dwarf mice), MR displayed a quite distinct pattern at the level of functional enrichment (FIG. 1C). It shared some common signatures with CR and GH-associated mutants, including upregulation of glutathione metabolism, drug metabolism by cytochrome P450 and regulation of telomere maintenance and downregulation of complement and coagulation cascades. However, it also exhibited upregulation of PI3K, insulin receptor and mTOR pathways and downregulation of oxidative phosphorylation and genes coding structural constituents of ribosome, which was distinct from CR and most other interventions. Data from other tissues, once they become available, may add to the understanding of similarities and differences across these interventions.

To get a more global view on the similarities among interventions in terms of regulation of cellular pathways, we built a heatmap of normalized enrichment scores (NES) of all functions enriched by at least one intervention and clustered the data using an average hierarchical approach (FIG. 1C). MR clustered separately from other interventions, but together with acarbose in females (unfortunately, we lacked tissues of female mice subjected to MR). Not surprisingly, GHRKO and Snell dwarf mice, which are both associated with growth hormone deficiency, showed a very similar pattern of gene expression both at gene and functional enrichment levels, in accordance with previous studies that examined gene expression response of GH deficiency (Amador-Noguez et al., 2004). Finally, females and males showed a similar response to CR and 17-α-estradiol at the level of functional enrichment, whereas their responses to acarbose and Protandim were quite different. Interestingly, although both 17-α-estradiol and Protandim lead to statistically significant lifespan extension only in males, similarities in the response to them across sexes seem to be different at the level of cellular pathways. High similarity in the functional response to 17-α-estradiol between males and females together with its substantial effect on median lifespan in males (increase by 19%) and absence of effect in females (Strong et al., 2016) point to the role of gender in the lifespan effect even when molecular changes induced by interventions are similar across sexes.

### Example 11

### Feminizing Effect of Lifespan-Extending Interventions

The finding of sex-specific gene expression changes in response to longevity interventions allowed us to examine this question in more detail. Several previous studies noted a feminizing effect of CR and GH deficiency on gene expression in males (Buckley and Klaassen, 2009; Estep et al., 2009; Fu and Klaassen, 2014; Li et al., 2013). To test if this effect is reproduced across different interventions, we first identified genes whose expression significantly differs between control males and females from UM-HET3 strains in both 6- and 12-month-old age groups. We then examined how lifespan-extending interventions affect these sex-associated differences. To analyze it in an unbiased way free of regression to the mean effect, for every intervention of a certain sex and age, we calculated the Spearman correlation of its gene expression response with the differences between males and females, calculated for another age group. In the case of Snell dwarf mice, GHRKO and MR, which had their own controls, we used both age groups for the calculation.

In males, we detected statistically significant feminizing-like patterns for genetic (GHRKO and Snell dwarf mice) and dietary (CR and MR) interventions at the gene expression level (FIG. 2B). In other words, each of these interventions led to upregulation of female-specific and downregulation of male-specific genes. For example, female- and male-associated expression patterns shared more than 66% of up- and 72% of downregulated genes, respectively, that were perturbed by GHRKO and 6-month-old CR in males and showed a statistically significant overlap with both (Fisher exact test adjust p-value <2.98·10⁻¹⁸) (FIG. 2A). The feminizing effect was especially strong for genetic mutants, reaching 80% correlation for GHRKO (Spearman correlation test adjusted p-value=6.1·10⁻⁵⁶; FIG. 8B). Besides mutants and diets, acarbose applied for 8 months in males also produced a significant feminizing effect (Spearman correlation=0.57, BH adjusted p-value=6.1·10⁻⁵⁶). Finally, weak feminization was produced by rapamycin applied for 4 months (Spearman correlation=0.19, BH adjusted p-value=4.1·10⁻³). Other drugs did not lead to a significant feminizing effect in males or even produced a slight negative effect, such as Protandim in 6-month-old mice (Spearman correlation=−0.11; BH adjusted p-value=0.088; FIG. 2B).

In females, the effect of interventions on sex-associated expression differences was mostly similar to that in males. For example, CR (Spearman correlation=0.12 and 0.2 and BH adjusted p-value=0.07 and 3.7·10⁻³ for 12- and 6-month age groups, respectively) and 12-month old acarbose (Spearman correlation=0.19 and BH adjusted p-value=4.7·10⁻³) females also exhibited a significant feminizing-like pattern (FIG. 2B). On the other hand, rapamycin females showed a significant anti-feminizing (“masculinizing”) pattern in both age groups (Spearman correlation=−0.49 and −0.14 and BH adjusted p-value=6.1·10⁻⁵⁶ and 0.04 for 12 and 6 months, respectively), upregulating male-specific and downregulating female-specific genes. Interestingly, one of the strongest masculinizing patterns in females was produced by 17-α-estradiol, which had no significant effect on sex-associated genes in males, hinting that its selective effect on males is not due to simple recapitulation of the female hormonal profile. Based on our data, feminization does not explain the effect of interventions on lifespan extension. Indeed, 17-α-estradiol does not lead to any feminizing changes in males but increases their median (by 19%) and maximum (by 12%) lifespan (Strong et al., 2016). Besides, in females rapamycin and 17-α-estradiol showed a similar and significant masculinizing effect, although the first drug extended lifespan in females even more strongly than in males (Miller et al., 2014), whereas the second compound did not lead to lifespan extension in females (Strong et al., 2016). Therefore, it seems that feminization or masculinization are neither necessary nor sufficient for lifespan extension, although a number of interventions, including GH mutants and diets, influence some of the genes associated with gender-specific differences.

Although various interventions had a different effect on feminizing genes across sexes, we observed a consistently stronger feminizing effect in males compared to females for every individual intervention and age group (Spearman correlation test BH adjusted p-value <2.6·10⁻⁶), except for Protandim, which showed the opposite trend (FIG. 2B). In other words, regardless of the direction and size of the effect of specific interventions on sex-associated genes in males, most of them lead to relatively more masculinizing changes in females. To test if such pattern results in the convergence of gender-associated gene expression profiles to some hypothetical neutral state, we calculated pairwise distances between expression of these genes in male and female samples for all experimental groups (FIG. 2C). Indeed, we found that sex-associated differences were significantly reduced by all interventions, except for 6-month Protandim and 12-month rapamycin (Mann-Whitney test BH adjusted p-value <0.024) treatments. This finding suggests that the survival state, induced by lifespan-extending interventions, is broadly shared across sexes, with differences between males and females becoming less prominent as a result. Therefore, we believe that different sexes share at least some general mechanisms of lifespan extension, although they have distinct initial states defined by gender-specific features.

To validate our findings at the level of metabolome, we performed metabolite profiling of 39 12-month-old male and female mice subjected to control diet, acarbose and rapamycin (at least 5 biological replicates in each experimental group). We further aggregated this data with our previous dataset, which included female and male mice of the same age subjected to control diet, CR, acarbose and rapamycin as well as male GHRKO and Snell dwarf mice (Ma et al., 2015). Using a similar procedure, we identified metabolites that significantly differ between control males and females in each of the datasets and then used them to calculate the feminizing effect at the metabolome level. In agreement with the gene expression results, we observed a significant feminizing effect of genetic interventions (GHRKO and Snell dwarf mice), CR, and acarbose in males (FIG. 2D). Rapamycin produced a significant feminizing effect only in one of the datasets. Applied to females, the same interventions also resulted in a significantly more masculinizing effect compared to males, except for rapamycin from the previously obtained dataset (Spearman correlation test BH adjusted p-value <0.098). Finally, in agreement with the gene expression findings, all interventions, except for rapamycin from the new dataset, showed a reduction of sex-related differences at the metabolome levels following introduction of lifespan-extending interventions (Mann-Whitney test BH adjusted p-value <0.011) (FIG. 2E). Therefore, metabolome data are consistent both with the feminizing effect of interventions in males and the convergence of sex-associated phenotypes identified at the level of gene expression, pointing to the congruence of these processes at different molecular levels.

To better understand the nature of the feminizing pattern, we identified sex-associated genes which change in response to interventions is, at the same time, associated with the feminizing effect. With the FDR threshold of 0.05 and FC threshold of 1.5, we detected 355 sex-associated genes differentially expressed at a higher level in females and 282 genes expressed at a lower level (FIG. 2F). Among them, 153 (out of 355) and 164 (out of 282) genes were positively and negatively associated with the feminizing effect, respectively. Functional enrichment of these genes using DAVID (Huang et al., 2009a, 2009b) revealed upregulation of drug metabolism (Fisher exact test BH adjusted p-value=1.5·10⁻⁹) and fatty acid metabolism (Fisher exact test BH adjusted p-value=0.026) (FIG. 2G). Cytochrome P450 genes, involved in drug metabolism, are well known to be differentially expressed between sexes in mice and regulated by GH and its sex-specific daily pulse frequency and amplitude (Waxman and Holloway, 2009). However, it was previously unclear whether the same xenobiotic metabolizing enzyme (XME) genes are altered in response to different lifespan-extending interventions. Here, we show that this is indeed the case. Interestingly, male rodents also demonstrate female-like alteration of some other sex-specific cytochrome P450s with age, both at the level of gene expression and enzymatic activity (Imaoka et al., 1991; Kamataki et al., 1985). This appears to be, at least partly, due to the change of their GH secretion profile (Imaoka et al., 1991; Wauthier et al., 2007). Therefore, feminization of the drug metabolism system in males seems to be an example of the pattern positively associated with both aging and response to several lifespan-extending interventions.

Among downregulated sex-associated genes, we detected enrichment of complement and coagulation cascades (Fisher exact test BH adjusted p-value=9.8·10⁻³) and major urinary proteins (MUP) genes (Fisher exact test BH adjusted p-value=0.021) (FIG. 2G). MUP expression is highly sex-specific, and the high concentration of total and specific MUP proteins in male mouse urine appears to influence male attractiveness to females (Garratt et al., 2011; Roberts et al., 2010), suggesting a possible link between lifespan extension and reproductive fitness. Interestingly, sexually dimorphic expression of most MUP isoforms is also known to be regulated by growth hormone. Indeed, Mup genes are significantly downregulated in GH-deficient mutants, but their level can be restored to control levels by GH injection (Knopf et al., 1983). Moreover, injection of GH was also able to masculinize MUP mRNA levels in female mice (al-Shawi et al., 1992). Therefore, it seems that gene expression changes associated with the feminizing effect across interventions are generally linked to GH as a key upstream regulator.

Overall, the data show that the feminizing effect is shared by genetic and dietary lifespan-extending interventions in males at both gene expression and metabolome levels, and that this effect is achieved through perturbations of common genes and molecular pathways including those regulated by GH. The feminizing effect does not explain lifespan extension but is consistently higher in males compared to females subjected to the same intervention, regardless of its direction and size. It also appears to reduce gender-associated differences at the gene expression and metabolite levels, pointing to the converging effect of lifespan-extending interventions on hepatic transcriptome and metabolome across sexes.

### Example 12

### Signatures of CR, Rapamycin and Growth Hormone Deficiency

To obtain a comprehensive picture of gene expression responses to interventions, we collected all publicly available microarray datasets for mouse liver and conducted a meta-analysis across aggregated data. We first focused on 3 interventions: CR, rapamycin and interventions related to GH deficiency (GHRKO, Little mice, Snell and Ames dwarf mice). The latter group was combined, because these interventions, although targeting different genes involved in GH production and sensing, result in a similar effect on liver due to inability to activate GHR. In addition to this mechanistic notion, similarity among these interventions could also be seen at the level of hepatic gene expression as demonstrated by other groups (Amador-Noguez et al., 2004) and our results (FIGS. 3F and 4C). As these 3 intervention groups seem to be the most well-studied at both experimental and gene expression levels, we were interested in the identification of consistent gene expression signatures associated with them. For this reason, we combined all data across different sexes, strains, ages, durations of interventions and doses. In total, we aggregated data from 29 CR datasets (across 13 different studies), 9 rapamycin datasets (across 3 different studies) and 20 GH deficiency datasets (across 7 different studies). Such heterogeneity in terms of sex, age, strains and dose may introduce variability of gene expression responses and, therefore, complicate direct comparison of interventions. Because we sought to identify robust signatures associated with lifespan extension, the heterogeneity, however, presents some advantages, supporting robustness of the identified patterns and allowing interpretation across a wide range of different genetic strains, sexes and ages.

To overcome issues associated with differences in platforms across different studies, along with batch effects, we developed an integrative method, based on independent preprocessing and normalization of individual datasets and following aggregation of means and standard deviations of logFC for all genes detected in our RNAseq data (resulting in 12,861 genes). Importantly, to account for possible differences in the general effect of interventions on mouse transcriptome, we did not normalize distributions of logFC across datasets. To include information about standard deviations of logFC and account for possible batch effects due to the use of several datasets sharing the same control (e.g., if several doses were tested), we applied a mixed-effect model, considering shared control as a random term. We used this method to identify genes up- or downregulated across datasets associated with the same type of intervention. Our approach, contrary to the comparison of lists of differentially expressed genes used in previous meta-analyses (Plank et al., 2012; Swindell, 2008), accounts for the size of the effect and variance of gene expression change within each individual dataset and, therefore, provides a more accurate and sensitive analysis. Besides standard p-value, obtained from the mixed-effect model test, we calculated “leave-one-out” (LOO) p-value as the largest (least significant) p-value after removal of every study one by one.

In this procedure, genes were designated statistically significant if their BH adjusted p-value was <0.01 and LOO p-value was <0.01. With these thresholds, we identified 419 up- and 370 downregulated genes for CR, 894 up- and 879 downregulated genes for GH deficiency, and 127 up- and 100 downregulated genes for rapamycin (FIG. 3A). Interestingly, CR and GH interventions significantly overlapped (37% of CR upregulated and 26.3% of CR downregulated genes were shared with GH interventions; Fisher exact test p-value <10⁻²⁸ for both up- and downregulated genes), whereas rapamycin did not show a statistically significant overlap with either of them. Upregulated genes shared by CR and GH deficiency were enriched for oxidative phosphorylation (Fisher exact test BH adjusted p-value=1.52·10⁻⁹), and downregulated genes were enriched for complement and coagulation cascades (Fisher exact test BH adjusted p-value=5.21·10⁻⁸). The difference in the gene expression response between CR and rapamycin was previously noted (Fok et al., 2014b; Miller et al., 2014), but is not well understood. Our data provide a clear case for largely distinct mechanisms by which these interventions act in the liver. Not surprisingly, all GH deficiency interventions showed downregulation of Igf1 (FIG. 9A) and its stabilizer lgfals along with upregulation of 2 genes encoding its inhibitors, IGF-binding proteins Igfbp1 and Igfbp2 (FIG. 9B). Interestingly, Igf1 expression showed no consistent significant changes in response to CR and was even slightly upregulated in response to rapamycin (FIG. 9A). Moreover, we did not detect an association of Igf1 logFC in response to CR with any other feature, including age of mice, duration of treatment, level of restriction and even the effect on lifespan. On the other hand, IGF-1 plasma levels are known to be decreased by CR in various mouse models differing in sex, strains and energy intake (Mitchell et al., 2016). However, even the same models integrated in our meta-analysis didn't show consistent downregulation of hepatic Igf1. On the other hand, we detected upregulation of two IGF-1 binding partners (Igfbp1 and Igfbp2) in response to CR (FIG. 9B). Therefore, inhibition of the IGF1 pathway by CR is not mediated by the direct regulation of hepatic Igf1 expression, but may be associated with elevated levels of its inhibitors. GH-deficient mutants, on the other hand, appear to both downregulate Igf1 (along with its stabilizer lgfals) (FIG. 9A) and upregulate its binding proteins (FIG. 9B).

By applying GSEA, we further identified several pathways shared by 2 or all 3 analyzed interventions (FIG. 3B). Interestingly, rapamycin was found to share perturbed molecular pathways with the other interventions, in agreement with our own RNAseq data. Thus, oxidative phosphorylation was commonly upregulated across all three interventions (q-value <0.008 for each of them). Other shared upregulated functions included TCA cycle, ribosome and genes associated with age-related diseases (Parkinson's and Huntington's).

To obtain further details on the regulation of molecular pathways by CR and GH deficiency, we used the iPANDA algorithm (Ozerov et al., 2016), which is another method of functional enrichment analysis that utilizes the sign of the effect of each specific gene on pathway activation or inhibition. We applied it to every individual dataset included in our meta-analysis and calculated an aggregated pathway activation score (PAS) along with its statistical significance using the mixed effect model described previously. In agreement with the GSEA output, we observed activation of TCA cycle, respiratory electron transport chain, urea cycle and PPAR pathways along with inhibition of alternative complement, interferon and insulin processing pathways in both CR (FIG. 10A) and GH deficiency (FIG. 10B) (BH adjusted p-values <0.068 for all specified functions). Pathways activated in response to CR also included transcriptional activation of mitochondrial biogenesis, triglyceride biosynthesis and circadian clock as well as downregulation of translational initiation regulated by mTOR signaling (BH adjusted p-value <0.032) (FIG. 10A). GH deficiency interventions, in turn, showed activation of the caspase cascade and GSK3 signaling pathways together with inhibition of IGF1R signaling and MAPK, biosynthesis of mineralocorticoids and cholesterol, mTOR and estrogen pathways (BH adjusted p-value <9.4·10⁻⁶) (FIG. 10B).

To identify upstream regulators of observed gene expression changes, we analyzed enrichment of transcription factors associated with differentially expressed genes using the Biobase Transfac platform (Matys, 2006). First, for each individual dataset we identified transcription factors binding to sequences enriched in promoters of genes differentially expressed in the corresponding dataset. We then applied a binomial statistical test to identify factors whose enrichment was overrepresented across datasets within the same type of intervention. A permutation FDR threshold of 0.01 resulted in the identification of 161 transcription factor IDs enriched for CR, 213 IDs enriched for GH-deficient interventions and 17 IDs enriched for rapamycin (FIG. 3C). As at the level of individual genes, CR and GH-deficient interventions shared many transcription factors (>50% of their enriched transcription factors were shared; Fisher exact test p-value <10⁻²⁶). However, in this case rapamycin also showed significant overlap with other interventions (58.8% and 47.1% of enriched transcriptional factors were shared with CR and GH deficiency, respectively; Fisher exact test p-value <0.002 in both cases). Interestingly, 8 factors shared by all 3 interventions included receptors related to glucose sensitivity and sterol metabolism, such as glucocorticoid receptor NR3C1 and sterol regulatory element binding transcription factor SREBP-1. Factors shared by CR and GH deficiency included NRF2, PPARα, PPARγ and a number of interferon regulatory factors, in accordance with the results of functional enrichment. Therefore, it appears that even though rapamycin exhibits a distinct pattern at the level of individual genes, its effect partly converges with the other interventions at the level of molecular pathways and transcriptional regulation. A non-significant overlap of the perturbed genes may also be explained by high variability of the response to rapamycin across different experimental settings (FIG. 4C) that is later reduced at the level of functional and transcriptional enrichment.

### Example 13

### Mutual Organization of Gene Expression Profiles of Lifespan-Extending Interventions

We next performed a meta-analysis of the dataset that included, in addition to the gene expression data we generated, all publicly available microarray data on lifespan-extending interventions in mouse liver. We also included resveratrol and metformin, which are interventions that did not increase lifespan in the ITP mouse cohort at the concentrations used (Miller et al., 2011; Strong et al., 2013, 2016), but are known to share some molecular mechanisms with lifespan-extending CR (Barger et al., 2008; Dhahbi et al., 2005; Martin-Montalvo et al., 2013; Pearson et al., 2008), increase healthspan of mammals, including improvement of cardiovascular function and physical performance along with inhibition of inflammation (Baur and Sinclair, 2006; Martin-Montalvo et al., 2013; Pearson et al., 2008), and lead to increased longevity of the nematode Caenorhabditis elegans (De Haes et al., 2014; Viswanathan et al., 2005; Wood et al., 2004), short-lived fish Nothobranchius furzeri in case of resveratrol (Valenzano et al., 2006), and mice under certain conditions (Baur et al., 2006; Martin-Montalvo et al., 2013; Pearson et al., 2008). After integration of all available data, our dataset included 17 different interventions and 77 control-intervention comparisons across 22 different sources (FIG. 3D). Importantly, our list of analyzed interventions included multiple representatives of each of the different intervention types, i.e. dietary, genetic (mutations, overexpression) and pharmacological.

Aggregation of data was performed using the approach discussed above. Interestingly, comparison of standard deviations of gene expression fold change distributions in response to different interventions showed that genetic manipulations had the largest effects on gene expression profile (Mann-Whitney test p-value=0.003 between dietary and genetic intervention groups), whereas pharmacological interventions had the smallest effect (Mann-Whitney test p-value=1.71·10⁻⁶ between pharmacological and dietary intervention groups) and dietary interventions were in the middle (FIG. 11A). As control, we examined possible differences between medians of gene fold change distributions, and did not observe significant differences between any pair of intervention groups (FIG. 11B). As expected, genetic manipulations caused more significant changes of transcriptome profiles compared to diets and, especially, to drugs. Therefore, it was particularly important to avoid normalization of mean fold change distributions across different datasets, as in that case the described important features of different interventions would be lost.

To examine how similar various interventions are in terms of gene expression signatures identified for CR, GH deficiency and rapamycin, we created a heatmap representing aggregated gene expression data across interventions for the identified genes (FIG. 3E). Not surprisingly, interventions associated with GH deficiency formed a tight cluster, indicating convergence of their molecular mechanisms in the liver. In general, we found that interventions resemble changes induced by CR, GH deficiency and rapamycin. Indeed, we observed positive Spearman correlation between aggregated gene expression changes for most interventions (FIG. 3F). However, some interventions turned out to show distinct gene expression patterns. For example, we observed a small separate cluster formed by rapamycin, Protandim and 17-α-estradiol, in which only the latter intervention showed positive correlation with the interventions representing the main cluster. Furthermore, acarbose and 17-α-estradiol showed positive correlation with both major and minor clusters, pointing to the existence of certain gene expression patterns within each of them, which do not necessarily conflict with each other. To see if different interventions recapitulate the gene expression changes separately induced by CR, rapamycin and GH deficiency, we performed GSEA for every intervention, using genes identified as signatures of the 3 specified interventions as input subsets. Using a BH adjusted permutation test p-value threshold of 0.1, we identified interventions with statistically significant positive association with CR, rapamycin and GH deficiency (FIG. 4A). Interestingly, the majority of interventions, including all GH deficiency interventions, all diets (CR, every-other-day feeding (EOD) and MR), acarbose, FGF21 overexpression, 17-α-estradiol and resveratrol, shared the changes induced by CR and GH deficiency, pointing to the commonality of gene expression responses to these interventions. On the other hand, rapamycin again showed a distinct pattern, which was, however, partially shared by some interventions (acarbose, GHRKO, Snell dwarf mice, 17-α-estradiol and Protandim). This approach, however, may include some batch effects resulting from comparison of datasets from the same source and even because of the use of the same, shared, controls (e.g., resveratrol and EOD along with Protandim and 17-α-estradiol obtained from the same data and compared against common controls).

To overcome the batch effect and investigate mutual organization of gene expression profiles of different interventions at the level of whole transcriptomes, we compared interventions pairwise, considering, for every pair of interventions, only pairs of control-intervention comparisons from different sources. For each of them, we calculated the Spearman correlation coefficient using the 250 most statistically significant differentially expressed genes. We then examined the distribution of these correlation coefficients among all pairs of control-intervention comparisons. Using this approach, we could get rid of the batch effect in that datasets from the same study were not compared when calculating the correlation coefficient. We also used the same unbiased procedure to obtain the distribution of correlation coefficients between different datasets of the same intervention. This let us investigate how consistent gene expression response to certain intervention is across different studies and experimental design settings.

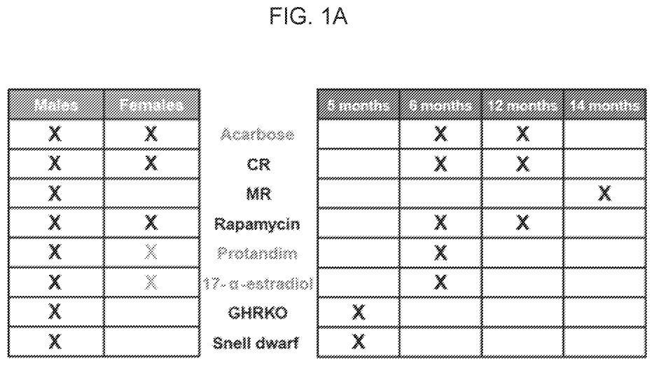

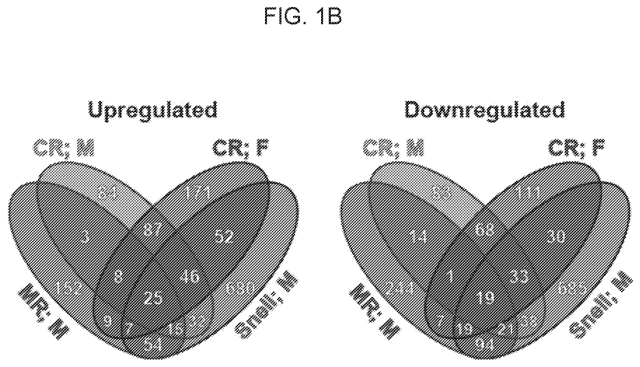

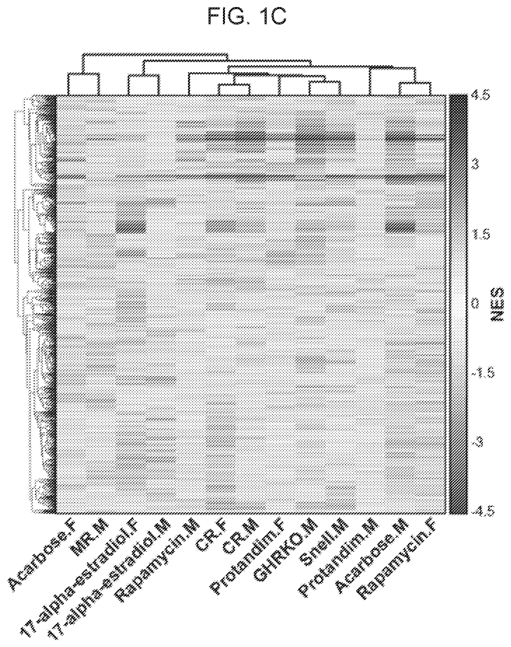

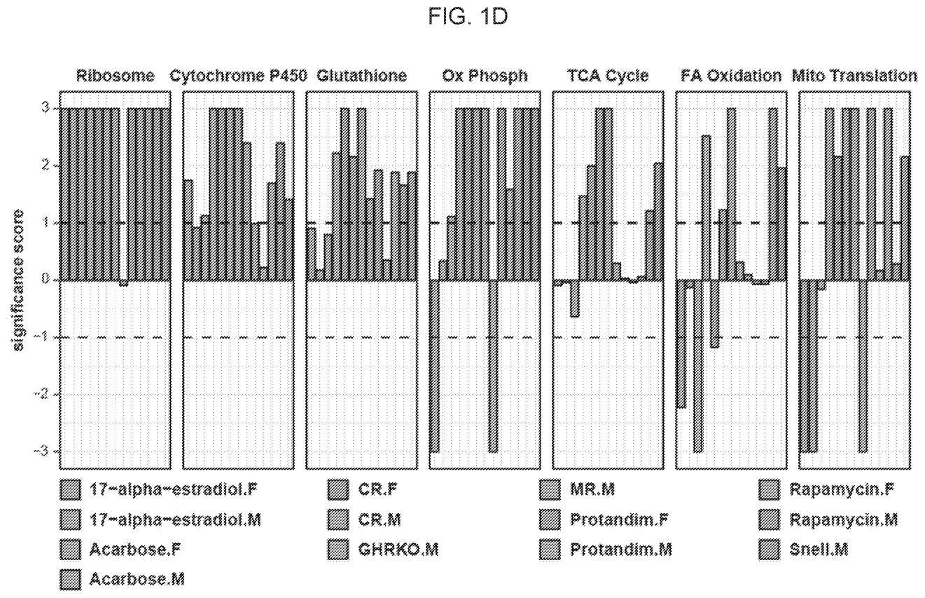

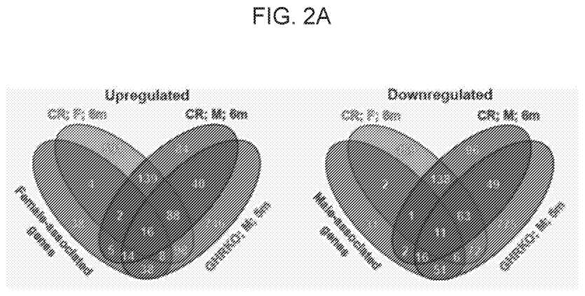

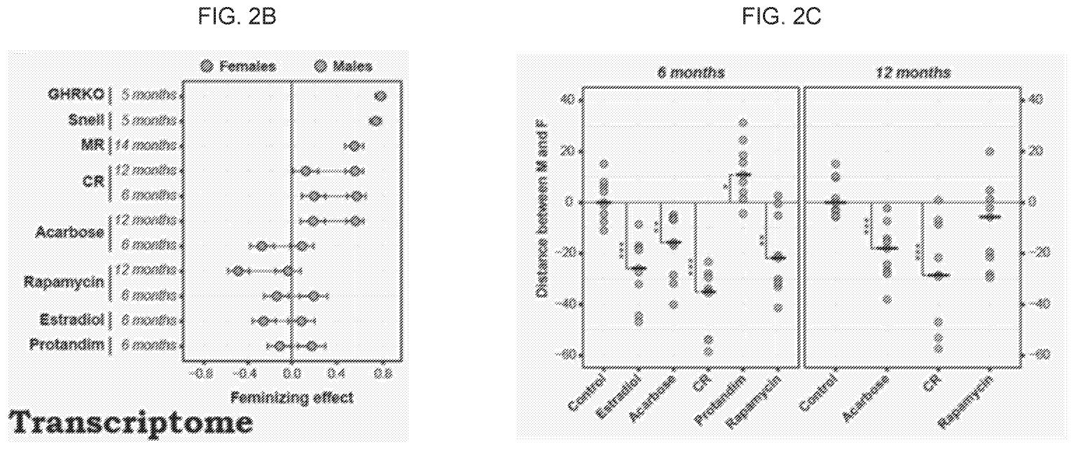

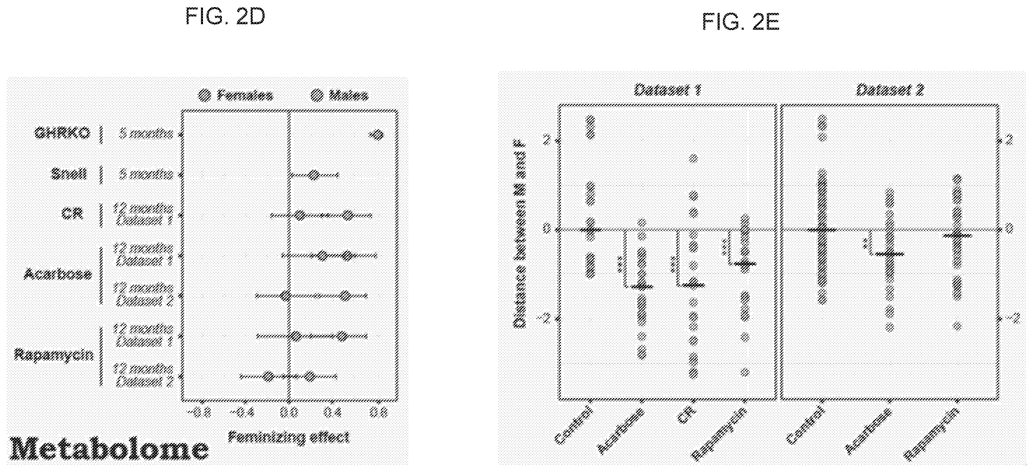

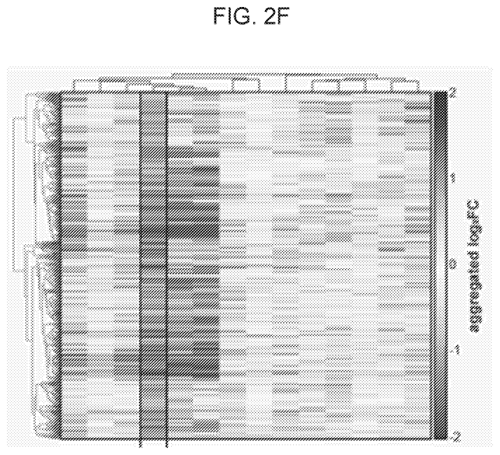

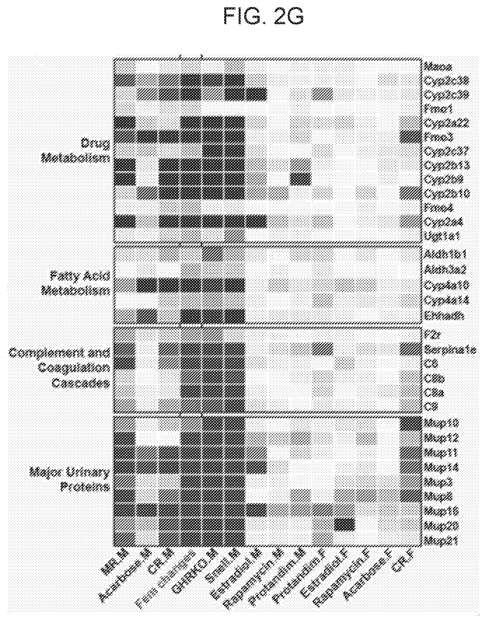

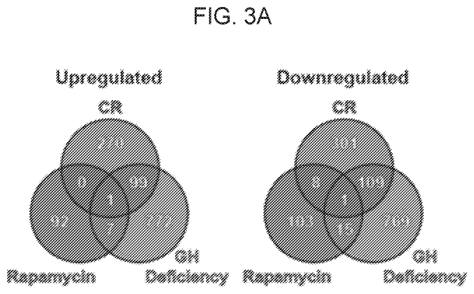

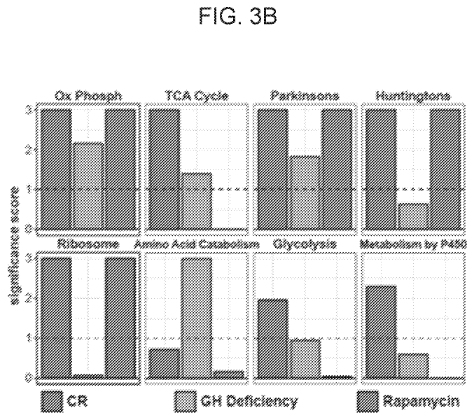

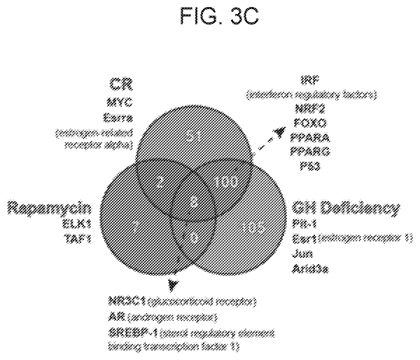

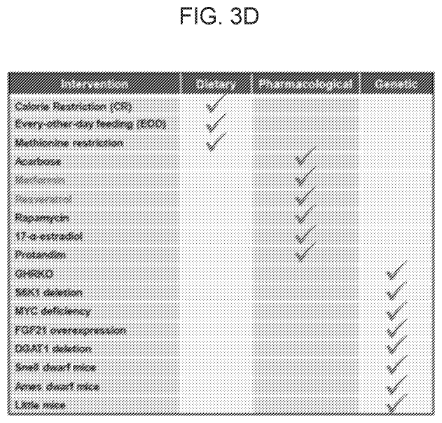

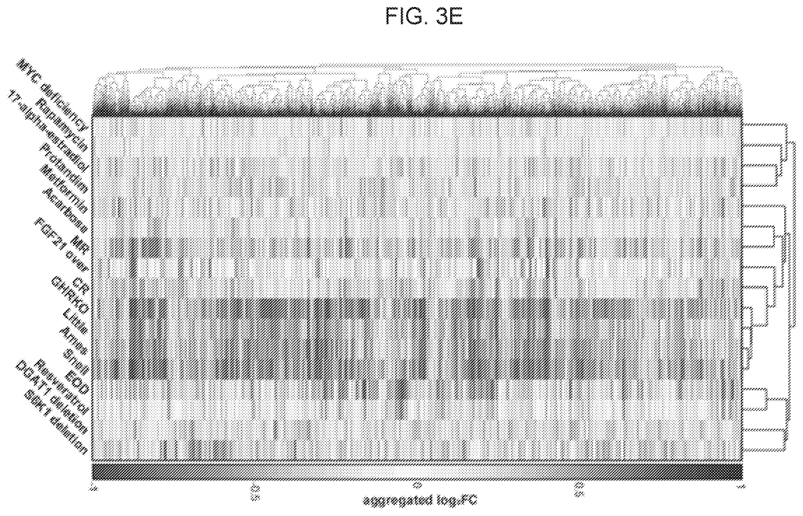

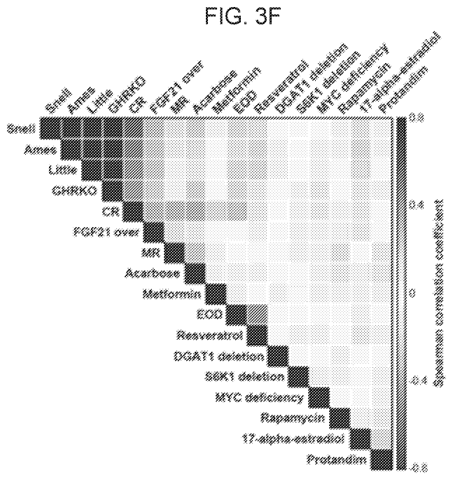

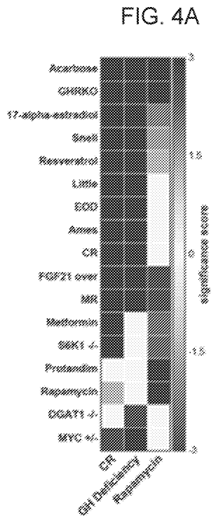

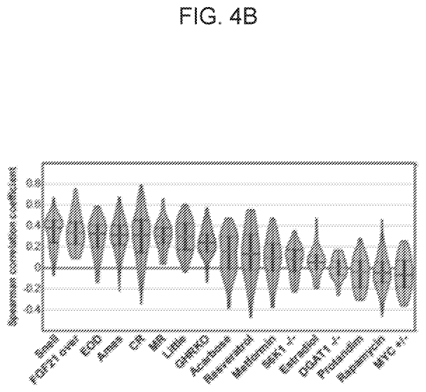

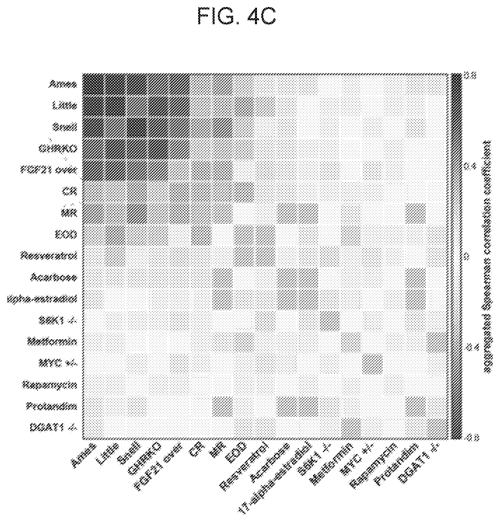

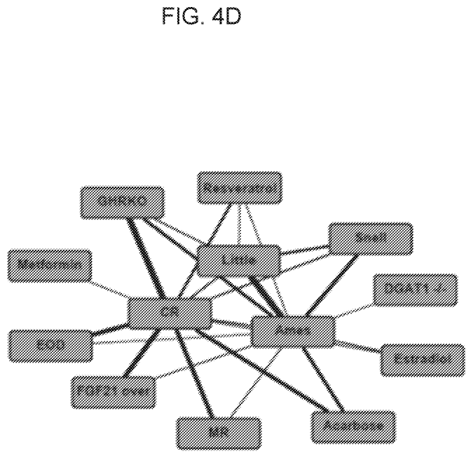

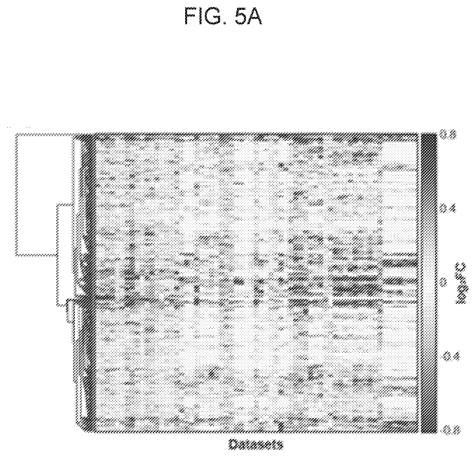

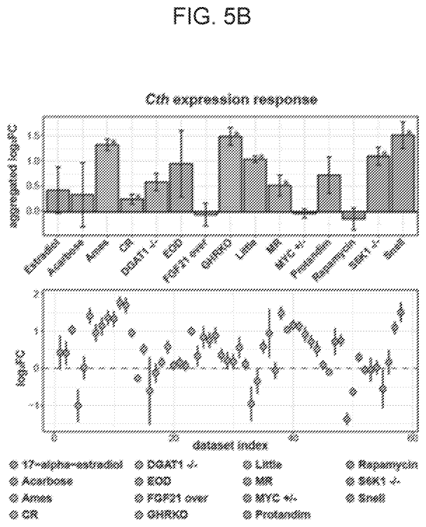

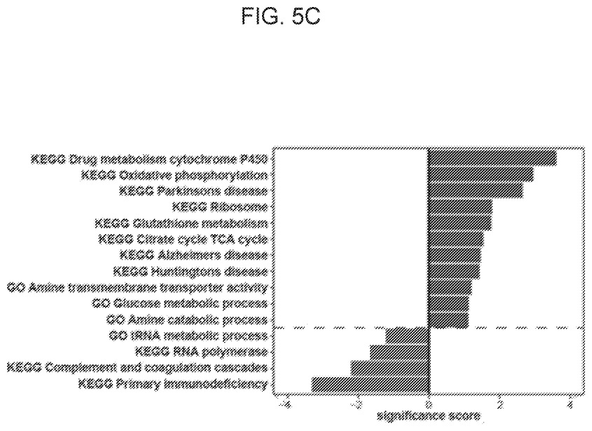

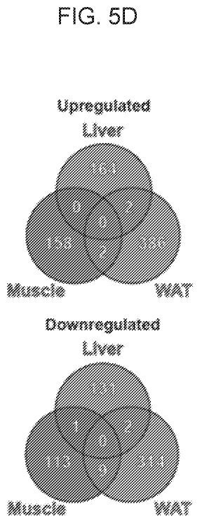

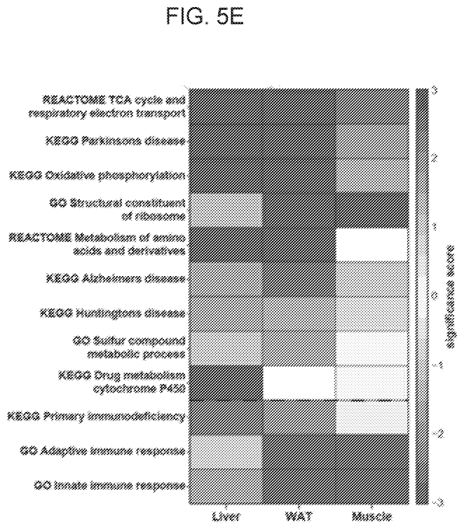

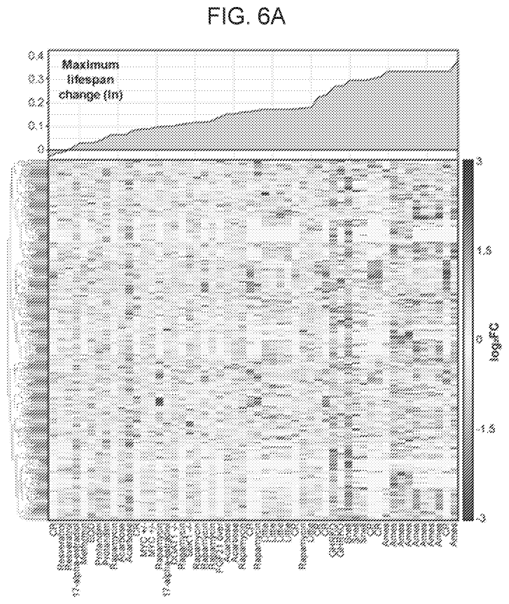

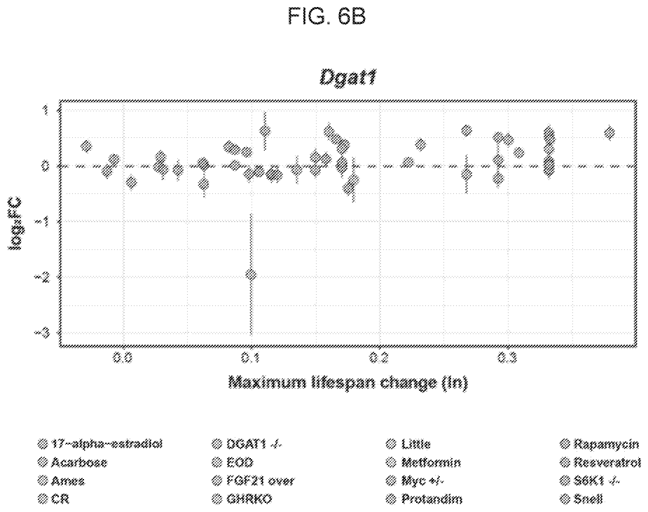

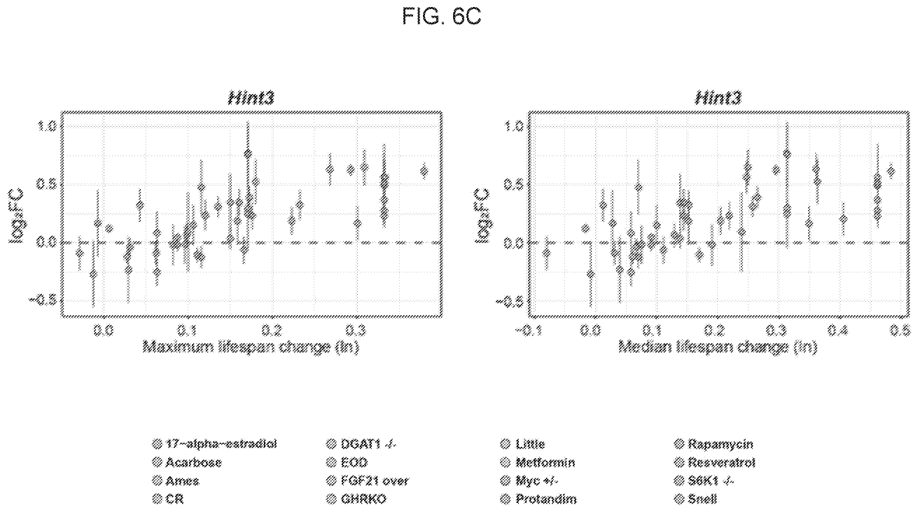

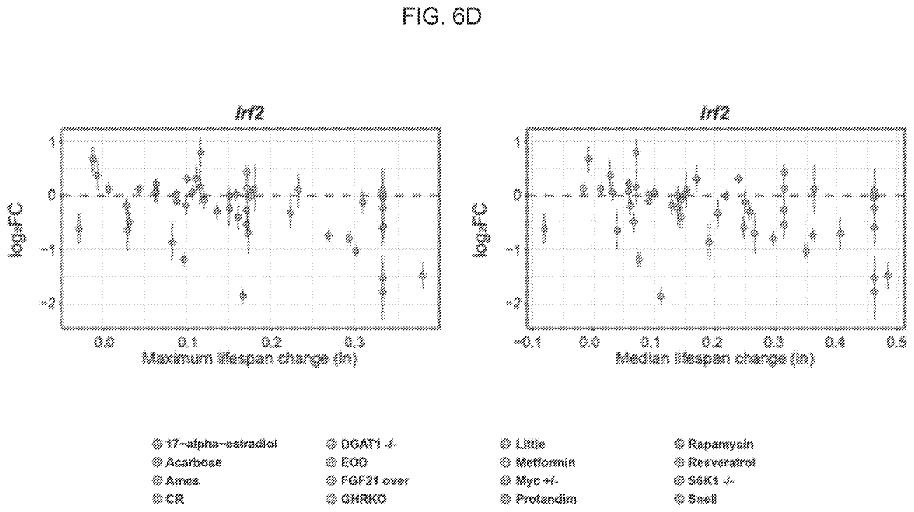

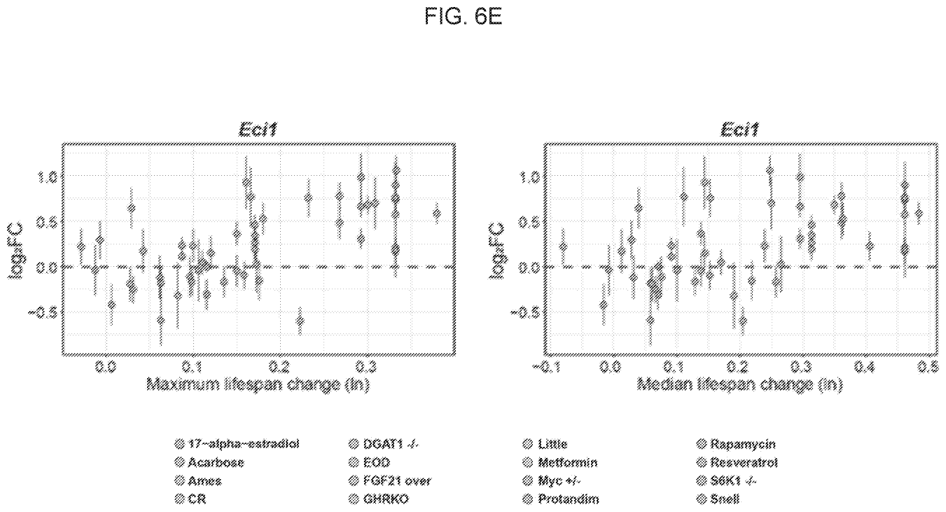

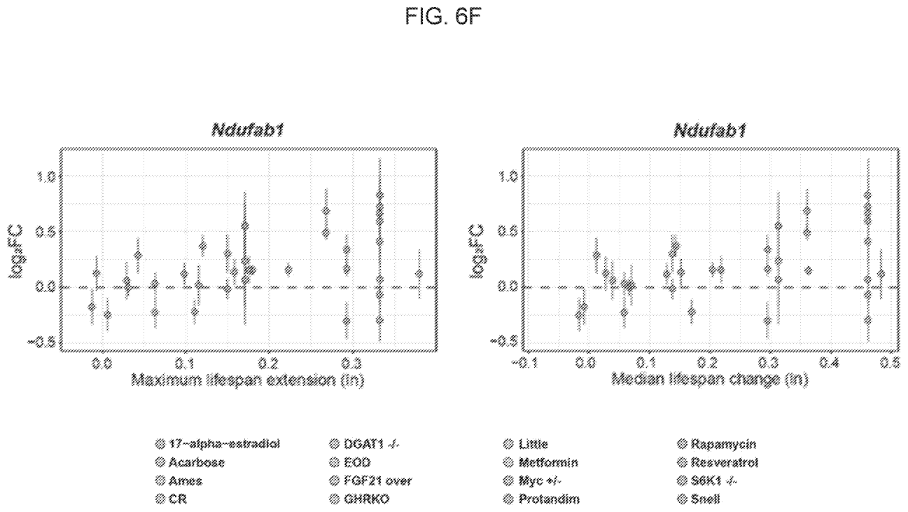

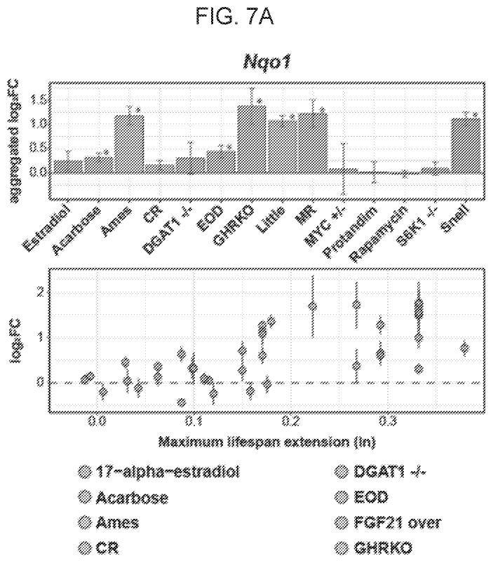

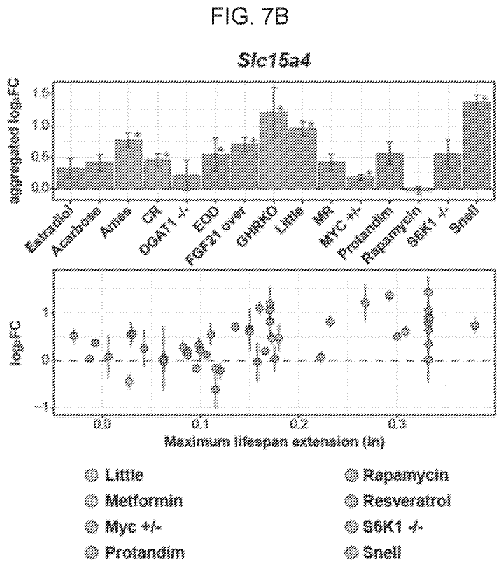

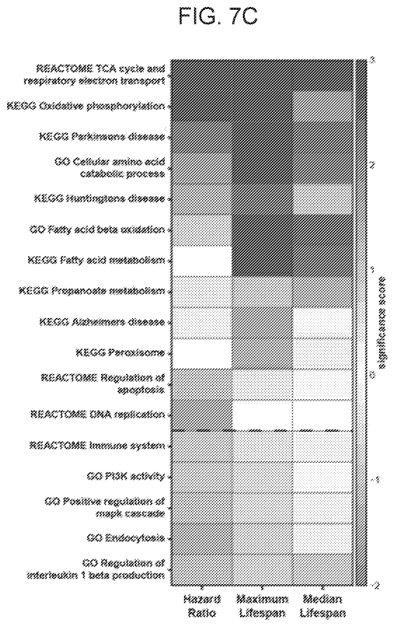

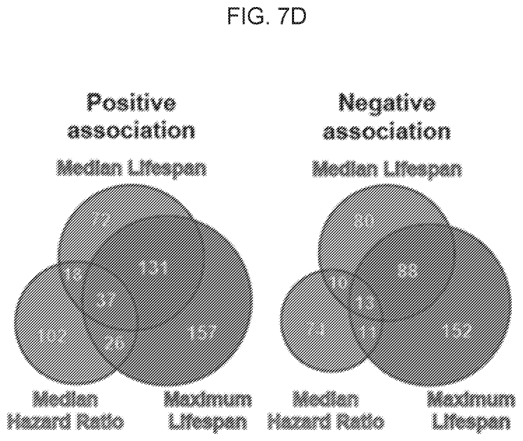

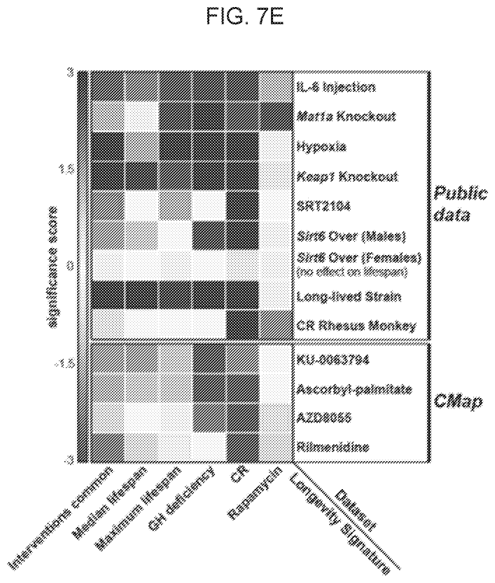

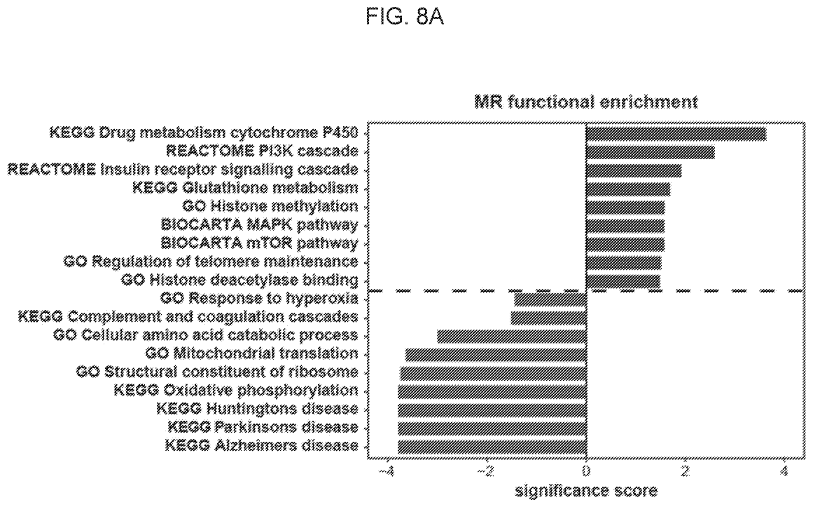

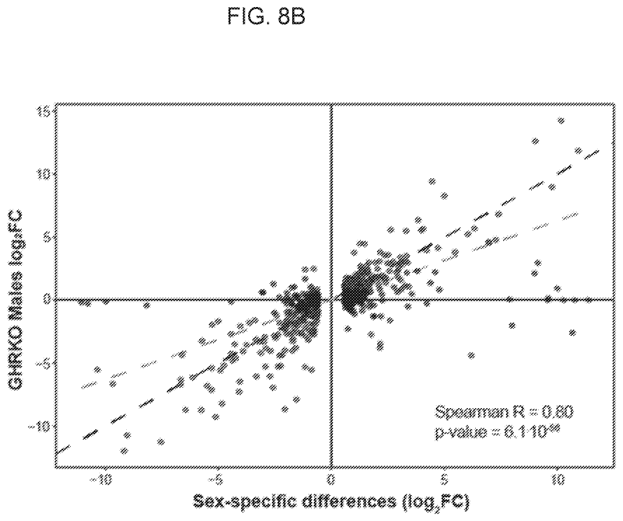

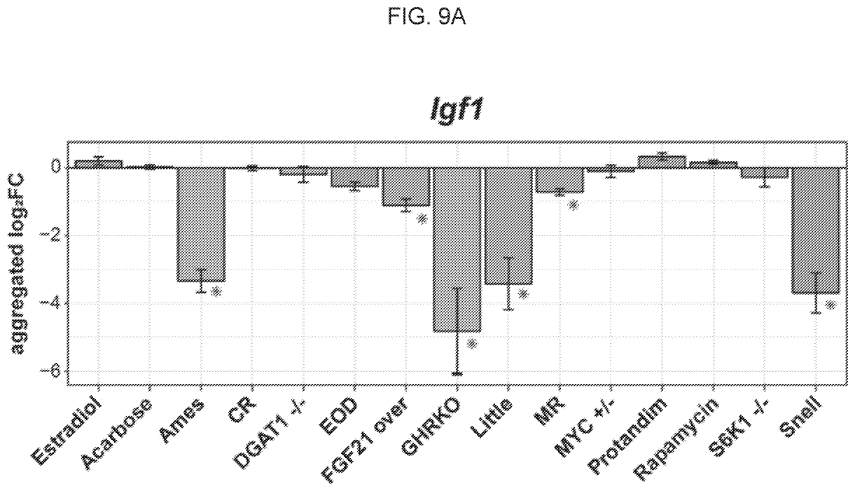

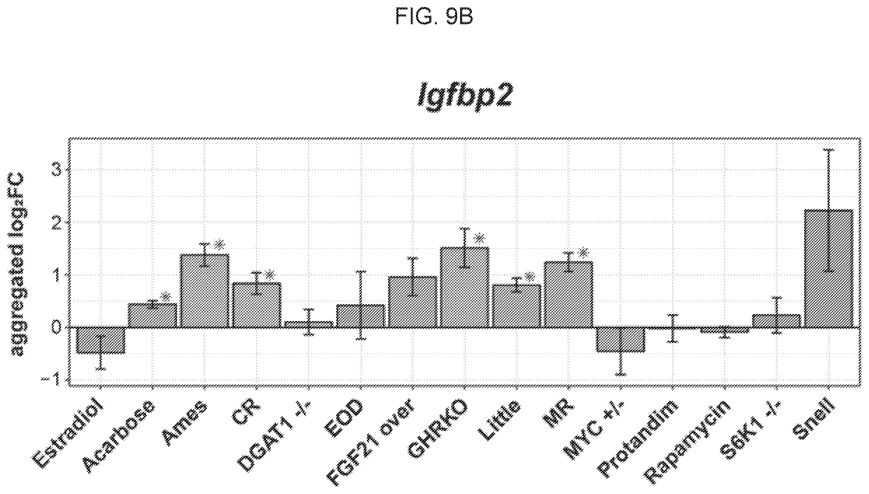

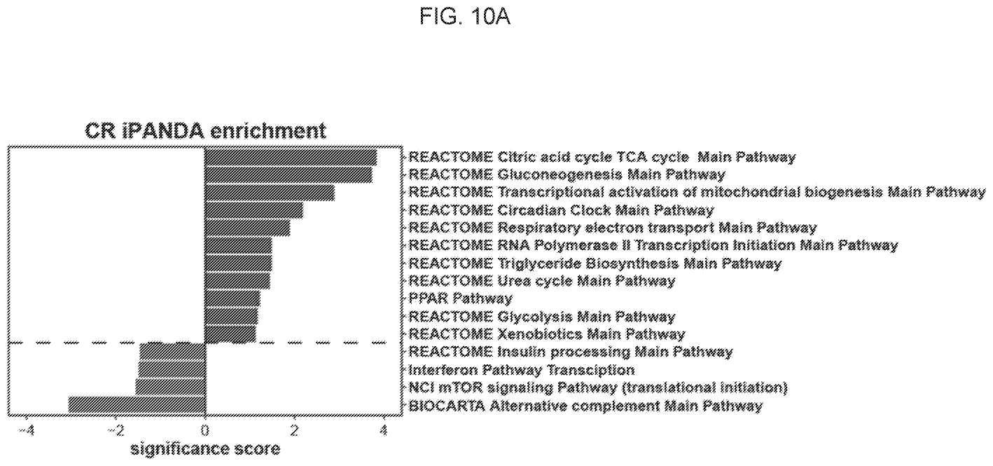

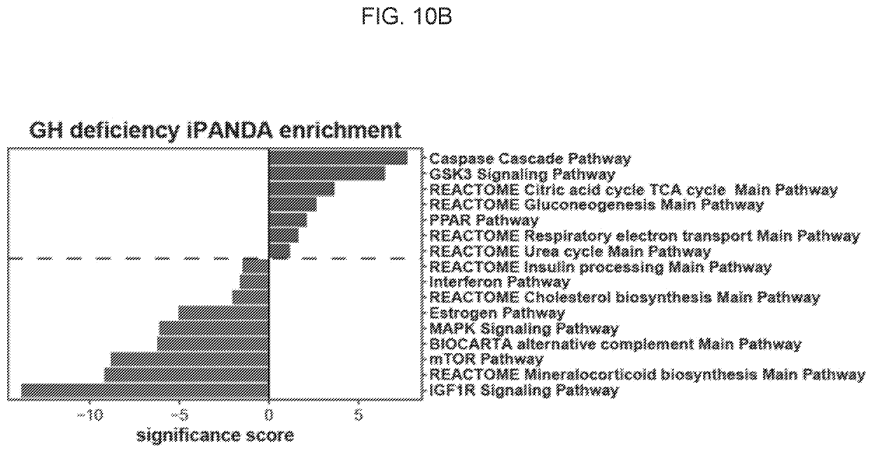

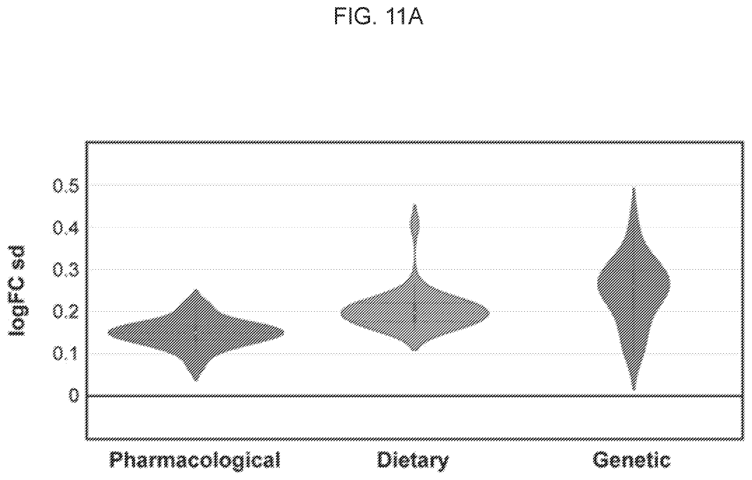

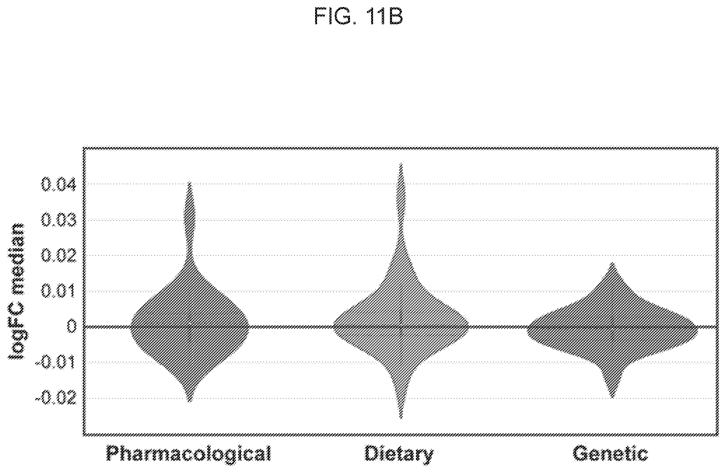

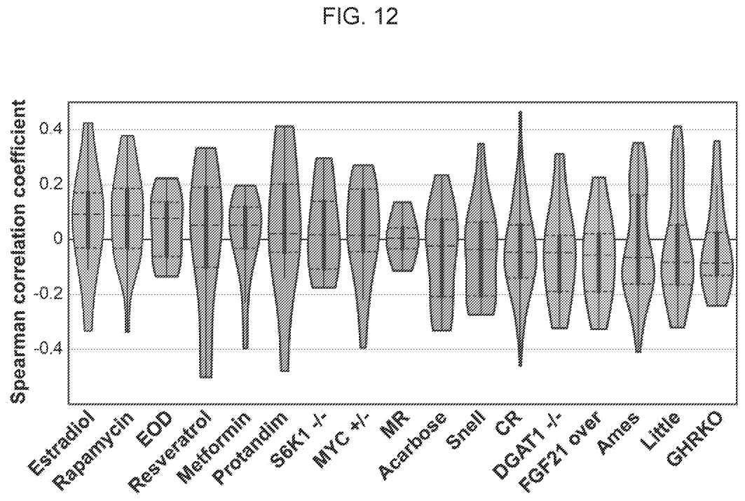

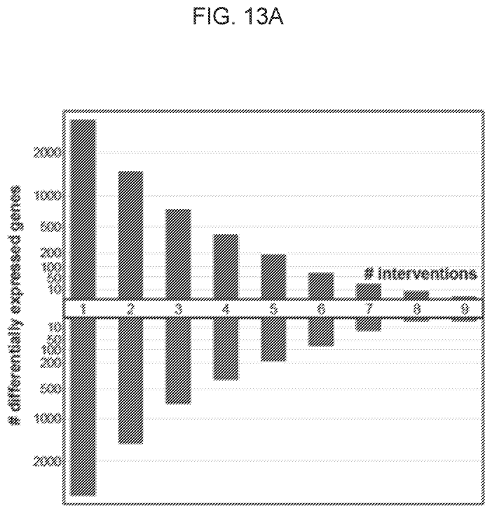

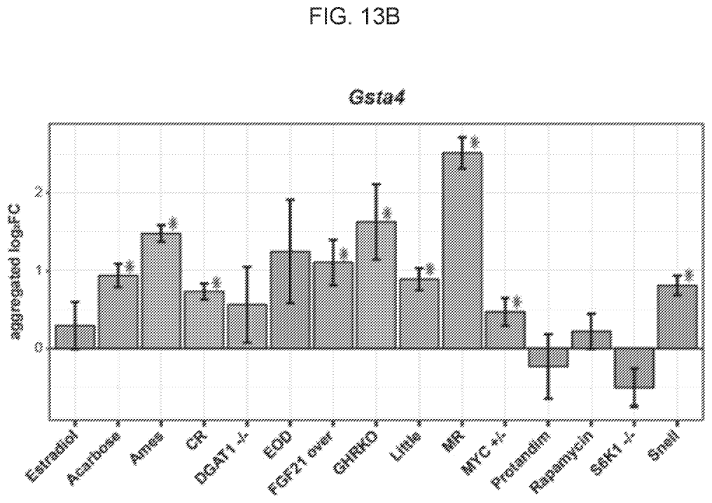

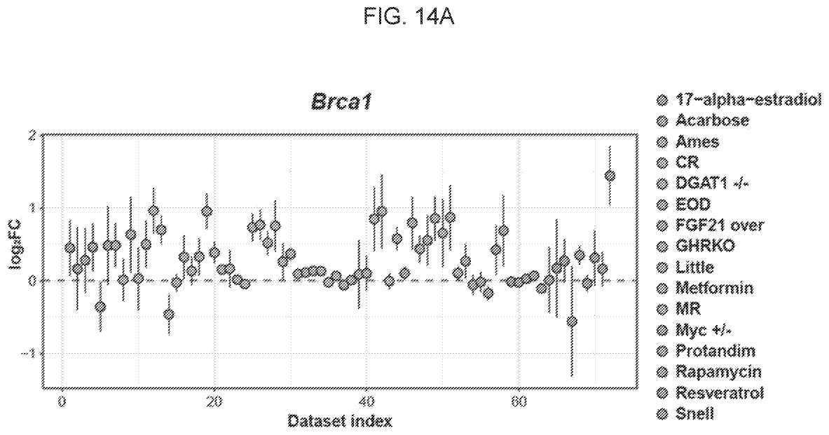

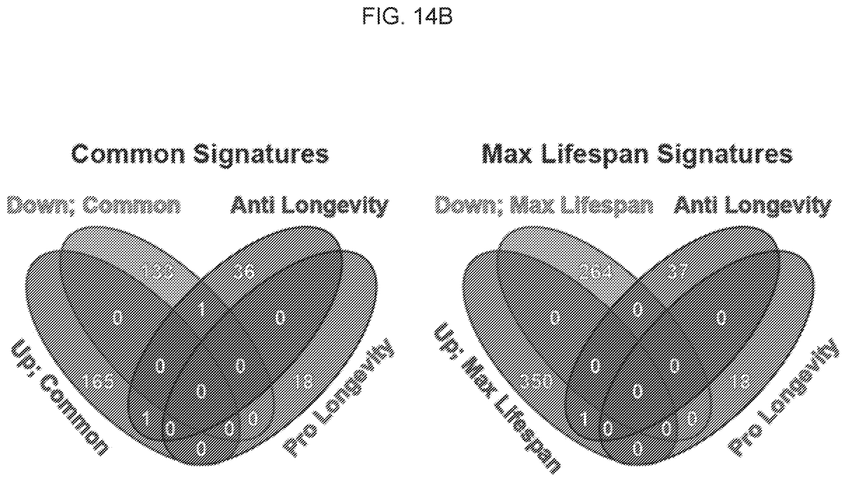

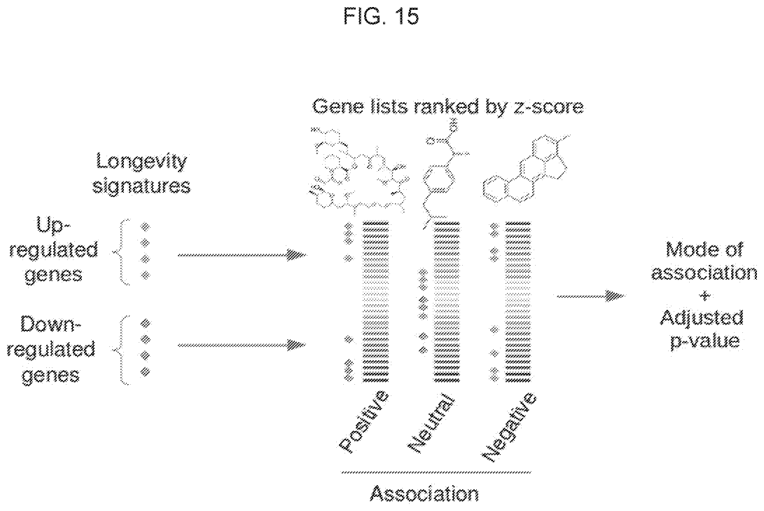

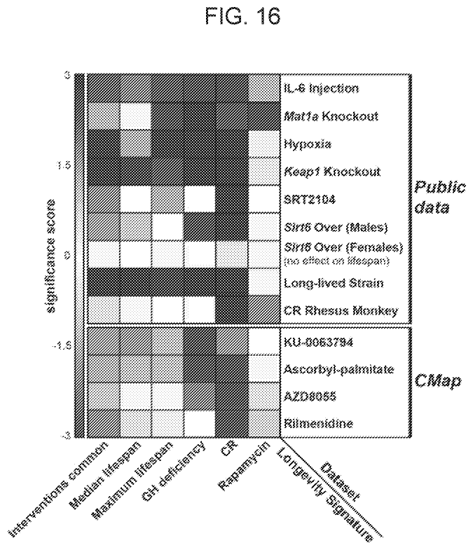

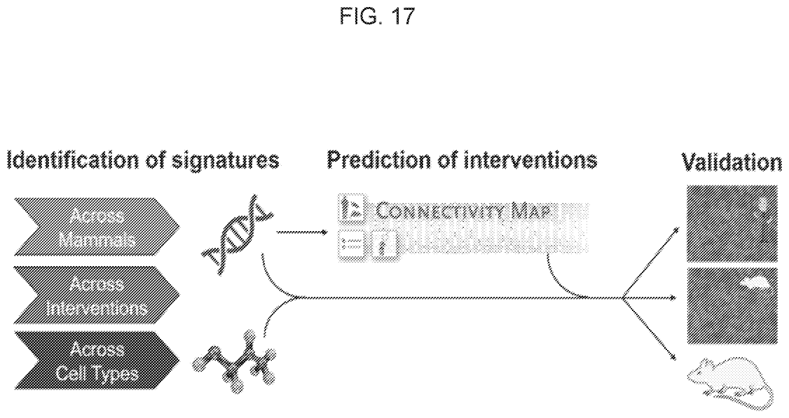

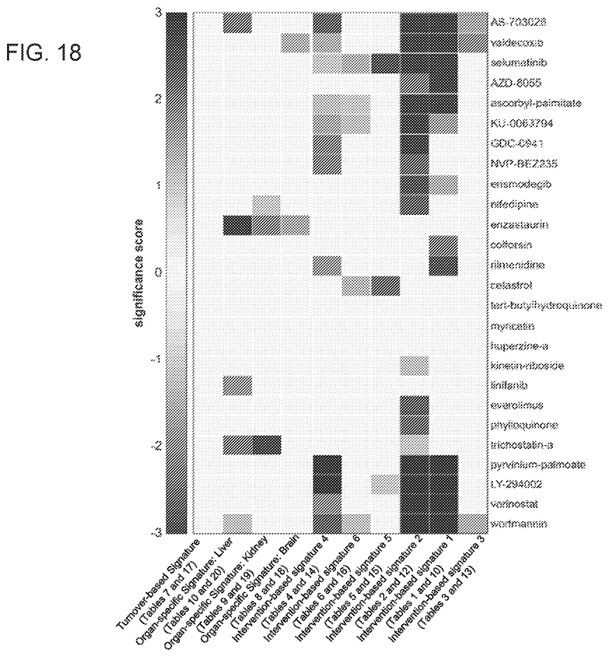

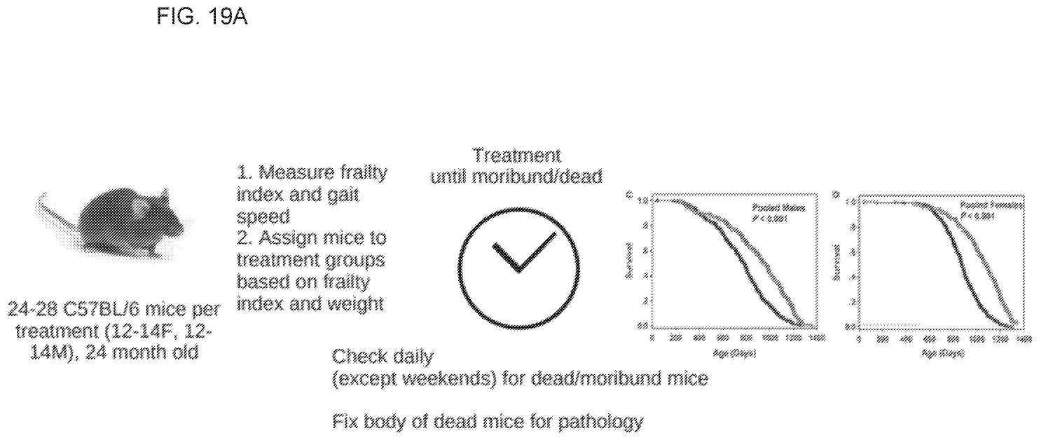

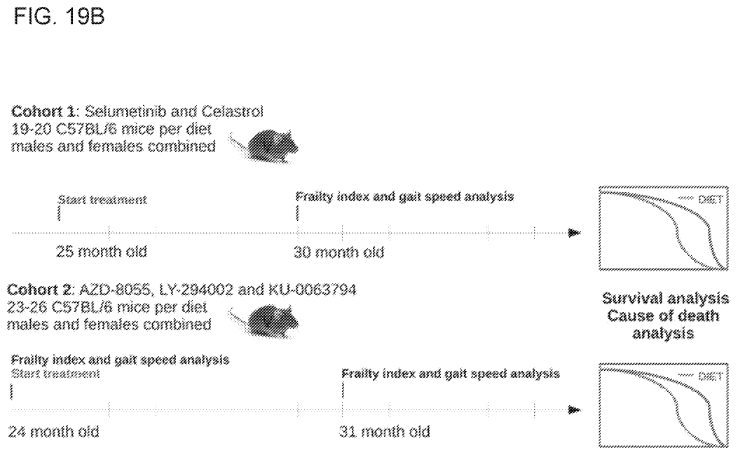

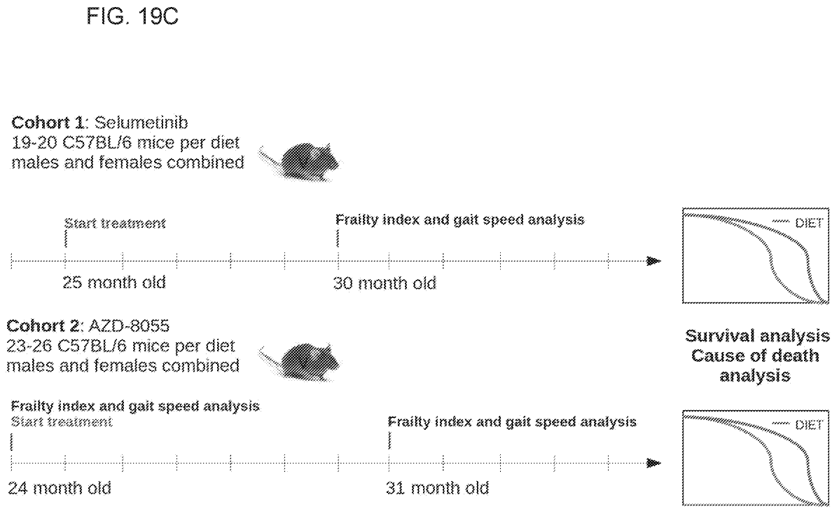

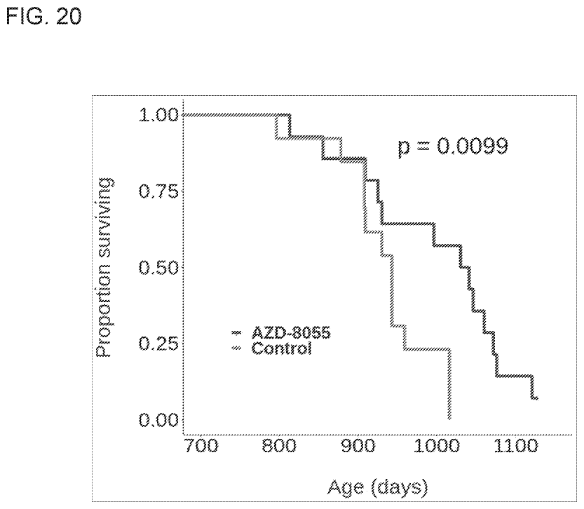

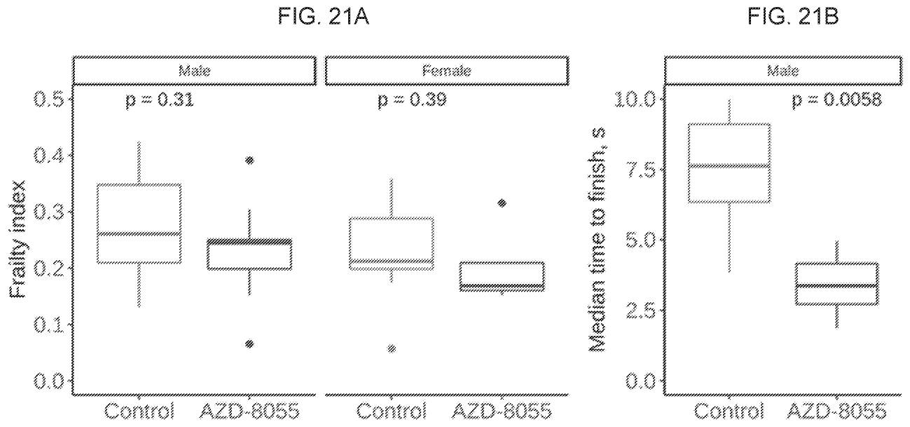

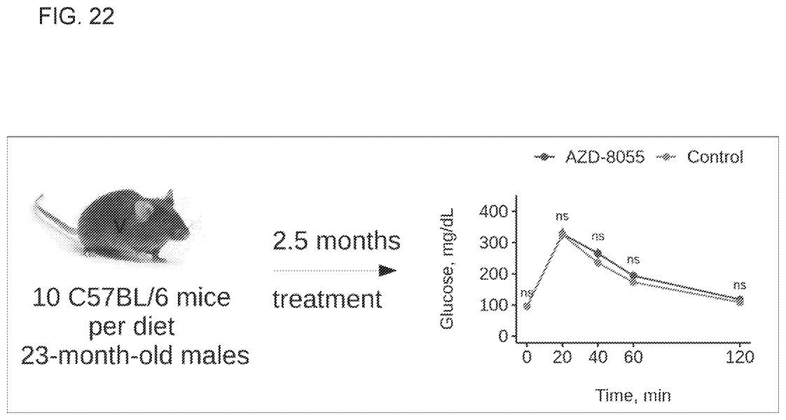

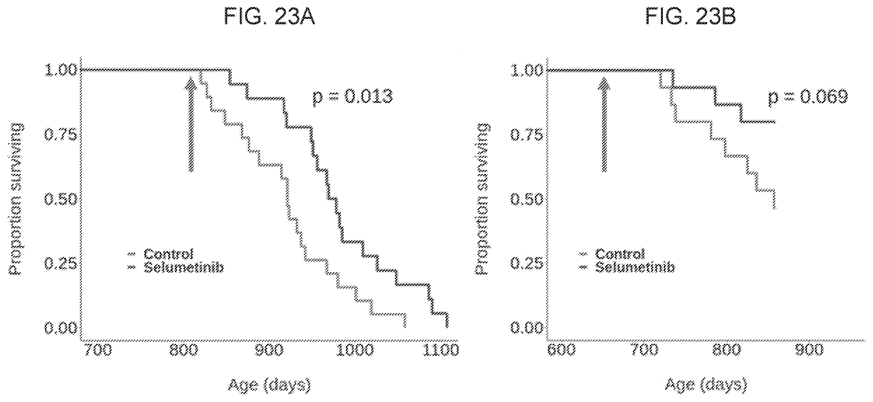

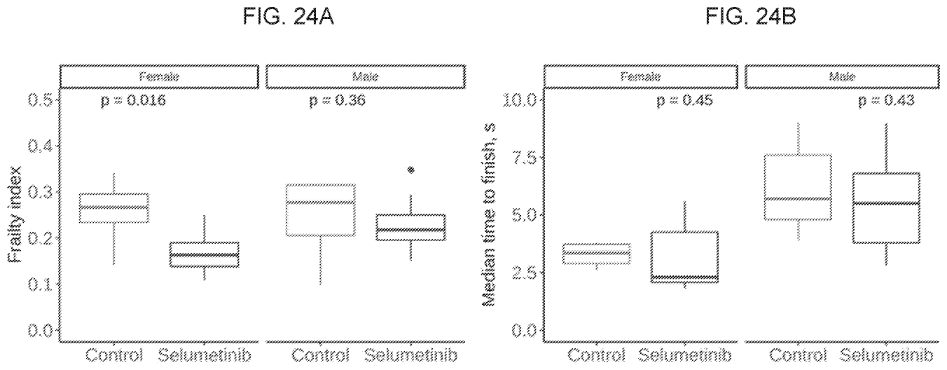

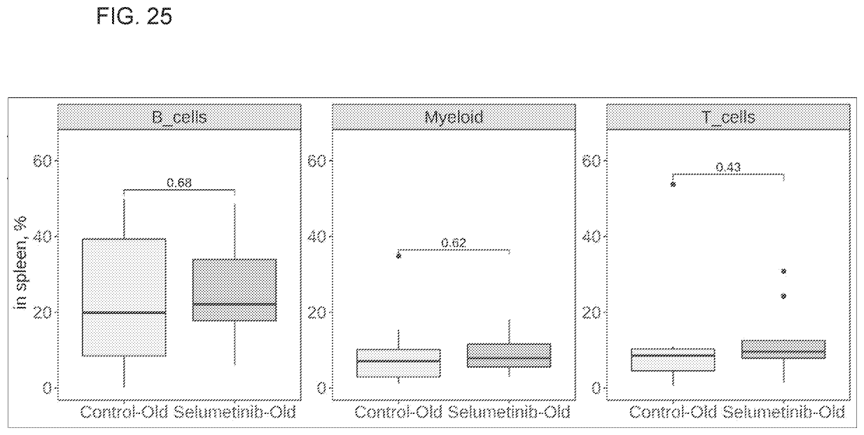

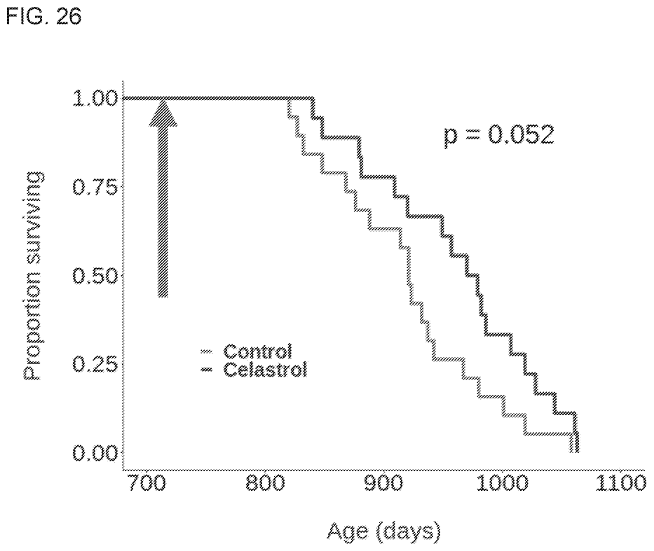

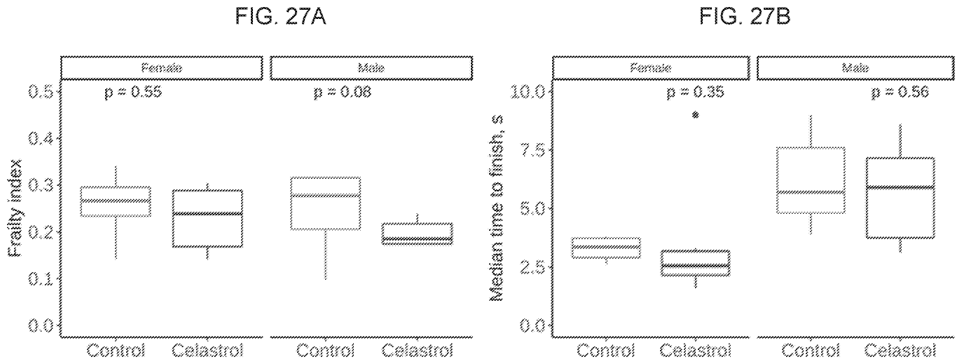

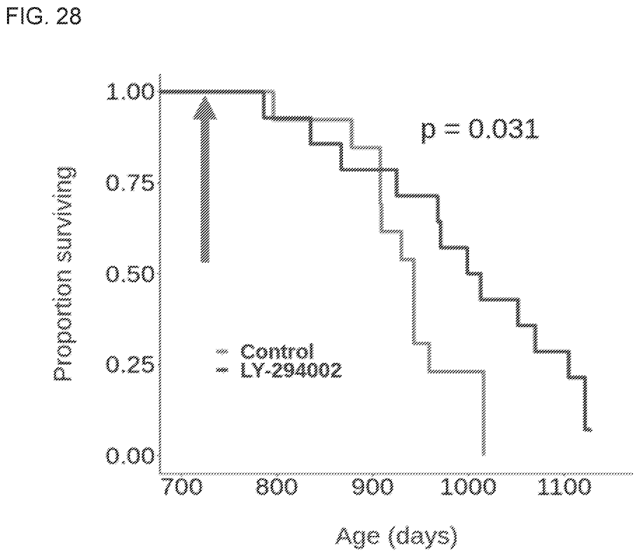

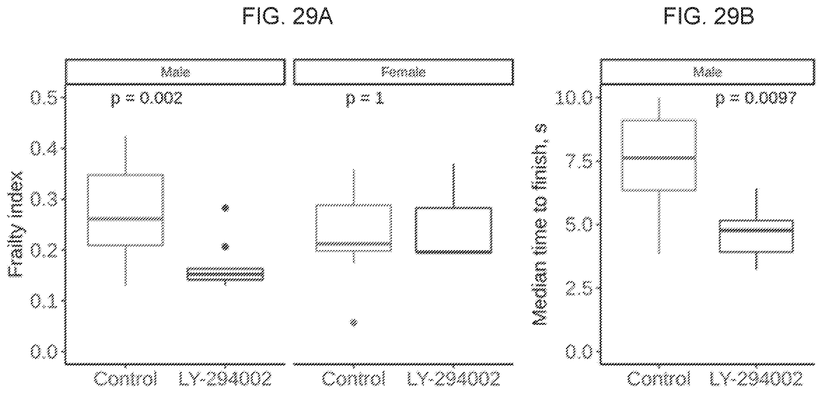

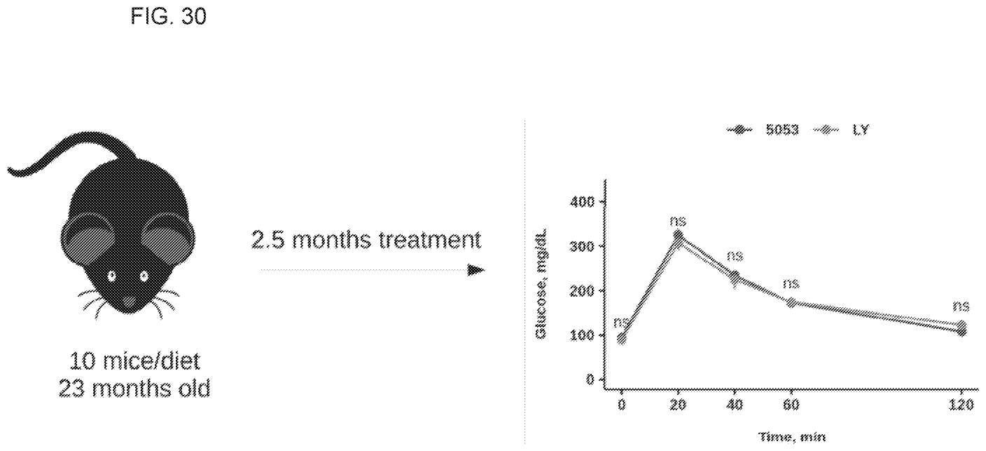

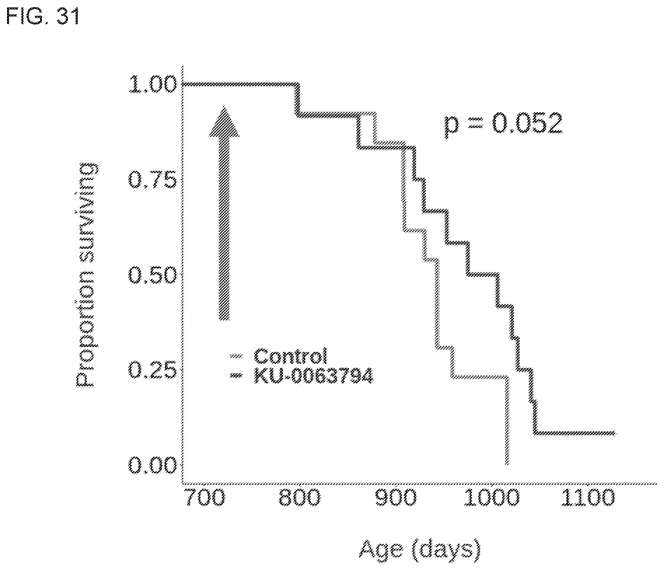

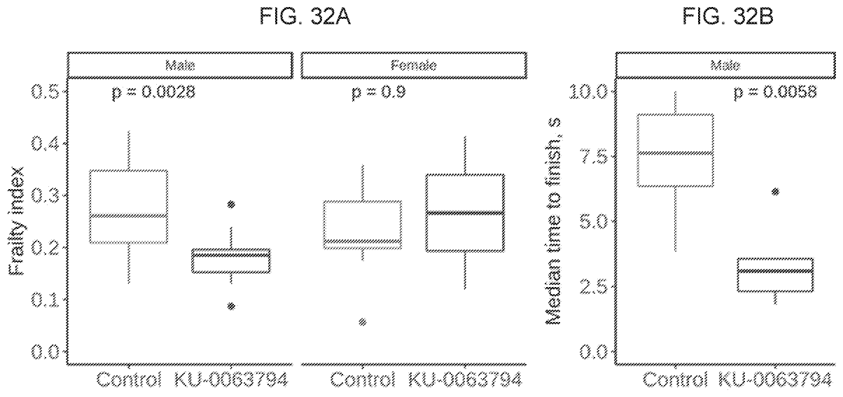

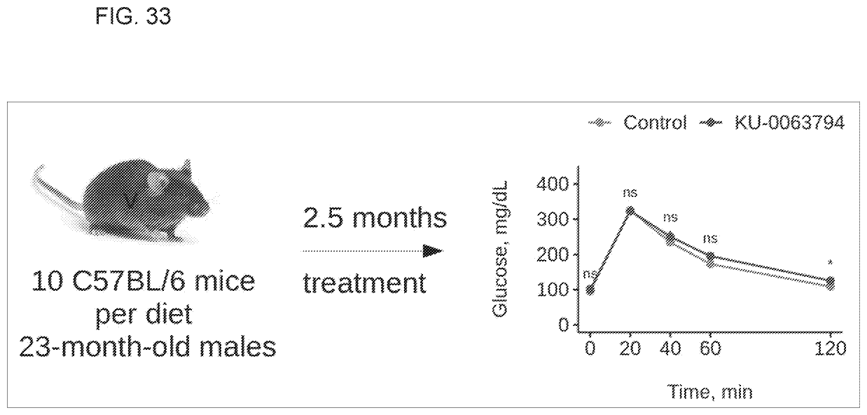

For CR, this method resulted in statistically significant positive correlations with the majority of interventions, including all GH deficiency interventions (BH adjusted Mann-Whitney p-value <6.1·10⁻¹⁰ for all of them), dietary interventions, such as CR itself (BH adjusted Mann-Whitney p-value=1.2·10⁻⁹⁵), MR and EOD (BH adjusted Mann-Whitney p-values <1.95·10⁻⁵), as well as FGF21 overexpression, acarbose, 17-α-estradiol, metformin and resveratrol (BH adjusted Mann-Whitney p-values <3.2·10⁻³) (FIG. 4B). Interestingly, although rapamycin was originally thought to be a CR mimetic, it instead showed a slight (median Spearman correlation coefficient=−0.049) but significant (BH adjusted Mann-Whitney p-value=8.9·10⁻⁵) negative correlation with CR when compared at the gene expression level, in accordance with the other results (FIGS. 3A, 3F, and 4A). The same analysis applied to rapamycin revealed its significant positive correlation only with itself (median Spearman correlation coefficient=0.088; BH adjusted Mann-Whitney p-value=2.8·10⁻³) (FIG. 12).

Using the same approach, we prepared a matrix with median Spearman correlation coefficients for every pair of interventions aggregated across all control-intervention comparisons from different sources (FIG. 4C). We detected a tight cluster formed by GH deficiency interventions and Fgf21 overexpression. Dietary interventions, including CR, MR and EOD, showed positive correlation with this cluster and each other. Other interventions showed either week positive correlation with the main cluster (resveratrol, 17-α-estradiol, acarbose, metformin and S6K1 −/−) or quite distinct gene expression patterns with no significant positive correlation with the group of highly correlated interventions defined by CR and GH deficiency (DGAT1 −/−, MYC +/−, Protandim and rapamycin). To clearly visualize similarity between gene expression profiles of different interventions, we built a network where the width of an edge connecting a pair of interventions reflected the level of statistical significance of Spearman correlation between them across datasets (FIG. 4D). Here, only the edges with statistically significant positive associations (BH adjusted Mann-Whitney p-value <0.1) are shown. In accordance with the results discussed above (FIG. 4A), most interventions shared similarity with CR and GH deficiency interventions (such as Ames and Little dwarf mice) at the level of gene expression (FIG. 4D). The lack of statistically significant associations between many interventions may reflect an insufficient number of independent datasets. The relatively high value of median Spearman correlation among interventions forming the main cluster (FIG. 4C) suggests that increase in the number of datasets may fill many edges missing in the network.

Overall, most lifespan-extending interventions showed similar gene expression patterns both at the level of whole transcriptomes and particular genes. However, some interventions, such as rapamycin, Protandim, S6K1 −/− and MYC +/−, showed quite distinct transcriptional patterns in liver, and did not demonstrate statistically significant positive correlation with any other intervention (FIG. 4D). This was especially interesting in the case of rapamycin, which, although originally thought to be a CR mimetic, showed positive correlation neither with CR nor with GH deficiency interventions, in accordance with the results of other groups (Fok et al., 2014b; Miller et al., 2014), and even showed statistically significant negative correlation with CR (FIG. 7). The data suggest distinct molecular mechanisms at the level of gene expression that mediate the effects of rapamycin and other interventions. However, based on the unbiased comparison of rapamycin datasets, we detected low (although significant) positive correlation of this intervention with itself (median Spearman correlation coefficient=0.088) (FIG. 7), which may point to high variability of the response to rapamycin at the level of gene expression across different experimental settings. The higher level of noise observed in the transcriptome response to rapamycin may also be a consequence of the generally lower extent of gene expression changes in response to drugs compared to diets and genetic manipulations (FIG. 11A). Therefore, a more comprehensive study of the rapamycin effect on the transcriptome is needed to validate our findings and better understand cellular mechanisms responsible for this unique pattern.

### Example 14

### Common Signatures Across Lifespan-Extending Interventions

To identify gene signatures commonly up- or downregulated by lifespan-extending interventions, which could serve as an approximation of ‘necessary’ features and qualitative predictors of lifespan extension, we first identified statistically significant genes regulated by each individual intervention using the same approach as in case of CR, rapamycin and GH deficiency interventions, where datasets from several independent sources were present. To account for possible differences of the intervention effect on lifespan across doses, ages, strains and sexes, introduced by heterogeneity of our data, here we only considered the datasets, whose experimental conditions were shown to produce statistically significant extension of lifespan.

Using the intervention-wise approach, for every gene we calculated the number of interventions, where it was up- or downregulated (FIG. 13A). One gene (Gsta4) was significantly upregulated in 9 different interventions (out of 15) (FIG. 12) and 7 genes (Gstt3, Abcb1a, Slc22a29, Slc15a4, Ak4, Serpina6 and Cers6) were upregulated in 8 interventions (BH adjusted p-value <0.1). These genes are involved in xenobiotic (Gsta4, Gstt3, Abcb1a and Slc22a29), glucocorticoid (Serpina6) and sphingolipid (Cers6) metabolism. In addition, 2 genes (C9 and C8a, both of which are complement components) were identified as significantly downregulated in 9 and 8 interventions, respectively. However, this approach has several disadvantages. First, it does not account for the difference in the number of datasets associated with every intervention along with the difference in quality of individual datasets (e.g., number of samples). Second, it does not consider possible similarity of different interventions at the level of global gene expression (as in case of GH deficiency interventions which showed very similar effects on the hepatic transcriptome). Therefore, this method leads to overfitting of common signatures by genes differentially expressed in response to GH deficiency.

To overcome this problem, we searched for genes shared by different interventions using a single mixed-effect model with an additional random term corresponding to intervention type and correlation matrix for this term composed from means of correlation coefficients of gene expression changes between the corresponding interventions across all possible pairs of datasets (FIG. 4C). This approach addresses the shortcomings of the previous method by increasing the weight of well-represented interventions and decreasing the weight of similar interventions (e.g., GH deficient mutants). Therefore, gene expression changes induced only by GH deficiency will have a higher probability of being realized by null hypothesis and, as a consequence, higher p-values. Using this method, we detected only 7 up- and 5 downregulated genes shared by all interventions with BH adjusted p-value <0.05. In other words, although there may be shared molecular mechanisms among different interventions, they are usually supported by different gene expression changes.

To detect genes commonly shared by most interventions, we weakened the criteria by letting one intervention to be an outlier. We accomplished this by removing each intervention one by one and taking the best remaining p-value (“robust p-value” approach). Using the BH adjusted robust p-value threshold of 0.05, we identified 166 upregulated and 134 downregulated genes (FIG. 5A). Interestingly, one of the most significant commonly upregulated genes was Cth (BH adjusted robust p-value=0.0033) (FIG. 5B). Cth encodes cystathionine gamma-lyase, which participates in glutathione synthesis and H₂S production (Kabil et al., 2011). H₂S by itself was demonstrated to extend lifespan in worms (Miller and Roth, 2007), and its production increased in response to CR in both sexes in different mouse strains (Mitchell et al., 2016). Cth was also shown to be upregulated in response to short-term 50% CR and to mediate oxidative stress resistance under conditions of sulfur amino acid restriction (Hine et al., 2015). Unexpectedly, its expression was increased in response to high-protein diet, which seems to be negatively associated with lifespan (Gokarn et al., 2018), although molecular mechanisms remain largely unknown. Except for this case, our data suggest that the hepatic expression of Cth is increased by most lifespan-extending interventions and could be used as a simple molecular biomarker associated with longevity.

Another interesting example of a gene commonly upregulated across lifespan-extending interventions is Brca1 (BH adjusted p-value=0.04) (FIG. 14A). This well-known tumor suppressor, whose loss-of-functions mutations are associated with breast and ovary cancer in humans with frequency of 80% and 40%, respectively (Narod and Foulkes, 2004), has also been found in several studies to be related to longevity in mice. In particular, its haploinsufficiency (Brca1+/−) led to shortened lifespan (by 8% in mean lifespan) with 70% tumor incidence vs about 10% in wild-type animals (Cao et al., 2003). Interestingly, besides being related to DNA repair, BRCA1 was also shown to physically interact with NRF2 and increase its stability and activation (Gorrini et al., 2013). Consequently, it may act by activating the NRF2-dependent antioxidant response. Thus, the common upregulation of Brca1 may be due to activation of NRF2 signaling, which is one of the shared signatures of lifespan-extending interventions (FIGS. 3C and 5D).

Several glutathione S-transferase genes were also significantly upregulated across lifespan-extending interventions, including Gstt2 (BH adjusted robust p-value=0.014), Gsto1 (BH adjusted robust p-value=0.037) and Gsta4 (BH adjusted robust p-value=0.013) (FIG. 13B). All of them are involved in glutathione metabolism, known to be activated at the gene expression level in response to CR (Fu and Klaassen, 2014) and several GH deficiency states (Sun et al., 2013; Tsuchiya et al., 2004). Administration of GH was shown to decrease GST activity in several tissues including liver (Brown-Borg et al., 2005). Overall, upregulation of Gst genes is a common signature of lifespan-extending interventions and they are significantly changed not only by GH deficiency and CR, but also by FGF21 overexpression, acarbose, MR, MYC deficiency and others (FIG. 13B).

To identify pathways associated with common up- and downregulated gene signatures, we performed functional GSEA (FIG. 5C). In accordance with the RNAseq findings, the most significant upregulated functions included metabolism of xenobiotics by cytochrome P450 (q-value=0.0055) and glutathione metabolism (q-value=0.017) mainly regulated by the NRF2 pathway, oxidative phosphorylation (q-value=0.001), ribosome (q-value=0.016), TCA cycle (q-value=0.028), glucose (q-value=0.074) and amino acid metabolism (q-value=0.075). Downregulated functions included primary immunodeficiency (q-value=4.8·10⁻⁴), RNA polymerase (q-value=0.022) and tRNA metabolic process (q-value=0.061). Interestingly, several age-related diseases associated at the molecular level with age-dependent changes in regulation of many cellular pathways, including mitochondrial function, oxidative phosphorylation, apoptosis and proteolysis, such as Alzheimer's (q-value=0.034), Parkinson's (q-value=2.2·10⁻³) and Huntington's (q-value=0.036) diseases, were enriched for common signatures, pointing to the connection between changes induced by aging and lifespan-extending interventions.

To generalize our findings across tissues, we aggregated publicly available data on gene expression responses to lifespan-extending interventions in two additional tissues, skeletal muscle and white adipose tissue (WAD. Using the same methods and threshold criteria, we examined this dataset for common longevity signatures in each tissue. We identified 160 and 390 upregulated along with 123 and 325 downregulated genes for the muscle and WAT, respectively. Interestingly, there was almost no overlap between common gene expression signatures across different tissues (FIG. 5D). On the other hand, GSEA resulted in the number of shared molecular functions enriched by these signatures (FIG. 5E). Thus, oxidative phosphorylation (q-value <0.024), amino acid metabolism (q-value <10⁻³ for liver and WAT), and ribosome structural genes (q-value <0.061) along with age-related diseases such as Parkinson's (q-value <0.012) and Alzheimer's (q-value <0.083) turned out to be commonly upregulated across tissues, while immune response genes were commonly downregulated (FIG. 5E). Some other functions, such as drug metabolism by cytochrome P450 discussed previously, appeared to be tissue-specific. Therefore, lifespan-extending interventions seem to affect different individual genes across tissues. However, in general, these signatures converge to the same molecular pathways, although some functions appear to be restricted to a particular tissue.

### Example 15

Signatures Associated with the Degree of Lifespan Extension

To identify genes positively and negatively associated with the degree of lifespan extension, potentially serving as quantitative predictors of longevity, we integrated a previously described mixed-effect regression model with 3 commonly used metrics of lifespan extension obtained from published survival data on corresponding interventions: median lifespan ratio, maximum lifespan ratio, calculated as the ratio of average lifespan of 10% longest-surviving individuals, and median hazard ratio, calculated as the ratio of slopes of survival curves at the timepoint where 50% of cohort is alive. We used these metrics as they seem to be the most consistent and robust to the effects of sampling size (Moorad et al., 2012). To account for heterogeneity of the data, we integrated gene expression and the longevity data only if they were associated with the same experimental design in terms of sex, strain, dose and the age at which the intervention started. As in the case of common signatures, we considered source and type of intervention as random terms and used the correlation matrix of interventions to account for similarity between them.

We designated genes as statistically significant if their BH adjusted p-value and LOO p-value, obtained after removal of every intervention one by one, were both <0.05. With these thresholds, we detected 351, 258 and 183 genes with positive and 264, 191 and 108 genes with negative association with maximum lifespan ratio, median lifespan ratio and median hazard ratio, respectively (FIGS. 6A and 7D). These gene sets showed a significant overlap (Fisher exact test p-value <10⁻¹⁸ in all cases), which was especially large between median and maximum lifespan. Indeed, 65.1% and 47.9% of genes with positive and 52.9% and 38.3% with negative association with median and maximum lifespan, respectively, were shared between them. As the median hazard ratio is more volatile compared to other metrics, median and maximum lifespan provide more reliable sets of genes. One of the strongest positive associations with maximum and median lifespan was found for Hint3 (BH adjusted p-value=3.2·10⁻′ and 2.5·10⁻⁴, respectively) encoding nucleotide hydrolase (FIG. 6C). On the other hand, Irf2 encoding interferon regulatory transcription factor showed a significant negative association with these metrics (BH adjusted p-value=2.57·10⁻⁶ and 1.2·10⁻⁵, respectively) (FIG. 6D).

Other genes positively associated with changes in both maximum and median lifespan included members of fatty acid metabolism, including acyl-coenzyme A dehydrogenase Acadm (BH adjusted p-value=0.001 and 0.005 for maximum and median lifespan, respectively) and enoyl-coenzyme A delta isomerase Eci1 (BH adjusted p-value=2.2·10⁻⁶ and 6.4·10⁻⁶) (FIG. 6E), and oxidative phosphorylation pathway, including the b subunit of ATP synthase Atp5f1 (BH adjusted p-value=5.3·10⁻⁴ and 0.004), cytochrome c oxidase assembly protein Cox17 (BH adjusted p-value=5·10⁻⁴ and 0.01) along with dehydrogenase 1 subcomplexes Ndufb3 (BH adjusted p-value=2.6·10⁻⁵ and 0.048) and Ndufab1 (BH adjusted p-value=0.005 and 0.003) (FIG. 6F).

Interestingly, the fat synthesis enzyme Dgat1, those knockout is associated with extension of mean and maximum lifespan in female mice by 23% and 8%, respectively (Streeper et al., 2012), was found to be slightly positively associated with median and maximum lifespan effects across interventions (slope coefficient=0.38 and 0.29 and BH adjusted p-value=0.007 and 0.04 for maximum and median lifespan, respectively) (FIG. 6B). However, the change of Dgat1 expression appears to be relatively small in response to all lifespan-extending interventions, except for Dgat1 deletion. Similar pattern was observed for Fgf21, whose expression was significantly increased only in response to Fgf21 overexpression. These examples demonstrate that longevity can be achieved through alteration of different master regulators, but these perturbations may result in the same downstream systemic responses, which are related to the lifespan extension effect.

To check if such pattern is universal for different genes, we compared the identified genes shared across signatures and associated with the degree of lifespan effect with the genes whose perturbation was demonstrated to extend mouse lifespan, obtained from GenAge (18 pro- and 38 anti-longevity genes) (De Magalhães and Toussaint, 2004). Indeed, we observed almost no overlap between these gene sets (Fisher exact test p-value >0.33 for all pairwise comparisons) (FIG. 14B). Therefore, the identified gene signatures appear to reflect the response of the whole molecular system and are associated with the longevity when altered together as a group, whereas lifespan-increasing genes represent upstream regulators, whose perturbations, in the end, lead to these systemic changes, similarly to dietary and pharmacological interventions.

To identify pathways enriched by genes positively and negatively associated with the lifespan extension effect, we ran GSEA for all 3 metrics of lifespan extension and observed general consistency among them in terms of functional enrichment (FIG. 7C). Thus, genes related to TCA cycle (q-value <10⁻³ for all metrics), oxidative phosphorylation (q-value <0.015 for all metrics), amino acid catabolism (q-value <0.02 for all metrics) and Huntington's (q-value <0.093 for all metrics) and Parkinson's diseases (q-value <0.004 for all metrics) were significantly positively associated among all three metrics used in the analysis, whereas fatty acid (q-value <0.003) and propanoate metabolism (q-value <0.081) genes showed significant positive association with maximum and median lifespan changes. On the other hand, regulation of interleukin 1 beta production showed significant negative associations with specified metrics (q-value <0.096 for median lifespan and median hazard ratio) (FIG. 7C). However, some functions, such as peroxisome (q-value=0.03 for maximum lifespan) and DNA replication (q-value=0.026 for median hazard ratio), were specific to single lifespan extension metrics. This may explain how certain interventions increase specific lifespan characteristics without affecting others.

Interestingly, some of the hepatic genes and pathways could be used for the prediction of both lifespan extension per se (qualitative estimate) as well as degree of this effect (quantitative estimate), being both common signatures and signatures associated with the lifespan extension effect. We identified 26 genes being both commonly changed across interventions and associated with either median or maximum lifespan extension effect in the same direction. 17 of them were upregulated and positively associated with lifespan extension, while 9 were downregulated and negatively associated. The identified genes are involved in regulation of apoptosis (Aatk, Net1, Rb1, Sgms1), immune response (C4 bp, P2ry14, Slc15a4, Tap2, Rb1), transcription (Pir, Sall1), stress response (Net1, Nqo1, Pck2, Rb1), glucose metabolism (Pck2, Pgm1) and cellular transport (Ldirad3, Slc15a4, Slc25a30 and Tap2).

For example, Nqo1, encoding NAD(P)H-dependent quinone oxidoreductase involved in oxidative stress response, showed a significant positive association with maximum and median lifespan (BH adjusted p-value=0.002 and 7.74·10⁻⁵, respectively) and was also commonly upregulated across lifespan-extending interventions (BH adjusted robust p-value=0.011) (FIG. 7A). Interestingly, this gene is one of the well-known targets of the transcription factor NRF2, an upstream regulator of gene expression response to various lifespan-extending interventions (Leiser and Miller, 2010; Mutter et al., 2015) (FIG. 3C).

Another interesting example is Slc15a4, which codes for lysosome-based proton-coupled amino-acid transporter of histidine and oligopeptides from lysosome to cytosol. In dendritic cells, this protein regulates the immune response by transporting bacterial muramyl dipeptide (MDP) to cytosol and, therefore, activating the NOD2-dependent innate immune response (Nakamura et al., 2014). In addition, its activity affects endolysosomal pH regulation and probably v-ATPase integrity, required for mTOR activation (Kobayashi et al., 2014). Our data show that Slc15a4 is a common signature of lifespan-extending interventions (BH adjusted robust p-value=0.008) along with some other transporters (FIG. 5C) and is positively associated with maximum lifespan (BH adjusted p-value=0.02) (FIG. 7B), pointing to the possible importance of lysosomal integrity and amino acid transport in lifespan extension.

As for the pathways, oxidative phosphorylation showed positive association with both common and lifespan effect associated signatures, and some functions involved in liver regulation of immune response showed negative association (FIGS. 5C, 5E, and 7C). Interestingly, downregulation of electron transport chain was also shown to be the only common signature of aging at the level of gene expression across different species including humans, mice and flies (Zahn et al., 2006). Therefore, contrary to the feminization effect, this pattern seems to demonstrate the opposite behavior during aging and in response to lifespan-extending interventions.

To make our data and tools available to the research community, we developed a web application, GENtervention, based on the R package shiny (Chang et al., 2016). It allows interrogation of gene expression data and, for every gene, it offers (i) expression change across different datasets related to every individual intervention (e.g. FIGS. 5B (upper), 7A (upper), 7B (upper), 9, and 13B), (ii) expression change in all available datasets across lifespan-extending interventions (common signatures) (FIGS. 5B (lower) and 14A), and (iii) the association of expression change with metrics of the longevity effect (signatures associated with the lifespan extension effect) (FIGS. 6B-6F, 7A (lower), and 7B (lower)). Within every section one can choose whether to include only interventions with confirmed lifespan-extending effect for the certain dose and regime or all analyzed regimes and interventions. Other available options include modes of randomization in mixed-effect model, statistical thresholds, lifespan extension effect metrics, filtering and coloring modes. GENtervention may be accessed through http://gladyshevlab.org/GENtervention/.

### Example 16

### Application of Longevity Signatures for the Identification of New Candidates for Lifespan Extension

In this work, we obtained gene expression patterns (signatures) associated with the response to particular well-studied interventions (CR, rapamycin and GH deficiency interventions), as well as signatures based on gene sets commonly regulated across different interventions and associated with the degree of lifespan extension. We considered the possibility that these ‘longevity signatures’ could be used as predictors of new lifespan-extending interventions at the gene expression level. We examined this possibility with two approaches. First, we checked if the signatures can be used to predict potential association of interventions of interest with the longevity gene expression response using publicly available datasets. Second, we tested their capability to predict new candidates for lifespan extension using the Connectivity Map (CMap) platform (Lamb et al., 2006; Subramanian et al., 2017).

For the first study, we preprocessed 6 publicly available datasets on hepatic gene expression in response to certain in vivo interventions in mouse models, including injection of interleukin 6 (IL-6) (Ramadoss et al., 2010), knockout of methionine adenosyltransferase gene (Mat1a) (Alonso et al., 2017), hypoxia conditions (Baze et al., 2010), knockout of Keap1 coding for an inhibitor of acute stress regulator NRF2 (Osburn et al., 2008), supplementation of SIRT1 activator SRT2104 (Mercken et al., 2014b) and overexpression of the sirtuin gene Sirt6 (Kanfi et al., 2012). We then ran a GSEA-based association test using longevity signatures as input subsets (FIG. 7E).

Interleukin-6 (IL-6) is one of the best studied pro-inflammatory cytokines secreted by T cells and macrophages to support the immune response. It was shown to stimulate the inflammatory and auto-immune response during progression of diseases, including diabetes (Kristiansen and Mandrup-Poulsen, 2005), Alzheimer's disease (Swardfager et al., 2010), multiple myeloma (Gadó et al., 2000) and others. Moreover, IL-6 was shown to induce insulin resistance directly by inhibiting insulin receptor signal transduction (Senn et al., 2002). Finally, functions related to liver regulation of the immune response stimulated by IL-6 were enriched for genes both commonly downregulated and negatively associated with the lifespan extension effect of longevity interventions. We tested if the intraperitoneal injection of interleukin-6 into mouse bloodstream leads to hepatic gene expression changes associated with longevity signatures and detected a significant negative association with all longevity signatures (BH adjusted p-value <0.025) (FIG. 7E), pointing to a potential negative effect of IL-6 on mouse lifespan.

Methionine adenosyltransferase 1A (Matta) is an enzyme that catalyzes conversion of methionine to S-adenosylmethionine. This gene plays a crucial role in methionine and glutathione metabolism. Its activity in liver is increased 205% in Ames dwarf mice compared to wild-type animals (Uthus and Brown-Borg, 2003), and the introduction of GH to these mice led to ˜40% decrease in MAT activity in liver (Brown-Borg et al., 2005). Moreover, due to the role of MAT in methionine metabolism, MAT deficiency in liver leads to persistent hypermethioninemia (Ubagai et al., 1995), which can be thought of as the opposite of MR. Therefore, we expected that knockout of Mat1a could be negatively associated with longevity signatures. Indeed, the test for longevity association revealed a negative association of this intervention with 4 out of 6 longevity signatures, the exceptions being GH deficiency and median lifespan effect signatures (BH adjusted p-value <0.02) (FIG. 7E). Therefore, Mat1a knockout leads to the changes in gene expression opposite to those caused by longevity signatures and is expected to diminish mouse longevity.

Hypoxia, a reduction in oxygen levels, has suggestive associations with longevity that are not yet well understood. First, aging is associated with hypoxia, e.g. showing 38% reduction in oxygen levels in adipose tissue (Zhang et al., 2011). Second, studies investigating the effect of hypoxia on longevity show contrasting results. Thus, one group showed that, in C. elegans, growth in low oxygen and mutation of VHL-1, a negative regulator of the main modulator of hypoxia HIF-1, extended worm lifespan up to 40% (Mehta et al., 2009). However, another group reported an increased lifespan in C. elegans following the deletion of HIF-1 gene under slightly different conditions (Chen et al., 2009). Also, by generating reactive oxygen species (ROS), hypoxia leads to activation of NRF2, one of the upstream regulators associated with the response to lifespan-extending interventions (FIG. 3C). Finally, hypoxia was found to be among the most effective protectors against mitochondrial disfunction associated with virtually all age-related degenerative diseases (Balaban et al., 2005; Jain et al., 2016). In mammals, chronic hypoxia leads not only to a compensatory increase in oxygen delivery due to increased production and affinity to hemoglobin, decreased weight, higher ventilation rate and capillary density and larger mass of lung, liver and left ventricle (Aaron and Powell, 1993; Baze et al., 2010), but also to a decrease in demand for oxygen through alterations in metabolism, including increased rate of anaerobic metabolism (glycolysis) along with decreased whole animal metabolic rate and body temperature (Gautier, 1996; Steiner and Branco, 2002). Therefore, we were particularly interested to investigate whether chronic hypoxia would affect hepatic gene expression in mice in ways that were correlated to lifespan gene expression signatures. We examined changes in gene expression in mice subjected to 11.5 kPa Poe hypoxia (11.8% oxygen in the air) for 32 days, and detected a significant positive association of hypoxia with all longevity signatures, except for rapamycin (BH adjusted p-value <0.034) (FIG. 7E), suggesting a potential positive effect of this intervention on mouse healthspan and/or lifespan.

NRF2 is one of the key acute stress regulators, which, among others, activates XMEs (Baird and Dinkova-Kostova, 2011) commonly upregulated at the level of hepatic gene expression across different lifespan-extending interventions (FIG. 5C). Overexpression of the NRF2 ortholog SKN-1 in C. elegans leads to a 5-20% increase in average lifespan (Tullet et al., 2008), whereas mutation of its inhibitor, Keap1, was shown to increase median lifespan by 8-10% in Drosophila melanogaster males (Sykiotis and Bohmann, 2008). Moreover, Protandim, a mixture of 5 botanical extracts known to stimulate Nrf2 activation, was proved to increase median lifespan in male mice by 7% (Strong et al., 2016). However, whether Nrf2 directly affects longevity of mammals remain unclear. We examined how hepatic gene expression is changed by hepatocyte-specific conditional knockout of Keap1 in mice and identified statistically significant positive association with almost all longevity signatures, except for rapamycin (BH adjusted p-value <0.0015) (FIG. 7E). This finding points to a potential positive effect of NRF2 activation on mouse healthspan and lifespan.

We also analyzed the association of sirtuin activation with longevity signatures using two mouse models, SIRT1 activator SRT2104 in males (Mercken et al., 2014b) and Sirt6 overexpression in both sexes (Kanfi et al., 2012). Both of these models were shown to extend lifespan of males, but the effect was modest (˜10% increase in median and maximum lifespan). Accordingly, we detected significant positive associations of these models in males with CR and signatures shared by lifespan-extending interventions. However, there was no consistent positive association with longevity signatures associated with the quantitative effect of lifespan extension, and we even observed a weak negative association for one of them (FIG. 7E). Interestingly, in the case of Sirt6 overexpression in females, which did not result affect lifespan (Kanfi et al., 2012), there was no significant associations with the lifespan-extension signature (FIG. 7E).

To test if the longevity gene expression signatures may be translated across species, we analyzed their association with the hepatic response to CR in rhesus monkey (Macaca mulatta) males (Rhoads et al., 2018). We observed a strong significant association with the CR signature, pointing to the occurrence of the shared gene expression response to this intervention in mammals (FIG. 7E). However, we did not detect an association of CR in the monkey with either common longevity signatures or signatures associated with the degree of lifespan extension. This may point to a weaker longevity effect of CR in primates or to the differences in the signatures of lifespan extension across species. This may also be due to statistical issues related to a limited sampling size. An extensive analysis of the primate response to lifespan-extending interventions may shed the light on this problem.

Finally, we tested if longevity signatures could be used to predict the difference in lifespan between different mouse strains, which may also be considered as genetic interventions. The GSE10421 dataset includes gene expression of for livers of male mice of 2 mouse strains tested at the same chronological age (7 weeks old): C57BL/6 and DBA/2 (Kautz et al., 2008). We ran a statistical model testing for genes with significant difference between these strains and subjected them to the longevity association test. All longevity signatures except for rapamycin showed a significant positive association with C57BL/6 gene expression profile compared to that of DBA/2 (BH adjusted p-value <5.3·10⁻⁴) (FIG. 7E). Lifespan of C57BL/6 mice (median lifespan=901 days) is significantly higher than that of DBA/2 (median lifespan=701 days) (Yuan et al., 2009). This difference was, therefore, captured by the longevity signatures, which were able to identify the strain with greater lifespan. These findings further support the notion that the longevity signatures can be used for the assessment of differences in expected lifespan.

For the second study, to test if such approach may be used for the identification of new lifespan-extending drugs, we utilized the CMap platform developed by the Broad Institute (Lamb et al., 2006; Subramanian et al., 2017). This platform contains gene expression profiles of different human cell lines, subjected to more than 1,500 chemical compounds, and allows searching for perturbagens producing gene expression changes similar to the genetic signature of interest. To identify drugs with significant longevity effects, we ranked them based on their association with the maximum lifespan signature. We then chose four compounds from the top of the ranking, prepared diets with them and applied these diets to UM-HET3 male mice for 1 month. These drugs included two mTOR inhibitors KU-0063794 (García-Martínez et al., 2009) and AZD-8055 (Chresta et al., 2010), antioxidant ascorbyl-palmitate (Cort, 1974) and antihypertensive agent rilmenidine (Mpoy et al., 1988).

We performed RNAseq on the liver samples of mice subjected to the drugs, together with the corresponding controls. To check if the hits predicted based on human cell lines are reproduced in mouse tissues, we calculated a gene expression response to each of these drugs and ran an association test as described earlier (FIG. 7E). In agreement with the predictions, all compounds demonstrated positive associations with the common gene signature across lifespan-extending interventions. Moreover, KU-0063794 and ascorbyl-palmitate demonstrated a consistent positive association with all lifespan-extending interventions, except for rapamycin (BH adjusted p-value <0.08 and <0.097 for KU-0063794 and ascorbyl-palmitate, respectively). AZD-8055 and rilmenidine showed a positive association with some of the signatures, including CR and GH deficiency, but not with the signatures associated with the lifespan extension effect. This inconsistency may be explained by imperfect translation of gene expression responses from human cell lines to mouse in vivo models or due to the insufficient sampling size. In general, however, this pilot study demonstrates the applicability of such approach for the identification of new interventions with a desirable effect on gene expression and identifies appealing candidates for further studies. A more extensive analysis of longevity-associated features in mouse models will be of high interest.

### Example 17

### Identification of a Turnover-Based Longevity Signature

We identified genes whose expression correlated with cell and tissue turnover. Available turnover times fora number of tissues and cell types (in days) were supplemented with estimates from the literature and used as a bona fide measure of lifespan (‘lifespan trait’). We applied generalized least squares regression, tested different evolutionary models and selected the best fit model by maximum likelihood.

Two hundred eight out of 12,044 genes showed significant correlation with turnover at a false discovery rate (Q-value) of 0.05, with 75% (155 genes, including those shown in Table 17) in negative correlation and 25% (53 genes, including those shown in Table 7) in positive correlation. Notable genes with a positive correlation included the complex SNRPN-SNURF locus, which gives rise to a number of proteins and short non-coding RNAs. We visualized the protein—protein interaction network represented by these 208 genes, revealing significant enrichment for genes involved in cell cycle, immune signaling (NF-κB) and p53 signaling. In our data set, hematopoietic tissues (bone marrow and spleen) and monocytes constituted the samples with the shortest turnover. Removal of these data points in the regression analysis retained the ‘turnover signature’, with the overlapping gene set comprising critical cell cycle and apoptosis associated genes, such as CHEK1, CHEK2, MKI67, FOXM1, TP53 and BCL10, while a correlation with immune signaling-associated genes was lost.

The procedures used to determine the turnover-based longevity signatures are described in Seim et al., Aging and Mechanisms of Disease 2:16014 (2016), the disclosure of which is incorporated herein by reference in its entirety.

### Example 18

### Identification of Organ-Specific Longevity Signatures by Analysis of Gene Expression Profiles Across Various Species

An analysis of gene expression divergence was carried out on 41 species of mammals having different lifespans, including terrestrial mammals of young adult age belonging to Euungulata (n=4), Carnivora (n=4), Chiroptera (n=2), Didelphimorphia (n=1), Diprotodoncia (n=1), Erinaceomorpha (n=1), Lagomorpha (n=1), Monotremata (n=1), Primate (n=8), Rodentia (n=9) and Soricomorpha (n=1) lineages. The total divergence of examined lineages corresponded to a period of about 160 million years. Evolution of these mammals yielded widespread variation in life histories, such as time to maturity, maximum lifespan and oxygen consumption (as a measure of basal metabolic rate, BMR). The relationship between these life histories defines a set of lineage-specific functional tradeoffs and adaptive investments developed during environmental specialization. For example, most primates are characterized by longevity, slow growth and reduced BMR, whereas muroid species (Eumuroida) often use opportunistic-type strategies characterized by rapid development and growth, low body mass and short lifespan. Moreover, some organisms such as representatives of Chiroptera and Histriocognathi, feature Eumuroida-sized species, but possess life history attributes of larger, longer-lived mammals.

Gene expression in three organs (i.e., liver (Tables 10 and 20), kidney (Tables 9 and 19) and brain (Tables 8 and 18)) was analyzed because of their easier availability, dominance of one cell type (e.g., liver), difference in metabolic functions, size of organs (which is a limitation for smaller animals) and compatibility with previous data from other labs. The majority of the examined species was represented by duplicated (52-60% of species) or triplicated (30-42% of species) biological replicates to account for within species gene expression variation. 25-60 million of 51-bp paired-and RNA-seq reads for each biological replicate were generated (data not shown).

Reads were then mapped to genomic sequences of organisms from Ensembl and NCBI databases. Database gene model annotations were used and 1-1 orthologous sequence relationships for these organisms were precomputed to calculate gene expression values defined as fragments per kilobase of transcript per million RNA-seq reads mapped (FPKM). Depending on species, RNA-seq read alignment efficiency varied from 55-99% (data not shown). For 12 species with no available genome sequences, full-length transcriptomic contigs using RNA-seq reads were de novo assembled (data not shown), encoded peptides were ab initio predicted (data not shown), and orthologous relationships with database proteins were inferred. Analyses on the expression of protein coding genes with a 1:1 orthologous relationship were further focused, derived from the dataset of 19,643 unique groups of sequences (data not shown).

In arriving at the gene signatures set forth in Tables 8-10 (up-regulated genes in long-living mammals) and Tables 18-20 (down-regulated genes in long-living mammals), the most relevant genes and biological pathways associated with life histories were examined. Gene set enrichment analysis revealed statistically significant label overrepresentation in the central energy metabolism combining numerous associated pathways such as pyruvate metabolism, carbohydrate degradation pathways, catabolism of tryptophan, lysine and valine oxidation and biosynthesis of fatty acids, Ppar, peroxisome, Ampk, growth hormone signaling and others. Interestingly, divergent evolution of marine vertebrates led to adaptive variation in growth and lifespan (St-Cyr et al., 2008) associated with expression signatures closely related to those observed in the studied mammals, indicating fundamental relatedness of strategies governing parallel life history and transcriptome evolution in vertebrates.

The full set of procedures used to determine the organ-specific longevity signatures set forth in Tables 8-10 and 18-20 are described in US 2016/0333407, the disclosure of which is incorporated herein by reference in its entirety.

### Example 19

### Listing of Intervention-Based Longevity Signatures, Turnover-Based Longevity Signature, and Organ-Specific Longevity Signatures

The various intervention-based longevity signatures, turnover-based longevity signature, and organ-specific longevity signatures described herein are listed in Tables 1-20, below.

TABLE 1
Intervention-based gene signature 1 (Calorie
restriction, up-regulated genes)

| Entry No. | Gene |
|---|---|
| 1 | Fmo3 |
| 2 | Gm4477 |
| 3 | Slc22a27 |
| 4 | Gm14420 |
| 5 | Acmsd |
| 6 | Slc22a29 |
| 7 | Eif4ebp3 |
| 8 | Nrg4 |
| 9 | Ctgf |
| 10 | Cyp39a1 |
| 11 | Orm2 |
| 12 | Per1 |
| 13 | Fmo2 |
| 14 | Por |
| 15 | Slc51b |
| 16 | Slco1a4 |
| 17 | Etnppl |
| 18 | Coq10b |
| 19 | Pde6c |
| 20 | Cyp2c39 |
| 21 | Akr1c19 |
| 22 | Abcc4 |
| 23 | Mthfd1l |
| 24 | Per2 |
| 25 | Rdh9 |
| 26 | Zbtb16 |
| 27 | Tef |
| 28 | Cyp2a4 |
| 29 | Cox7a1 |
| 30 | Rorc |
| 31 | Gstt2 |
| 32 | Cbr1 |
| 33 | Igfbp1 |
| 34 | Gde1 |
| 35 | Mgst3 |
| 36 | Txnip |
| 37 | Igfbp2 |
| 38 | Tbc1d8 |
| 39 | Akr1b7 |
| 40 | Cdkn1c |
| 41 | Aldoc |
| 42 | Idh2 |
| 43 | Gas2l3 |
| 44 | Gsta4 |
| 45 | Steap2 |
| 46 | Gstt3 |
| 47 | E130012A19Rik |
| 48 | Rnf145 |
| 49 | Nampt |
| 50 | Fam84b |
| 51 | Tsc22d3 |
| 52 | Mdh2 |
| 53 | Slc37a4 |
| 54 | Map2k6 |
| 55 | Cnst |
| 56 | Irs2 |
| 57 | Ces1b |
| 58 | Hspa2 |
| 59 | Gm5621 |
| 60 | Lonrf1 |
| 61 | Zpr1 |
| 62 | Fmo5 |
| 63 | Enpp1 |
| 64 | Dynll1 |
| 65 | D630033O11Rik |
| 66 | Lrp4 |
| 67 | Crym |
| 68 | Mknk2 |
| 69 | Tat |
| 70 | Lpin2 |
| 71 | Otud1 |
| 72 | Ppl |
| 73 | Morc3 |
| 74 | Rbm3 |
| 75 | BC023829 |
| 76 | Sall1 |
| 77 | Ccbl2 |
| 78 | Bmper |
| 79 | Ripk4 |
| 80 | Stard5 |
| 81 | Pls1 |
| 82 | Glrx |
| 83 | Ldhb |
| 84 | Nudt19 |
| 85 | Fmo4 |
| 86 | Ndel1 |
| 87 | Nhlrc2 |
| 88 | Cry2 |
| 89 | Acy1 |
| 90 | Lmo4 |
| 91 | Itpr1 |
| 92 | Adamts7 |
| 93 | Esrra |
| 94 | Vldlr |
| 95 | Etnk2 |
| 96 | Asl |
| 97 | Marveld3 |
| 98 | Klhl3 |
| 99 | Hspa9 |
| 100 | Pdk1 |
| 101 | 0610031O16Rik |
| 102 | Baiap2l1 |
| 103 | Tk1 |
| 104 | Gmnn |
| 105 | Dnajb6 |
| 106 | Car2 |
| 107 | Pck1 |
| 108 | Gab1 |
| 109 | Sestd1 |
| 110 | Cnn3 |
| 111 | Dctpp1 |
| 112 | Tm4sf4 |
| 113 | Scarf1 |
| 114 | Rev1 |
| 115 | Chchd7 |
| 116 | Slc15a4 |
| 117 | Hmgn5 |
| 118 | Cd163 |
| 119 | Man2a1 |
| 120 | Sult1a1 |
| 121 | Rpl22l1 |
| 122 | Rps9 |
| 123 | Slc6a8 |
| 124 | Timm8a1 |
| 125 | Fam73a |
| 126 | 2410131K14Rik |

TABLE 2
Intervention-based gene signature 2 (Growth hormone
deficient mutants, up-regulated genes)

| Entry No. | Gene |
|---|---|
| 1 | Sult1e1 |
| 2 | Cyp2b13 |
| 3 | Spink1 |
| 4 | Hao2 |
| 5 | Krt23 |
| 6 | Lrtm2 |
| 7 | Cyp4a14 |
| 8 | Cyp39a1 |
| 9 | Igfbp1 |
| 10 | Cyp2b9 |
| 11 | Atp6v0d2 |
| 12 | Ppp1r3g |
| 13 | Pcp4l1 |
| 14 | Serpina7 |
| 15 | Chil1 |
| 16 | Scd2 |
| 17 | Vldlr |
| 18 | Abcc4 |
| 19 | Lpl |
| 20 | Abcd2 |
| 21 | Pde6c |
| 22 | 5330417C22Rik |
| 23 | BC089597 |
| 24 | Rcan2 |
| 25 | Robo1 |
| 26 | Slc16a5 |
| 27 | Cyp2b10 |
| 28 | Fam126a |
| 29 | Pls1 |
| 30 | Defb1 |
| 31 | Abcb1a |
| 32 | Gstt3 |
| 33 | Nr4a1 |
| 34 | Col4a5 |
| 35 | Tceal8 |
| 36 | 8430408G22Rik |
| 37 | Lgals1 |
| 38 | Slco1a4 |
| 39 | Cyp4a31 |
| 40 | Slc16a7 |
| 41 | Il1m |
| 42 | Parp16 |
| 43 | Pparg |
| 44 | Aldh1b1 |
| 45 | Adora1 |
| 46 | Orm2 |
| 47 | Igfbp2 |
| 48 | Gsta2 |
| 49 | Usp18 |
| 50 | Cables1 |
| 51 | Adssl1 |
| 52 | Serpina6 |
| 53 | Ppargc1a |
| 54 | Tmem237 |
| 55 | Rnf145 |
| 56 | Cdkn1c |
| 57 | Cth |
| 58 | Crym |
| 59 | Tstd1 |
| 60 | Cxcl1 |
| 61 | Hexb |
| 62 | Tmtc2 |
| 63 | Cd83 |
| 64 | Idh2 |
| 65 | Gstm3 |
| 66 | Gsta4 |
| 67 | Card10 |
| 68 | 1810046K07Rik |
| 69 | Sh2d4a |
| 70 | Cpe |
| 71 | Dclre1a |
| 72 | Tcea3 |
| 73 | Cdpf1 |
| 74 | Ldhb |
| 75 | Mfsd7c |
| 76 | Rdh9 |
| 77 | Vnn3 |
| 78 | Pla2g12a |
| 79 | Tenm3 |
| 80 | As3mt |
| 81 | Gbp2 |
| 82 | Arrdc4 |
| 83 | Gadd45b |
| 84 | Mycl |
| 85 | Nqo1 |
| 86 | Arhgap18 |
| 87 | Ldlrad3 |
| 88 | Wee1 |
| 89 | Dqx1 |
| 90 | Rab30 |
| 91 | Smpd3 |
| 92 | Rbp1 |
| 93 | Enpp2 |
| 94 | Tmem98 |
| 95 | Slc25a48 |
| 96 | Rhbg |
| 97 | Slc15a4 |
| 98 | Mtmr11 |
| 99 | Dusp6 |
| 100 | Pigp |
| 101 | Sult1a1 |
| 102 | Btg2 |
| 103 | Meis1 |
| 104 | Agt |
| 105 | Slc7a2 |
| 106 | Cox7a1 |
| 107 | Nhlrc2 |
| 108 | Afp |
| 109 | Echdc3 |
| 110 | Nudt19 |
| 111 | Rassf3 |
| 112 | Cers6 |
| 113 | Btg1 |
| 114 | Acad10 |
| 115 | Ugp2 |
| 116 | Lcn2 |
| 117 | Fam134b |
| 118 | Ropn1l |

TABLE 3
Intervention-based gene signature
3 (Rapamycin, up-regulated genes)

| Entry No. | Gene |
|---|---|
| 1 | 2700060E02Rik |
| 2 | Acsf3 |
| 3 | Adh1 |
| 4 | Alg2 |
| 5 | Alyref |
| 6 | Apopt1 |
| 7 | Arl14ep |
| 8 | Arpc3 |
| 9 | Arpp19 |
| 10 | Atp5c1 |
| 11 | Atp5j |
| 12 | Atp5s |
| 13 | Bola3 |
| 14 | Btbd1 |
| 15 | Btbd3 |
| 16 | Btf3l4 |
| 17 | Car14 |
| 18 | Cdc14b |
| 19 | Ces1f |
| 20 | Chtop |
| 21 | Churc1 |
| 22 | Cnbp |
| 23 | Ctnnd1 |
| 24 | Ctps |
| 25 | D630033O11Rik |
| 26 | Dctn6 |
| 27 | Ddhd1 |
| 28 | Dnttip1 |
| 29 | Dtymk |
| 30 | Echdc3 |
| 31 | Eif3l |
| 32 | Elac1 |
| 33 | Erlin1 |
| 34 | Erp44 |
| 35 | Extl2 |
| 36 | Fez2 |
| 37 | Frat2 |
| 38 | G6pc3 |
| 39 | Gm5621 |
| 40 | Gnai3 |
| 41 | Gnpnat1 |
| 42 | Hdac2 |
| 43 | Hdgfrp2 |
| 44 | Helb |
| 45 | Hnrnph3 |
| 46 | Hprt |
| 47 | Ier3ip1 |
| 48 | Ifrd1 |
| 49 | Ift52 |
| 50 | Igf1 |
| 51 | Igsf5 |
| 52 | Iscu |
| 53 | Isy1 |
| 54 | Kctd5 |
| 55 | Klkb1 |
| 56 | Lamc1 |
| 57 | Lasp1 |
| 58 | Lrrfip1 |
| 59 | Lum |
| 60 | Maob |
| 61 | Mapk1ip1 |
| 62 | Med6 |
| 63 | Med9 |
| 64 | Mgat4b |
| 65 | Mien1 |
| 66 | Mks1 |
| 67 | Mphosph6 |
| 68 | Mpp6 |
| 69 | Mrpl30 |
| 70 | Mrpl42 |
| 71 | Mrpl57 |
| 72 | Mrps16 |
| 73 | Mrps25 |
| 74 | Mrps27 |
| 75 | Mterf4 |
| 76 | Ndufa4 |
| 77 | Ndufaf7 |
| 78 | Ndufb3 |
| 79 | Nsun4 |
| 80 | Nucks1 |
| 81 | Nudt7 |
| 82 | Nudt9 |
| 83 | Nxt1 |
| 84 | Ola1 |
| 85 | Oma1 |
| 86 | Oxnad1 |
| 87 | Pdzd11 |
| 88 | Pkp2 |
| 89 | Polr2h |
| 90 | Ppp3cb |
| 91 | Ppp3r1 |
| 92 | Psmb7 |
| 93 | Rab4a |
| 94 | Rad23a |
| 95 | Rbm22 |
| 96 | Rdh7 |
| 97 | Rhoa |
| 98 | Rnaseh1 |
| 99 | Rnf7 |
| 100 | Rpl27 |
| 101 | Rpl36al |
| 102 | Rpl9 |
| 103 | Rps17 |
| 104 | Rrbp1 |
| 105 | Slc12a6 |
| 106 | Smim11 |
| 107 | Snrpg |
| 108 | Sod2 |
| 109 | Spop |
| 110 | Stxbp3 |
| 111 | Taf11 |
| 112 | Tmed10 |
| 113 | Tmem125 |
| 114 | Tmem216 |
| 115 | Trabd |
| 116 | Ttf2 |
| 117 | Txnl4a |
| 118 | Ube2d2a |
| 119 | Ufc1 |
| 120 | Ugt2b36 |
| 121 | Ugt3a1 |
| 122 | Utp11l |
| 123 | Vamp4 |
| 124 | Wwc1 |
| 125 | Xkr9 |
| 126 | Yipf4 |
| 127 | Zfp938 |

TABLE 4
Intervention-based gene signature 4 (Common
to all interventions, up-regulated genes)

| Entry No. | Gene |
|---|---|
| 1 | 1600020E01Rik |
| 2 | 1810030O07Rik |
| 3 | 2310010J17Rik |
| 4 | Aass |
| 5 | Acmsd |
| 6 | Actr6 |
| 7 | Adcy3 |
| 8 | Adrb2 |
| 9 | Agmo |
| 10 | Agpat9 |
| 11 | Akr1b7 |
| 12 | Arpc3 |
| 13 | B4galt6 |
| 14 | Bambi |
| 15 | Bbs2 |
| 16 | Bckdhb |
| 17 | Brap |
| 18 | Brca1 |
| 19 | Cblb |
| 20 | Ccdc152 |
| 21 | Cep78 |
| 22 | Cep97 |
| 23 | Cggbp1 |
| 24 | Chrac1 |
| 25 | Cluap1 |
| 26 | Cmklr1 |
| 27 | Cnn3 |
| 28 | Cnst |
| 29 | Cps1 |
| 30 | Crebrf |
| 31 | Cth |
| 32 | Cul4b |
| 33 | Cyp3a59 |
| 34 | Cyp4a14 |
| 35 | Dab2 |
| 36 | Dbt |
| 37 | Ddr1 |
| 38 | Dhrs11 |
| 39 | Dynlt1b |
| 40 | Ecscr |
| 41 | Efcab2 |
| 42 | Ehhadh |
| 43 | Epor |
| 44 | Erf |
| 45 | Exosc2 |
| 46 | F13b |
| 47 | Fam105a |
| 48 | Fam167b |
| 49 | Fbxw9 |
| 50 | Fhit |
| 51 | Fmo4 |
| 52 | Gab1 |
| 53 | Gch1 |
| 54 | Ggta1 |
| 55 | Gm10639 |
| 56 | Gm16124 |
| 57 | Gm29376 |
| 58 | Gm5621 |
| 59 | Gpc6 |
| 60 | Grb7 |
| 61 | Gsta4 |
| 62 | Gsto1 |
| 63 | Gstt2 |
| 64 | Gtf2a2 |
| 65 | Hacd2 |
| 66 | Hmgn5 |
| 67 | Hspa9 |
| 68 | Inpp5a |
| 69 | Kcnn2 |
| 70 | Kctd12b |
| 71 | Klf15 |
| 72 | Las1l |
| 73 | Ldlrad3 |
| 74 | Lrrc8c |
| 75 | Maoa |
| 76 | Maob |
| 77 | Map2k6 |
| 78 | Map3k15 |
| 79 | Map4k2 |
| 80 | Mat2b |
| 81 | Mdh2 |
| 82 | Med14 |
| 83 | Megf9 |
| 84 | Mertk |
| 85 | Mier1 |
| 86 | Mospd2 |
| 87 | Msr1 |
| 88 | Mtmr7 |
| 89 | ND4 |
| 90 | Ndc1 |
| 91 | Ndel1 |
| 92 | Ndufa12 |
| 93 | Net1 |
| 94 | Neurl3 |
| 95 | Nmnat1 |
| 96 | Npc1 |
| 97 | Npr3 |
| 98 | Nqo1 |
| 99 | Nsmce2 |
| 100 | Nsmce4a |
| 101 | Ocln |
| 102 | Orc6 |
| 103 | P2ry14 |
| 104 | Pck2 |
| 105 | Pde7a |
| 106 | Peli1 |
| 107 | Pgm1 |
| 108 | Phtf2 |
| 109 | Pigu |
| 110 | Pir |
| 111 | Plekhb2 |
| 112 | Plekhg3 |
| 113 | Plk3 |
| 114 | Postn |
| 115 | Ppic |
| 116 | Ppt1 |
| 117 | Prkag1 |
| 118 | Psmd9 |
| 119 | Qk |
| 120 | Qpct |
| 121 | Rab4a |
| 122 | Rab9 |
| 123 | Rbm22 |
| 124 | Rbm3 |
| 125 | Rdh16 |
| 126 | Rdh9 |
| 127 | Rilpl1 |
| 128 | Rnase4 |
| 129 | Rnaseh2b |
| 130 | Rpl10a |
| 131 | Rpusd1 |
| 132 | Rragc |
| 133 | Rsph3a |
| 134 | Sall1 |
| 135 | Sept8 |
| 136 | Sertad2 |
| 137 | Sestd1 |
| 138 | Sfxn2 |
| 139 | Sgk1 |
| 140 | Sgms1 |
| 141 | Slc15a4 |
| 142 | Slc25a36 |
| 143 | Slc2a2 |
| 144 | Slc35a5 |
| 145 | Slc51b |
| 146 | Smc4 |
| 147 | Snhg20 |
| 148 | Snx16 |
| 149 | Snx6 |
| 150 | Sorl1 |
| 151 | Sos2 |
| 152 | Stat3 |
| 153 | Susd2 |
| 154 | Tax1bp3 |
| 155 | Tfap4 |
| 156 | Timm8a1 |
| 157 | Tmem50a |
| 158 | Tob2 |
| 159 | Tpd52l1 |
| 160 | Trim24 |
| 161 | Txnrd1 |
| 162 | Xpot |
| 163 | Zfp429 |
| 164 | Zfp764 |
| 165 | Zfp938 |
| 166 | Zzz3 |

TABLE 5
Intervention-based gene signature 5 (Association
with maximum lifespan change, up-regulated genes)

| Entry No. | Gene |
|---|---|
| 1 | Fam19a2 |
| 2 | Sult2a7 |
| 3 | Ppp1r3g |
| 4 | AA465934 |
| 5 | Serpina7 |
| 6 | Slc16a5 |
| 7 | Lpl |
| 8 | Nipal1 |
| 9 | Robo1 |
| 10 | Sybu |
| 11 | Col4a5 |
| 12 | Gm26684 |
| 13 | Ildr2 |
| 14 | Clec4a1 |
| 15 | Fam126a |
| 16 | ND3 |
| 17 | Ldhb |
| 18 | Utp14b |
| 19 | Tstd1 |
| 20 | Ndrg1 |
| 21 | Cox7a1 |
| 22 | Mfsd7c |
| 23 | Rassf3 |
| 24 | 2810013P06Rik |
| 25 | Nqo1 |
| 26 | Igfbp2 |
| 27 | Gys2 |
| 28 | Il17rb |
| 29 | Hexa |
| 30 | Ldlrad3 |
| 31 | Pla2g16 |
| 32 | Chkb |
| 33 | Arrdc4 |
| 34 | 5730508B09Rik |
| 35 | Agpat9 |
| 36 | Tnfrsf12a |
| 37 | Ropn1l |
| 38 | Hsd17b1l |
| 39 | Hunk |
| 40 | Wee1 |
| 41 | Rcn2 |
| 42 | Gabarapl1 |
| 43 | Rpa2 |
| 44 | Fads6 |
| 45 | Cbln3 |
| 46 | Zfp820 |
| 47 | Sptssa |
| 48 | Slc16a10 |
| 49 | Gskip |
| 50 | Ppm1k |
| 51 | Mettl10 |
| 52 | Parp16 |
| 53 | Sardh |
| 54 | Tcea3 |
| 55 | Ddah2 |
| 56 | Tmem25 |
| 57 | Eci1 |
| 58 | Acsm3 |
| 59 | Dcxr |
| 60 | Dnajb2 |
| 61 | Polr3gl |
| 62 | ND6 |
| 63 | Galk1 |
| 64 | Ttc38 |
| 65 | Agxt2 |
| 66 | Nudt16 |
| 67 | Aldh3a2 |
| 68 | Neurl2 |
| 69 | Pgap3 |
| 70 | Slc25a4 |
| 71 | Megf9 |
| 72 | Haus1 |
| 73 | Macrod1 |
| 74 | Lgals4 |
| 75 | Spg21 |
| 76 | Clk1 |
| 77 | Zfp960 |
| 78 | Cpt2 |
| 79 | 5031425E22Rik |
| 80 | Siva1 |
| 81 | Pdk2 |
| 82 | Hook2 |
| 83 | Vegfb |
| 84 | Ppat |
| 85 | Immp2l |
| 86 | Postn |
| 87 | Acot7 |
| 88 | Apoc2 |
| 89 | Tceal8 |
| 90 | Osgep |
| 91 | Mcee |
| 92 | Dhtkd1 |
| 93 | Kctd12 |
| 94 | Hpd |
| 95 | Cyp4f15 |
| 96 | Ppp3cc |
| 97 | Mnat1 |
| 98 | P2ry14 |
| 99 | Fam167b |
| 100 | Adck3 |
| 101 | Gldc |
| 102 | Gpr108 |
| 103 | Fam195a |
| 104 | Spopl |
| 105 | Cbr4 |
| 106 | Hint3 |
| 107 | Cep85 |
| 108 | Acadm |
| 109 | Dtnbp1 |
| 110 | Aldh4a1 |
| 111 | Kansl3 |
| 112 | Mrpl42 |
| 113 | Ddit3 |
| 114 | Aaed1 |
| 115 | Cdip1 |
| 116 | Clasp2 |
| 117 | Akr1b10 |
| 118 | Lhfpl2 |
| 119 | Emc8 |
| 120 | Bfar |
| 121 | Mapkapk2 |
| 122 | Fam118a |
| 123 | Abcb6 |
| 124 | Ndufb3 |
| 125 | Cmtm8 |
| 126 | Rbks |
| 127 | Cul9 |
| 128 | Il17ra |
| 129 | 1190005I06Rik |
| 130 | Rbm15 |
| 131 | Slc25a34 |
| 132 | Tmem106b |
| 133 | Med20 |
| 134 | Catsper2 |
| 135 | Zfp7 |
| 136 | Fh1 |
| 137 | Ntan1 |
| 138 | Sbds |
| 139 | Gm5621 |
| 140 | Rab9 |
| 141 | Nmnat1 |
| 142 | Mospd2 |
| 143 | Orc6 |
| 144 | Cblb |
| 145 | Cnst |
| 146 | Crebrf |
| 147 | Maob |
| 148 | Ehhadh |
| 149 | Pir |
| 150 | Brca1 |
| 151 | Map2k6 |
| 152 | Cth |
| 153 | Ecscr |
| 154 | Adcy3 |
| 155 | Exosc2 |
| 156 | Cmklr1 |

TABLE 6
Intervention-based gene signature 6 (Association
with median lifespan change, up-regulated genes)

| Entry No. | Gene |
|---|---|
| 1 | Sult2a7 |
| 2 | Fam19a2 |
| 3 | AA465934 |
| 4 | Serpinb1a |
| 5 | Serpina7 |
| 6 | Col4a5 |
| 7 | Fst |
| 8 | Nipal1 |
| 9 | Cyp2b10 |
| 10 | Pls1 |
| 11 | Fam126a |
| 12 | Ldhb |
| 13 | Mfsd7c |
| 14 | Ndrg1 |
| 15 | Tstd1 |
| 16 | Nqo1 |
| 17 | Igfbp2 |
| 18 | Il17rb |
| 19 | As3mt |
| 20 | Ldlrad3 |
| 21 | C330018D20Rik |
| 22 | Gys2 |
| 23 | Arrdc4 |
| 24 | Parp16 |
| 25 | Rdh9 |
| 26 | Rassf3 |
| 27 | 2810013P06Rik |
| 28 | Rnf186 |
| 29 | Agpat9 |
| 30 | Hsd17b11 |
| 31 | Pla2g16 |
| 32 | Wwtr1 |
| 33 | Hexa |
| 34 | Chkb |
| 35 | Grtp1 |
| 36 | Sptssa |
| 37 | Slc1a4 |
| 38 | Fam151b |
| 39 | Ppm1k |
| 40 | ND6 |
| 41 | Vnn3 |
| 42 | Aldh1b1 |
| 43 | Slc25a4 |
| 44 | Aig1 |
| 45 | Ttc38 |
| 46 | Tsc22d4 |
| 47 | Postn |
| 48 | Cbln3 |
| 49 | Sult1a1 |
| 50 | Kctd12 |
| 51 | Tcea3 |
| 52 | Mettl10 |
| 53 | Ropn1l |
| 54 | Zfp820 |
| 55 | Megf9 |
| 56 | Rcn2 |
| 57 | Ugcg |
| 58 | 9330175E14Rik |
| 59 | H2-M3 |
| 60 | Aaed1 |
| 61 | Polr3gl |
| 62 | Nudt16 |
| 63 | Clk1 |
| 64 | Rpa2 |
| 65 | Agxt2 |
| 66 | Ppat |
| 67 | Macrod1 |
| 68 | Gls2 |
| 69 | Eci1 |
| 70 | Gpr108 |
| 71 | Haus1 |
| 72 | Rell1 |
| 73 | Mcee |
| 74 | Tmem106b |
| 75 | Tmem25 |
| 76 | Vldlr |
| 77 | Spopl |
| 78 | Rnf170 |
| 79 | Acadm |
| 80 | Cyp4f15 |
| 81 | Adcy6 |
| 82 | Ppp3cc |
| 83 | Spg21 |
| 84 | Zfp960 |
| 85 | Ntan1 |
| 86 | P2ry14 |
| 87 | Pgap3 |
| 88 | Acot7 |
| 89 | Mnat1 |
| 90 | Aldh4a1 |
| 91 | Tnnc1 |
| 92 | Cbr4 |
| 93 | Spred1 |
| 94 | Vbp1 |
| 95 | Cdip1 |
| 96 | Adck3 |
| 97 | Khdrbs3 |
| 98 | Zbtb44 |
| 99 | Acsm3 |
| 100 | Dcxr |
| 101 | Crcp |
| 102 | Immp2l |
| 103 | Tmem123 |
| 104 | Kansl3 |
| 105 | Nrros |
| 106 | Ube2m |
| 107 | Anpep |
| 108 | Abcg1 |
| 109 | Dnajb2 |
| 110 | Fam118a |
| 111 | Plekhm3 |
| 112 | Vamp2 |
| 113 | Il16 |
| 114 | Ndufab1 |
| 115 | Sgms1 |
| 116 | Crym |
| 117 | Far1 |
| 118 | Ptpn6 |
| 119 | Dcbld1 |
| 120 | H2afv |
| 121 | Hint3 |
| 122 | Sh3bp5l |
| 123 | Lyrm5 |
| 124 | Gnpnat1 |
| 125 | Ctnnd1 |
| 126 | Pitrm1 |
| 127 | Akr1b10 |
| 128 | Marcks |
| 129 | Ppa1 |
| 130 | Kdsr |
| 131 | Slc22a18 |
| 132 | Spg7 |
| 133 | Mtap |
| 134 | Tmem53 |
| 135 | Polk |
| 136 | Mmachc |
| 137 | Ctsf |
| 138 | Nhlrc2 |
| 139 | Gins1 |
| 140 | Rbm26 |
| 141 | Pgm1 |
| 142 | Zfp36l2 |
| 143 | Zfp961 |
| 144 | Gm5621 |
| 145 | Rab9 |
| 146 | Nmnat1 |
| 147 | Mospd2 |
| 148 | Orc6 |
| 149 | Cblb |
| 150 | Cnst |
| 151 | Crebrf |
| 152 | Maob |
| 153 | Ehhadh |
| 154 | Pir |
| 155 | Brca1 |
| 156 | Map2k6 |
| 157 | Cth |
| 158 | Ecscr |
| 159 | Adcy3 |
| 160 | Exosc2 |
| 161 | Cmklr1 |

TABLE 7
Cell turnover-based gene signature (up-regulated genes)

| Entry No. | Gene |
|---|---|
| 1 | Ankrd34a |
| 2 | Bcl6b |
| 3 | Ccdc92 |
| 4 | Celf2 |
| 5 | Creld1 |
| 6 | Cry2 |
| 7 | Eif5b |
| 8 | Hey1 |
| 9 | Pcdh9 |
| 10 | Plekhg1 |
| 11 | Shank3 |
| 12 | Snrpn |
| 13 | Stoml1 |
| 14 | Ttyh2 |
| 15 | Zbtb46 |

TABLE 8
Organ-specific gene signature 1 (Brain, up-regulated genes)

| Entry No. | Gene |
|---|---|
| 1 | Arl2 |
| 2 | Arrb2 |
| 3 | Atf3 |
| 4 | AU022252 |
| 5 | Bahcc1 |
| 6 | Bcap31 |
| 7 | Cdk5rap1 |
| 8 | Chrne |
| 9 | Ciita |
| 10 | Cndp2 |
| 11 | Creb5 |
| 12 | Cxcr4 |
| 13 | Cyr61 |
| 14 | Ddx41 |
| 15 | Dennd3 |
| 16 | Dysf |
| 17 | Efna1 |
| 18 | Eif2b5 |
| 19 | Erh |
| 20 | Esyt1 |
| 21 | Fam178b |
| 22 | Fam195b |
| 23 | Fam229a |
| 24 | Fer1l4 |
| 25 | Fignl1 |
| 26 | Fxyd6 |
| 27 | Gchfr |
| 28 | Gpr179 |
| 29 | Gsap |
| 30 | Hagh |
| 31 | Higd1a |
| 32 | Hnrnpdl |
| 33 | Lamtor5 |
| 34 | Lgals3 |
| 35 | Litaf |
| 36 | Mapk12 |
| 37 | Mbd4 |
| 38 | Metrnl |
| 39 | Morn2 |
| 40 | Mrps11 |
| 41 | Mst1 |
| 42 | Myom2 |
| 43 | Narfl |
| 44 | Nfkbia |
| 45 | Nlrc5 |
| 46 | Npm2 |
| 47 | Parp1 |
| 48 | Pcsk7 |
| 49 | Pex10 |
| 50 | Pfkfb3 |
| 51 | Phf1 |
| 52 | Plac8l1 |
| 53 | Plcb2 |
| 54 | Prdx3 |
| 55 | Prx |
| 56 | Psmf1 |
| 57 | Psmg3 |
| 58 | Qrich2 |
| 59 | Rabl6 |
| 60 | Rhbdl2 |
| 61 | Slain1 |
| 62 | Slc44a3 |
| 63 | Srp14 |
| 64 | Stag3 |
| 65 | Syngr2 |
| 66 | Tagln |
| 67 | Tap1 |
| 68 | Timm50 |
| 69 | Tmem14c |
| 70 | Top3a |
| 71 | Tssk3 |
| 72 | Txnip |
| 73 | Ung |
| 74 | Zfp36 |

TABLE 9
Organ-specific gene signature 2 (Kidney, up-regulated genes)

| Entry No. | Gene |
|---|---|
| 1 | 2510039O18Rik |
| 2 | Ackr1 |
| 3 | Alkbh7 |
| 4 | Apex1 |
| 5 | Apitd1 |
| 6 | Arl2 |
| 7 | B4galt7 |
| 8 | Bcap31 |
| 9 | Capg |
| 10 | Ccdc50 |
| 11 | Cct7 |
| 12 | Cel |
| 13 | Ciz1 |
| 14 | Clip3 |
| 15 | Cln3 |
| 16 | Cnp |
| 17 | Cpsf31 |
| 18 | Csrp1 |
| 19 | Cyr61 |
| 20 | Dbndd1 |
| 21 | Dgcr14 |
| 22 | Eef1g |
| 23 | Efna1 |
| 24 | Eif3k |
| 25 | Eif4b |
| 26 | Fam195b |
| 27 | Fis1 |
| 28 | Finc |
| 29 | Flot1 |
| 30 | Fosl2 |
| 31 | Fus |
| 32 | Gltscr2 |
| 33 | Gnptg |
| 34 | Gypc |
| 35 | Hdac10 |
| 36 | Hmg20b |
| 37 | Hnrnpd |
| 38 | Hnrnpdl |
| 39 | Hoxa5 |
| 40 | Iqck |
| 41 | Itga5 |
| 42 | Krt18 |
| 43 | Larp6 |
| 44 | Lgi2 |
| 45 | LOC100862468 |
| 46 | Lsm3 |
| 47 | Map6d1 |
| 48 | Meiob |
| 49 | Mrps15 |
| 50 | Naca |
| 51 | Necab3 |
| 52 | Nol4l |
| 53 | Nop56 |
| 54 | Nr2f1 |
| 55 | Nsl1 |
| 56 | P4htm |
| 57 | Pcbp2 |
| 58 | Pcgf2 |
| 59 | Pdlim1 |
| 60 | Plcb2 |
| 61 | Psmf1 |
| 62 | Rnase1 |
| 63 | Rpa2 |
| 64 | Rpl22 |
| 65 | Rpl28 |
| 66 | Rpl30 |
| 67 | Rpl37 |
| 68 | Rps9 |
| 69 | Rtfdc1 |
| 70 | Sdc1 |
| 71 | Sh3kbp1 |
| 72 | Skap1 |
| 73 | Slc41a3 |
| 74 | Slx1b |
| 75 | Smarcb1 |
| 76 | Smyd3 |
| 77 | Spata20 |
| 78 | Ssbp3 |
| 79 | Styxl1 |
| 80 | Tbc1d8 |
| 81 | Tppp3 |
| 82 | Trub2 |
| 83 | Ttc21a |
| 84 | Tub |
| 85 | Tubgcp6 |
| 86 | Vps28 |
| 87 | Wash1 |
| 88 | Wdr13 |
| 89 | Yipf3 |

TABLE 10
Organ-specific gene signature 3 (Liver, up-regulated genes)

| Entry No. | Gene |
|---|---|
| 1 | 2510039O18Rik |
| 2 | 6820408C15Rik |
| 3 | Afap1l2 |
| 4 | Agbl2 |
| 5 | Akap12 |
| 6 | Arhgap15 |
| 7 | Arhgdib |
| 8 | Basp1 |
| 9 | Blvrb |
| 10 | Bok |
| 11 | Cdh23 |
| 12 | Cep112 |
| 13 | Cited2 |
| 14 | Col6a2 |
| 15 | Crispld2 |
| 16 | Ctgf |
| 17 | Cxcr4 |
| 18 | Cyba |
| 19 | Cytip |
| 20 | Ddx41 |
| 21 | Dkk3 |
| 22 | Eef1d |
| 23 | Eef1g |
| 24 | Egr1 |
| 25 | Eif3l |
| 26 | Erbb2 |
| 27 | Evc2 |
| 28 | Fam129b |
| 29 | Fam149a |
| 30 | Fanca |
| 31 | Fkbp11 |
| 32 | Flna |
| 33 | Fos |
| 34 | Fosl2 |
| 35 | Fus |
| 36 | Glipr2 |
| 37 | Gltscr2 |
| 38 | Gm7102 |
| 39 | Gnptg |
| 40 | H2afv |
| 41 | Hebp2 |
| 42 | Hmgb2 |
| 43 | Hnrnpd |
| 44 | Hnrnpdl |
| 45 | Jun |
| 46 | Junb |
| 47 | Krtcap2 |
| 48 | Larp6 |
| 49 | Ldb2 |
| 50 | Lgals1 |
| 51 | Lmod1 |
| 52 | Mbd4 |
| 53 | Mbp |
| 54 | Med4 |
| 55 | Mrps15 |
| 56 | Myc |
| 57 | Naa20 |
| 58 | Ncapd2 |
| 59 | Ncf2 |
| 60 | Nfkbia |
| 61 | Nr2f1 |
| 62 | Nsmce1 |
| 63 | Pdlim3 |
| 64 | Pfkp |
| 65 | Plekho2 |
| 66 | Ptprc |
| 67 | Ramp2 |
| 68 | Rasl11a |
| 69 | Relt |
| 70 | Rpa2 |
| 71 | Rpl10a |
| 72 | Rpl11 |
| 73 | Rpl22 |
| 74 | Rpl24 |
| 75 | Rpl28 |
| 76 | Rpl30 |
| 77 | Rpl35a |
| 78 | Rpl37 |
| 79 | Rpl38 |
| 80 | Rpl5 |
| 81 | Rpl6 |
| 82 | Rps15a |
| 83 | Rps17 |
| 84 | Rps19 |
| 85 | Slc27a3 |
| 86 | Slx1b |
| 87 | Stx11 |
| 88 | Styxl1 |
| 89 | Sumo2 |
| 90 | Tagln |
| 91 | Tagln2 |
| 92 | Thbs1 |
| 93 | Tm6sf2 |
| 94 | Trpv2 |
| 95 | Tubgcp6 |
| 96 | U2af1 |
| 97 | Wbscr22 |
| 98 | Wdr13 |

TABLE 11
Intervention-based gene signature 1 (Calorie
restriction, down-regulated genes)

| Entry No. | Gene |
|---|---|
| 1 | Mup14 |
| 2 | Ifit1 |
| 3 | Elovl3 |
| 4 | Serpina12 |
| 5 | Saa1 |
| 6 | Adh6-ps1 |
| 7 | Spon2 |
| 8 | C6 |
| 9 | Csrp3 |
| 10 | Hsd3b7 |
| 11 | Saa2 |
| 12 | C9 |
| 13 | Ifi47 |
| 14 | Irgm2 |
| 15 | Rsad2 |
| 16 | Igtp |
| 17 | Ugt3a1 |
| 18 | Paqr9 |
| 19 | Nudt7 |
| 20 | Fitm1 |
| 21 | Pdilt |
| 22 | Cyp7b1 |
| 23 | A230050P20Rik |
| 24 | Slc30a10 |
| 25 | Ifit3 |
| 26 | Slc22a7 |
| 27 | Cyp2u1 |
| 28 | Slc25a30 |
| 29 | Isg15 |
| 30 | Trim12c |
| 31 | Alas2 |
| 32 | Car3 |
| 33 | Klhdc7a |
| 34 | Insig2 |
| 35 | Arsg |
| 36 | Insc |
| 37 | Sult5a1 |
| 38 | Iigp1 |
| 39 | C8b |
| 40 | Gna14 |
| 41 | Oasl1 |
| 42 | Ly6a |
| 43 | 3010026O09Rik |
| 44 | Ifit3b |
| 45 | Sdr42e1 |
| 46 | Cmpk2 |
| 47 | Aox3 |
| 48 | Psmb9 |
| 49 | Cyp2d40 |
| 50 | Tlcd2 |
| 51 | Gdf15 |
| 52 | Chn1os3 |
| 53 | Adck5 |
| 54 | Irgm1 |
| 55 | Dhx58 |
| 56 | Srd5a1 |
| 57 | Mikl |
| 58 | Tsc22d1 |
| 59 | Hsd17b2 |
| 60 | Apon |
| 61 | Pctp |
| 62 | Dct |
| 63 | Tgtp1 |
| 64 | Plcxd2 |
| 65 | Eps8l2 |
| 66 | MbH |
| 67 | Exoc4 |
| 68 | Nsmf |
| 69 | Stat1 |
| 70 | Cyp8b1 |
| 71 | Irf9 |
| 72 | Ldhd |
| 73 | S100a10 |
| 74 | St3gal3 |
| 75 | Lgals3bp |
| 76 | Fam89a |
| 77 | Apol9a |
| 78 | Plin2 |
| 79 | Mx2 |
| 80 | Nudt1 |
| 81 | Lonp2 |
| 82 | Tmem19 |
| 83 | Parp14 |
| 84 | Necab1 |
| 85 | Slc15a3 |
| 86 | LOC102640772 |
| 87 | Smagp |
| 88 | Serpina10 |
| 89 | Kynu |
| 90 | Parp9 |
| 91 | Scamp5 |
| 92 | Lasp1 |
| 93 | Mapk15 |
| 94 | Gsdmd |
| 95 | Cyp2f2 |
| 96 | Zc3h12d |
| 97 | Nrp1 |
| 98 | Irf5 |
| 99 | St6gal1 |
| 100 | Dpp7 |
| 101 | Cldn2 |
| 102 | Acy3 |
| 103 | Cox19 |
| 104 | Bcl3 |
| 105 | Mocos |
| 106 | Fabp2 |
| 107 | Trim34a |
| 108 | C4bp |
| 109 | Fpgs |
| 110 | Cxcl10 |
| 111 | Acsf2 |
| 112 | Fam114a1 |
| 113 | Gbp7 |
| 114 | Glo1 |
| 115 | Ifi35 |
| 116 | Rab29 |
| 117 | Cmtm6 |
| 118 | Pdcd4 |
| 119 | Dock8 |
| 120 | Aox1 |
| 121 | 3830406C13Rik |
| 122 | Csf2rb |
| 123 | Sept9 |
| 124 | Tap1 |

TABLE 12
Intervention-based gene signature 2 (Growth hormone
deficient mutants, down-regulated genes)

| Entry No. | Gene |
|---|---|
| 1 | Hsd3b5 |
| 2 | Slco1a1 |
| 3 | Wfdc21 |
| 4 | Elovl3 |
| 5 | Igf1 |
| 6 | Serpina3k |
| 7 | C8a |
| 8 | Igfals |
| 9 | Det |
| 10 | Keg1 |
| 11 | Mup3 |
| 12 | Susd4 |
| 13 | Ppp1r14a |
| 14 | Serpina12 |
| 15 | Cyp7b1 |
| 16 | C6 |
| 17 | Scara5 |
| 18 | Dpy19l3 |
| 19 | Nudt7 |
| 20 | Csrp3 |
| 21 | Egfr |
| 22 | Ces3a |
| 23 | Srd5a1 |
| 24 | Crygn |
| 25 | Csad |
| 26 | C8b |
| 27 | Sdr9c7 |
| 28 | Dmrta1 |
| 29 | Ugt2b38 |
| 30 | Onecut1 |
| 31 | Socs2 |
| 32 | Nat8 |
| 33 | Cyp2u1 |
| 34 | Slc30a10 |
| 35 | F11 |
| 36 | Gna14 |
| 37 | Nrep |
| 38 | Pdilt |
| 39 | Slc10a2 |
| 40 | Npr2 |
| 41 | Cmah |
| 42 | Cyp4f14 |
| 43 | Fabp5 |
| 44 | Serpina11 |
| 45 | Neb |
| 46 | Zap70 |
| 47 | Cela1 |
| 48 | Sult2a8 |
| 49 | Fabp2 |
| 50 | Ablim3 |
| 51 | Derl3 |
| 52 | Apcs |
| 53 | Sdf2l1 |
| 54 | Gpc1 |
| 55 | Phlda1 |
| 56 | Celsr1 |
| 57 | C9 |
| 58 | Hsd17b2 |
| 59 | Cadm4 |
| 60 | Aox3 |
| 61 | Slc25a30 |
| 62 | Irf6 |
| 63 | E2f8 |
| 64 | Trp53inp2 |
| 65 | Lift |
| 66 | Alas2 |
| 67 | Pnpla7 |
| 68 | Bmyc |
| 69 | Sntg2 |
| 70 | Ugt2b1 |
| 71 | Hspb1 |
| 72 | Gadd45g |
| 73 | Ttc39c |
| 74 | Rapgef4 |
| 75 | Arhgap44 |
| 76 | Hsd3b2 |
| 77 | Me1 |
| 78 | Apoa4 |
| 79 | Iigp1 |
| 80 | A230050P20Rik |
| 81 | Cadps2 |
| 82 | Creld2 |
| 83 | Aacs |
| 84 | Ero1lb |
| 85 | Omd |
| 86 | Cish |
| 87 | Tmem19 |
| 88 | Tars |
| 89 | Hes6 |
| 90 | Ifi47 |
| 91 | Slc25a33 |
| 92 | Slc41a2 |
| 93 | Cfh |
| 94 | Lrg1 |
| 95 | Manf |
| 96 | Syvn1 |
| 97 | Enho |
| 98 | Inhbe |
| 99 | Rdh11 |
| 100 | Dpp7 |
| 101 | Prlr |
| 102 | Sipa1l3 |
| 103 | Slc22a30 |
| 104 | Ifi47 |
| 105 | Pdia6 |
| 106 | Errfi1 |
| 107 | Ccnf |
| 108 | Tnfaip8l1 |
| 109 | Ifi35 |
| 110 | Cyp2f2 |
| 111 | Plekhb1 |
| 112 | Zfand4 |
| 113 | Orm1 |
| 114 | D16Ertd472e |
| 115 | Mkx |
| 116 | Insc |
| 117 | Tspan33 |
| 118 | Al661453 |
| 119 | Mcm10 |
| 120 | Sdr42e1 |
| 121 | Sec11c |
| 122 | Hao1 |
| 123 | Acsm1 |
| 124 | Dnajb11 |
| 125 | Tnk2 |
| 126 | Slc34a2 |
| 127 | Ctsc |
| 128 | Aatk |
| 129 | Zfp445 |
| 130 | Dpy19l1 |
| 131 | Pms1 |
| 132 | Ldhd |

TABLE 13
Intervention-based gene signature
3 (Rapamycin, down-regulated genes)

| Entry No. | Gene |
|---|---|
| 1 | 2310033P09Rik |
| 2 | Abcb4 |
| 3 | Abcc3 |
| 4 | Abhd13 |
| 5 | Acnat2 |
| 6 | Adcy9 |
| 7 | Aqp1 |
| 8 | Arhgap23 |
| 9 | Arhgap30 |
| 10 | Atff7p |
| 11 | Atg2a |
| 12 | Atp13a1 |
| 13 | Baz1b |
| 14 | Cactin |
| 15 | Casp3 |
| 16 | Ccdc97 |
| 17 | Cdk13 |
| 18 | Col4a3bp |
| 19 | Coro1c |
| 20 | Cpn2 |
| 21 | Crim1 |
| 22 | Cyb561d2 |
| 23 | Cyp2b10 |
| 24 | Ddhd2 |
| 25 | Dkk3 |
| 26 | Dnajc14 |
| 27 | Dpy19l1 |
| 28 | Egln2 |
| 29 | Elmo3 |
| 30 | Emp2 |
| 31 | Endod1 |
| 32 | Esyt1 |
| 33 | Fam160a2 |
| 34 | Fbxo18 |
| 35 | Foxk2 |
| 36 | Frk |
| 37 | Gga2 |
| 38 | Hiatl1 |
| 39 | Hif1an |
| 40 | Irs2 |
| 41 | Iws1 |
| 42 | Kank3 |
| 43 | Keap1 |
| 44 | Klhl18 |
| 45 | Lrrk1 |
| 46 | Man2b2 |
| 47 | Mapk4 |
| 48 | Mb21d2 |
| 49 | Mybbp1a |
| 50 | Myo6 |
| 51 | Naa25 |
| 52 | Naa30 |
| 53 | Naip2 |
| 54 | Nek4 |
| 55 | Neurl4 |
| 56 | Nfic |
| 57 | Nhlrc3 |
| 58 | Nipal3 |
| 59 | Os9 |
| 60 | P2rx4 |
| 61 | Pear1 |
| 62 | Phactr4 |
| 63 | Pi4ka |
| 64 | Plekhm1 |
| 65 | Pofut2 |
| 66 | Prpf4 |
| 67 | Psmd2 |
| 68 | Ptpra |
| 69 | Rab3gap1 |
| 70 | Rapgef5 |
| 71 | Sart3 |
| 72 | Scarb2 |
| 73 | Selo |
| 74 | Serpinb6b |
| 75 | Sf3b3 |
| 76 | Slc1a2 |
| 77 | Slc35g1 |
| 78 | Slc6a8 |
| 79 | Slc7a5 |
| 80 | Snx8 |
| 81 | Stim1 |
| 82 | Tcf25 |
| 83 | Tfpi |
| 84 | Thsd1 |
| 85 | Tle4 |
| 86 | Tmem44 |
| 87 | Tnfaip1 |
| 88 | Tor1b |
| 89 | Tpp1 |
| 90 | Traf7 |
| 91 | Tstd2 |
| 92 | Tubgcp6 |
| 93 | Ubac1 |
| 94 | Vgll4 |
| 95 | Vstm4 |
| 96 | Wfdc2 |
| 97 | Wnt2 |
| 98 | Zbtb11 |
| 99 | Zfp58 |
| 100 | Zfyve27 |

TABLE 14
Intervention-based gene signature 4 (Common
to all interventions, down-regulated genes)

| Entry No. | Gene |
|---|---|
| 1 | 1110037F02Rik |
| 2 | 1600012H06Rik |
| 3 | 1700049G17Rik |
| 4 | 2610015P09Rik |
| 5 | 4933421O10Rik |
| 6 | 9130023H24Rik |
| 7 | Aasdh |
| 8 | Aatk |
| 9 | Acp5 |
| 10 | Adam17 |
| 11 | Adh6-ps1 |
| 12 | Alkbh8 |
| 13 | Apol9b |
| 14 | Arhgef12 |
| 15 | Bbx |
| 16 | BC017158 |
| 17 | Bcdin3d |
| 18 | Braf |
| 19 | C4bp |
| 20 | Ccnj |
| 21 | Ccpg1os |
| 22 | Cdk17 |
| 23 | Cenpj |
| 24 | Cfi |
| 25 | Cldn3 |
| 26 | Cmtr1 |
| 27 | Commd7 |
| 28 | Cpn2 |
| 29 | Cyb561 |
| 30 | Cyb561a3 |
| 31 | Cyb5d2 |
| 32 | Cyp4f17 |
| 33 | Cyp4v3 |
| 34 | Ddost |
| 35 | E2f8 |
| 36 | Ercc6 |
| 37 | Erp29 |
| 38 | Exoc1 |
| 39 | Exoc2 |
| 40 | Ext2 |
| 41 | Fabp1 |
| 42 | Fadd |
| 43 | Fam219b |
| 44 | Fem1a |
| 45 | Gatc |
| 46 | Gbp7 |
| 47 | Ghr |
| 48 | Gorasp1 |
| 49 | Gpat2 |
| 50 | Gpc4 |
| 51 | Gpr107 |
| 52 | Gtf3c1 |
| 53 | H2-T23 |
| 54 | Hc |
| 55 | Hipk2 |
| 56 | Hps4 |
| 57 | Ikbkap |
| 58 | Il6st |
| 59 | Irf3 |
| 60 | Itsn1 |
| 61 | Kbtbd3 |
| 62 | Kctd17 |
| 63 | Klhl18 |
| 64 | Larp1 |
| 65 | Litaf |
| 66 | Lmbr1 |
| 67 | Lmf1 |
| 68 | LOC102631757 |
| 69 | Lrrc14 |
| 70 | Lysmd4 |
| 71 | Malat1 |
| 72 | Mfsd3 |
| 73 | Mgmt |
| 74 | Mllt4 |
| 75 | Mrps18a |
| 76 | Mrs2 |
| 77 | Nbas |
| 78 | Nupl2 |
| 79 | Oasl1 |
| 80 | Onecut1 |
| 81 | Osbpl9 |
| 82 | Otud4 |
| 83 | Oxr1 |
| 84 | P4hb |
| 85 | Parp9 |
| 86 | Pdcd4 |
| 87 | Pdia6 |
| 88 | Pgpep1 |
| 89 | Pik3r4 |
| 90 | Pnn |
| 91 | Polb |
| 92 | Prelp |
| 93 | Proz |
| 94 | Psmb9 |
| 95 | Qprt |
| 96 | Rai14 |
| 97 | Rb1 |
| 98 | Rec114 |
| 99 | Rfwd2 |
| 100 | Rplp0 |
| 101 | Saa4 |
| 102 | Serpina10 |
| 103 | Slc25a30 |
| 104 | Smarcal1 |
| 105 | Snap47 |
| 106 | Snapc5 |
| 107 | Snx17 |
| 108 | Sox5 |
| 109 | Spsb4 |
| 110 | St3gal3 |
| 111 | Stk16 |
| 112 | Stk19 |
| 113 | Tap2 |
| 114 | Tbc1d5 |
| 115 | Tfr2 |
| 116 | Tlr5 |
| 117 | Tmem261 |
| 118 | Tmem8b |
| 119 | Trmt5 |
| 120 | Ttc30b |
| 121 | Ttc41 |
| 122 | Tuft1 |
| 123 | Ube3a |
| 124 | Vcpip1 |
| 125 | Vps18 |
| 126 | Vwa5a |
| 127 | Wbp1 |
| 128 | Wfdc2 |
| 129 | Zfp507 |
| 130 | Zfp595 |
| 131 | Zfp729b |
| 132 | Zkscan7 |
| 133 | Zmym2 |
| 134 | Znfx1 |

TABLE 15
Intervention-based gene signature 5 (Association with
maximum lifespan change, down-regulated genes)

| Entry No. | Gene |
|---|---|
| 1 | Mup16 |
| 2 | Mup19 |
| 3 | Mup15 |
| 4 | Mup14 |
| 5 | Mup11 |
| 6 | Nuggc |
| 7 | Ugt2b37 |
| 8 | Tnik |
| 9 | Mfhas1 |
| 10 | Npr2 |
| 11 | B4galnt3 |
| 12 | Srd5a1 |
| 13 | Apcs |
| 14 | Slc3a1 |
| 15 | Gm17296 |
| 16 | Lrp2bp |
| 17 | Cmah |
| 18 | Sort1 |
| 19 | Piezo1 |
| 20 | Ablim3 |
| 21 | Cml2 |
| 22 | Errfi1 |
| 23 | Ston1 |
| 24 | Nup210 |
| 25 | Zbtb20 |
| 26 | Slc25a30 |
| 27 | Cfh |
| 28 | C8a |
| 29 | Wdr91 |
| 30 | Gm15446 |
| 31 | Socs3 |
| 32 | Hsph1 |
| 33 | Mug2 |
| 34 | Prex1 |
| 35 | Whsc1 |
| 36 | Tspan33 |
| 37 | Fkbp5 |
| 38 | Zdhhc14 |
| 39 | Crp |
| 40 | Fan1 |
| 41 | Ccbl1 |
| 42 | Proca1 |
| 43 | Tnk2 |
| 44 | Fam135a |
| 45 | Orm1 |
| 46 | Irf2 |
| 47 | Stard4 |
| 48 | Tbc1d4 |
| 49 | Dock8 |
| 50 | 6430548M08Rik |
| 51 | Tmem45b |
| 52 | Slc35b1 |
| 53 | Scfd2 |
| 54 | Bhlhe40 |
| 55 | Slc40a1 |
| 56 | Crlf2 |
| 57 | Ubr2 |
| 58 | Fam89a |
| 59 | Coro1c |
| 60 | Slc25a23 |
| 61 | Slc16a2 |
| 62 | Kif13b |
| 63 | Dirc2 |
| 64 | Ahsa2 |
| 65 | Lrg1 |
| 66 | Surf4 |
| 67 | Itih3 |
| 68 | Mgat5 |
| 69 | Cdc42bpa |
| 70 | Frmd8 |
| 71 | Simc1 |
| 72 | Cipc |
| 73 | Bahcc1 |
| 74 | Tango6 |
| 75 | Kng1 |
| 76 | Slco2b1 |
| 77 | C1ra |
| 78 | Kdm4a |
| 79 | Adi1 |
| 80 | Wipi1 |
| 81 | Sccpdh |
| 82 | Lpgat1 |
| 83 | Apof |
| 84 | Itih1 |
| 85 | C4bp |
| 86 | Fn1 |
| 87 | Gorasp1 |
| 88 | Tap2 |
| 89 | Cux1 |
| 90 | Kynu |
| 91 | Ptpre |
| 92 | Xbp1 |
| 93 | Ywhab |
| 94 | Sox13 |
| 95 | Slc10a1 |
| 96 | Brpf3 |
| 97 | Lman1 |
| 98 | Fbxl20 |
| 99 | Ginm1 |
| 100 | Acad9 |
| 101 | Aars |
| 102 | 4930453N24Rik |
| 103 | Tmed9 |
| 104 | Tpst2 |
| 105 | Aldh2 |
| 106 | Tiam1 |
| 107 | Dhx36 |
| 108 | Ppib |
| 109 | Gucd1 |
| 110 | Rps6ka1 |
| 111 | Golgb1 |
| 112 | Trim26 |
| 113 | Adh6-ps1 |
| 114 | Itsn1 |
| 115 | 1600012H06Rik |
| 116 | Exoc2 |
| 117 | Ercc6 |
| 118 | Fam219b |
| 119 | Mrps18a |
| 120 | Zfp729b |
| 121 | St3gal3 |
| 122 | Vps18 |
| 123 | Apol9b |

TABLE 16
Intervention-based gene signature 6 Association with
median lifespan change, down-regulated genes)

| Entry No. | Gene |
|---|---|
| 1 | Mup16 |
| 2 | Mup19 |
| 3 | Mup14 |
| 4 | Tnik |
| 5 | Mfhas1 |
| 6 | Cfb |
| 7 | Srd5a1 |
| 8 | Rpl35a |
| 9 | Serpina1d |
| 10 | Apcs |
| 11 | B4galnt3 |
| 12 | Slc6a9 |
| 13 | Npr2 |
| 14 | C8a |
| 15 | Bhlhe40 |
| 16 | Neb |
| 17 | Gm17296 |
| 18 | 1810064F22Rik |
| 19 | Gnai1 |
| 20 | Coq2 |
| 21 | Aatk |
| 22 | Gadd45g |
| 23 | Tspan33 |
| 24 | Homer2 |
| 25 | Gm15446 |
| 26 | Cadm4 |
| 27 | Wdr91 |
| 28 | Sort1 |
| 29 | Zdhhc14 |
| 30 | F11 |
| 31 | Prex1 |
| 32 | Zbtb20 |
| 33 | Stard4 |
| 34 | Rap2a |
| 35 | Irf2 |
| 36 | 6430548M08Rik |
| 37 | 2200002D01Rik |
| 38 | Crp |
| 39 | Crlf2 |
| 40 | Slc17a2 |
| 41 | Tnk2 |
| 42 | Tmem45b |
| 43 | Simc1 |
| 44 | Scfd2 |
| 45 | Ubr2 |
| 46 | Surf4 |
| 47 | Slc35b1 |
| 48 | Zfp324 |
| 49 | Phlpp2 |
| 50 | C1ra |
| 51 | Dirc2 |
| 52 | Syt1 |
| 53 | Tigar |
| 54 | Dpp7 |
| 55 | Txndc11 |
| 56 | D3Ertd254e |
| 57 | Slc25a23 |
| 58 | Kynu |
| 59 | Sox13 |
| 60 | Epb41l4b |
| 61 | Mgat5 |
| 62 | Gorasp1 |
| 63 | Kif13b |
| 64 | Ahsa2 |
| 65 | Tmem19 |
| 66 | Tango6 |
| 67 | Slc38a2 |
| 68 | Tbcel |
| 69 | Bahcc1 |
| 70 | Slc6a13 |
| 71 | Adamts5 |
| 72 | Dhx36 |
| 73 | Cdc42bpa |
| 74 | Serpinc1 |
| 75 | Gne |
| 76 | Aldh2 |
| 77 | Abtb1 |
| 78 | Fbxl20 |
| 79 | Trim26 |
| 80 | Ppib |
| 81 | Coro1c |
| 82 | Slco2b1 |
| 83 | Fyco1 |
| 84 | Nr1h4 |
| 85 | Ciz1 |
| 86 | Myo6 |
| 87 | Thada |
| 88 | Zfp335 |
| 89 | Kng1 |
| 90 | Edrf1 |
| 91 | Spop |
| 92 | Gbf1 |
| 93 | Cys1 |
| 94 | Gucd1 |
| 95 | Cdk5rap3 |
| 96 | Capn1 |
| 97 | Ctnnb1 |
| 98 | Acad9 |
| 99 | Adarb1 |
| 100 | Ttc7b |
| 101 | Brpf3 |
| 102 | Fgd6 |
| 103 | Rabggta |
| 104 | Eif4ebp2 |
| 105 | Lgals8 |
| 106 | Tmed9 |
| 107 | Cpn2 |
| 108 | Adh6-ps1 |
| 109 | Itsn1 |
| 110 | 1600012H06Rik |
| 111 | Exoc2 |
| 112 | Ercc6 |
| 113 | Fam219b |
| 114 | Mrps18a |
| 115 | Zfp729b |
| 116 | St3gal3 |
| 117 | Vps18 |
| 118 | Apol9b |

TABLE 17
Cell turnover-based gene signature (down-regulated genes)

| Entry No. | Gene |
|---|---|
| 1 | Ano10 |
| 2 | Arfip1 |
| 3 | Bcl10 |
| 4 | Brca2 |
| 5 | Bub1b |
| 6 | Ccnb2 |
| 7 | Cdca3 |
| 8 | Cdca8 |
| 9 | Cenpf |
| 10 | Cenpw |
| 11 | Chek1 |
| 12 | Chek2 |
| 13 | Cnot1 |
| 14 | Ddb2 |
| 15 | Ddx52 |
| 16 | Ect2 |
| 17 | Eif6 |
| 18 | Exo1 |
| 19 | Fancd2 |
| 20 | Foxm1 |
| 21 | Gdi2 |
| 22 | Hnrnpf |
| 23 | Kif11 |
| 24 | Kif23 |
| 25 | Mki67 |
| 26 | Msh5 |
| 27 | Nadsyn1 |
| 28 | Ncapg |
| 29 | Ncaph |
| 30 | Net1 |
| 31 | Nuf2 |
| 32 | Orc1 |
| 33 | Parpbp |
| 34 | Pdcd6ip |
| 35 | Plk4 |
| 36 | Rars |
| 37 | Rcc1 |
| 38 | Rccd1 |
| 39 | Samd9l |
| 40 | Scyl2 |
| 41 | Slc25a43 |
| 42 | Spata18 |
| 43 | Stk38 |
| 44 | Trp53 |
| 45 | Zwint |

TABLE 18
Organ-specific gene signature 1 (Brain, down-regulated genes)

| Entry No. | Gene |
|---|---|
| 1 | 1810030O07Rik |
| 2 | A830018L16Rik |
| 3 | Actr2 |
| 4 | Adarb1 |
| 5 | Adprh |
| 6 | Agap2 |
| 7 | Ankrd55 |
| 8 | Ankrd63 |
| 9 | Arfgef1 |
| 10 | Atad1 |
| 11 | Atp11b |
| 12 | Atp2b1 |
| 13 | Atp6ap1l |
| 14 | Atp6v1c1 |
| 15 | Atxn7l3 |
| 16 | Cacnb3 |
| 17 | Ccdc39 |
| 18 | Cdadc1 |
| 19 | Cds1 |
| 20 | Cep120 |
| 21 | Col1a1 |
| 22 | Col4a1 |
| 23 | Ctps2 |
| 24 | Cyb5r4 |
| 25 | Dclk3 |
| 26 | Dgkb |
| 27 | Dlst |
| 28 | Dnajb5 |
| 29 | Dnajc27 |
| 30 | Dnal1 |
| 31 | Dnm1l |
| 32 | Dtl |
| 33 | Dync2li1 |
| 34 | Egflam |
| 35 | Fam13b |
| 36 | Fam83f |
| 37 | Fbxw2 |
| 38 | Fmnl1 |
| 39 | Fmo1 |
| 40 | Gad1 |
| 41 | Gapvd1 |
| 42 | Gdap2 |
| 43 | Gfra1 |
| 44 | Gnal |
| 45 | Gng12 |
| 46 | Gpcpd1 |
| 47 | Gpld1 |
| 48 | Gria3 |
| 49 | Hapln1 |
| 50 | Htr1f |
| 51 | Il1rap |
| 52 | Inpp5j |
| 53 | Ipmk |
| 54 | Jak2 |
| 55 | Kcnj6 |
| 56 | Kcnk2 |
| 57 | Kcnt2 |
| 58 | Klhdc7a |
| 59 | Lclat1 |
| 60 | Mas1 |
| 61 | Mb21d2 |
| 62 | Mtmr3 |
| 63 | Nckap1 |
| 64 | Ndrg4 |
| 65 | Opa1 |
| 66 | Oxsr1 |
| 67 | Palmd |
| 68 | Paqr9 |
| 69 | Pcsk2 |
| 70 | Pde1b |
| 71 | Pdyn |
| 72 | Pik3ca |
| 73 | Pitpna |
| 74 | Plxdc2 |
| 75 | Pold3 |
| 76 | Ppfia3 |
| 77 | Ppm1e |
| 78 | Ppme1 |
| 79 | Ppp1r9a |
| 80 | Ppp2r5a |
| 81 | Ppp3r1 |
| 82 | Prkci |
| 83 | Purb |
| 84 | Rala |
| 85 | Rap2c |
| 86 | Rbm46 |
| 87 | Ric1 |
| 88 | Rock2 |
| 89 | Rragc |
| 90 | Senp7 |
| 91 | Sfmbt1 |
| 92 | Slc22a8 |
| 93 | Slc30a5 |
| 94 | Slc35b4 |
| 95 | Snx13 |
| 96 | Sppl3 |
| 94 | Src |
| 98 | Stk38 |
| 99 | Strbp |
| 100 | Stx6 |
| 101 | Sugt1 |
| 102 | Susd2 |
| 103 | Tbata |
| 104 | Tbc1d8b |
| 105 | Tenm4 |
| 106 | Tgds |
| 107 | Tmem229a |
| 108 | Tomm70a |
| 109 | Trpc4 |
| 110 | Tspan2 |
| 111 | Ube3b |
| 112 | Ubr5 |
| 113 | Vps54 |
| 114 | Vti1a |
| 115 | Wdr36 |
| 116 | Wnt6 |
| 117 | Xkr4 |
| 118 | Xpr1 |
| 119 | Zfc3h1 |
| 120 | Zfp106 |

TABLE 19
Organ-specific gene signature 2 (Kidney, down-regulated genes)

| Entry No. | Gene |
|---|---|
| 1 | 1110059E24Rik |
| 2 | 1700067K01Rik |
| 3 | 4930402H24Rik |
| 4 | 4930453N24Rik |
| 5 | Abcd3 |
| 6 | Abce1 |
| 7 | Aco1 |
| 8 | Aco2 |
| 9 | Actl6a |
| 10 | Adprh |
| 11 | Adra2b |
| 12 | Agpat3 |
| 13 | Arfgef2 |
| 14 | Arhgap11a |
| 15 | Arid2 |
| 16 | Arih1 |
| 17 | Atad2b |
| 18 | Atg7 |
| 19 | Atl2 |
| 20 | Atp11a |
| 21 | Atp2b1 |
| 22 | Atp5a1 |
| 23 | Atp5b |
| 24 | Avpr2 |
| 25 | Birc6 |
| 26 | Brdt |
| 27 | Brwd1 |
| 28 | Cab39l |
| 29 | Cacul1 |
| 30 | Camk2n2 |
| 31 | Camsap2 |
| 32 | Casd1 |
| 33 | Cep19 |
| 34 | Cgrrf1 |
| 35 | Chac2 |
| 36 | Cisd1 |
| 37 | Clock |
| 38 | Cmip |
| 39 | Cnot6l |
| 40 | Col4a3 |
| 41 | Cul4b |
| 42 | Cyp2e1 |
| 43 | D15Ertd621e |
| 44 | Dbt |
| 45 | Ddb1 |
| 46 | Dgkq |
| 47 | Dlat |
| 48 | Dnajc13 |
| 49 | Dpp9 |
| 50 | Dynd1li1 |
| 51 | Efr3a |
| 52 | Eif4g3 |
| 53 | Etfa |
| 54 | Fam135a |
| 55 | Fam20b |
| 56 | Fam210a |
| 57 | Fam8a1 |
| 58 | Fbxo33 |
| 59 | Fetub |
| 60 | Fkbpl |
| 61 | Fmo2 |
| 62 | Fmo5 |
| 63 | Ghitm |
| 64 | Gxylt1 |
| 65 | Hectd2 |
| 66 | Igf1 |
| 67 | Immt |
| 68 | Ipmk |
| 69 | Kbtbd8 |
| 70 | Kcnj16 |
| 71 | Kidins220 |
| 72 | Kif20a |
| 73 | Klhl24 |
| 74 | Kmt2a |
| 75 | Kras |
| 76 | Lace1 |
| 77 | Lekr1 |
| 78 | Lin7c |
| 79 | Lmod2 |
| 80 | Lpgat1 |
| 81 | Ltn1 |
| 82 | Mapt |
| 83 | Mars2 |
| 84 | Mgat3 |
| 85 | Mier3 |
| 86 | Mon2 |
| 87 | Mvb12a |
| 88 | Ncbp1 |
| 89 | Nckap1 |
| 90 | Ndufa9 |
| 91 | Ndufab1 |
| 92 | Ndufs1 |
| 93 | Ndufs2 |
| 94 | Nek2 |
| 95 | Neurl1b |
| 96 | Nsf |
| 97 | Nuak2 |
| 98 | Odf3 |
| 99 | Opa1 |
| 100 | Oplah |
| 101 | Pank1 |
| 102 | Papd5 |
| 103 | Paqr9 |
| 104 | Parg |
| 105 | Pcdh17 |
| 106 | Pcsk6 |
| 107 | Pcyt1a |
| 108 | Pigu |
| 109 | Pik3ca |
| 110 | Pitpnb |
| 111 | Pkn2 |
| 112 | Pkp3 |
| 113 | Ppp2ca |
| 114 | Ppp4r1 |
| 115 | Ppp4r4 |
| 116 | Rab18 |
| 117 | Rbm46 |
| 118 | Rfx7 |
| 119 | Rhebl1 |
| 120 | Rock2 |
| 121 | Sacm1l |
| 122 | Sbk1 |
| 123 | Sclt1 |
| 124 | Sdhb |
| 125 | Sel1l |
| 126 | Slc16a13 |
| 127 | Slc16a7 |
| 128 | Slc34a1 |
| 129 | Slc39a10 |
| 130 | Slc5a1 |
| 131 | Slc5a8 |
| 132 | Slc6a6 |
| 133 | Smagp |
| 134 | Snx13 |
| 135 | Socs7 |
| 136 | Srp54b |
| 137 | Stxbp5 |
| 138 | Synj1 |
| 139 | Taok1 |
| 140 | Tcp11l2 |
| 141 | Tert |
| 142 | Tgfbr1 |
| 143 | Tm9sf3 |
| 144 | Tmppe |
| 145 | Tmx3 |
| 146 | Tnfaip8 |
| 147 | Tnks2 |
| 148 | Tns1 |
| 149 | Top2a |
| 150 | Trpc3 |
| 151 | Trpm7 |
| 152 | Tulp4 |
| 153 | Ubr5 |
| 154 | Uhrf1 |
| 155 | Wdr26 |
| 156 | Wdr35 |
| 157 | Wdr36 |
| 158 | Xylb |
| 159 | Zbtb43 |
| 160 | Zc3h12c |
| 161 | Zfp706 |

TABLE 20
Organ-specific gene signature 3 (Liver, down-regulated genes)

| Entry No. | Gene |
|---|---|
| 1 | 1110059E24Rik |
| 2 | 1700006E09Rik |
| 3 | 1700066M21Rik |
| 4 | Abcd3 |
| 5 | Abce1 |
| 6 | Abcf2 |
| 7 | Acbd5 |
| 8 | Acox1 |
| 9 | Acpp |
| 10 | Ado |
| 11 | Agpat3 |
| 12 | Ankrd52 |
| 13 | Arid2 |
| 14 | Arl5a |
| 15 | Arl5b |
| 16 | Armc1 |
| 17 | Asb8 |
| 18 | Asun |
| 19 | Atad2b |
| 20 | Atl2 |
| 21 | Atp11b |
| 22 | Atp5a1 |
| 23 | Atp5b |
| 24 | BC004004 |
| 25 | Bmf |
| 26 | Bpnt1 |
| 27 | Brpf1 |
| 28 | Btbd8 |
| 29 | Cacul1 |
| 30 | Carnmt1 |
| 31 | Cdip1 |
| 32 | Cep152 |
| 33 | Cgrrf1 |
| 34 | Chac2 |
| 35 | Chmp7 |
| 36 | Chuk |
| 37 | Cisd1 |
| 38 | Clock |
| 39 | Clpx |
| 40 | Cluap1 |
| 41 | Cluh |
| 42 | Cps1 |
| 43 | Cul4b |
| 44 | Cyb5r4 |
| 45 | Dbt |
| 46 | Derl1 |
| 47 | Dnajc3 |
| 48 | Dtnbp1 |
| 49 | Ehmt2 |
| 50 | Erap1 |
| 51 | Evi5 |
| 52 | Fam175b |
| 53 | Fam214a |
| 54 | Fbxo45 |
| 55 | Gan |
| 56 | Gmfb |
| 57 | Gnpnat1 |
| 58 | Gpc4 |
| 59 | Hectd1 |
| 60 | Hsdl1 |
| 61 | Igf1 |
| 62 | Immt |
| 63 | Ipmk |
| 64 | Kbtbd8 |
| 65 | Kif21a |
| 66 | Klhl11 |
| 67 | Klhl24 |
| 68 | Lace1 |
| 69 | Larp4b |
| 70 | Lpar3 |
| 71 | Lppr5 |
| 72 | Lyrm2 |
| 73 | Mafg |
| 74 | Map2k4 |
| 75 | Mapt |
| 76 | Minpp1 |
| 77 | Mtf1 |
| 78 | Naa15 |
| 79 | Nanp |
| 80 | Ndufa10 |
| 81 | Nmt1 |
| 82 | Nr3c1 |
| 83 | Ogdh |
| 84 | Opa1 |
| 85 | Opcml |
| 86 | Oprm1 |
| 87 | Pafah1b1 |
| 88 | Papss2 |
| 89 | Parp16 |
| 90 | Pcyt1a |
| 91 | Pitpnb |
| 92 | Pkp3 |
| 93 | Plaa |
| 94 | Ppm1a |
| 95 | Ppp2r5e |
| 96 | Psmd1 |
| 97 | Psmd11 |
| 98 | Pvrl1 |
| 99 | Rmnd1 |
| 100 | Rnf4 |
| 101 | Rragc |
| 102 | Sacm1l |
| 103 | Sbno1 |
| 104 | Scai |
| 105 | Slc25a13 |
| 106 | Slc25a15 |
| 107 | Slc25a20 |
| 108 | Slc25a23 |
| 109 | Slc25a44 |
| 110 | Slc25a46 |
| 111 | Slc33a1 |
| 112 | Slc38a7 |
| 113 | Slc4a4 |
| 114 | Slmap |
| 115 | Smim8 |
| 116 | Smurf2 |
| 117 | Snx13 |
| 118 | Socs4 |
| 119 | Sox6 |
| 120 | Spef1 |
| 121 | Sppl3 |
| 122 | Stk35 |
| 123 | Stx17 |
| 124 | Sucla2 |
| 125 | Suds3 |
| 126 | Suv420h2 |
| 127 | Synj2bp |
| 128 | Taok1 |
| 129 | Tcp11l2 |
| 130 | Tex2 |
| 131 | Tm9sf3 |
| 132 | Tmem106b |
| 133 | Tmem170b |
| 134 | Tmem63b |
| 135 | Tmppe |
| 136 | Trappc13 |
| 137 | Ttc7 |
| 138 | Tulp4 |
| 139 | Uba3 |
| 140 | Ube2a |
| 141 | Ube2w |
| 142 | Ubr5 |
| 143 | Ufm1 |
| 144 | Umad1 |
| 145 | Usp14 |
| 146 | Usp47 |
| 147 | Vwa8 |
| 148 | Wdr36 |
| 149 | Wdtc1 |
| 150 | Zbtb41 |
| 151 | Zbtb44 |
| 152 | Zfp740 |

### Example 20

Procedure for Identifying Candidate Interventions Based on Association with Longevity Signatures

There are a variety of protocols that can be implemented in order to use the longevity gene signatures described herein to identify new interventions capable of extending lifespan, reducing frailty, improving learning ability, and/or preventing/delaying the onset of a geriatric syndrome. An exemplary protocol that can be used for this purpose is described below:

1. Download longevity signatures. Every signature contains two sets of genes. One of them includes genes positively associated with a certain longevity metric, and the other includes genes with the negative association.
2. Prepare dataset of interest. For every gene in the gene expression data of interest, calculate fold changes and corresponding p-values between intervention and control groups. For every gene, calculate significance score, defined as −log₁₀(p. value)×sgn(logFC). Sort genes based on the significance score (from the highest value to the lowest).
3. Filter out excess genes. From particular longevity signature gene sets, filter out all genes that are not represented in the sorted list corresponding to gene expression dataset of interest.
4. Calculate connectivity score (metric of the effect size). Calculate connectivity scores separately for gene sets positively and negatively associated with longevity metric as described in (Lamb et al., 2006). First, calculate Kolmogorov-Smirnov enrichment statistics (ES) separately for positively and negatively associated genes. Then, calculate the final connectivity score as an average between the two:

$$connectivity score = \frac{E {S}_{+} - {ES}_{-}}{2} .$$

5. Calculate p-value (metric of statistical significance). To calculate statistical significance of obtained connectivity score, apply permutation test. Randomly choose genes from the sorted list so that they form gene sets of the same size as longevity gene sets. Then calculate the connectivity score for these randomized signatures using the same algorithm as described above. Repeat this algorithm (e.g., 3,000 times). Then calculate p-value as the proportion of cases when the absolute value of random connectivity score is bigger than the absolute value of the real connectivity score:

$$p . value = \frac{\# ( \vert random connectivity score \vert > \vert connectivity score \vert )}{\# permutations}$$

6. Adjust p-values for multiple hypotheses. Adjust obtained p-values corresponding to different longevity signatures using multiple hypothesis correction techniques (e.g., Benjamini-Hochberg method). The resulting connectivity scores and adjusted p-values may be used as a metric of association between longevity signatures and gene expression response to the intervention of interest.

### Example 21

### Testing Longevity Interventions in Mice: Effects of Various Agents on Lifespan, Gait Speed, Frailty Index, and Muscle Function

### Materials and Methods

Interventions were predicted in a screen based on the gene expression longevity signatures that we developed. The predicted interventions were then verified for gene expression responses in human and mouse primary cell culture (hepatocytes) and in live mice (after mice were fed for 1 month with the diets containing these interventions). The interventions that passed these tests were further assessed for the effect on lifespan of 2-year-old C57BI/6 mice. Older mice (2-year-old) were chosen for this experiment in order to mimic the effect of giving interventions to human subjects in their second half of life.

The basic scheme of the experiment is shown in FIGS. 19A-19C. Briefly, 24-28 mice per intervention were used, with an approximately equal number of males and females. They were first assessed with regard to frailty index and gait speed, and then randomized to make sure the experimental and control groups had the same average frailty index and gait speed. Mice were then given a diet containing a compound of interest. Control mice were treated identically, except that their diet did not have the compound of interest. Mice were monitored daily until they died. A separate cohort of old mice was assessed for frailty index and gait speed. Compounds that exhibited a lifespan-extending effect are discussed below.

### Results: AZD-8055

AZD-8055 was given to mice ad libitum in the amount of 20 mg/kg of food. This agent extends the lifespan of male mice (FIG. 20). Gait speed was also assessed and was found to be improved in old males compared to controls, suggesting that AZD-8055 helps to preserve muscle function in old males (FIG. 21). We found no effect of this compound on frailty index. AZD-8055 did not compromise glucose tolerance at the dose given (FIG. 22).

### Results: Selumetinib

Selumetinib was given ad libitum at the concentration of 100 mg/kg of diet. We found that it extends lifespan of C57BI/6 mice (FIG. 23, left). We also set up an independent cohort of female mice, and again found that Selumetinib extends lifespan (FIG. 23, right). Selumetinib also improves frailty index (FIG. 24) and does not alter the relative populations of immune cells in the spleen (FIG. 25).

### Results: Celastrol

Celastrol was given ad libitum at the concentration of 8 mg/kg of food. We found that it has a lifespan-extending effect (p=0.052) (FIG. 26). It does not affect frailty index and gait speed (FIG. 27).

### Results: LY294002

LY294002 was given ad libitum at the level of 600 mg/kg of diet. We found that it extends lifespan of male mice (FIG. 28). It also improves gait speed and frailty index in males (FIG. 29). LY294002 did not have a significant effect on glucose tolerance (FIG. 30).

### Results: KU-0063794

KU-0063794 was given ad libitum at the concentration of 10 mg/kg of diet. This agent was found to extend lifespan of male mice (p=0.052) (FIG. 31) as well as frailty index and gait speed (FIG. 32). KU-0063794 did not have a significant effect on glucose tolerance (FIG. 33).

### REFERENCES

Aaron, E. A., and Powell, F. L. (1993). Effect of chronic hypoxia on hypoxic ventilatory response in awake rats. J. Appl. Physiol. 74, 1635-1640.
Ables, G. P., Perrone, C. E., Orentreich, D., and Orentreich, N. (2012). Methionine-Restricted C57BL/6J Mice Are Resistant to Diet-Induced Obesity and Insulin Resistance but Have Low Bone Density. PLoS One 7, 1-12.
Ables, G. P., Ouattara, A., Hampton, T. G., Cooke, D., Perodin, F., Augie, I., and Orentreich, D. S. (2015). Dietary methionine restriction in mice elicits an adaptive cardiovascular response to hyperhomocysteinemia. Sci. Rep. 5, 1-10.
   al-Shawi, R., Wallace, H., Harrison, S., Jones, C., Johnson, D., and Bishop, J. O. (1992). Sexual dimorphism and growth hormone regulation of a hybrid gene in transgenic mice. Mol. Endocrinol. 6, 181-190.
Alonso, C., Fernández-Ramos, D., Varela-Rey, M., Martinez-Arranz, I., Navasa, N., Van Liempd, S. M., Lavin Trueba, J. L., Mayo, R., Ilisso, C. P., de Juan, V. G., et al. (2017). Metabolomic Identification of Subtypes of Nonalcoholic Steatohepatitis. Gastroenterology 152, 1449-1461.e7.
Amador-Noguez, D., Yagi, K., Venable, S., and Darlington, G. (2004). Gene expression profile of long-lived Ames dwarf mice and Little mice. Aging Cell 3, 423-441.
Baird, L., and Dinkova-Kostova, A. T. (2011). The cytoprotective role of the Keap1-Nrf2 pathway. Arch. Toxicol. 85, 241-272.
Balaban, R. S., Nemoto, S., and Finkel, T. (2005). Mitochondria, oxidants, and aging. Cell 120, 483-495.
Barger, J. L., Kayo, T., Vann, J. M., Arias, E. B., Wang, J., Hacker, T. A., Wang, Y., Raederstorff, D., Morrow, J. D., Leeuwenburgh, C., et al. (2008). A low dose of dietary resveratrol partially mimics caloric restriction and retards aging parameters in mice. PLoS One 3.
Baur, J. A., and Sinclair, D. A. (2006). Therapeutic potential of resveratrol: The in vivo evidence. Nat. Rev. Drug Discov. 5, 493-506.
Baur, J. A., Pearson, K. J., Price, N. L., Jamieson, H. A., Lerin, C., Kalra, A., Prabhu, V. V, Allard, J. S., Lopez-Lluch, G., Lewis, K., et al. (2006). Resveratrol improves health and survival of mice on a high-calorie diet. Nature 444, 337-342.
Baze, M. M., Schlauch, K., and Hayes, J. P. (2010). Gene expression of the liver in response to chronic hypoxia. 275-288.
Boylston, W. H., DeFord, J. H., and Papaconstantinou, J. (2006). Identification of longevity-associated genes in long-lived Snell and Ames dwarf mice. Age (Omaha). 28, 125-144.
Brown-borg, H. M. (2007). Hormonal regulation of longevity in mammals. Ageing Res. Rev. 6, 28-45.
Brown-Borg, H. M., Rakoczy, S. G., and Uthus, E. O. (2005). Growth hormone alters methionine and glutathione metabolism in Ames dwarf mice. Mech. Ageing Dev. 126, 389-398.
Buckley, D. B., and Klaassen, C. D. (2009). Mechanism of Gender-Divergent UDP-Glucuronosyltransferase mRNA Expression in Mouse Liver and Kidney. 37, 834-840.
Cao, L., Li, W., Kim, S., Brodie, S. G., and Deng, C. X. (2003). Senescence, aging, and malignant transformation mediated by p53 in mice lacking the brca1 full-length isoform. Genes Dev. 17, 201-213.
Chang, W., Cheng, J., Allaire, J., Xie, Y., and McPherson, J. (2016). shiny: Web Application Framework for R. R Packag. Version 0.14.2. Https//CRAN.R-Project.Org/Package=shiny.
Chen, D., Thomas, E. L., and Kapahi, P. (2009). HIF-1 modulates dietary restriction-mediated lifespan extension via IRE-1 in Caenorhabditis elegans. PLoS Genet. 5.
Chresta, C. M., Davies, B. R., Hickson, I., Harding, T., Cosulich, S., Critchlow, S. E., Vincent, J. P., Ellston, R., Jones, D., Sini, P., et al. (2010). AZD8055 is a potent, selective, and orally bioavailable ATP-competitive mammalian target of rapamycin kinase inhibitor with in vitro and in vivo antitumor activity. Cancer Res. 70, 288-298.
Cort, W. M. (1974). Antioxidant activity of tocopherols, ascorbyl palmitate, and ascorbic acid and their mode of action. J. Am. Oil Chem. Soc. 51, 321-325.
Coschigano, K. T., Clemmons, D., Bellush, L. L., and Kopchick, J. J. (2000). Assessment of growth parameters and lifespan of GHR/BP gene-disrupted mice. Endocrinology 141, 2608-26β.
Coschigano, K. T., Holland, A. N., Riders, M. E., List, E. O., Flyvbjerg, A., and Kopchick, J. J. (2003). Deletion, but not antagonism, of the mouse growth hormone receptor results in severely decreased body weights, insulin, and insulin-like growth factor I levels and increased life span. Endocrinology 144, 3799-3810.
David, J., Van Herrewege, J., and Fouillet, P. (1971). Quantitative under-feeding of drosophila: Effects on adult longevity and fecundity. Exp. Gerontol. 6, 249-257.
Dhahbi, J. M., Mote, P. L., Fahy, G. M., and Spindler, S. R. (2005). Identification of potential caloric restriction mimetics by microarray profiling. Am. Physiol. Soc. 23, 343-350.
Estep, P. W., Warner, J. B., and Bulyk, M. L. (2009). Short-term calorie restriction in male mice feminizes gene expression and alters key regulators of conserved aging regulatory pathways. PLoS One 4.
Fok, W. C., Chen, Y., Bokov, A., Zhang, Y., Salmon, A. B., Diaz, V., Javors, M., Wood, W. H., Zhang, Y., Becker, K. G., et al. (2014a). Mice fed rapamycin have an increase in lifespan associated with major changes in the liver transcriptome. PLoS One 9.
Fok, W. C., Bokov, A., Gelfond, J., Yu, Z., Zhang, Y., Doderer, M., Chen, Y., Javors, M., Wood, W. H., Zhang, Y., et al. (2014b). Combined treatment of rapamycin and dietary restriction has a larger effect on the transcriptome and metabolome of liver. Aging Cell 13, 311-319.
Fontana, L., Partridge, L., and Longo, V. D. (2010). Extending Healthy Life Span-From Yeast to Humans. Science (80-.). 328, 321-326.
Fu, Z. D., and Klaassen, C. D. (2014). Short-term calorie restriction feminizes the mRNA profiles of drug metabolizing enzymes and transporters in livers of mice. Toxicol. Appl. Pharmacol. 274, 137-146.
Gadó, K., Domján, G., Hegyesi, H., and Falus, A. (2000). Role of interleukin-6 in the pathogenesis of multiple myeloma. Cell Biol. Int. 24, 195-209.
García-Martínez, J. M., Alessi, D. R., Moran, J., Cosulich, S. C., Clarke, R. G., Gray, A., and Chresta, C. M. (2009). Ku-0063794 is a specific inhibitor of the mammalian target of rapamycin (mTOR). Biochem. J. 421, 29-42.
Garratt, M., Stockley, P., Armstrong, S. D., Beynon, R. J., and Hurst, J. L. (2011). The scent of senescence: Sexual signalling and female preference in house mice. J. Evol. Biol. 24, 2398-2409.
Gautier, H. (1996). Interactions and control among metabolic of breathing rate, hypoxia, and control of breathing. 521-527.
Gertz, M., Nguyen, G. T. T., Fischer, F., Suenkel, B., Schlicker, C., Fränzel, B., Tomaschewski, J., Aladini, F., Becker, C., Wolters, D., et al. (2012). A Molecular Mechanism for Direct Sirtuin Activation by Resveratrol. PLoS One 7, 1-12.
Gokarn, R., Solon-Biet, S. M., Cogger, V. C., Cooney, G. J., Wahl, D., McMahon, A. C., Mitchell, J. R., Mitchell, S. J., Hine, C., De Cabo, R., et al. (2018). Long-term Dietary Macronutrients and Hepatic Gene Expression in Aging Mice. J Gerontol A Biol Sci Med Sci 00, 1-8.
Gorrini, C., Baniasadi, P. S., Harris, I. S., Silvester, J., Inoue, S., Snow, B., Joshi, P. A., Wakeham, A., Molyneux, S. D., Martin, B., et al. (2013). BRCA1 interacts with Nrf2 to regulate antioxidant signaling and cell survival. J. Exp. Med. 210, 1529-1544.
Grandison, R. C., Piper, M. D. W., and Partridge, L. (2009). Amino acid imbalance explains extension of lifespan by dietary restriction in Drosophila. Nature 462, 1061-1064.
De Haes, W., Frooninckx, L., Van Assche, R., Smolders, A., Depuydt, G., Billen, J., Braeckman, B. P., Schoofs, L., and Temmerman, L. (2014). Metformin promotes lifespan through mitohormesis via the peroxiredoxin PRDX-2. Proc. Natl. Acad. Sci. 111, E2501-E2509.
Harrison, D. E., Strong, R., Sharp, Z. D., Nelson, J. F., Astle, C. M., Flurkey, K., Nadon, N. L., Wilkinson, J. E., Frenkel, K., Carter, C. S., et al. (2009). Rapamycin fed late in life extends lifespan in genetically heterogeneous mice. Nature 460, 392-395.
Harrison, D. E., Strong, R., Allison, D. B., Ames, B. N., Astle, C. M., Atamna, H., Fernandez, E., Flurkey, K.,
Javors, M. A., Nadon, N. L., et al. (2014). Acarbose, 17-α-estradiol, and nordihydroguaiaretic acid extend mouse lifespan preferentially in males. Aging Cell 13, 273-282.
Hine, C., Harputlugil, E., Zhang, Y., Ruckenstuhl, C., Lee, B. C., Brace, L., Longchamp, A., Trevino-Villarreal, J. H., Mejia, P., Ozaki, C. K., et al. (2015). Endogenous hydrogen sulfide production is essential for dietary restriction benefits. Cell 160, 132-144.
Hofmann, J. W., Zhao, X., De Cecco, M., Peterson, A. L., Pagliaroli, L., Manivannan, J., Hubbard, G. B., Ikeno, Y., Zhang, Y., Feng, B., et al. (2015). Reduced expression of MYC increases longevity and enhances healthspan. Cell 160, 477-488.
Houthoofd, K., and Vanfleteren, J. R. (2006). The longevity effect of dietary restriction in Caenorhabditis elegans. Exp. Gerontol. 41, 1026-1031.
Huang, D. W., Sherman, B. T., and Lempicki, R. A. (2009a). Bioinformatics enrichment tools: Paths toward the comprehensive functional analysis of large gene lists. Nucleic Acids Res. 37, 1-13.
Huang, D. W., Lempicki, R. a, and Sherman, B. T. (2009b). Systematic and integrative analysis of large gene lists using DAVID bioinformatics resources. Nat. Protoc. 4, 44-57.
Imaoka, S., Fujita, S., and Funae, Y. (1991). Age-dependent exression of cytochrome P-450s in rat liver. BBA—Mol. Basis Dis. 1097, 187-192.
Jain, I. H., Zazzeron, L., Goli, R., Alexa, K., Schatzman-Bone, S., Dhillon, H., Goldberger, O., Peng, J., Shalem, O., Sanjana, N. E., et al. (2016). Hypoxia as a therapy for mitochondrial disease. Science (80-.). 352, 54-61.
Kabil, O., Vitvitsky, V., Xie, P., and Banerjee, R. (2011). The quantitative significance of the transsulfuration enzymes for H2S production in murine tissues. Antioxid. Redox Signal. 15, 363-372.
Kamataki, T., Maeda, K., Shimada, M., Kitani, K., Nagai, T., and Kato, R. (1985). Age-Related Alteration in the Activities of Drug-Metabolizing Enzymes and Contents of Sex-Specific Forms of Cytochrome P-450 in Liver Microsomes from Male and Female Rats1. J. Pharmacol. Exp. Ther. 233, 222-228.
Kanfi, Y., Naiman, S., Amir, G., Peshti, V., Zinman, G., Nahum, L., Bar-Joseph, Z., and Cohen, H. Y. (2012). The sirtuin SIRT6 regulates lifespan in male mice. Nature 483, 218-221.
Kapahi, P., Zid, B. M., and Harper, T. (2004). Regulation of Lifespan in Drosophila by Modulation of Genes in the TOR Signaling Pathway. Curr. Biol. 14, 885-890.
Kautz, L., Meynard, D., Monnier, A., Darnaud, V., Bouvet, R., Wang, R. H., Deng, C., Vaulont, S., Mosser, J., Coppin, H., et al. (2008). Iron regulates phosphorylation of Smad1/5/8 and gene expression of Bmp6, Smad7, Id1, and Atoh8 in the mouse liver. Blood 112, 1503-1509.
Knopf, J. L., Gallagher, J. F., and Held, W. A. (1983). Differential, multihormonal regulation of the mouse major urinary protein gene family in the liver. Mol. Cell. Biol. 3, 2232-2240.
Kobayashi, T., Shimabukuro-Demoto, S., Yoshida-Sugitani, R., Furuyama-Tanaka, K., Karyu, H., Sugiura, Y., Shimizu, Y., Hosaka, T., Goto, M., Kato, N., et al. (2014). The histidine transporter SLC15A4 coordinates mTOR-dependent inflammatory responses and pathogenic antibody production. Immunity 41, 375-388.
Kristiansen, O. P., and Mandrup-Poulsen, T. (2005). Interleukin-6 and Diabetes. Diabetes 54, S114 LP-S124.
Lakowski, B., and Hekimi, S. (1998). The genetics of caloric restriction in Caenorhabditis elegans. Proc. Natl. Acad. Sci. 95, 13091-13096.
Lamb, J., Crawford, E. D., Peck, D., Modell, J. W., Blat, I. C., Wrobel, M. J., Lerner, J., Brunet, J., Subramanian, A., Ross, K. N., et al. (2006). The Connectivity Map: Using Gene-Expression Signatures to Connect Small Molecules, Genes, and Disease. Science (80-.). 313, 1929-1935.
Leiser, S. F., and Miller, R. A. (2010). Nrf2 Signaling, a Mechanism for Cellular Stress Resistance in Long-Lived Mice. Mol. Cell. Biol. 30, 871-884.
Li, X., Bartke, A., Berryman, D. E., Funk, K., Kopchick, J. J., List, E. O., Sun, L., and Miller, R. A. (2013). Direct and indirect effects of growth hormone receptor ablation on liver expression of xenobiotic metabolizing genes. AJP Endocrinol. Metab. 305, E942-E950.
Lin, A. S., Defossez, P., Guarente, L., Lin, S., Defossez, P., and Guarentet, L. (2000). Requirement of NAD and SIR2 for Life-Span Extension by Calorie Restriction in Saccharomyces cerevisiae. Science (80-.). 289, 2126-2128.
Lopez-Otin, C., Blasco, M. A., Partridge, L., Serrano, M., and Kroemer, G. (2013). The hallmarks of aging. Cell 153.
Ma, S., Yim, S. H., Lee, S.-G., Kim, E. B., Lee, S.-R., Chang, K.-T., Buffenstein, R., Lewis, K. N., Park, T. J., Miller, R. A., et al. (2015). Organization of the Mammalian Metabolome according to Organ Function, Lineage Specialization, and Longevity. Cell Metab. 22, 332-343.
De Magalhães, J. P., and Toussaint, 0. (2004). GenAge: A genomic and proteomic network map of human ageing. FEBS Lett. 571, 243-247.
Martin-Montalvo, A., Mercken, E. M., Mitchell, S. J., Palacios, H. H., Mote, P. L., Scheibye-Knudsen, M., Gomes, A. P., Ward, T. M., Minor, R. K., Blouin, M.-J., et al. (2013). Metformin improves healthspan and lifespan in mice. Nat Commun. 4, 2192.
Matys, V. (2006). TRANSFAC® and its module TRANSCompel®: transcriptional gene regulation in eukaryotes. Nucleic Acids Res. 34, D108-D110.
Mehta, R., Steinkraus, K. A., Sutphin, G. L., Ramos, F. J., S, L., Huh, A., Davis, C., Chandler-brown, D., and Kaeberlein, M. (2009). Proteasomal Regulation of the Hypoxic Response Modulates Aging in C. elegans.Science (80-.). 324, 1196-1198.
Mercken, E. M., Hu, J., Krzysik-Walker, S., Wei, M., Li, Y., Mcburney, M. W., de Cabo, R., and Longo, V. D. (2014a). SIRT1 but not its increased expression is essential for lifespan extension in caloric-restricted mice. Aging Cell 13, 193-196.
Mercken, E. M., Mitchell, S. J., Martin-, A., Minor, R. K., Almeida, M., Gomes, A. P., Scheibye-knudsen, M., Hector, H., Licata, J. J., Zhang, Y., et al. (2014b). SRT2104 extends survival of male mice on a standard diet and preserves bone and muscle mass. 787-796.
Miller, D. L., and Roth, M. B. (2007). Hydrogen sulfide increases thermotolerance and lifespan in Caenorhabditis elegans. Proc. Natl. Acad. Sci. 104, 20618-20622.
Miller, R. A., Harrison, D. E., Astle, C. M., Floyd, R. A., Flurkey, K., Hensley, K. L., Javors, M. A., Leeuwenburgh, C., Nelson, J. F., Ongini, E., et al. (2007). An aging Interventions Testing Program: Study design and interim report. Aging Cell 6, 565-575.
Miller, R. A., Harrison, D. E., Astle, C. M., Baur, J. A., Boyd, A. R., De Cabo, R., Fernandez, E., Flurkey, K., Javors, M. A., Nelson, J. F., et al. (2011). Rapamycin, but not resveratrol or simvastatin, extends life span of genetically heterogeneous mice. Journals Gerontol.—Ser. A Biol. Sci. Med. Sci. 66 A, 191-201.
Miller, R. A., Harrison, D. E., Astle, C. M., Fernandez, E., Flurkey, K., Han, M., Javors, M. A., Li, X., Nadon, N. L., Nelson, J. F., et al. (2014). Rapamycin-mediated lifespan increase in mice is dose and sex dependent and metabolically distinct from dietary restriction. Aging Cell 13, 468-477.
Mitchell, S. J., Madrigal-Matute, J., Scheibye-Knudsen, M., Fang, E., Aon, M., Gonzalez-Reyes, J. A., Cortassa, S., Kaushik, S., Gonzalez-Freire, M., Patel, B., et al. (2016). Effects of Sex, Strain, and Energy Intake on Hallmarks of Aging in Mice. Cell Metab. 23, 1093-1112.
Moorad, J. A., Promislow, D. E. L., Nate, F., and Miller Richard A. (2012). A comparative assessment of univariate longevity measures using zoological animal records. Aging Cell 11, 940-948.
Mpoy, M., Vandeleene, B., Ketelslegers, J. M., and Lambert, A. E. (1988). Treatment of systemic hypertension in insulin-treated diabetes mellitus with rilmenidine. Am. J. Cardiol. 61, 5-8.
Mutter, F. E., Park, B. K., and Copple, I. M. (2015). Value of monitoring Nrf2 activity for the detection of chemical and oxidative stress. Biochem. Soc. Trans. 43, 657-662.
Nakamura, N., Lill, J. R., Phung, Q., Jiang, Z., Bakalarski, C., De Mazière, A., Klumperman, J., Schlatter, M., Delamarre, L., and Mellman, I. (2014). Endosomes are specialized platforms for bacterial sensing and NOD2 signalling. Nature 509, 240-244.
Narod, S. A., and Foulkes, W. D. (2004). BRCA1 and BRCA2: 1994 and beyond. Nat. Rev. Cancer 4, 665-676.
Osburn, W. O., Yates, M. S., Dolan, P. D., Chen, S., Liby, K. T., Sporn, M. B., Taguchi, K., Yamamoto, M., and Kensler, T. W. (2008). Genetic or pharmacologic amplification of Nrf2 signaling inhibits acute inflammatory liver injury in mice. Toxicol. Sci. 104, 218-227.
Ozerov, I. V., Lezhnina, K. V., lzumchenko, E., Artemov, A. V., Medintsev, S., Vanhaelen, Q., Aliper, A., Vijg, J., Osipov, A. N., Labat, I., et al. (2016). In silico Pathway Activation Network Decomposition Analysis (iPANDA) as a method for biomarker development. Nat. Commun. 7, 1-11.
Pearson, K. J., Baur, J. A., Lewis, K. N., Peshkin, L., Price, N. L., Labinskyy, N., Swindell, W. R., Kamara, D., Minor, R. K., Perez, E., et al. (2008). Resveratrol Delays Age-Related Deterioration and Mimics Transcriptional Aspects of Dietary Restriction without Extending Life Span. Cell Metab. 8, 157-168.
Plank, M., Wuttke, D., van Dam, S., Clarke, S. A., and de Magalhães, J. P. (2012). A meta-analysis of caloric restriction gene expression profiles to infer common signatures and regulatory mechanisms. Mol. Biosyst. 8, 1339.
Ramadoss, P., Chiappini, F., Bilban, M., and Hollenberg, A. N. (2010). Regulation of hepatic six transmembrane epithelial antigen of prostate 4 (STEAP4) expression by STAT3 and CCAAT/enhancer-binding protein α. J. Biol. Chem. 285, 16453-16466.
Rhoads, T. W., Burhans, M. S., Chen, V. B., Coon, J. J., Colman, R. J., Anderson, R. M., Rhoads, T. W., Burhans, M. S., Chen, V. B., Hutchins, P. D., et al. (2018). Caloric Restriction Engages Hepatic RNA Processing Mechanisms in Rhesus Monkeys Resource Caloric Restriction Engages Hepatic RNA Processing Mechanisms in Rhesus Monkeys. Cell Metab. 27, 677-688.e5.
Richie, J. P., Leutzinger, Y., Parthasarathy, S., Malloy, V., Orentreich, N., and Zimmerman, J. a (1994). Methionine restriction increases blood glutathione and longevity in F344 rats. FASEB J. 8, 1302-1307. Roberts, S. A., Simpson, D. M., Armstrong, S. D., Davidson, A. J., Robertson, D. H., McLean, L., Beynon, R. J., and Hurst, J. L. (2010). Darcin: A male pheromone that stimulates female memory and sexual attraction to an individual male's odour. BMC Biol. 8.
Rowland, J. E., Lichanska, A. M., Linda, M., White, M., Aniello, E. M., Maher, S. L., Brown, R., Teasdale, R. D., Noakes, P. G., Waters, M. J., et al. (2005). In Vivo Analysis of Growth Hormone Receptor Signaling Domains and Their Associated Transcripts In Vivo Analysis of Growth Hormone Receptor Signaling Domains and Their Associated Transcripts. Mol. Cell. Biol. 25, 66-77.
Rusli, F., Boekschoten, M. V., Zubia, A. A., Lute, C., Müller, M., and Steegenga, W. T. (2015). A weekly alternating diet between caloric restriction and medium fat protects the liver from fatty liver development in middle-aged C57BL/6J mice. Mol. Nutr. Food Res. 59, 533-543.
Senn, J. J., Klover, P. J., Nowak, I. A., and Mooney, R. A. (2002). Interleukin-6 induces cellular insulin resistance in hepatocytes. Diabetes 51, 3391-3399.
Steinbaugh, M. J., Sun, L. Y., Bartke, A., and Miller, R. A. (2012). Activation of genes involved in xenobiotic metabolism is a shared signature of mouse models with extended lifespan. Am. J. Physiol. Endocrinol. Metab. 303, E488-95.
Steiner, A. A., and Branco, L. G. S. (2002). Hypoxia-Induced Anapyrexia: Implications and Putative Mediators. Annu. Rev. Physiol. 64, 263-288.
Streeper, R. S., Grueter, C. A., Salomonis, N., Cases, S., Levin, M. C., Koliwad, S. K., Zhou, P., Hirschey, M. D., Verdin, E., and Farese, R. V. (2012). Deficiency of the lipid synthesis enzyme, DGAT1, extends longevity in mice. Aging (Albany. N.Y.). 4, 13-27.
Strong, R., Miller, R. A., Astle, C. M., Floyd, R. A., Flurkey, K., Hensley, K. L., Javors, M. A., Leeuwenburgh, C., Nelson, J. F., Ongini, E., et al. (2008). Nordihydroguaiaretic acid and aspirin increase lifespan of genetically heterogeneous male mice. Aging Cell 7, 641-650.
Strong, R., Miller, R. A., Astle, C. M., Baur, J. A., De Cabo, R., Fernandez, E., Guo, W., Javors, M., Kirkland, J. L., Nelson, J. F., et al. (2013). Evaluation of resveratrol, green tea extract, curcumin, oxaloacetic acid, and medium-chain triglyceride oil on life span of genetically heterogeneous mice. Journals Gerontol.—Ser. A Biol. Sci. Med. Sci. 68, 6-16.
Strong, R., Miller, R. A., Antebi, A., Astle, C. M., Bogue, M., Denzel, M. S., Fernandez, E., Flurkey, K., Hamilton, K. L., Lamming, D. W., et al. (2016). Longer lifespan in male mice treated with a weakly estrogenic agonist, an antioxidant, an α-glucosidase inhibitor or a Nrf2-inducer. Aging Cell 15, 872-884.

Subramanian, A., Tamayo, P., Mootha, V. K., Mukherjee, S., Ebert, B. L., Gillette, M. a, Paulovich, A., Pomeroy, S. L., Golub, T. R., Lander, E. S., et al. (2005). Gene set enrichment analysis: a knowledge-based approach for interpreting genome-wide expression profiles. Proc. Natl. Acad. Sci. U.S.A 102, 15545-15550.
Subramanian, A., Narayan, R., Corsello, S. M., Peck, D. D., Natoli, T. E., Lu, X. L., Gould, J., Doench, J. G., Bittker, J. A., Root, D. E., et al. (2017). A Next Generation Connectivity Map: L1000 platform and the first 1,000,000 profiles. Cell 171, 1437-1452.
Sun, L. Y., Spong, A., Swindell, W. R., Fang, Y., Hill, C., Huber, J. A., Boehm, J. D., Westbrook, R., Salvatori, R., and Bartke, A. (2013). Growth hormone-releasing hormone disruption extends lifespan and regulates response to caloric restriction in mice. Elife 2, e01098.
Swardfager, W., Lanctt, K., Rothenburg, L., Wong, A., Cappell, J., and Herrmann, N. (2010). A meta-analysis of cytokines in Alzheimer's disease. Biol. Psychiatry 68, 930-941.
Swindell, W. R. (2008). Comparative analysis of microarray data identifies common responses to caloric restriction among mouse tissues. Mech. Ageing Dev. 129, 138-153.
Sykiotis, G. P., and Bohmann, D. (2008). Keap1/Nrf2 Signaling Regulates Oxidative Stress Tolerance and Lifespan in Drosophila. Dev. Cell 14, 76-85.
Sziráki, A., Tyshkovskiy, A., and Gladyshev, V. N. (2018). Global remodeling of the mouse DNA methylome during aging and in response to calorie restriction. Aging Cell e12738.
Tissenbaum, H. a, and Guarente, L. (2001). Increased dosage of a sir-2 gene extends lifespan in Caenorhabditis elegans. Nature 410, 227-230.
Tsuchiya, T., Dhahbi, J. M., Cui, X., Mote, P. L., Bartke, A., and Spindler, S. R. (2004). Additive regulation of hepatic gene expression by dwarfism and caloric restriction. Physiol. Genomics 17, 307-315.
Tullet, J. M. A., Hertweck, M., An, J. H., Baker, J., Hwang, J. Y., Liu, S., Oliveira, R. P., Baumeister, R., and Blackwell, T. K. (2008). Direct Inhibition of the Longevity-Promoting Factor SKN-1 by Insulin-like Signaling in C. elegans. Cell 132, 1025-1038.
Ubagai, T., Lei, K. J., Huang, S., Mudd, S. H., Levy, H. L., and Chou, J. Y. (1995). Molecular mechanisms of an inborn error of methionine pathway. Methionine adenosyltransferase deficiency. J. Clin. Invest. 96, 1943-1947.
Uthus, E. O., and Brown-Borg, H. M. (2003). Altered methionine metabolism in long living Ames dwarf mice. Exp. Gerontol. 38, 491-498.
Valenzano, D. R., Terzibasi, E., Genade, T., Cattaneo, A., Domenici, L., and Cellerino, A. (2006). Resveratrol prolongs lifespan and retards the onset of age-related markers in a short-lived vertebrate. Curr. Biol. 16, 296-300.
Vellai, T., Takacs-Vellai, K., Zhang, Y., Kovacs, A. L., Orosz, L., and Müller, F. (2003). Genetics: influence of TOR kinase on lifespan in C. elegans. Nature 426, 620.
Viswanathan, M., Kim, S. K., Berdichevsky, A., and Guarente, L. (2005). A role for SIR-2.1 regulation of ER stress response genes in determining C. elegans life span. Dev. Cell 9, 605-615.
Wauthier, V., Verbeeck, R., and Buc Calderon, P. (2007). The Effect of Ageing on Cytochrome P450 Enzymes: Consequences for Drug Biotransformation in the Elderly. Curr. Med. Chem. 14, 745-757.
Waxman, D. J., and Holloway, M. G. (2009). Sex Differences in the Expression of Hepatic Drug Metabolizing Enzymes. Mol Pharmacol 76, 215-228.
Weindruch, R., Walford, R. L., Fligiel, S., and Guthrie, D. (1986). The retardation of aging in mice by dietary restriction: longevity, cancer, immunity and lifetime energy intake. J. Nutr. 116, 641-654.
Wood, J. G., Regina, B., Lavu, S., Hewitz, K., Helfand, S. L., Tatar, M., and Sinclair, D. (2004). Sirtuin activators mimic caloric restriction and delay ageing in metazoans. Nature 430, 686-689.
Yuan, R., Tsaih, S., Petkova, S. B., Evsikova, C. M. De, Marion, M. A., Bogue, M. A., Mills, K. D., Peters, L. L., Bult, C. J., Rosen, C. J., et al. (2009). Aging in inbred strains of mice: Study design and interim report on median lifespan and circulating IGF1 levels. Aging Cell 8, 277-287.
Zahn, J. M., Sonu, R., Vogel, H., Crane, E., Mazan-Mamczarz, K., Rabkin, R., Davis, R. W., Becker, K. G., Owen, A. B., and Kim, S. K. (2006). Transcriptional profiling of aging in human muscle reveals a common aging signature. PLoS Genet. 2, 1058-1069.
Zhang, L., Ebenezer, P. J., Dasuri, K., Fernandez-Kim, S. O., Francis, J., Mariappan, N., Gao, Z., Ye, J., Bruce-Keller, A. J., and Keller, J. N. (2011). Aging is associated with hypoxia and oxidative stress in adipose tissue: implications for adipose function. Am. J. Physiol. Endocrinol. Metab. 301, E599-607.
Zhang, Y., Xie, Y., Berglund, E. D., Colbert Coate, K., He, T. T., Katafuchi, T., Xiao, G., Potthoff, M. J., Wei, W., Wan, Y., et al. (2012). The starvation hormone, fibroblast growth factor-21, extends lifespan in mice. Elife 2012, 1-14.
Zhao, Y., Tyshkovskiy, A., Muñoz-Espin, D., Tian, X., Serrano, M., de Magalhaes, J. P., Nevo, E., Gladyshev, V. N., Seluanov, A., and Gorbunova, V. (2018). Naked mole rats can undergo developmental, oncogene-induced and DNA damage-induced cellular senescence. Proc. Natl. Acad. Sci. 115, 1801-1806.
Zhou, Y., Xu, B. C., Maheshwari, H. G., He, L., Reed, M., Lozykowski, M., Okada, S., Cataldo, L., Coschigamo, K., Wagner, T. E., et al. (1997). A mammalian model for Laron syndrome produced by targeted disruption of the mouse growth hormone receptor/binding protein gene (the Laron mouse). Proc. Natl. Acad. Sci. U.S.A 94, 13215-13220.
Ables, G. P., Perrone, C. E., Orentreich, D., and Orentreich, N. (2012). Methionine-Restricted C57BL/6J Mice Are Resistant to Diet-Induced Obesity and Insulin Resistance but Have Low Bone Density. PLoS One 7, 1-12.
Ables, G. P., Ouattara, A., Hampton, T. G., Cooke, D., Perodin, F., Augie, I., and Orentreich, D. S. (2015). Dietary methionine restriction in mice elicits an adaptive cardiovascular response to hyperhomocysteinemia. Sci. Rep. 5, 1-10.
Alonso, C., Fernández-Ramos, D., Varela-Rey, M., Martinez-Arranz, I., Navasa, N., Van Liempd, S. M., Lavin Trueba, J. L., Mayo, R., Ilisso, C. P., de Juan, V. G., et al. (2017). Metabolomic Identification of Subtypes of Nonalcoholic Steatohepatitis. Gastroenterology 152, 1449-1461.e7.
Anders, S., and Huber, W. (2010). Differential expression analysis for sequence count data. Genome Biol. 11.
Baze, M. M., Schlauch, K., and Hayes, J. P. (2010). Gene expression of the liver in response to chronic hypoxia. Physiol Genomics 41, 275-288.
Benjamini, Y., and Hochberg, Y. (1995). Controlling the False Discovery Rate: A Practical and Powerful Approach to Multiple Testing. J. R. Stat. Soc. Ser. B 57, 289-300.
Coschigano, K. T., Holland, A. N., Riders, M. E., List, E. O., Flyvbjerg, A., and Kopchick, J. J. (2003). Deletion, but not antagonism, of the mouse growth hormone receptor results in severely decreased body weights, insulin, and insulin-like growth factor I levels and increased life span. Endocrinology 144, 3799-3810.
Dobin, A., Davis, C. A., Schlesinger, F., Drenkow, J., Zaleski, C., Jha, S., Batut, P., Chaisson, M., and Gingeras, T. R. (2013). STAR: Ultrafast universal RNA-seq aligner. Bioinformatics 29, 15-21.
Edgar, R. (2002). Gene Expression Omnibus: NCBI gene expression and hybridization array data repository. Nucleic Acids Res. 30, 207-210.
Flurkey, K., Papaconstantinou, J., Miller, R. A., and Harrison, D. E. (2001). Lifespan extension and delayed immune and collagen aging in mutant mice with defects in growth hormone production. Proc. Natl. Acad. Sci. 98, 6736-6741.
García-Martínez, J. M., Wullschleger, S., Preston, G., Guichard, S., Fleming, S., Alessi, D. R., and Duce, S. L. (2011). Effect of PI3K- and mTOR-specific inhibitors on spontaneous B-cell follicular lymphomas in PTEN/LKB1-deficient mice. Br. J. Cancer 104, 1116-1125.
Harrison, D. E., Strong, R., Allison, D. B., Ames, B. N., Astle, C. M., Atamna, H., Fernandez, E., Flurkey, K., Javors, M. A., Nadon, N. L., et al. (2014). Acarbose, 17-α-estradiol, and nordihydroguaiaretic acid extend mouse lifespan preferentially in males. Aging Cell 13, 273-282.
Hashimshony, T., Senderovich, N., Avital, G., Klochendler, A., de Leeuw, Y., Anavy, L., Gennert, D., Li, S., Livak, K. J., Rozenblatt-Rosen, O., et al. (2016). CEL-Seq2: Sensitive highly-multiplexed single-cell RNA-Seq. Genome Biol. 17, 1-7.
Huang, D. W., Sherman, B. T., and Lempicki, R. A. (2009a). Bioinformatics enrichment tools: Paths toward the comprehensive functional analysis of large gene lists. Nucleic Acids Res. 37, 1-13.
Huang, D. W., Lempicki, R. a, and Sherman, B. T. (2009b). Systematic and integrative analysis of large gene lists using DAVID bioinformatics resources. Nat. Protoc. 4, 44-57.
Jackson, K. L., Palma-Rigo, K., Nguyen-Huu, T. P., Davern, P. J., and Head, G. A. (2014). Actions of rilmenidine on neurogenic hypertension in BPH/2J genetically hypertensive mice. J. Hypertens. 32, 575-586.
Kanfi, Y., Naiman, S., Amir, G., Peshti, V., Zinman, G., Nahum, L., Bar-Joseph, Z., and Cohen, H. Y. (2012). The sirtuin SIRT6 regulates lifespan in male mice. Nature 483, 218-221.
Kautz, L., Meynard, D., Monnier, A., Darnaud, V., Bouvet, R., Wang, R. H., Deng, C., Vaulont, S., Mosser, J., Coppin, H., et al. (2008). Iron regulates phosphorylation of Smad1/5/8 and gene expression of Bmp6, Smad7, Id1, and Atoh8 in the mouse liver. Blood 112, 1503-1509.
Kolesnikov, N., Hastings, E., Keays, M., Melnichuk, O., Tang, Y. A., Williams, E., Dylag, M., Kurbatova, N., Brandizi, M., Burdett, T., et al. (2015). ArrayExpress update-simplifying data submissions. Nucleic Acids Res. 43, D1113-D1116.
Lamb, J., Crawford, E. D., Peck, D., Modell, J. W., Blat, I. C., Wrobel, M. J., Lerner, J., Brunet, J., Subramanian, A., Ross, K. N., et al. (2006). The Connectivity Map: Using Gene-Expression Signatures to Connect Small Molecules, Genes, and Disease. Science (80-.). 313, 1929-1935.
Liao, Y., Smyth, G. K., and Shi, W. (2014). FeatureCounts: An efficient general purpose program for assigning sequence reads to genomic features. Bioinformatics 30, 923-930.
Ma, S., Yim, S. H., Lee, S.-G., Kim, E. B., Lee, S.-R., Chang, K.-T., Buffenstein, R., Lewis, K. N., Park, T. J., Miller, R. A., et al. (2015). Organization of the Mammalian Metabolome according to Organ Function, Lineage Specialization, and Longevity. Cell Metab. 22, 332-343.
De Magalhães, J. P., and Toussaint, 0. (2004). GenAge: A genomic and proteomic network map of human ageing. FEBS Lett. 571, 243-247.
Matys, V. (2006). TRANSFAC® and its module TRANSCompel®: transcriptional gene regulation in eukaryotes. Nucleic Acids Res. 34, D108-D110.
Mercken, E. M., Mitchell, S. J., Martin-, A., Minor, R. K., Almeida, M., Gomes, A. P., Scheibye-knudsen, M., Hector, H., Licata, J. J., Zhang, Y., et al. (2014). SRT2104 extends survival of male mice on a standard diet and preserves bone and muscle mass. 787-796.
Miller, R. A., Harrison, D. E., Astle, C. M., Baur, J. A., Boyd, A. R., De Cabo, R., Fernandez, E., Flurkey, K., Javors, M. A., Nelson, J. F., et al. (2011). Rapamycin, but not resveratrol or simvastatin, extends life span of genetically heterogeneous mice. Journals Gerontol.—Ser. A Biol. Sci. Med. Sci. 66 A, 191-201.
Miller, R. A., Harrison, D. E., Astle, C. M., Fernandez, E., Flurkey, K., Han, M., Javors, M. A., Li, X., Nadon, N. L., Nelson, J. F., et al. (2014). Rapamycin-mediated lifespan increase in mice is dose and sex dependent and metabolically distinct from dietary restriction. Aging Cell 13, 468-477.
Mitchell, S. J., Madrigal-Matute, J., Scheibye-Knudsen, M., Fang, E., Aon, M., Gonzalez-Reyes, J. A., Cortassa, S., Kaushik, S., Gonzalez-Freire, M., Patel, B., et al. (2016). Effects of Sex, Strain, and Energy Intake on Hallmarks of Aging in Mice. Cell Metab. 23, 1093-1112.
Osburn, W. O., Yates, M. S., Dolan, P. D., Chen, S., Liby, K. T., Sporn, M. B., Taguchi, K., Yamamoto, M., and Kensler, T. W. (2008). Genetic or pharmacologic amplification of Nrf2 signaling inhibits acute inflammatory liver injury in mice. Toxicol. Sci. 104, 218-227.
Ozerov, I. V., Lezhnina, K. V., lzumchenko, E., Artemov, A. V., Medintsev, S., Vanhaelen, Q., Aliper, A., Vijg, J., Osipov, A. N., Labat, I., et al. (2016). In silico Pathway Activation Network Decomposition Analysis (iPANDA) as a method for biomarker development. Nat. Commun. 7, 1-11.
Paynter, N. P., Balasubramanian, R., Giulianini, F., Wang, D. D., Tinker, L. F., Gopal, S., Deik, A. A., Albert, C. M., Clish, C. B., and Rexrode, K. M. (2018). Metabolic Predictors of Incident Coronary Heart Disease in Women. Circulation 137, 841-853.
Plank, M., Wuttke, D., van Dam, S., Clarke, S. A., and de Magalhaes, J. P. (2012). A meta-analysis of caloric restriction gene expression profiles to infer common signatures and regulatory mechanisms. Mol. Biosyst. 8, 1339.
Ramadoss, P., Chiappini, F., Bilban, M., and Hollenberg, A. N. (2010). Regulation of hepatic six transmembrane epithelial antigen of prostate 4 (STEAP4) expression by STAT3 and CCAAT/enhancer-binding protein α. J. Biol. Chem. 285, 16453-16466.
Rhoads, T. W., Burhans, M. S., Chen, V. B., Coon, J. J., Colman, R. J., Anderson, R. M., Rhoads, T. W., Burhans, M. S., Chen, V. B., Hutchins, P. D., et al. (2018). Caloric Restriction Engages Hepatic RNA Processing Mechanisms in Rhesus Monkeys. Cell Metab. 27, 677-688.e5.
Ritchie, M. E., Phipson, B., Wu, D., Hu, Y., Law, C. W., Shi, W., and Smyth, G. K. (2015). Limma powers differential expression analyses for RNA-sequencing and microarray studies. Nucleic Acids Res. 43, e47.
Robinson, M. D., McCarthy, D. J., and Smyth, G. K. (2009). edgeR: A Bioconductor package for differential expression analysis of digital gene expression data. Bioinformatics 26, 139-140.
Shannon, P., Markiel, A., Owen Ozier, 2, Baliga, N. S., Wang, J. T., Ramage, D., Amin, N., Schwikowski, B., and Ideker, T. (2003). Cytoscape: a software environment for integrated models of biomolecular interaction networks. Genome Res. 2498-2504.
St-Cyr, J., Derome, N. & Bernatchez, L. (2008) The transcriptomics of life-history trade-offs in whitefish species pairs (Coregonus sp.). Mol. Ecol. 17, 1850-1870.
Strong, R., Miller, R. A., Antebi, A., Astle, C. M., Bogue, M., Denzel, M. S., Fernandez, E., Flurkey, K., Hamilton, K. L., Lamming, D. W., et al. (2016). Longer lifespan in male mice treated with a weakly estrogenic agonist, an antioxidant, an α-glucosidase inhibitor or a Nrf2-inducer. Aging Cell 15, 872-884.
Subramanian, A., Tamayo, P., Mootha, V. K., Mukherjee, S., Ebert, B. L., Gillette, M. a, Paulovich, A., Pomeroy, S. L., Golub, T. R., Lander, E. S., et al. (2005). Gene set enrichment analysis: a knowledge-based approach for interpreting genome-wide expression profiles. Proc. Natl. Acad. Sci. U.S.A 102, 15545-15550.
Veurink, G., Liu, D., Taddei, K., Perry, G., Smith, M. A., Robertson, T. A., Hone, E., Groth, D. M., Atwood, C. S., and Martins, R. N. (2003). Reduction of inclusion body pathology in ApoE-deficient mice fed a combination of antioxidants. Free Radic. Biol. Med. 34, 1070-1077.
Viechtbauer, W. (2010). Conducting Meta-Analyses in R with the metafor Package. J. Stat. Softw. 36, 1-48.
Yongxi, T., Haijun, H., Jiaping, Z., Guoliang, S., and Hongying, P. (2015). Autophagy inhibition sensitizes KU-0063794-mediated anti-HepG2 hepatocellular carcinoma cell activity in vitro and in vivo. Biochem. Biophys. Res. Commun. 465, 494-500.

### Enumerated Embodiments of the Invention

The invention is also characterized by the following enumerated embodiments:

1. A method of identifying an agent capable of increasing the lifespan of a mammalian subject, the method comprising contacting the agent with a cell comprising one or more genes set forth in any of Tables 1-20, wherein a finding that the agent (i) increases expression of one or more genes in any of Tables 1-10 and/or (ii) decreases expression of one or more genes in any of Tables 11-20 identifies the agent as being capable of increasing the lifespan of a mammalian subject.

2. The method of embodiment 1, wherein the subject is a human.

3. The method of embodiment 1 or 2, wherein the cell comprises one or more genes set forth in any of Tables 1-6 or Tables 11-16, wherein a finding that the agent (i) increases expression of one or more genes in any of Tables 1-6 and/or (ii) decreases expression of one or more genes in any of Tables 11-16 identifies the agent as being capable of increasing the lifespan of the mammalian subject.

4. The method of embodiment 3, wherein the cell comprises two, three, four, five, six, seven, eight, nine, ten, or more genes set forth in Table 1 and/or Table 11.

5. The method of embodiment 3 or 4, wherein the cell comprises two, three, four, five, six, seven, eight, nine, ten, or more genes set forth in Table 2 and/or Table 12.

6. The method of any one of embodiments 3-5, wherein the cell comprises two, three, four, five, six, seven, eight, nine, ten, or more genes set forth in Table 3 and/or Table 13.

7. The method of any one of embodiments 3-6, wherein the cell comprises two, three, four, five, six, seven, eight, nine, ten, or more genes set forth in Table 4 and/or Table 14.

8. The method of any one of embodiments 3-7, wherein the cell comprises two, three, four, five, six, seven, eight, nine, ten, or more genes set forth in Table 5 and/or Table 15.

9. The method of any one of embodiments 3-8, wherein the cell comprises two, three, four, five, six, seven, eight, nine, ten, or more genes set forth in Table 6 and/or Table 16.

10. The method of any one of embodiments 1-9, wherein the cell comprises one or more genes set forth in Table 7 or Table 17, wherein a finding that the agent (i) increases expression of one or more genes in Table 7 and/or (ii) decreases expression of one or more genes in Table 17 identifies the agent as being capable of increasing the lifespan of the mammalian subject.

11. The method of embodiment 10, wherein the cell comprises two, three, four, five, six, seven, eight, nine, ten, or more genes set forth in Table 7 and/or Table 17.

12. The method of any one of embodiments 1-11, wherein the cell comprises one or more genes set forth in any of Tables 8-10 or Tables 18-20, wherein a finding that the agent (i) increases expression of one or more genes in any of Tables 8-10 and/or (ii) decreases expression of one or more genes in any of Tables 18-20 identifies the agent as being capable of increasing the lifespan of the mammalian subject.

13. The method of embodiment 12, wherein the cell comprises two, three, four, five, six, seven, eight, nine, ten, or more genes set forth in Table 8 and/or Table 18.

14. The method of embodiment 12 or 13, wherein the cell comprises two, three, four, five, six, seven, eight, nine, ten, or more genes set forth in Table 9 and/or Table 19.

15. The method of any one of embodiments 12-14, wherein the cell comprises two, three, four, five, six, seven, eight, nine, ten, or more genes set forth in Table 10 and/or Table 20.

16. The method of any one of embodiments 1-15, wherein the agent is contacted with the cell by administering the agent to a test subject comprising the cell.

17. The method of embodiment 16, wherein the test subject is a mammal.

18. The method of embodiment 17, wherein the test subject is a mouse.

19. The method of any one of embodiments 1-18, wherein expression of the one or more genes in the cell is determined by RNA-seq.

20. The method of any one of embodiments 1-19, the method further comprising administering the identified agent to a mammalian subject to increase the lifespan of the subject and/or to treat an age-related disease.

21. A collection of (i) gene expression signatures as set forth in any of Tables 1-10, or a combination thereof, that are upregulated, and (ii) gene expression signatures as set forth in any of Tables 11-20, or a combination thereof, that are downregulated.

22. A composition comprising a biological sample and a plurality of nucleic acid primers suitable for amplification of one or more genes set forth in any of Tables 1-10 and/or Tables 11-20.

23. The composition of embodiment 22, wherein the nucleic acid primers are at least 85% complementary to a portion of one or more of the genes set forth in any of Tables 1-10 and/or Tables 11-20.

24. The composition of embodiment 23, wherein the nucleic acid primers are at least 90% complementary to a portion of one or more of the genes set forth in any of Tables 1-10 and/or Tables 11-20.

25. The composition of embodiment 24, wherein the nucleic acid primers are at least 95% complementary to a portion of one or more of the genes set forth in any of Tables 1-10 and/or Tables 11-20.

26. The composition of embodiment 25, wherein the nucleic acid primers are 100% complementary to a portion of one or more of the genes set forth in any of Tables 1-10 and/or Tables 11-20.

27. The composition of any one of embodiments 22-26, wherein the nucleic acid primers are suitable for amplification of one or more genes set forth in any of Tables 1-6 or Tables 11-16.

28. The composition of embodiment 27, wherein the nucleic acid primers are suitable for amplification of two, three, four, five, six, seven, eight, nine, ten, or more genes set forth in Table 1 and/or Table 11.

29. The composition of embodiment 27 or 28, wherein the nucleic acid primers are suitable for amplification of two, three, four, five, six, seven, eight, nine, ten, or more genes set forth in Table 2 and/or Table 12.

30. The composition of any one of embodiments 27-29, wherein the nucleic acid primers are suitable for amplification of two, three, four, five, six, seven, eight, nine, ten, or more genes set forth in Table 3 and/or Table 13.

31. The composition of any one of embodiments 27-30, wherein the nucleic acid primers are suitable for amplification of two, three, four, five, six, seven, eight, nine, ten, or more genes set forth in Table 4 and/or Table 14.

32. The composition of any one of embodiments 27-31, wherein the nucleic acid primers are suitable for amplification of two, three, four, five, six, seven, eight, nine, ten, or more genes set forth in Table 5 and/or Table 15.

33. The composition of any one of embodiments 27-32, wherein the nucleic acid primers are suitable for amplification of two, three, four, five, six, seven, eight, nine, ten, or more genes set forth in Table 6 and/or Table 16.

34. The composition of any one of embodiments 22-33, wherein the nucleic acid primers are suitable for amplification of one or more genes set forth in Table 7 or Table 17.

35. The composition of embodiment 34, wherein the nucleic acid primers are suitable for amplification of two, three, four, five, six, seven, eight, nine, ten, or more genes set forth in Table 7 and/or Table 17.

36. The composition of any one of embodiments 22-35, wherein the nucleic acid primers are suitable for amplification of one or more genes set forth in any of Tables 8-10 or Tables 18-20.

37. The composition of embodiment 36, wherein the nucleic acid primers are suitable for amplification of two, three, four, five, six, seven, eight, nine, ten, or more genes set forth in Table 8 and/or Table 18.

38. The composition of embodiment 36 or 37, wherein the nucleic acid primers are suitable for amplification of two, three, four, five, six, seven, eight, nine, ten, or more genes set forth in Table 9 and/or Table 19.

39. The composition of any one of embodiments 36-38, wherein the nucleic acid primers are suitable for amplification of two, three, four, five, six, seven, eight, nine, ten, or more genes set forth in Table 10 and/or Table 20.

40. A method of increasing the lifespan of a mammalian subject, the method comprising providing the subject with a treatment that (i) increases expression of one or more genes set forth in any of Tables 1-10 and/or (ii) decreases expression of one or more genes set forth in any of Tables 11-20.

41. A method of reducing the frailty index in a mammalian subject, the method comprising providing the subject with a treatment that (i) increases expression of one or more genes set forth in any of Tables 1-10 and/or (ii) decreases expression of one or more genes set forth in any of Tables 11-20.

42. A method of improving learning ability in a mammalian subject, the method comprising providing the subject with a treatment that (i) increases expression of one or more genes set forth in any of Tables 1-10 and/or (ii) decreases expression of one or more genes set forth in any of Tables 11-20.

43. A method of delaying onset of a geriatric syndrome in a mammalian subject, the method comprising providing the subject with a treatment that (i) increases expression of one or more genes set forth in any of Tables 1-10 and/or (ii) decreases expression of one or more genes set forth in any of Tables 11-20.

44. A method of increasing the lifespan of a mammalian subject, the method comprising administering to the subject a therapeutically effective amount of Selumetinib (6-(4-Bromo-2-chloroanilino)-7-fluoro-N-(2-hydroxyethoxy)-3-methylbenzimidazole-5-carboxamide), LY294002 (2-Morpholin-4-yl-8-phenylchromen-4-one), AZD-8055 (5-[2,4-bis[(3S)-3-methyl-4-morpholinyl]pyrido[2,3-d]pyrimidin-7-yl]-2-methoxy-benzenemethanol), KU-0063794 (rel-5-[2-[(2R,6S)-2,6-dimethyl-4-morpholinyl]-4-(4-morpholinyl)pyrido[2,3-d]pyrimidin-7-yl]-2-methoxybenzenemethanol), Celastrol (3-Hydroxy-9β,13α-dimethyl-2-oxo-24,25,26-trinoroleana-1(10),3,5,7-tetraen-29-oic acid), Ascorbyl Palmitate ([(2S)-2-[(2R)-4,5-Dihydroxy-3-oxo-2-furyl]-2-hydroxy-ethyl] hexadecanoate), Oligomycin-a ((1R,4E,5'S,6S,6'S,7R,8S,10R,11R,12S,14R,15S,16R,18E,20E,22R,25S,27R,28S,29R)-22-ethyl-7,11,14,15-tetrahydroxy-6′-[(2R)-2-hydroxypropyl]-5′,6,8,10,12,14,16,28,29-nonamethyl-3′,4′,5′,6′-tetrahydro-3H,9H,13H-spiro[2,26-dioxabicyclo[23.3.1]nonacosa-4,18,20-triene-27,2′-pyran]-3,9,13-trione), NVP-BEZ235 (2-Methyl-2-{4-[3-methyl-2-oxo-8-(quinolin-3-yl)-2,3-dihydro-1H-imidazo[4,5-c]quinolin-1-yl]phenyl}propanenitrile), Importazole (N-(1-Phenylethyl)-2-(pyrrolidin-1-yl)quinazolin-4-amine), Ryuvidine (2-methyl-5-[(4-methylphenyl)amino]-4,7-benzothiazoledione), NSC-663284 (6-Chloro-7-[[2-(4-morpholinyl)ethyl]amino]-5,8-quinolinedione), P1-828 (2-(4-Morpholinyl)-8-(4-aminopheny)l-4H-1-benzopyran-4-one), Pyrvinium pamoate (6-(Dimethylamino)-2-[2-(2,5-dimethyl-1-phenyl-1H-pyrrol-3-yl)ethenyl]-1-methyl-4,4′-methylenebis[3-hydroxy-2-naphthalenecarboxylate] (2:1)-quinolinium), P1-103 (3-[4-(4-morpholinyl)pyrido[3′,2′:4,5]furo[3,2-d]pyrimidin-2-yl]-phenol), YM-155 (4,9-dihydro-1-(2-methoxyethyl)2-methyl-4,9-dioxo-3-(2-pyrazinylmethyl)-1H-naphth[2,3-d]imidazolium, bromide), Prostratin ((1aR,1bS,4aR,7aS,7bR,8R,9aS)-4a,7b-dihydroxy-3-(hydroxymethyl)-1,1,6,8-tetramethyl-5-oxo-1,1a,1b,4,4a,5,7a,7b,8,9-decahydro-9aH-cyclopropa[3,4]benzo[1,2-e]azulen-9a-yl acetate), BCI hydrochloride (3-(cyclohexylamino)-2,3-dihydro-2-(phenylmethylene)-1H-inden-1-one, monohydrochloride), Dorsomorphin-Compound C (6-[4-[2-(1-Piperidinyl)ethoxy]phenyl]-3-(4-pyridinyl)pyrazolo[1,5-a]pyrimidine), VU-0418947-2 (6-Phenyl-N-[(3-phenylphenyl)methyl]-3-pyridin-2-yl-1,2,4-triazin-5-amine), JNK-9L (4-[3-fluoro-5-(4-morpholinyl)phenyl]-N-[4-[3-(4-morpholinyl)-1,2,4-triazol-1-yl]phenyl]-2-pyrimidinamine), Phloretin (3-(4-Hydroxyphenyl)-1-(2,4,6-trihydroxyphenyl)propan-1-one), ZG-10 ((E)-4-(4-(dimethylamino)but-2-enamido)-N-(3-((4-(pyridin-3-yl)pyrimidin-2-yl)amino)phenyl)benzamide), Proscillaridin (5-[(3S,8R,9S,10R,13R,14S,17R)-14-Hydroxy-10,13-dimethyl-3-((2R,3R,4R,5R,6R)-3,4,5-trihydroxy-6-methyltetrahydro-2H-pyran-2-yloxy)-2,3,6,7,8,9,10,11,12,13,14,15,16,17-tetradecahydro-1H-cyclopenta[a]phenanthren-17-yl]-2H-pyran-2-one), YC-1 (3-(5′-Hydroxymethyl-2′-furyl)-1-benzyl indazole), IKK-2-inhibitor-V (N-(3,5-Bis-trifluoromethylphenyl)-5-chloro-2-hydroxybenzamide), Anisomycin ((2R,3S,4S)-4-hydroxy-2-(4-methoxybenzyl)-pyrrolidin-3-yl acetate), Colforsin ([(3R,4aR,5S,6S,6aS,10S,10aR,10bS)-5-acetyloxy-3-ethenyl-10,10b-dihydroxy-3,4a,7,7,10a-Pentamethyl-1-oxo-5,6,6a,8,9,10-hexahydro-2H-benzo[f]chromen-6-yl] 3-d imethylaminopropanoate), Rilmenidine (N-(Dicyclopropylmethyl)-4,5-dihydro-1,3-oxazol-2-amine), GDC-0941 (Pictilisib, 4-(2-(1H-Indazol-4-yl)-6-((4-(methylsulfonyl)piperazin-1-yl)methyl)thieno[3,2-d]pyrimidin-4-yl)morpholine), Valdecoxib (4-(5-methyl-3-phenylisoxazol-4-yl)benzenesulfonamide), Myricetin (3,5,7-Trihydroxy-2-(3,4,5-trihydroxyphenyl)-4-chromenone), Cyproheptadine (4-(5H-Dibenzo[a,d]cyclohepten-5-ylidene)-1-methylpiperidine), Vorinostat (N-Hydroxy-N′-phenyloctanediamide), Nifedipine (3,5-Dimethyl 2,6-dimethyl-4-(2-nitrophenyl)-1,4-dihydropyridine-3,5-dicarboxylate), Phylloquinone (2-Methyl-3-[(E)-3,7,11,15-tetramethylhexadec-2-enyl]naphthalene-1,4-dione), Withaferin-A ((4β,5β,6β,22R)-4,27-Dihydroxy-5,6:22,26-diepoxyergosta-2,24-diene-1,26-dione), Temsirolimus ((1R,2R,4S)-4-{(2R)-2-[(3S,6R,7E,9R,10R,12R,14S,15E,17E,19E,21S,23S,26R,27R,34aS)-9,27-dihydroxy-10,21-dimethoxy-6,8,12,14,20,26-hexamethyl-1,5,11,28,29-pentaoxo-1,4,5,6,9,10,11,12,13,14,21,22,23,24,25,26,27,28,29,31,32,33,34,34a-tetracosahydro-3H-23,27-epoxypyrido[2,1-c][1,4]oxazacyclohentriacontin-3-yl]propyl}-2-methoxycyclohexyl 3-hydroxy-2-(hydroxymethyl)-2-methylpropanoate), SN-38 (4,11-diethyl-4,9-dihydroxy-(4S)-1H-pyrano[3′,4′:6,7]indolizino[1,2-b]quinoline-3,14(4H,12H)-dione), GSK-1059615 (5-[[4-(4-Pyridinyl)-6-quinolinyl]methylene]-2,4-thiazolidenedione), Tipifarnib (6-[(R)-amino-(4-chlorophenyl)-(3-methylimidazol-4-yl)methyl]-4-(3-chlorophenyl)-1-methylquinolin-2-one), Linifanib (1-[4-(3-amino-1H-indazol-4-yl)phenyl]-3-(2-fluoro-5-methylphenyl)urea), WYE-354 (4-[6-[4-[(methoxycarbonyl)amino]phenyl]-4-(4-morpholinyl)-1H-pyrazolo[3,4-d]pyrimidin-1-yl]methyl ester-1-piperidinecarboxylic acid), MK-212 (6-Chloro-2-(1-piperazinyl)pyrazine hydrochloride), and/or Enzastaurin (3-(1-Methylindol-3-yl)-4-[1-[1-(pyridin-2-ylmethyl)piperidin-4-yl]indol-3-yl]pyrrole-2,5-dione), thereby increasing the lifespan of the subject.

45. A method of reducing the frailty index of a mammalian subject, the method comprising administering to the subject a therapeutically effective amount of Selumetinib, LY294002, AZD-8055, KU-0063794, Celastrol, Ascorbyl Palmitate, Oligomycin-a, NVP-BEZ235, Importazole, Ryuvidine, NSC-663284, P1-828, Pyrvinium pamoate, P1-103, YM-155, Prostratin, BCI hydrochloride, Dorsomorphin-Compound C, VU-0418947-2, JNK-9L, Phloretin, ZG-10, Proscillaridin, YC-1, IKK-2-inhibitor-V, Anisomycin, Colforsin, Rilmenidine, GDC-0941, Valdecoxib, Myricetin, Cyproheptadine, Vorinostat, Nifedipine, Phylloquinone, Withaferin-A, Temsirolimus, SN-38, GSK-1059615, Tipifarnib, Linifanib, WYE-354, MK-212, and/or Enzastaurin, thereby reducing the frailty index of the subject.

46. A method of improving learning ability in a mammalian subject, the method comprising administering to the subject a therapeutically effective amount of Selumetinib, LY294002, AZD-8055, KU-0063794, Celastrol, Ascorbyl Palmitate, Oligomycin-a, NVP-BEZ235, Importazole, Ryuvidine, NSC-663284, P1-828, Pyrvinium pamoate, P1-103, YM-155, Prostratin, BCI hydrochloride, Dorsomorphin-Compound C, VU-0418947-2, JNK-9L, Phloretin, ZG-10, Proscillaridin, YC-1, IKK-2-inhibitor-V, Anisomycin, Colforsin, Rilmenidine, GDC-0941, Valdecoxib, Myricetin, Cyproheptadine, Vorinostat, Nifedipine, Phylloquinone, Withaferin-A, Temsirolimus, SN-38, GSK-1059615, Tipifarnib, Linifanib, WYE-354, MK-212, and/or Enzastaurin, thereby improving the learning ability of the subject.

47. A method of delaying onset of a geriatric syndrome in a mammalian subject, the method comprising administering to the subject a therapeutically effective amount of Selumetinib, LY294002, AZD-8055, Celastrol, KU-0063794, Ascorbyl Palmitate, Oligomycin-a, NVP-BEZ235, Importazole, Ryuvidine, NSC-663284, PI-828, Pyrvinium pamoate, PI-103, YM-155, Prostratin, BCI hydrochloride, Dorsomorphin-Compound C, VU-0418947-2, JNK-9L, Phloretin, ZG-10, Proscillaridin, YC-1, IKK-2-inhibitor-V, Anisomycin, Colforsin, Rilmenidine, GDC-0941, Valdecoxib, Myricetin, Cyproheptadine, Vorinostat, Nifedipine, Phylloquinone, Withaferin-A, Temsirolimus, SN-38, GSK-1059615, Tipifarnib, Linifanib, WYE-354, MK-212, and/or Enzastaurin, thereby delaying the onset of a geriatric syndrome in the subject.

48. The method of any one of embodiments 40-47, wherein the subject is a human.

49. The method of any one of embodiments 40-43, wherein the treatment comprises administration of an agent, a lifestyle change, a change in disease status, or a combination thereof.

50. The method of embodiment 49, wherein the treatment comprises administration of an agent.

51. The method of embodiment 50, wherein the agent comprises a small molecule, a peptide, a peptidomimetic, an interfering ribonucleic acid (RNA), an antibody, an aptamer, or a gene therapy.

52. The method of embodiment 51, wherein the agent comprises a small molecule.

53. The method of embodiment 52, wherein the agent comprises a compound represented by formula (I)

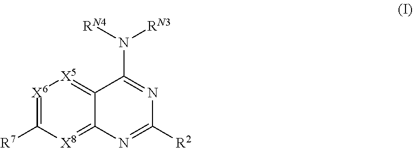

wherein one or two of X⁵, X⁶ and k is N, and the other(s) is/are CH;

R⁷ is selected from halo, OR⁰¹, SR^{S1} NR^{N1}R^{N2}, NR^{N7a}C(═O)R^{C1}, NR^{N7b}SO₂R^{s2a}, an optionally substituted C₅₋₂₀ heteroaryl group, and an optionally substituted C₅₋₂₀ aryl group;

R⁰¹ and R^{S1} are selected from H, an optionally substituted C₅₋₂₀ aryl group, an optionally substituted C₅₋₂₀ heteroaryl group, and an optionally substituted C₁₋₇ alkyl group;

R^{N1} and R^{N2} are independently selected from H, an optionally substituted C₁₋₇ alkyl group, an optionally substituted C₅₋₂₀ heteroaryl group, and an optionally substituted C₅₋₂₀ aryl group, or R^{N1} and R^{N2}, together with the nitrogen to which they are bound, form a heterocyclic ring comprising from 3 to 8 ring atoms;

R^{C1} is selected from H, an optionally substituted C₅₋₂₀ aryl group, an optionally substituted C₅₋₂₀ heteroaryl group, an optionally substituted C₁₋₇ alkyl group;

NR^{N8}R^{N9}, wherein R^{N8} and R^{N9} are independently selected from H, an optionally substituted C₁₋₇ alkyl group, an optionally substituted C₅₋₂₀ heteroaryl group, and an optionally substituted C₅₋₂₀ aryl group, or R^{N8} and R^{N9}, together with the nitrogen to which they are bound, form a heterocyclic ring comprising from 3 to 8 ring atoms; R^{S2a} is selected from H, an optionally substituted C₅₋₂₀ aryl group, an optionally substituted C₅₋₂₀ heteroaryl group, and an optionally substituted C₁₋₇ alkyl group;

R^{N7a} and R^{N7b} are selected from H and a C₁₋₄ alkyl group;

R^{N3} and R^{N4}, together with the nitrogen to which they are bound, form an optionally substituted heterocyclic ring comprising from 3 to 8 ring atoms;

R² is selected from H, halo, OR⁰², SR^{S2b}, NR^{N5}R^{N6}, an optionally substituted C₅₋₂₀ heteroaryl group, and an optionally substituted C₅₋₂₀ aryl group, wherein R⁰² and R^{S2b} are selected from H, an optionally substituted C₅₋₂₀ aryl group, an optionally substituted C₅₋₂₀ heteroaryl group, and an optionally substituted C₁₋₇ alkyl group; and

R^{N5} and R^{N6} are independently selected from H, an optionally substituted C₁₋₇ alkyl group, an optionally substituted C₅₋₂₀ heteroaryl group, and an optionally substituted C₅₋₂₀ aryl group, or R^{N5} and R^{N6}, together with the nitrogen to which they are bound, form an optionally substituted heterocyclic ring comprising from 3 to 8 ring atoms,

or a pharmaceutically acceptable salt thereof.

54. The method of embodiment 53, wherein the agent comprises KU-0063794, represented by formula (1)

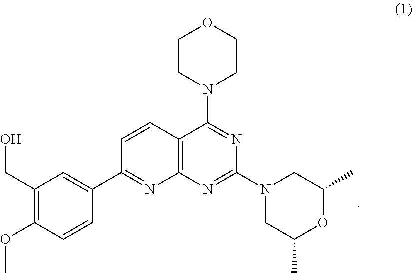 [SMILES: COc1ccc(-c2ccc3c(N4CCOCC4)nc(N4C[C@@H](C)O[C@@H](C)C4)nc3n2)cc1CO]

55. The method of any one of embodiments 52-54, wherein the agent comprises Selumetinib, LY294002, AZD-8055, Celastrol, or ascorbyl palmitate.

56. The method of any one of embodiments 49-55, wherein the treatment comprises a lifestyle change.

57. The method of embodiment 56, wherein the lifestyle change comprises a dietary change.

58. The method of any one of embodiments 49-57, wherein the agent is administered to the subject orally, intraarticularly, intravenously, intramuscularly, rectally, cutaneously, subcutaneously, topically, transdermally, sublingually, nasally, intravesicularly, intrathecally, epidurally, or transmucosally.

59. The method of embodiment 58, wherein the agent is administered to the subject orally.

60. The method of any one of embodiments 49-59, wherein the agent is formulated as a tablet, capsule, gel cap, powder, liquid solution, or liquid suspension.

61. The method of any one of embodiments 40-60, further comprising monitoring the subject for (i) an increase in expression of one or more genes set forth in Tables 1-10 and/or (ii) a decrease in expression of one or more genes set forth in Tables 11-20 following the treatment.

62. A pharmaceutical composition comprising a compound represented by formula (I)

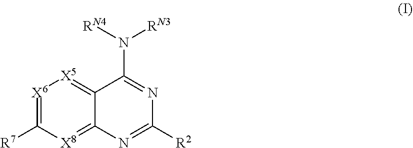

wherein one or two of X⁵, X⁶ and X⁸ is N, and the other(s) is/are CH;

R⁷ is selected from halo, OR⁰¹, SR^{S1}, NR^{N1}R^{N2}, NR^{N7a}C(═O)R^{C1}, NR^{N7b}SO₂R^{S2a}, an optionally substituted C₅₋₂₀ heteroaryl group, and an optionally substituted C₅₋₂₀ aryl group;

R⁰¹ and R^{S1} are selected from H, an optionally substituted C₅₋₂₀ aryl group, an optionally substituted C₅₋₂₀ heteroaryl group, and an optionally substituted C₁₋₇ alkyl group;

R^{N1} and R^{N2} are independently selected from H, an optionally substituted C₁₋₇ alkyl group, an optionally substituted C₅₋₂₀ heteroaryl group, and an optionally substituted C₅₋₂₀ aryl group, or R^{N1} and R^{N2}, together with the nitrogen to which they are bound, form a heterocyclic ring comprising from 3 to 8 ring atoms;

R^{C1} is selected from H, an optionally substituted C₅₋₂₀ aryl group, an optionally substituted C₅₋₂₀ heteroaryl group, an optionally substituted C₁₋₇ alkyl group;

NR^{N8}R^{N9}, wherein R^{N8} and R^{N9} are independently selected from H, an optionally substituted C₁₋₇ alkyl group, an optionally substituted C₅₋₂₀ heteroaryl group, and an optionally substituted C₅₋₂₀ aryl group, or R^{N8} and R^{N9}, together with the nitrogen to which they are bound, form a heterocyclic ring comprising from 3 to 8 ring atoms;

R^{S2a} is selected from H, an optionally substituted C₅₋₂₀ aryl group, an optionally substituted C₅₋₂₀ heteroaryl group, and an optionally substituted C₁₋₇ alkyl group;

R^{N7a} and R^{N7b} are selected from H and a C₁₋₄ alkyl group; R^{N3} and R^{N4}, together with the nitrogen to which they are bound, form an optionally substituted heterocyclic ring comprising from 3 to 8 ring atoms;

R² is selected from H, halo, OR⁰², SR^{S2b}, NR^{N5}R^{N6}, an optionally substituted C₅₋₂₀ heteroaryl group, and an optionally substituted C₅₋₂₀ aryl group, wherein R⁰² and R^{S2b} are selected from H, an optionally substituted C₅₋₂₀ aryl group, an optionally substituted C₅₋₂₀ heteroaryl group, and an optionally substituted C₁₋₇ alkyl group; and

R^{N5} and R^{N6} are independently selected from H, an optionally substituted C₁₋₇ alkyl group, an optionally substituted C₅₋₂₀ heteroaryl group, and an optionally substituted C₅₋₂₀ aryl group, or R^{N5} and R^{N6}, together with the nitrogen to which they are bound, form an optionally substituted heterocyclic ring comprising from 3 to 8 ring atoms,

or a pharmaceutically acceptable salt thereof,

wherein the composition comprises one or more pharmaceutically acceptable excipients and is formulated for administration to a subject in combination with a meal.

63. The pharmaceutical composition of embodiment 62, wherein the compound is KU-0063794, represented by formula (1)

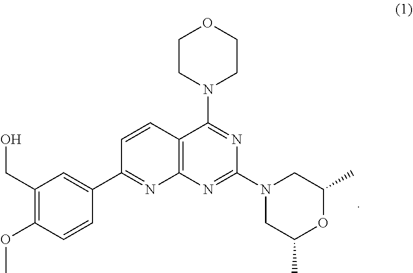 [SMILES: COc1ccc(-c2ccc3c(N4CCOCC4)nc(N4C[C@@H](C)O[C@@H](C)C4)nc3n2)cc1CO]

64. A pharmaceutical composition comprising Selumetinib, LY294002, AZD-8055, Celastrol, or ascorbyl palmitate, and one or more pharmaceutically acceptable excipients, wherein the composition is formulated for administration to a subject in combination with a meal.

65. A pharmaceutical composition comprising Selumetinib, LY294002, AZD-8055, KU-0063794, Celastrol, Ascorbyl Palmitate, Oligomycin-a, NVP-BEZ235, Importazole, Ryuvidine, NSC-663284, PI-828, Pyrvinium pamoate, PI-103, YM-155, Prostratin, BCI hydrochloride, Dorsomorphin-Compound C, VU-0418947-2, JNK-9L, Phloretin, ZG-10, Proscillaridin, YC-1, IKK-2-inhibitor-V, Anisomycin, Colforsin, Rilmenidine, GDC-0941, Valdecoxib, Myricetin, Cyproheptadine, Vorinostat, Nifedipine, Phylloquinone, Withaferin-A, Temsirolimus, SN-38, GSK-1059615, Tipifarnib, Linifanib, WYE-354, MK-212, and/or Enzastaurin, and one or more pharmaceutically acceptable excipients, wherein the composition is formulated for administration to a subject in combination with a meal.

66. The pharmaceutical composition of any one of embodiments 62-65, wherein the composition is a tablet, capsule, gel cap, powder, liquid solution, or liquid suspension.

67. The pharmaceutical composition of any one of embodiments 62-66, wherein the subject is a mammal.

68. The pharmaceutical composition of embodiment 67, wherein the mammal is a human. 69. A dietary supplement comprising Selumetinib, LY294002, AZD-8055, KU-0063794, Celastrol, Ascorbyl Palmitate, Oligomycin-a, NVP-BEZ235, Importazole, Ryuvidine, NSC-663284, PI-828, Pyrvinium pamoate, PI-103, YM-155, Prostratin, BCI hydrochloride, Dorsomorphin-Compound C, VU-0418947-2, JNK-9L, Phloretin, ZG-10, Proscillaridin, YC-1, IKK-2-inhibitor-V, Anisomycin, Colforsin, Rilmenidine, GDC-0941, Valdecoxib, Myricetin, Cyproheptadine, Vorinostat, Nifedipine, Phylloquinone, Withaferin-A, Temsirolimus, SN-38, GSK-1059615, Tipifarnib, Linifanib, WYE-354, MK-212, or Enzastaurin, or a combination thereof.

70. The dietary supplement of embodiment 69, wherein the dietary supplement is a tablet, capsule, gel cap, powder, liquid solution, or liquid suspension.

71. The dietary supplement of embodiment 69 or 70, wherein the dietary supplement is formulated for administration to a subject in combination with a meal.

72. The dietary supplement of embodiment 71, wherein the subject is a mammal.

73. The dietary supplement of embodiment 72, wherein the mammal is a human.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

What is claimed is:

1. A method of increasing the lifespan of a mammalian subject, the method comprising administering to the subject a therapeutically effective amount of Selumetinib (6-(4-Bromo-2-chloroanilino)-7-fluoro-N-(2-hydroxyethoxy)-3-methylbenzimidazole-5-carboxamide), LY294002 (2-Morpholin-4-yl-8-phenylchromen-4-one), AZD-8055 (5-[2,4-bis[(3S)-3-methyl-4-morpholinyl]pyrido[2,3-d]pyrimidin-7-yl]-2-methoxy-benzenemethanol), KU-0063794 (rel-5-[2-[(2R,6S)-2,6-dimethyl-4-morpholinyl]-4-(4-morpholinyl)pyrido[2,3-d]pyrimidin-7-yl]-2-methoxybenzenemethanol), Celastrol (3-Hydroxy-9β,13α-dimethyl-2-oxo-24,25,26-trinoroleana-1(10),3,5,7-tetraen-29-oic acid), Ascorbyl Palmitate ([(2S)-2-[(2R)-4,5-Dihydroxy-3-oxo-2-furyl]-2-hydroxy-ethyl] hexadecanoate), Oligomycin-a ((1R,4E,5'S,6S,6'S,7R,8S,10R,11R,12S,14R,15S,16R,18E,20E,22R,25S,27R,28S,29R)-22-ethyl-7,11,14,15-tetrahydroxy-6′-[(2R)-2-hydroxypropyl]-5′,6,8,10,12,14,16,28,29-nonamethyl-3′,4′,5′,6′-tetrahydro-3H,9H,13H-spiro[2,26-dioxabicyclo[23.3.1]nonacosa-4,18,20-triene-27,2′-pyran]-3,9,13-trione), NVP-BEZ235 (2-Methyl-2-{4-[3-methyl-2-oxo-8-(quinolin-3-yl)-2,3-dihydro-1H-imidazo[4,5-c]quinolin-1-yl]phenyl}propanenitrile), Importazole (N-(1-Phenylethyl)-2-(pyrrolidin-1-yl)quinazolin-4-amine), Ryuvidine (2-methyl-5-[(4-methylphenyl)amino]-4,7-benzothiazoledione), NSC-663284 (6-Chloro-7-[[2-(4-morpholinyl)ethyl]amino]-5,8-quinolinedione), P1-828 (2-(4-Morpholinyl)-8-(4-aminopheny)l-4H-1-benzopyran-4-one), Pyrvinium pamoate (6-(Dimethylamino)-2-[2-(2,5-dimethyl-1-phenyl-1H-pyrrol-3-yl)ethenyl]-1-methyl-4,4′-methylenebis[3-hydroxy-2-naphthalenecarboxylate] (2:1)-quinolinium), P1-103 (3-[4-(4-morpholinyl)pyrido[3′,2′:4,5]furo[3,2-d]pyrimidin-2-yl]-phenol), YM-155 (4,9-dihydro-1-(2-methoxyethyl)2-methyl-4,9-dioxo-3-(2-pyrazinylmethyl)-1H-naphth[2,3-d]imidazolium, bromide), Prostratin ((1aR,1 bS,4aR,7aS,7bR,8R,9aS)-4a,7b-dihydroxy-3-(hydroxymethyl)-1,1,6,8-tetramethyl-5-oxo-1,1a,1b,4,4a,5,7a,7b,8,9-decahydro-9aH-cyclopropa[3,4]benzo[1,2-e]azulen-9a-yl acetate), BCI hydrochloride (3-(cyclohexylamino)-2,3-dihydro-2-(phenylmethylene)-1H-inden-1-one, monohydrochloride), Dorsomorphin-Compound C (6-[4-[2-(1-Piperidinyl)ethoxy]phenyl]-3-(4-pyridinyl)pyrazolo[1,5-a]pyrimidine), VU-0418947-2 (6-Phenyl-N-[(3-phenylphenyl)methyl]-3-pyridin-2-yl-1,2,4-triazin-5-amine), JNK-9L (4-[3-fluoro-5-(4-morpholinyl)phenyl]-N-[4-[3-(4-morpholinyl)-1,2,4-triazol-1-yl]phenyl]-2-pyrimidinamine), Phloretin (3-(4-Hydroxyphenyl)-1-(2,4,6-trihydroxyphenyl)propan-1-one), ZG-10 ((E)-4-(4-(dimethylamino)but-2-enamido)-N-(3-((4-(pyridin-3-yl)pyrimidin-2-yl)amino)phenyl)benzamide), Proscillaridin (5-[(3S,8R,9S,10R,13R,14S,17R)-14-Hydroxy-10,13-dimethyl-3-((2R,3R,4R,5R,6R)-3,4,5-trihydroxy-6-methyltetrahydro-2H-pyran-2-yloxy)-2,3,6,7,8,9,10,11,12,13,14,15,16,17-tetradecahydro-1H-cyclopenta[a]phenanthren-17-yl]-2H-pyran-2-one), YC-1 (3-(5′-Hydroxymethyl-2′-furyl)-1-benzyl indazole), IKK-2-inhibitor-V (N-(3,5-Bis-trifluoromethylphenyl)-5-chloro-2-hydroxybenzamide), Anisomycin ((2R,3S,4S)-4-hydroxy-2-(4-methoxybenzyl)-pyrrolid in-3-yl acetate), Colforsin ([(3R,4aR,5S,6S,6aS,10S,10aR,10bS)-5-acetyloxy-3-ethenyl-10,10b-dihydroxy-3,4a,7,7,10a-Pentamethyl-1-oxo-5,6,6a,8,9,10-hexahydro-2H-benzo[f]chromen-6-yl] 3-d imethylaminopropanoate), Rilmenidine (N-(Dicyclopropylmethyl)-4,5-dihydro-1,3-oxazol-2-amine), GDC-0941 (Pictilisib, 4-(2-(1H-Indazol-4-yl)-6-((4-(methylsulfonyl)piperazin-1-yl)methyl)thieno[3,2-d]pyrimidin-4-yl)morpholine), Valdecoxib (4-(5-methyl-3-phenylisoxazol-4-yl)benzenesulfonamide), Myricetin (3,5,7-Trihydroxy-2-(3,4,5-trihydroxyphenyl)-4-chromenone), Cyproheptadine (4-(5H-Dibenzo[a,d]cyclohepten-5-ylidene)-1-methylpiperidine), Vorinostat (N-Hydroxy-N′-phenyloctanediamide), Nifedipine (3,5-Dimethyl 2,6-dimethyl-4-(2-nitrophenyl)-1,4-dihydropyridine-3,5-dicarboxylate), Phylloquinone (2-Methyl-3-[(E)-3,7,11,15-tetramethylhexadec-2-enyl]naphthalene-1,4-dione), Withaferin-A ((4β,5β,6β,22R)-4,27-Dihydroxy-5,6:22,26-diepoxyergosta-2,24-diene-1,26-dione), Temsirolimus ((1R,2R,4S)-4-{(2R)-2-[(3S,6R,7E,9R,10R,12R,14S,15E,17E,19E,21S,23S,26R,27R,34aS)-9,27-dihydroxy-10,21-dimethoxy-6,8,12,14,20,26-hexamethyl-1,5,11,28,29-pentaoxo-1,4,5,6,9,10,11,12,13,14,21,22,23,24,25,26,27,28,29,31,32,33,34,34a-tetracosahydro-3H-23,27-epoxypyrido[2,1-c][1,4]oxazacyclohentriacontin-3-yl]propyl}-2-methoxycyclohexyl 3-hydroxy-2-(hydroxymethyl)-2-methylpropanoate), SN-38 (4,11-diethyl-4,9-dihydroxy-(4S)-1H-pyrano[3′,4′:6,7]indolizino[1,2-b]quinoline-3,14(4H,12H)-dione), GSK-1059615 (5-[[4-(4-Pyridinyl)-6-quinolinyl]methylene]-2,4-thiazolidenedione), Tipifarnib (6-[(R)-amino-(4-chlorophenyl)-(3-methylimidazol-4-yl)methyl]-4-(3-chlorophenyl)-1-methylquinolin-2-one), Linifanib (1-[4-(3-amino-1H-indazol-4-yl)phenyl]-3-(2-fluoro-5-methylphenyl)urea), WYE-354 (4-[6-[4-[(methoxycarbonyl)amino]phenyl]-4-(4-morpholinyl)-1H-pyrazolo[3,4-d]pyrimidin-1-yl]-methyl ester-1-piperidinecarboxylic acid), MK-212 (6-Chloro-2-(1-piperazinyl)pyrazine hydrochloride), and/or Enzastaurin (3-(1-Methylindol-3-yl)-4-[1-[1-(pyridin-2-ylmethyl)piperidin-4-yl]indol-3-yl]pyrrole-2,5-dione), thereby increasing the lifespan of the subject.

2. A method of reducing the frailty index of a mammalian subject, the method comprising administering to the subject a therapeutically effective amount of Selumetinib, LY294002, AZD-8055, KU-0063794, Celastrol, Ascorbyl Palmitate, Oligomycin-a, NVP-BEZ235, Importazole, Ryuvidine, NSC-663284, PI-828, Pyrvinium pamoate, PI-103, YM-155, Prostratin, BCI hydrochloride, Dorsomorphin-Compound C, VU-0418947-2, JNK-9L, Phloretin, ZG-10, Proscillaridin, YC-1, IKK-2-inhibitor-V, Anisomycin, Colforsin, Rilmenidine, GDC-0941, Valdecoxib, Myricetin, Cyproheptadine, Vorinostat, Nifedipine, Phylloquinone, Withaferin-A, Temsirolimus, SN-38, GSK-1059615, Tipifarnib, Linifanib, WYE-354, MK-212, and/or Enzastaurin, thereby reducing the frailty index of the subject.

3. A method of improving learning ability in a mammalian subject, the method comprising administering to the subject a therapeutically effective amount of Selumetinib, LY294002, AZD-8055, KU-0063794, Celastrol, Ascorbyl Palmitate, Oligomycin-a, NVP-BEZ235, Importazole, Ryuvidine, NSC-663284, PI-828, Pyrvinium pamoate, PI-103, YM-155, Prostratin, BCI hydrochloride, Dorsomorphin-Compound C, VU-0418947-2, JNK-9L, Phloretin, ZG-10, Proscillaridin, YC-1, IKK-2-inhibitor-V, Anisomycin, Colforsin, Rilmenidine, GDC-0941, Valdecoxib, Myricetin, Cyproheptadine, Vorinostat, Nifedipine, Phylloquinone, Withaferin-A, Temsirolimus, SN-38, GSK-1059615, Tipifarnib, Linifanib, WYE-354, MK-212, and/or Enzastaurin, thereby improving the learning ability of the subject.

4. A method of delaying onset of a geriatric syndrome in a mammalian subject, the method comprising administering to the subject a therapeutically effective amount of Selumetinib, LY294002, AZD-8055, Celastrol, KU-0063794, Ascorbyl Palmitate, Oligomycin-a, NVP-BEZ235, Importazole, Ryuvidine, NSC-663284, PI-828, Pyrvinium pamoate, PI-103, YM-155, Prostratin, BCI hydrochloride, Dorsomorphin-Compound C, VU-0418947-2, JNK-9L, Phloretin, ZG-10, Proscillaridin, YC-1, IKK-2-inhibitor-V, Anisomycin, Colforsin, Rilmenidine, GDC-0941, Valdecoxib, Myricetin, Cyproheptadine, Vorinostat, Nifedipine, Phylloquinone, Withaferin-A, Temsirolimus, SN-38, GSK-1059615, Tipifarnib, Linifanib, WYE-354, MK-212, and/or Enzastaurin, thereby delaying the onset of a geriatric syndrome in the subject.

5. The method of any one of claims 1-4, wherein the subject is a human.

6. A pharmaceutical composition comprising Selumetinib, LY294002, AZD-8055, Celastrol, or ascorbyl palmitate, and one or more pharmaceutically acceptable excipients, wherein the composition is formulated for administration to a human in combination with a meal.

7. A pharmaceutical composition comprising Selumetinib, LY294002, AZD-8055, KU-0063794, Celastrol, Ascorbyl Palmitate, Oligomycin-a, NVP-BEZ235, Importazole, Ryuvidine, NSC-663284, PI-828, Pyrvinium pamoate, PI-103, YM-155, Prostratin, BCI hydrochloride, Dorsomorphin-Compound C, VU-0418947-2, JNK-9L, Phloretin, ZG-10, Proscillaridin, YC-1, IKK-2-inhibitor-V, Anisomycin, Colforsin, Rilmenidine, GDC-0941, Valdecoxib, Myricetin, Cyproheptadine, Vorinostat, Nifedipine, Phylloquinone, Withaferin-A, Temsirolimus, SN-38, GSK-1059615, Tipifarnib, Linifanib, WYE-354, MK-212, and/or Enzastaurin, and one or more pharmaceutically acceptable excipients, wherein the composition is formulated for administration to a human in combination with a meal.

8. A dietary supplement comprising Selumetinib, LY294002, AZD-8055, KU-0063794, Celastrol, Ascorbyl Palmitate, Oligomycin-a, NVP-BEZ235, Importazole, Ryuvidine, NSC-663284, PI-828, Pyrvinium pamoate, PI-103, YM-155, Prostratin, BCI hydrochloride, Dorsomorphin-Compound C, VU-0418947-2, JNK-9L, Phloretin, ZG-10, Proscillaridin, YC-1, IKK-2-inhibitor-V, Anisomycin, Colforsin, Rilmenidine, GDC-0941, Valdecoxib, Myricetin, Cyproheptadine, Vorinostat, Nifedipine, Phylloquinone, Withaferin-A, Temsirolimus, SN-38, GSK-1059615, Tipifarnib, Linifanib, WYE-354, MK-212, or Enzastaurin, or a combination thereof.

9. The dietary supplement of claim 8, wherein the dietary supplement is formulated for administration to a human in combination with a meal.

10. A method of increasing the lifespan of a mammalian subject, the method comprising providing the subject with a treatment that (i) increases expression of one or more genes set forth in any of Tables 1-10 and/or (ii) decreases expression of one or more genes set forth in any of Tables 11-20.

11. A method of reducing the frailty index in a mammalian subject, the method comprising providing the subject with a treatment that (i) increases expression of one or more genes set forth in any of Tables 1-10 and/or (ii) decreases expression of one or more genes set forth in any of Tables 11-20.

12. A method of improving learning ability in a mammalian subject, the method comprising providing the subject with a treatment that (i) increases expression of one or more genes set forth in any of Tables 1-10 and/or (ii) decreases expression of one or more genes set forth in any of Tables 11-20.

13. A method of delaying onset of a geriatric syndrome in a mammalian subject, the method comprising providing the subject with a treatment that (i) increases expression of one or more genes set forth in any of Tables 1-10 and/or (ii) decreases expression of one or more genes set forth in any of Tables 11-20.

14. The method of any one of claims 10-13, wherein the treatment comprises administration of an agent, a lifestyle change, a change in disease status, or a combination thereof.

15. The method of claim 14, wherein the treatment comprises administration of an agent.

16. The method of claim 15, wherein the agent comprises a small molecule, a peptide, a peptidomimetic, an interfering ribonucleic acid (RNA), an antibody, an aptamer, or a gene therapy.

17. The method of claim 16, wherein the agent comprises a small molecule.

18. The method of claim 17, wherein the agent comprises a compound represented by formula (I) 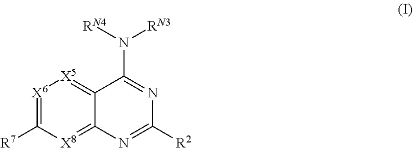
wherein one or two of X⁵, X⁶ and X⁸ is N, and the other(s) is/are CH;
R⁷ is selected from halo, OR⁰¹, SR^{S1}, NR^{N1}R^{N2}, NR^{N7a}C(═O)R^{C1}, NR^{N7b}SO₂R^{S2a}, an optionally substituted C₅₋₂₀ heteroaryl group, and an optionally substituted C₅₋₂₀ aryl group;
R⁰¹ and R^{S1} are selected from H, an optionally substituted C₅₋₂₀ aryl group, an optionally substituted C₅₋₂₀ heteroaryl group, and an optionally substituted C₁₋₇ alkyl group;
R^{N1} and R^{N2} are independently selected from H, an optionally substituted C₁₋₇ alkyl group, an optionally substituted C₅₋₂₀ heteroaryl group, and an optionally substituted C₅₋₂₀ aryl group, or R^{N1} and R^{N2}, together with the nitrogen to which they are bound, form a heterocyclic ring comprising from 3 to 8 ring atoms;
R^{C1} is selected from H, an optionally substituted C₅₋₂₀ aryl group, an optionally substituted C₅₋₂₀ heteroaryl group, an optionally substituted C₁₋₇ alkyl group;
NR^{N8}R^{N9}, wherein R^{N8} and R^{N9} are independently selected from H, an optionally substituted C₁₋₇ alkyl group, an optionally substituted C₅₋₂₀ heteroaryl group, and an optionally substituted C₅₋₂₀ aryl group, or R^{N8} and R^{N9}, together with the nitrogen to which they are bound, form a heterocyclic ring comprising from 3 to 8 ring atoms;
R^{S2a} is selected from H, an optionally substituted C₅₋₂₀ aryl group, an optionally substituted C₅₋₂₀ heteroaryl group, and an optionally substituted C₁₋₇ alkyl group;
R^{N7a} and R^{N7b} are selected from H and a C₁₋₄ alkyl group;
R^{N3} and R^{N4}, together with the nitrogen to which they are bound, form an optionally substituted heterocyclic ring comprising from 3 to 8 ring atoms;
R² is selected from H, halo, OR⁰², SR^{S2b}, NR^{N5}R^{N6}, an optionally substituted C₅₋₂₀ heteroaryl group, and an optionally substituted C₅₋₂₀ aryl group, wherein R⁰² and R^{S2b} are selected from H, an optionally substituted C₅₋₂₀ aryl group, an optionally substituted C₅₋₂₀ heteroaryl group, and an optionally substituted C₁₋₇ alkyl group; and
R^{N5} and R^{N6} are independently selected from H, an optionally substituted C₁₋₇ alkyl group, an optionally substituted C₅₋₂₀ heteroaryl group, and an optionally substituted C₅₋₂₀ aryl group, or R^{N5} and R^{N6}, together with the nitrogen to which they are bound, form an optionally substituted heterocyclic ring comprising from 3 to 8 ring atoms,
or a pharmaceutically acceptable salt thereof.

19. The method of claim 18, wherein the agent comprises KU-0063794, represented by formula (1)

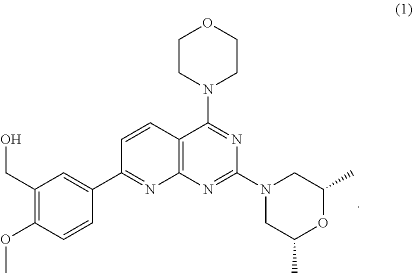 [SMILES: COc1ccc(-c2ccc3c(N4CCOCC4)nc(N4C[C@@H](C)O[C@@H](C)C4)nc3n2)cc1CO]

20. The method of any one of claims 17-19, wherein the agent comprises Selumetinib, LY294002, AZD-8055, Celastrol, or ascorbyl palmitate.

21. The method of any one of claims 14-20, wherein the treatment comprises a lifestyle change.

22. The method of claim 21, wherein the lifestyle change comprises a dietary change.

23. The method of any one of claims 14-22, wherein the agent is administered to the subject orally, intraarticularly, intravenously, intramuscularly, rectally, cutaneously, subcutaneously, topically, transdermally, sublingually, nasally, intravesicularly, intrathecally, epidurally, or transmucosally.

24. The method of claim 23, wherein the agent is administered to the subject orally.

25. The method of any one of claims 14-24, wherein the agent is formulated as a tablet, capsule, gel cap, powder, liquid solution, or liquid suspension.

26. The method of any one of claims 10-25, further comprising monitoring the subject for (i) an increase in expression of one or more genes set forth in Tables 1-10 and/or (ii) a decrease in expression of one or more genes set forth in Tables 11-20 following treatment.
